SEQUENCE LISTING (175 sequences)

SEQ ID NO: 1 - DNA, 363 bp, synthetic construct
  source 1..363: mol_type = other DNA
gaggtgcagctggtggagtctgggggaggcttggtacagcctggagggtccctgagactctcctgtgcagcctctggattcatcttcagtagttatgaaatgcattgggtccgccaggctccagggaaggggctggagtggatttcatacattagtagtagtggtactaccatatactacgcagactctgtgaagggccgattcaccatctccagagacaacgccaagaactcactgtatctgcacatgaacagcctgagagccgaggacacggctgtttattactgtacgagagcccgtataactggaactttcgatgtttttgatatctggggccaagggacaatggtcaccgtctcttca

SEQ ID NO: 2 - protein, 121 aa, synthetic construct
EVQLVESGGGLVQPGGSLRLSCAASGFIFSSYEMHWVRQAPGKGLEWISYISSSGTTIYYADSVKGRFTISRDNAKNSLYLHMNSLRAEDTAVYYCTRARITGTFDVFDIWGQGTMVTVSS

SEQ ID NO: 3 - DNA, 24 bp, synthetic construct
  source 1..24: mol_type = other DNA
ggattcatcttcagtagttatgaa

SEQ ID NO: 4 - protein, 8 aa, synthetic construct
GFIFSSYE

SEQ ID NO: 5 - DNA, 24 bp, synthetic construct
  source 1..24: mol_type = other DNA
attagtagtagtggtactaccata

SEQ ID NO: 6 - protein, 8 aa, synthetic construct
ISSSGTTI

SEQ ID NO: 7 - DNA, 42 bp, synthetic construct
  source 1..42: mol_type = other DNA
acgagagcccgtataactggaactttcgatgtttttgatatc

SEQ ID NO: 8 - protein, 14 aa, synthetic construct
TRARITGTFDVFDI

SEQ ID NO: 9 - DNA, 321 bp, synthetic construct
  source 1..321: mol_type = other DNA
gacatccagatgacccagtctccatcctccctgtctgcatctgtgggagacagagtcaccatcacttgccgggcaagtcagagcattagcagctatttaaattggtatcagcagaaaccagggaaagcccctaagctcctgatctttgctgcatccaatttacagagtggggtcccatcaaggttcagtggcagtagatctgggacagatttcactctcaccatcagcagtctgcaacctgaagattttgcaacttactactgtcaacagaattacaatatcccgtacacttttggccaggggaccaagctggagatcaaa

SEQ ID NO: 10 - protein, 107 aa, synthetic construct
DIQMTQSPSSLSASVGDRVTITCRASQSISSYLNWYQQKPGKAPKLLIFAASNLQSGVPSRFSGSRSGTDFTLTISSLQPEDFATYYCQQNYNIPYTFGQGTKLEIK

SEQ ID NO: 11 - DNA, 18 bp, synthetic construct
  source 1..18: mol_type = other DNA
cagagcattagcagctat

SEQ ID NO: 12 - protein, 6 aa, synthetic construct
QSISSY

SEQ ID NO: 13-14 - skipped (2 entries)

SEQ ID NO: 15 - DNA, 27 bp, synthetic construct
  source 1..27: mol_type = other DNA
caacagaattacaatatcccgtacact

SEQ ID NO: 16 - protein, 9 aa, synthetic construct
QQNYNIPYT

SEQ ID NO: 17 - DNA, 1347 bp, synthetic construct
  source 1..1347: mol_type = other DNA
gaggtgcagctggtggagtctgggggaggcttggtacagcctggagggtccctgagactctcctgtgcagcctctggattcatcttcagtagttatgaaatgcattgggtccgccaggctccagggaaggggctggagtggatttcatacattagtagtagtggtactaccatatactacgcagactctgtgaagggccgattcaccatctccagagacaacgccaagaactcactgtatctgcacatgaacagcctgagagccgaggacacggctgtttattactgtacgagagcccgtataactggaactttcgatgtttttgatatctggggccaagggacaatggtcaccgtctcttcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 18 - protein, 448 aa, synthetic construct
EVQLVESGGGLVQPGGSLRLSCAASGFIFSSYEMHWVRQAPGKGLEWISYISSSGTTIYYADSVKGRFTISRDNAKNSLYLHMNSLRAEDTAVYYCTRARITGTFDVFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - DNA, 645 bp, synthetic construct
  source 1..645: mol_type = other DNA
gacatccagatgacccagtctccatcctccctgtctgcatctgtgggagacagagtcaccatcacttgccgggcaagtcagagcattagcagctatttaaattggtatcagcagaaaccagggaaagcccctaagctcctgatctttgctgcatccaatttacagagtggggtcccatcaaggttcagtggcagtagatctgggacagatttcactctcaccatcagcagtctgcaacctgaagattttgcaacttactactgtcaacagaattacaatatcccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 20 - protein, 214 aa, synthetic construct
DIQMTQSPSSLSASVGDRVTITCRASQSISSYLNWYQQKPGKAPKLLIFAASNLQSGVPSRFSGSRSGTDFTLTISSLQPEDFATYYCQQNYNIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 22 - protein, 369 aa, synthetic construct
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQKLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 23 - DNA, 1480 bp, Homo sapiens
  source 1..1480: mol_type = other DNA
acagacagactacacccagggaatgaagagcaagcgccatgttgaagccatcattaccattcacatccctcttattcctgcagctgcccctgctgggagtggggctgaacacgacaattctgacgcccaatgggaatgaagacaccacagctgatttcttcctgaccactatgcccactgactccctcagtgtttccactctgcccctcccagaggttcagtgttttgtgttcaatgtcgagtacatgaattgcacttggaacagcagctctgagccccagcctaccaacctcactctgcattattggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctggtgatcccctgggctccagagaacctaacacttcacaaactgagtgaatcccagctagaactgaactggaacaacagattcttgaaccactgtttggagcacttggtgcagtaccggactgactgggaccacagctggactgaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaagagaatcctttcctgtttgcattggaagccgtggttatctctgttggctccatgggattgattatcagccttctctgtgtgtatttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctgaaccccaatcctctgacagaagaaccccagggtcctgtagccctaagtggtactaactttccttcattcaacccacctgcgtctcatactcacctcaccccactgtggctgatttggaattttgtgcccccatgtaagcaccccttcatttggcattccccacttgagaattacccttttgccccgaacatgtttttcttctccctcagtctggcccttccttttcgcaggattcttcctccctccctctttccctcccttcctctttccatctaccctccgattgttcctgaaccgatgagaaataaagtttctgttgataatcatcaaaaa

SEQ ID NO: 24 - DNA, 1110 bp, synthetic construct
  source 1..1110: mol_type = other DNA
atgttgaagccatcattaccattcacatccctcttattcctgcagctgcccctgctgggagtggggctgaacacgacaattctgacgcccaatgggaatgaagacaccacagctgatttcttcctgaccactatgcccactgactccctcagtgtttccactctgcccctcccagaggttcagtgttttgtgttcaatgtcgagtacatgaattgcacttggaacagcagctctgagccccagcctaccaacctcactctgcattattggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctggtgatcccctgggctccagagaacctaacacttcacaaactgagtgaatcccagctagaactgaactggaacaacagattcttgaaccactgtttggagcacttggtgcagtaccggactgactgggaccacagctggactgaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaagagaatcctttcctgtttgcattggaagccgtggttatctctgttggctccatgggattgattatcagccttctctgtgtgtatttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctga

SEQ ID NO: 25 - protein, 24 aa, synthetic construct
TFVVQLQDPREPRRQATQMLKLQN

SEQ ID NO: 26 - protein, 24 aa, synthetic construct
TFVVQLQDPQKPQRRAVQKLNLQN

SEQ ID NO: 27 - protein, 24 aa, synthetic construct
TFVVQLQDPQKPQRRAVQKLNLQN

SEQ ID NO: 28 - protein, 24 aa, synthetic construct
TFVVQLQDPREPRRQAVQKLNLQN

SEQ ID NO: 29 - protein, 24 aa, synthetic construct
TFVVQLQDPQKPQRRATQMLKLQN

SEQ ID NO: 30 - protein, 24 aa, synthetic construct
TFVVQLQDPQRRAVQKLNLQNLQN

SEQ ID NO: 31 - protein, 24 aa, synthetic construct
TFVVQLQDPQKPRRQATQMLKLQN

SEQ ID NO: 32 - protein, 24 aa, synthetic construct
TFVVQLQDPQRRATQMLKLQNLQN

SEQ ID NO: 33 - protein, 24 aa, synthetic construct
TFVVQLQDPQKPQRQATQMLKLQN

SEQ ID NO: 34 - protein, 24 aa, synthetic construct
TFVVQLQDPREPRRQAVQMLKLQN

SEQ ID NO: 35 - protein, 24 aa, synthetic construct
TFVVQLQDPREPRRQATQKLKLQN

SEQ ID NO: 36 - protein, 24 aa, synthetic construct
TFVVQLQDPREPRRQATQMLNLQN

SEQ ID NO: 37 - protein, 24 aa, synthetic construct
TFVVQLQDPREPRRQATQKLNLQN

SEQ ID NO: 38 - protein, 16 aa, synthetic construct
REPRRQATQMLKLQNL

SEQ ID NO: 39 - protein, 16 aa, synthetic construct
REPRRQATQKLKLQNL

SEQ ID NO: 40 - DNA, 27 bp, synthetic construct
  source 1..27: mol_type = other DNA
ggctacacagatgctaaaactgcagaa

SEQ ID NO: 41 - protein, 1391 aa, synthetic construct
MDKPKKKRKVKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 42 - DNA, 4176 bp, synthetic construct
  source 1..4176: mol_type = other DNA
atggacaagcccaagaaaaagcggaaagtgaagtacagcatcggcctggacatcggcaccaactctgtgggctgggccgtgatcaccgacgagtacaaggtgcccagcaagaaattcaaggtgctgggcaacaccgacaggcacagcatcaagaagaacctgatcggcgccctgctgttcgacagcggcgaaacagccgaggccaccagactgaagagaaccgccagaagaagatacaccaggcggaagaacaggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacaagagacctgccgccactaagaaggccggacaggccaaaaagaagaagtga

SEQ ID NO: 43 - protein, 7 aa, synthetic construct
PKKKRKV

SEQ ID NO: 44 - protein, 7 aa, synthetic construct
PKKKRRV

SEQ ID NO: 45 - protein, 16 aa, synthetic construct
KRPAATKKAGQAKKKK

SEQ ID NO: 46 - RNA, 16 nt, synthetic construct
  source 1..16: mol_type = other RNA
gttttagagctatgct

SEQ ID NO: 47 - RNA, 22 nt, synthetic construct
  source 1..22: mol_type = other RNA
gttttagagctatgctgttttg

SEQ ID NO: 48 - RNA, 67 nt, synthetic construct
  source 1..67: mol_type = other RNA
agcatagcaagttaaaataaggctagtccgttatcaacttgaaaaagtggcaccgagtcggtgcttt

SEQ ID NO: 49 - RNA, 72 nt, synthetic construct
  source 1..72: mol_type = other RNA
aaacagcatagcaagttaaaataaggctagtccgttatcaacttgaaaaagtggcaccgagtcggtgctttt

SEQ ID NO: 50 - RNA, 82 nt, synthetic construct
  source 1..82: mol_type = other RNA
gttggaaccattcaaaacagcatagcaagttaaaataaggctagtccgttatcaacttgaaaaagtggcaccgagtcggtgc

SEQ ID NO: 51 - RNA, 77 nt, synthetic construct
  source 1..77: mol_type = other RNA
gttttagagctagaaatagcaagttaaaataaggctagtccgttatcaacttgaaaaagtggcaccgagtcggtgct

SEQ ID NO: 52 - RNA, 82 nt, synthetic construct; identical to SEQ ID NO: 50
  source 1..82: mol_type = other RNA

SEQ ID NO: 53 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 54 - RNA, 86 nt, synthetic construct
  source 1..86: mol_type = other RNA
gtttaagagctatgctggaaacagcatagcaagtttaaataaggctagtccgttatcaacttgaaaaagtggcaccgagtcggtgc

SEQ ID NO: 55 - RNA, 83 nt, synthetic construct
  source 1..83: mol_type = other RNA
gttttagagctagaaatagcaagttaaaataaggctagtccgttatcaacttgaaaaagtggcaccgagtcggtgcttttttt

SEQ ID NO: 56 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 57 - RNA, 92 nt, synthetic construct
  source 1..92: mol_type = other RNA
gtttaagagctatgctggaaacagcatagcaagtttaaataaggctagtccgttatcaacttgaaaaagtggcaccgagtcggtgctttttt

SEQ ID NO: 58 - RNA, 68 nt, synthetic construct
  source 1..68: mol_type = other RNA
gttttagagctagaaatagcaagttaaaataaggctagtccgttatcaacttggcaccgagtcggtgc

SEQ ID NO: 59-61 - skipped (3 entries)

SEQ ID NO: 62 - protein, 11 aa, synthetic construct
LHYWYKNSDND

SEQ ID NO: 63 - protein, 11 aa, synthetic construct
LHYRYKVSDNN

SEQ ID NO: 64 - DNA, 20 bp, synthetic construct
  source 1..20: mol_type = other DNA
cctgtctcctgggttcccgt

SEQ ID NO: 65 - DNA, 20 bp, synthetic construct
  source 1..20: mol_type = other DNA
actgcagaatctgggtaatt

SEQ ID NO: 66 - DNA, 20 bp, synthetic construct
  source 1..20: mol_type = other DNA
gaacaacaaatgtttggtag

SEQ ID NO: 67 - DNA, 20 bp, synthetic construct
  source 1..20: mol_type = other DNA
aacatttgttgttcagctcc

SEQ ID NO: 68 - DNA, 20 bp, synthetic construct
  source 1..20: mol_type = other DNA
tccaggacccacgggaaccc

SEQ ID NO: 69 - DNA, 20 bp, synthetic construct
  source 1..20: mol_type = other DNA
tcctgggttcccgtgggtcc

SEQ ID NO: 70 - DNA, 20 bp, synthetic construct
  source 1..20: mol_type = other DNA
ttgttcagctccaggaccca

SEQ ID NO: 71 - DNA, 20 bp, synthetic construct
  source 1..20: mol_type = other DNA
ctgcagttttagcatctgtg

SEQ ID NO: 72 - DNA, 20 bp, synthetic construct
  source 1..20: mol_type = other DNA
tgttcagctccaggacccac

SEQ ID NO: 73 - DNA, 20 bp, synthetic construct
  source 1..20: mol_type = other DNA
gatgctaaaactgcagaatc

SEQ ID NO: 74 - DNA, 20 bp, synthetic construct
  source 1..20: mol_type = other DNA
gcctgtctcctgggttcccg

SEQ ID NO: 75 - DNA, 20 bp, synthetic construct
  source 1..20: mol_type = other DNA
ggagctgaacaacaaatgtt

SEQ ID NO: 76 - RNA, 20 nt, synthetic construct
  source 1..20: mol_type = other RNA
cctgtctcctgggttcccgt

SEQ ID NO: 77 - RNA, 20 nt, synthetic construct
  source 1..20: mol_type = other RNA
actgcagaatctgggtaatt

SEQ ID NO: 78 - RNA, 20 nt, synthetic construct
  source 1..20: mol_type = other RNA
gaacaacaaatgtttggtag

SEQ ID NO: 79 - RNA, 20 nt, synthetic construct
  source 1..20: mol_type = other RNA
aacatttgttgttcagctcc

SEQ ID NO: 80 - RNA, 20 nt, synthetic construct
  source 1..20: mol_type = other RNA
tccaggacccacgggaaccc

SEQ ID NO: 81 - RNA, 20 nt, synthetic construct
  source 1..20: mol_type = other RNA
tcctgggttcccgtgggtcc

SEQ ID NO: 82 - RNA, 20 nt, synthetic construct
  source 1..20: mol_type = other RNA
ttgttcagctccaggaccca

SEQ ID NO: 83 - RNA, 20 nt, synthetic construct
  source 1..20: mol_type = other RNA
ctgcagttttagcatctgtg

SEQ ID NO: 84 - RNA, 20 nt, synthetic construct
  source 1..20: mol_type = other RNA
tgttcagctccaggacccac

SEQ ID NO: 85 - RNA, 20 nt, synthetic construct
  source 1..20: mol_type = other RNA
gatgctaaaactgcagaatc

SEQ ID NO: 86 - RNA, 20 nt, synthetic construct
  source 1..20: mol_type = other RNA
gcctgtctcctgggttcccg

SEQ ID NO: 87 - RNA, 20 nt, synthetic construct
  source 1..20: mol_type = other RNA
ggagctgaacaacaaatgtt

SEQ ID NO: 88 - DNA, 120 bp, synthetic construct
  source 1..120: mol_type = other DNA
aggacccacgggaacccaggagacaggccacacagaagctaaaactgcagaatctgggtaatttgaaaagaaagggtcaagagaccagggatactgtgggacattggagtctacagagta

SEQ ID NO: 89 - DNA, 120 bp, synthetic construct
  source 1..120: mol_type = other DNA
tactctgtagactccaatgtcccacagtatccctggtctcttgaccctttcttttcaaattacccagattctgcagttttagcttctgtgtggcctgtctcctgggttcccgtgggtcct

SEQ ID NO: 90 - DNA, 90 bp, synthetic construct
  source 1..90: mol_type = other DNA
ccaggagacaggccacacagaagctaaaactgcagaatctgggtaatttgaaaagaaagggtcaagagaccagggatactgtgggacatt

SEQ ID NO: 91 - DNA, 90 bp, synthetic construct
  source 1..90: mol_type = other DNA
aatgtcccacagtatccctggtctcttgaccctttcttttcaaattacccagattctgcagttttagcttctgtgtggcctgtctcctgg

SEQ ID NO: 92 - DNA, 60 bp, synthetic construct
  source 1..60: mol_type = other DNA
cacagaagctaaaactgcagaatctgggtaatttgaaaagaaagggtcaagagaccaggg

SEQ ID NO: 93 - DNA, 60 bp, synthetic construct
  source 1..60: mol_type = other DNA
ccctggtctcttgaccctttcttttcaaattacccagattctgcagttttagcttctgtg

SEQ ID NO: 94 - DNA, 90 bp, synthetic construct
  source 1..90: mol_type = other DNA
cacagaagctaaaactgcagaatctgggtaatttgaaaagaaagggtcaagagaccagggatactgtgggacattggagtctacagagta

SEQ ID NO: 95 - DNA, 90 bp, synthetic construct
  source 1..90: mol_type = other DNA
tactctgtagactccaatgtcccacagtatccctggtctcttgaccctttcttttcaaattacccagattctgcagttttagcttctgtg

SEQ ID NO: 96 - DNA, 90 bp, synthetic construct
  source 1..90: mol_type = other DNA
aggacccacgggaacccaggagacaggccacacagaagctaaaactgcagaatctgggtaatttgaaaagaaagggtcaagagaccaggg

SEQ ID NO: 97 - DNA, 90 bp, synthetic construct
  source 1..90: mol_type = other DNA
ccctggtctcttgaccctttcttttcaaattacccagattctgcagttttagcttctgtgtggcctgtctcctgggttcccgtgggtcct

SEQ ID NO: 98 - DNA, 120 bp, synthetic construct
  source 1..120: mol_type = other DNA
tatccctggtctcttgaccctttctttccaaattacccagattctgcagttttagcttctgtgttgcctgtctcctgggttcccgtgggtcctggagctgaacaacaaatgtttggtaga

SEQ ID NO: 99 - DNA, 120 bp, synthetic construct
  source 1..120: mol_type = other DNA
tctaccaaacatttgttgttcagctccaggacccacgggaacccaggagacaggcaacacagaagctaaaactgcagaatctgggtaatttggaaagaaagggtcaagagaccagggata

SEQ ID NO: 100 - DNA, 90 bp, synthetic construct
  source 1..90: mol_type = other DNA
tgaccctttctttccaaattacccagattctgcagttttagcttctgtgttgcctgtctcctgggttcccgtgggtcctggagctgaaca

SEQ ID NO: 101 - DNA, 90 bp, synthetic construct
  source 1..90: mol_type = other DNA
tgttcagctccaggacccacgggaacccaggagacaggcaacacagaagctaaaactgcagaatctgggtaatttggaaagaaagggtca

SEQ ID NO: 102 - DNA, 60 bp, synthetic construct
  source 1..60: mol_type = other DNA
aaattacccagattctgcagttttagcttctgtgttgcctgtctcctgggttcccgtggg

SEQ ID NO: 103 - DNA, 60 bp, synthetic construct
  source 1..60: mol_type = other DNA
cccacgggaacccaggagacaggcaacacagaagctaaaactgcagaatctgggtaattt

SEQ ID NO: 104 - DNA, 30 bp, synthetic construct
  source 1..30: mol_type = other DNA
tgcagttttagcttctgtgttgcctgtctc

SEQ ID NO: 105 - DNA, 30 bp, synthetic construct
  source 1..30: mol_type = other DNA
gagacaggcaacacagaagctaaaactgca

SEQ ID NO: 106 - DNA, 90 bp, synthetic construct
  source 1..90: mol_type = other DNA
aaattacccagattctgcagttttagcttctgtgttgcctgtctcctgggttcccgtgggtcctggagctgaacaacaaatgtttggtag

SEQ ID NO: 107 - DNA, 90 bp, synthetic construct
  source 1..90: mol_type = other DNA
ctaccaaacatttgttgttcagctccaggacccacgggaacccaggagacaggcaacacagaagctaaaactgcagaatctgggtaattt

SEQ ID NO: 108 - DNA, 90 bp, synthetic construct
  source 1..90: mol_type = other DNA
tatccctggtctcttgaccctttctttccaaattacccagattctgcagttttagcttctgtgttgcctgtctcctgggttcccgtgggt

SEQ ID NO: 109 - DNA, 90 bp, synthetic construct
  source 1..90: mol_type = other DNA
acccacgggaacccaggagacaggcaacacagaagctaaaactgcagaatctgggtaatttggaaagaaagggtcaagagaccagggata

SEQ ID NO: 110 - DNA, 120 bp, synthetic construct
  source 1..120: mol_type = other DNA
tctttccaaattacccagattctgcagttttagcttctgtgtggcctgtctcctgggctctcgtgggtcctggagctgaacaacaaatgtttggtagaggtggatctcctttttttgcaa

SEQ ID NO: 111 - DNA, 120 bp, synthetic construct
  source 1..120: mol_type = other DNA
ttgcaaaaaaaggagatccacctctaccaaacatttgttgttcagctccaggacccacgagagcccaggagacaggccacacagaagctaaaactgcagaatctgggtaatttggaaaga

SEQ ID NO: 112 - DNA, 90 bp, synthetic construct
  source 1..90: mol_type = other DNA
cagattctgcagttttagcttctgtgtggcctgtctcctgggctctcgtgggtcctggagctgaacaacaaatgtttggtagaggtggat

SEQ ID NO: 113 - DNA, 90 bp, synthetic construct
  source 1..90: mol_type = other DNA
atccacctctaccaaacatttgttgttcagctccaggacccacgagagcccaggagacaggccacacagaagctaaaactgcagaatctg

SEQ ID NO: 114 - DNA, 60 bp, synthetic construct
  source 1..60: mol_type = other DNA
tagcttctgtgtggcctgtctcctgggctctcgtgggtcctggagctgaacaacaaatgt

SEQ ID NO: 115 - DNA, 60 bp, synthetic construct
  source 1..60: mol_type = other DNA
acatttgttgttcagctccaggacccacgagagcccaggagacaggccacacagaagcta

SEQ ID NO: 116 - DNA, 90 bp, synthetic construct
  source 1..90: mol_type = other DNA
tagcttctgtgtggcctgtctcctgggctctcgtgggtcctggagctgaacaacaaatgtttggtagaggtggatctcctttttttgcaa

SEQ ID NO: 117 - DNA, 90 bp, synthetic construct
  source 1..90: mol_type = other DNA
ttgcaaaaaaaggagatccacctctaccaaacatttgttgttcagctccaggacccacgagagcccaggagacaggccacacagaagcta

SEQ ID NO: 118 - DNA, 20 bp, synthetic construct
  source 1..20: mol_type = other DNA
tcgagtacatgaattgcact

SEQ ID NO: 119 - DNA, 20 bp, synthetic construct
  source 1..20: mol_type = other DNA
cagagtgaggttggtaggct

SEQ ID NO: 120 - DNA, 20 bp, synthetic construct
  source 1..20: mol_type = other DNA
taatgcagagtgaggttggt

SEQ ID NO: 121 - DNA, 20 bp, synthetic construct
  source 1..20: mol_type = other DNA
ggtcaggaagaaatctagat

SEQ ID NO: 122 - DNA, 20 bp, synthetic construct
  source 1..20: mol_type = other DNA
gcagagtgaggttggtaggc

SEQ ID NO: 123 - DNA, 20 bp, synthetic construct
  source 1..20: mol_type = other DNA
gaaacactgagggagtcagt

SEQ ID NO: 124 - DNA, 20 bp, synthetic construct
  source 1..20: mol_type = other DNA
agtcagtgggcatagtggtc

SEQ ID NO: 125 - DNA, 20 bp, synthetic construct
  source 1..20: mol_type = other DNA
caaaacactgaacctctggg

SEQ ID NO: 126 - DNA, 20 bp, synthetic construct
  source 1..20: mol_type = other DNA
tcaggaagaaatctagattg

SEQ ID NO: 127 - DNA, 20 bp, synthetic construct
  source 1..20: mol_type = other DNA
gtcaggaagaaatctagatt

SEQ ID NO: 128 - DNA, 20 bp, synthetic construct
  source 1..20: mol_type = other DNA
aacacaaaacactgaacctc

SEQ ID NO: 129 - DNA, 20 bp, synthetic construct
  source 1..20: mol_type = other DNA
gagggagtcagtgggcatag

SEQ ID NO: 130 - DNA, 20 bp, synthetic construct
  source 1..20: mol_type = other DNA
aaaacactgaacctctggga

SEQ ID NO: 131 - DNA, 20 bp, synthetic construct
  source 1..20: mol_type = other DNA
aaacactgaacctctgggag

SEQ ID NO: 132 - DNA, 20 bp, synthetic construct
  source 1..20: mol_type = other DNA
agagtgaggttggtaggctg

SEQ ID NO: 133 - DNA, 20 bp, synthetic construct
  source 1..20: mol_type = other DNA
gggcagagtggaaacactga

SEQ ID NO: 134 - DNA, 20 bp, synthetic construct
  source 1..20: mol_type = other DNA
acacaaaacactgaacctct

SEQ ID NO: 135 - DNA, 20 bp, synthetic construct
  source 1..20: mol_type = other DNA
ggaaacactgagggagtcag

SEQ ID NO: 136 - RNA, 20 nt, synthetic construct
  source 1..20: mol_type = other RNA
tcgagtacatgaattgcact

SEQ ID NO: 137 - RNA, 20 nt, synthetic construct
  source 1..20: mol_type = other RNA
cagagtgaggttggtaggct

SEQ ID NO: 138 - RNA, 20 nt, synthetic construct
  source 1..20: mol_type = other RNA
taatgcagagtgaggttggt

SEQ ID NO: 139 - RNA, 20 nt, synthetic construct
  source 1..20: mol_type = other RNA
ggtcaggaagaaatctagat

SEQ ID NO: 140 - RNA, 20 nt, synthetic construct
  source 1..20: mol_type = other RNA
gcagagtgaggttggtaggc

SEQ ID NO: 141 - RNA, 20 nt, synthetic construct
  source 1..20: mol_type = other RNA
gaaacactgagggagtcagt

SEQ ID NO: 142 - RNA, 20 nt, synthetic construct
  source 1..20: mol_type = other RNA
agtcagtgggcatagtggtc

SEQ ID NO: 143 - RNA, 20 nt, synthetic construct
  source 1..20: mol_type = other RNA
caaaacactgaacctctggg

SEQ ID NO: 144 - RNA, 20 nt, synthetic construct
  source 1..20: mol_type = other RNA
tcaggaagaaatctagattg

SEQ ID NO: 145 - RNA, 20 nt, synthetic construct
  source 1..20: mol_type = other RNA
gtcaggaagaaatctagatt

SEQ ID NO: 146 - RNA, 20 nt, synthetic construct
  source 1..20: mol_type = other RNA
aacacaaaacactgaacctc

SEQ ID NO: 147 - RNA, 20 nt, synthetic construct
  source 1..20: mol_type = other RNA
gagggagtcagtgggcatag

SEQ ID NO: 148 - RNA, 20 nt, synthetic construct
  source 1..20: mol_type = other RNA
aaaacactgaacctctggga

SEQ ID NO: 149 - RNA, 20 nt, synthetic construct
  source 1..20: mol_type = other RNA
aaacactgaacctctgggag

SEQ ID NO: 150 - RNA, 20 nt, synthetic construct
  source 1..20: mol_type = other RNA
agagtgaggttggtaggctg

SEQ ID NO: 151 - RNA, 20 nt, synthetic construct
  source 1..20: mol_type = other RNA
gggcagagtggaaacactga

SEQ ID NO: 152 - RNA, 20 nt, synthetic construct
  source 1..20: mol_type = other RNA
acacaaaacactgaacctct

SEQ ID NO: 153 - RNA, 20 nt, synthetic construct
  source 1..20: mol_type = other RNA
ggaaacactgagggagtcag

SEQ ID NO: 154 - DNA, 120 bp, synthetic construct
  source 1..120: mol_type = other DNA
cttcttcatcccctccccctcgtcccttctcatactgataatgcagagtgaggttggtaggctgtggctcagagctgctgttccaagtgcaattcatgtactcgacattgaacacaaaac

SEQ ID NO: 155 - DNA, 120 bp, synthetic construct
  source 1..120: mol_type = other DNA
gttttgtgttcaatgtcgagtacatgaattgcacttggaacagcagctctgagccacagcctaccaacctcactctgcattatcagtatgagaagggacgagggggaggggatgaagaag

SEQ ID NO: 156 - DNA, 90 bp, synthetic construct
  source 1..90: mol_type = other DNA
cccctcgtcccttctcatactgataatgcagagtgaggttggtaggctgtggctcagagctgctgttccaagtgcaattcatgtactcga

SEQ ID NO: 157 - DNA, 90 bp, synthetic construct
  source 1..90: mol_type = other DNA
tcgagtacatgaattgcacttggaacagcagctctgagccacagcctaccaacctcactctgcattatcagtatgagaagggacgagggg

SEQ ID NO: 158 - DNA, 60 bp, synthetic construct
  source 1..60: mol_type = other DNA
catactgataatgcagagtgaggttggtaggctgtggctcagagctgctgttccaagtgc

SEQ ID NO: 159 - DNA, 60 bp, synthetic construct
  source 1..60: mol_type = other DNA
gcacttggaacagcagctctgagccacagcctaccaacctcactctgcattatcagtatg

SEQ ID NO: 160 - DNA, 90 bp, synthetic construct
  source 1..90: mol_type = other DNA
catactgataatgcagagtgaggttggtaggctgtggctcagagctgctgttccaagtgcaattcatgtactcgacattgaacacaaaac

SEQ ID NO: 161 - DNA, 90 bp, synthetic construct
  source 1..90: mol_type = other DNA
gttttgtgttcaatgtcgagtacatgaattgcacttggaacagcagctctgagccacagcctaccaacctcactctgcattatcagtatg

SEQ ID NO: 162 - DNA, 90 bp, synthetic construct
  source 1..90: mol_type = other DNA
cttcttcatcccctccccctcgtcccttctcatactgataatgcagagtgaggttggtaggctgtggctcagagctgctgttccaagtgc

SEQ ID NO: 163 - DNA, 90 bp, synthetic construct
  source 1..90: mol_type = other DNA
gcacttggaacagcagctctgagccacagcctaccaacctcactctgcattatcagtatgagaagggacgagggggaggggatgaagaag

SEQ ID NO: 164 - DNA, 120 bp, synthetic construct
  source 1..120: mol_type = other DNA
cacctcttcttcatcccctccccctcgtcccttctcatactgatagtgtagtgttaggttggtaggctggggctcagagctgctgttccaagtgcaattcatgtactcgacattgaacac

SEQ ID NO: 165 - DNA, 120 bp, synthetic construct
  source 1..120: mol_type = other DNA
gtgttcaatgtcgagtacatgaattgcacttggaacagcagctctgagccccagcctaccaacctaacactacactatcagtatgagaagggacgagggggaggggatgaagaagaggtg

SEQ ID NO: 166 - DNA, 90 bp, synthetic construct
  source 1..90: mol_type = other DNA
ccctccccctcgtcccttctcatactgatagtgtagtgttaggttggtaggctggggctcagagctgctgttccaagtgcaattcatgta

SEQ ID NO: 167 - DNA, 90 bp, synthetic construct
  source 1..90: mol_type = other DNA
tacatgaattgcacttggaacagcagctctgagccccagcctaccaacctaacactacactatcagtatgagaagggacgagggggaggg

SEQ ID NO: 168 - DNA, 60 bp, synthetic construct
  source 1..60: mol_type = other DNA
cttctcatactgatagtgtagtgttaggttggtaggctggggctcagagctgctgttcca

SEQ ID NO: 169 - DNA, 60 bp, synthetic construct
  source 1..60: mol_type = other DNA
tggaacagcagctctgagccccagcctaccaacctaacactacactatcagtatgagaag

SEQ ID NO: 170 - DNA, 90 bp, synthetic construct
  source 1..90: mol_type = other DNA
cttctcatactgatagtgtagtgttaggttggtaggctggggctcagagctgctgttccaagtgcaattcatgtactcgacattgaacac

SEQ ID NO: 171 - DNA, 90 bp, synthetic construct
  source 1..90: mol_type = other DNA
gtgttcaatgtcgagtacatgaattgcacttggaacagcagctctgagccccagcctaccaacctaacactacactatcagtatgagaag

SEQ ID NO: 172 - DNA, 90 bp, synthetic construct
  source 1..90: mol_type = other DNA
cacctcttcttcatcccctccccctcgtcccttctcatactgatagtgtagtgttaggttggtaggctggggctcagagctgctgttcca

SEQ ID NO: 173 - DNA, 90 bp, synthetic construct
  source 1..90: mol_type = other DNA
tggaacagcagctctgagccccagcctaccaacctaacactacactatcagtatgagaagggacgagggggaggggatgaagaagaggtg

SEQ ID NO: 174 - protein, 12 aa, synthetic construct
NLTLHYWYKNSD

SEQ ID NO: 175 - protein, 12 aa, synthetic construct
NLTLHYQYKNSD

(19) United States
(12) Patent Application Publication
(10) Pub. No.: US 2024/0390460 A1
(43) Pub. Date: Nov. 28, 2024

(54) ANTIBODY RESISTANT MODIFIED RECEPTORS TO ENHANCE CELL-BASED THERAPIES

(71) Applicant: Regeneron Pharmaceuticals, Inc., Tarrytown, NY (US)

(72) Inventors: Michael Moore, New York, NY (US); Casandra Panea, Tenafly, NJ (US); Matthew Sleeman, Yorktown Heights, NY (US); Andrew J. Murphy, Croton-on-Hudson, NY (US); George D. Yancopoulos, Yorktown Heights, NY (US); Liang Zhang, Hartsdale, NY (US); Annabel Romero Hernández, White Plains, NY (US)

(73) Assignee: Regeneron Pharmaceuticals, Inc., Tarrytown, NY (US)

(21) Appl. No.: 18/799,190

(22) Filed: Aug. 9, 2024

Related U.S. Application Data
(63) Continuation of application No. PCT/US2023/075431, filed on Sep. 28, 2023.
(60) Provisional application No. 63/578,729, filed on Aug. 25, 2023.
(60) Provisional application No. 63/377,444, filed on Sep. 28, 2022.

(51) Int. Cl. A61K 38/17; A61K 35/28; A61K 35/545; A61K 39/00; A61K 39/395; A61P 35/00; C07K 14/715; C12N 5/0783; C12N 9/22; C12N 15/11; C12N 15/90

(52) U.S. Cl. CPC A61K 38/1793; A61K 35/28; A61K 35/545; A61K 39/3955; A61K 39/4611; A61K 39/4612; A61K 39/4613; A61K 39/4631; A61K 39/4632; A61P 35/00; C07K 14/7155; C12N 5/0636; C12N 9/22; C12N 15/11; C12N 15/907; C12N 2310/20; C12N 2510/00

(57) ABSTRACT

Methods for improving engraftment of donor cells in a subject thereof are provided. Such methods can comprise providing donor cells that have been modified to express a first isoform of a target protein (e.g., interleukin-2 receptor subunit gamma (IL2RG)) that is functionally indistinguishable but immunologically distinguishable from a second isoform of the target protein, administering the donor cells to the subject, and then selectively inhibiting host cells in the subject based on their expression of the second isoform of the target protein, thereby improving engraftment of donor cells in the subject. Also provided are combinations for administration to a subject in need thereof, wherein the combination comprises (1) a population of donor cells modified to express a first isoform of a target protein (e.g., IL2RG) and (2) an antagonist (e.g., anti-IL2RG antigen-binding protein) that specifically binds to a second isoform of the target protein but does not specifically bind to the first isoform of the target protein.

### CROSS-REFERENCE TO RELATED APPLICATIONS

This application is a continuation of International Application No. PCT/US2023/075431, filed Sep. 28, 2023, which claims the benefit of U.S. Application No. 63/377,444, filed Sep. 28, 2022, and U.S. Application No. 63/578,729, filed Aug. 25, 2023, each of which is herein incorporated by reference in its entirety for all purposes.

### REFERENCE TO A SEQUENCE LISTING SUBMITTED AS AN XML FILE

The Sequence Listing written in file 611344SEQLIST.xml is 163 kilobytes, was created on Aug. 9, 2024, and is hereby incorporated by reference.

### BACKGROUND

Host conditioning is an essential step prior to transplant or adoptive cell therapies in which immunosuppressive agents are administered to a transplant recipient to support donor graft cell uptake. Conditioning treatments serve several key purposes, including clearance of niche space for grafted immune cells, suppression of allogeneic graft rejection, and depletion or debulking of diseased cells (e.g., hematologic and solid tumors or defective immune cells). However, standard of care agents for pre-transplant conditioning and post-transplant immune suppression are genotoxic drugs that cause damage to many tissues, leave patients highly immunocompromised, and can induce secondary malignancies, thus excluding many patients from potentially curative transplant treatments.

Non-genotoxic lymphosuppressive agents, such as monoclonal antibodies targeting lymphocytes, are an alternative conditioning treatment. An obstacle to use of lymphosuppressive agents as conditioning therapies, however, is the susceptibility of grafted cells, in addition to the target host cells, to their effects.

### SUMMARY

Methods for improving engraftment of donor cells in a subject in need thereof are provided. Also provided are combinations or combination medicaments for administration to a subject in need thereof. Also provided are isolated cells or populations of cells modified to express a first isoform of interleukin-2 receptor subunit gamma (IL2RG) that is different from a second isoform of IL2RG. Also provided are methods of making the isolated cells or populations of cells. Also provided are genetically engineered interleukin-2 receptor subunit gamma (IL2RG) proteins and nucleic acids encoding the proteins.

Methods for improving engraftment of donor cells in a subject in need thereof are provided. Some such methods comprise: (a) providing donor cells that have been modified to express a first isoform of a target protein, wherein the target protein is a protein expressed on the cell surface of hematopoietic cells, wherein the first isoform of the target protein is different from a second isoform of the target protein, wherein the second isoform is expressed in host cells of the subject; (b) administering the donor cells to the subject, and (c) selectively inhibiting host cells in the subject based on their expression of the second isoform of the target protein, thereby improving engraftment of donor cells in the subject.

In some such methods, the target protein is a receptor. In some such methods, the target protein is a cytokine receptor or a chemokine receptor, optionally wherein the target protein is the cytokine receptor. In some such methods, the target protein is a protein expressed on the cell surface of lymphocytes. In some such methods, the target protein is a cytokine receptor sub-unit of an interleukin-2 (IL-2) receptor, an IL-4 receptor, an IL-7 receptor, an IL-9 receptor, an IL-15 receptor, or an IL-21 receptor. In some such methods, the target protein is interleukin-2 receptor subunit gamma (IL2RG).

In some such methods, the selective inhibition of host cells in step (c) does not comprise ablation of host cells or does not comprise ablation of host cells by an active killing mechanism. In some such methods, the selective inhibition in step (c) comprises: (1) blocking growth of the host cells to provide a competitive growth advantage to the donor cells; (2) blocking localization or trafficking of the host cells to provide a competitive homing advantage to the donor cells; (3) blocking a cell-cell interaction or adhesion of the host cells to provide a competitive tissue infiltration advantage to the donor cells; or (4) blocking immune cell activation in the host cells to provide a competitive advantage to the donor cells. In some such methods, the selective inhibition in step (c) comprises blocking growth (i.e., blocking proliferation) of the host cells and/or blocking immune cell activation in the host cells to provide a competitive growth advantage to the donor cells.

In some such methods, the first isoform and the second isoform are functionally indistinguishable but immunologically distinguishable. In some such methods, the donor cells express both the first isoform of the target protein and the second isoform of the target protein. In some such methods, the donor cells express only the first isoform of the target protein.

In some such methods, the first isoform of the target protein is expressed from an expression vector in the donor cells. In some such methods, a genomic locus has been edited to express the first isoform of the target protein in the donor cells. In some such methods, the genomic locus is an endogenous genomic locus encoding the target protein, optionally wherein the target protein is IL2RG and the genomic locus is an IL2RG genomic locus. In some such methods, the genomic locus is not an endogenous genomic locus encoding the target protein, optionally wherein the target protein is IL2RG, and the genomic locus is not an IL2RG genomic locus.

In some such methods, the first isoform of the target protein is a genetically engineered isoform of the target protein. In some such methods, the first isoform of the target protein is genetically engineered to comprise a mutation to provide an altered epitope, optionally wherein the mutation is an artificial mutation. In some such methods, the altered epitope is a binding region of a target protein antagonist (e.g., antigen-binding protein) such that the target protein antagonist exhibits reduced or abolished ability to bind and/or inhibit the first isoform of the target protein (e.g., compared to its ability to bind and/or inhibit the second isoform of the target protein). In some such methods, both the first isoform of the target protein and the second isoform of the target protein retain the ability to bind to an endogenous ligand, optionally wherein the target protein antagonist (e.g., antigen-binding protein) blocks the binding of the endogenous ligand to the second isoform of the target protein but not the first isoform of the target protein.

In some such methods, the target protein is IL2RG, the altered epitope is in a binding region of the target protein antagonist, the target protein antagonist is an antibody comprising an immunoglobulin light chain or variable region thereof comprising three light chain CDRs and an immunoglobulin heavy chain or variable region thereof comprising three heavy chain CDRs, the three light chain CDRs comprise, consist essentially of, or consist of the sequences set forth in SEQ ID NOS: 12, 14, and 16, respectively, and the three heavy chain CDRs comprise, consist essentially of, or consist of the sequences set forth in SEQ ID NOS: 4, 6, and 8, respectively. In some such methods, the target protein is IL2RG, and the mutation comprises a mutation encoded by nucleotides within exon 2 and/or exon 3 of the IL2RG gene at the IL2RG genomic locus. In some such methods, the target protein is IL2RG, and the mutation comprises a mutation or substitution within a region from position T127 to position N150 and/or within a region from position L87 to position D97. In some such methods, the target protein is IL2RG, and the mutation comprises a mutation or substitution at position M145, position W90, position K92, position N93, position D95, position D97, position T127, position R139, position R140, position Q141, position T143, and/or position K147. In some such methods, the target protein is IL2RG, and the mutation comprises a mutation or substitution at position M145, optionally wherein the substitution is a M145K substitution, a M145D substitution, a M145E substitution, a M145P substitution, a M145W substitution, or an M145Y substitution. In some such methods, the target protein is IL2RG, and the mutation comprises a M145K substitution. In some such methods, the target protein is IL2RG, and the mutation comprises a mutation or substitution at position W90, optionally wherein the substitution is a W90V substitution, a W90R substitution, a W90Q substitution, a W90L substitution, a W90K substitution, a W90E substitution, or a W90D substitution, optionally wherein the mutation comprises a W90Q substitution. In some such methods, the target protein is IL2RG, and the mutation comprises a mutation or substitution at position M145 and a mutation or substitution at position W90.

In some such methods, the selective inhibition in step (c) comprises administering a target protein antagonist to the subject, wherein the target protein antagonist specifically binds to the second isoform of the target protein but does not specifically bind to the first isoform of the target protein, optionally wherein step (c) comprises multiple administrations of the target protein antagonist. In some such methods, the target protein antagonist is an antigen-binding protein. In some such methods, the antigen-binding protein is an antibody or an antigen-binding fragment thereof.

In some such methods, the target protein is IL2RG, the antigen-binding protein comprises an immunoglobulin light chain or variable region thereof comprising three light chain CDRs and an immunoglobulin heavy chain or variable region thereof comprising three heavy chain CDRs, the three light chain CDRs comprise, consist essentially of, or consist of sequences at least 90% identical to the sequences set forth in SEQ ID NOS: 12, 14, and 16, respectively, and the three heavy chain CDRs comprise, consist essentially of, or consist of sequences at least 90% identical to the sequences set forth in SEQ ID NOS: 4, 6, and 8, respectively. In some such methods, the three light chain CDRs comprise, consist essentially of, or consist of the sequences set forth in SEQ ID NOS: 12, 14, and 16, respectively, and the three heavy chain CDRs comprise, consist essentially of, or consist of the sequences set forth in SEQ ID NOS: 4, 6, and 8, respectively. In some such methods, the target protein is IL2RG, the antigen-binding protein comprises an immunoglobulin light chain variable region that comprises, consists essentially of, or consists of a sequence at least 90% identical to the sequence set forth in SEQ ID NO: 10, and the antigen-binding protein comprises an immunoglobulin heavy chain variable region that comprises, consists essentially of, or consists of a sequence at least 90% identical to the sequence set forth in SEQ ID NO: 2. In some such methods, the target protein is IL2RG, the immunoglobulin light chain variable region comprises, consists essentially of, or consists of the sequence set forth in SEQ ID NO: 10, and the immunoglobulin heavy chain variable region comprises, consists essentially of, or consists of the sequence set forth in SEQ ID NO: 2. In some such methods, the target protein is IL2RG, the antigen-binding protein comprises an immunoglobulin light chain that comprises, consists essentially of, or consists of the sequence set forth in SEQ ID NO: 20, and the antigen-binding protein comprises an immunoglobulin heavy chain that comprises, consists essentially of, or consists of the sequence set forth in SEQ ID NO: 18.

In some such methods, the donor cells and/or the host cells are hematopoietic cells. In some such methods, the donor cells and/or the host cells are immune cells. In some such methods, the donor cells and/or the host cells are lymphocytes or lymphoid progenitor cells. In some such methods, the donor cells and/or the host cells are T cells. In some such methods, the donor cells and/or the host cells are tumor infiltrating lymphocytes (TILs). In some such methods, the donor cells and/or the host cells are B cells. In some such methods, the donor cells and/or the host cells are NK cells. In some such methods, the donor cells and/or the host cells are hematopoietic stem and progenitor cells. In some such methods, the donor cells and/or the host cells are derived from hematopoietic stem cells or hematopoietic stem and progenitor cells. In some such methods, the donor cells are derived from induced pluripotent stem cells. In some such methods, the subject is a mammal or a non-human mammal, and the donor cells are mammalian cells or non-human mammalian cells. In some such methods, the subject is a human, and the donor cells are human cells.

In some such methods, the donor cells comprise or express a therapeutic molecule. In some such methods, the therapeutic molecule does not target the target protein. In some such methods, the donor cells comprise or express an immunoglobulin. In some such methods, the immunoglobulin does not target the target protein. In some such methods, the donor cells comprise a chimeric antigen receptor (CAR) or an exogenous T cell receptor (TCR). In some such methods, the CAR or the exogenous TCR does not target the target protein.

In some such methods, the donor cells are autologous. In some such methods, the donor cells are allogeneic or syngeneic.

In some such methods, the subject has a disease or disorder, and the method is for treating the disease or disorder. In some such methods, the subject has cancer. In some such methods, the cancer is a solid tumor cancer. In some such methods, the cancer is a hematologic cancer. In some such methods, the subject has a hematopoietic malignancy, and the method is for treating the hematopoietic malignancy in the subject. In some such methods, the subject has defective immune cells or a genetic deficiency in hematopoiesis. In some such methods, the genetic deficiency in hematopoiesis is sickle cell disease or severe combined immunodeficiency (SCID). In some such methods, the therapeutic molecule targets diseased cells.

In some such methods, steps (b) and (c) occur simultaneously. In some such methods, step (b) occurs prior to step (c), optionally wherein step (c) comprises multiple administrations of a target protein antagonist subsequent to step (b). In some such methods, step (b) occurs subsequent to step (c), optionally wherein step (c) comprises multiple administrations of a target protein antagonist prior to step (b). In some such methods, step (c) occurs both prior to and subsequent to step (b), optionally wherein step (c) comprises multiple administrations of a target protein antagonist prior to step (b) and/or multiple administrations of the target protein antagonist subsequent to step (b).

Some such methods further comprise generating the donor cells by modifying a population of cells to express the first isoform of the target protein prior to step (a). In some such methods, the population of cells is a population of induced pluripotent stem cells, and the method further comprises differentiating the induced pluripotent stem cells prior to step (a) into the donor cells that are administered in step (a), optionally wherein the induced pluripotent stem cells are differentiated into hematopoietic cells, lymphocytes or lymphoid progenitor cells, T cells, B cells, NK cells, hematopoietic stem cells, or hematopoietic stem and progenitor cells. In some such methods, the population of cells is a population of hematopoietic stem cells or hematopoietic stem and progenitor cells, and the method further comprises differentiating the hematopoietic stem cells or hematopoietic stem and progenitor cells prior to step (a) into the donor cells that are administered in step (a), optionally wherein the hematopoietic stem cells or hematopoietic stem and progenitor cells are differentiated into differentiated hematopoietic cells, lymphocytes or lymphoid progenitor cells, T cells, B cells, or NK cells.

In some such methods, generating the donor cells comprises introducing an expression vector encoding the first isoform of the target protein to express the first isoform of the target protein prior to step (a). In some such methods, generating the donor cells comprises editing a genomic locus in the population of cells to express the first isoform of the target protein prior to step (a). In some such methods, the genomic locus is an endogenous genomic locus encoding the target protein, optionally wherein the target protein is IL2RG, and the genomic locus is an IL2RG genomic locus. In some such methods, the genomic locus is not an endogenous genomic locus encoding the target protein, optionally wherein the target protein is IL2RG, and the genomic locus is not an IL2RG genomic locus.

In some such methods, the editing comprises introducing into the population of cells: (1) a nuclease agent or one or more nucleic acids encoding the nuclease agent, wherein the nuclease agent targets a nuclease target sequence in the genomic locus; and (2) an exogenous donor nucleic acid, wherein the nuclease agent cleaves the genomic locus and the exogenous donor nucleic acid is inserted into the genomic locus or recombines with the genomic locus to generate the donor cells that express the first isoform of the target protein. In some such methods, the nuclease agent comprises: (a) a zinc finger nuclease (ZFN); (b) a transcription activator-like effector nuclease (TALEN); or (c) (i) a Cas protein; and (ii) a guide RNA, wherein the guide RNA comprises a DNA-targeting segment that targets a guide RNA target sequence that is the nuclease target sequence, and wherein the guide RNA binds to the Cas protein and targets the Cas protein to the guide RNA target sequence. In some such methods, the nuclease agent comprises the Cas protein and the guide RNA, optionally wherein the target protein is IL2RG, and the DNA-targeting segment comprises the sequence set forth in any one of SEQ ID NOS: 76-87 or the guide RNA target sequence comprises the sequence set forth in any one of SEQ ID NOS: 64-75, or optionally wherein the target protein is IL2RG, and the DNA-targeting segment comprises the sequence set forth in any one of SEQ ID NOS: 136-153 or the guide RNA target sequence comprises the sequence set forth in any one of SEQ ID NOS: 118-135. In some such methods, the Cas protein is a Cas9 protein. In some such methods, the exogenous donor nucleic acid comprises homology arms. In some such methods, the exogenous donor nucleic acid is a single-stranded oligodeoxynucleotide (ssODN), optionally wherein the target protein is IL2RG, and the ssODN comprises the nucleic acid sequence set forth in any one of SEQ ID NOS: 88-117 or optionally wherein the target protein is IL2RG, and the ssODN comprises the nucleic acid sequence set forth in any one of SEQ ID NOS: 154-173.

In some such methods, the target protein is IL2RG, and (I) the DNA-targeting segment comprises the sequence set forth in SEQ ID NO: 77 or the guide RNA target sequence comprises the sequence set forth in SEQ ID NO: 65, and the ssODN comprises the nucleic acid sequence set forth in any one of SEQ ID NOS: 88-97; (II) the DNA-targeting segment comprises the sequence set forth in SEQ ID NO: 83 or the guide RNA target sequence comprises the sequence set forth in SEQ ID NO: 71, and the ssODN comprises the nucleic acid sequence set forth in any one of SEQ ID NOS: 98-10⁷; (III) the DNA-targeting segment comprises the sequence set forth in SEQ ID NO: 86 or the guide RNA target sequence comprises the sequence set forth in SEQ ID NO: 74, and the ssODN comprises the nucleic acid sequence set forth in any one of SEQ ID NOS: 108-117; (IV) the DNA-targeting segment comprises the sequence set forth in SEQ ID NO: 137 or the guide RNA target sequence comprises the sequence set forth in SEQ ID NO: 119, and the ssODN comprises the nucleic acid sequence set forth in any one of SEQ ID NOS: 154-163; or (V) the DNA-targeting segment comprises the sequence set forth in SEQ ID NO: 138 or the guide RNA target sequence comprises the sequence set forth in SEQ ID NO: 120, and the ssODN comprises the nucleic acid sequence set forth in any one of SEQ ID NOS: 164-173.

Some such methods further comprise isolating the population of cells from the subject or from a different subject prior to modifying the population of cells.

In another aspect, provide are combinations or combination medicaments for administration to a subject in need thereof. Some such combinations comprise: (a) a population of donor cells modified to express a first isoform of a target protein, wherein the target protein is a protein expressed on the cell surface of hematopoietic cells, wherein the first isoform of the target protein is different from a second isoform of the target protein; and (b) a target protein antagonist that specifically binds to the second isoform of the target protein but does not specifically bind to the first isoform of the target protein.

In some such combinations, the target protein is a receptor. In some such combinations, the target protein is a cytokine receptor or a chemokine receptor, optionally wherein the target protein is the cytokine receptor. In some such combinations, the target protein is a protein expressed on the cell surface of lymphocytes. In some such combinations, the target protein is a cytokine receptor sub-unit of an interleukin-2 (IL-2) receptor, an IL-4 receptor, an IL-7 receptor, an IL-9 receptor, an IL-15 receptor, or an IL-21 receptor. In some such combinations, the target protein is interleukin-2 receptor subunit gamma (IL2RG).

In some such combinations, the target protein antagonist selectively inhibits host cells in the subject based on their expression of the second isoform of the target protein. In some such combinations, the selective inhibition of host cells does not comprise ablation of host cells by an active killing mechanism. In some such combinations, the selective inhibition comprises: (1) blocking growth of the host cells to provide a competitive growth advantage to the donor cells; (2) blocking localization or trafficking of the host cells to provide a competitive homing advantage to the donor cells; (3) blocking a cell-cell interaction or adhesion of the host cells to provide a competitive tissue infiltration advantage to the donor cells; or (4) blocking immune cell activation in the host cells to provide a competitive advantage to the donor cells. In some such combinations, the selective inhibition comprises blocking growth (i.e., blocking proliferation) of the host cells and/or blocking immune cell activation in the host cells to provide a competitive growth advantage to the donor cells.

In some such combinations, the first isoform and the second isoform are functionally indistinguishable but immunologically distinguishable. In some such combinations, the donor cells express both the first isoform of the target protein and the second isoform of the target protein. In some such combinations, the donor cells express only the first isoform of the target protein.

In some such combinations, the first isoform of the target protein is expressed from an expression vector in the population of donor cells. In some such combinations, a genomic locus has been edited to express the first isoform of the target protein in the population of donor cells. In some such combinations, the genomic locus is an endogenous genomic locus encoding the target protein, optionally wherein the target protein is IL2RG, and the genomic locus is an IL2RG genomic locus. In some such combinations, the genomic locus is not an endogenous genomic locus encoding the target protein, optionally wherein the target protein is IL2RG, and the genomic locus is not an IL2RG genomic locus.

In some such combinations, the first isoform of the target protein is a genetically engineered isoform of the target protein. In some such combinations, the first isoform of the target protein is genetically engineered to comprise a mutation to provide an altered epitope, optionally wherein the mutation is an artificial mutation. In some such combinations, the altered epitope is a binding region of the target protein antagonist (e.g., antigen-binding protein) such that the target protein antagonist exhibits reduced or abolished ability to bind and/or inhibit the first isoform of the target protein (e.g., compared to its ability to bind and/or inhibit the second isoform of the target protein). In some such combinations, both the first isoform of the target protein and the second isoform of the target protein retain the ability to bind to an endogenous ligand, optionally wherein the target protein antagonist (e.g., antigen-binding protein) blocks the binding of the endogenous ligand to the second isoform of the target protein but not the first isoform of the target protein.

In some such combinations, the target protein is IL2RG, the altered epitope is in a binding region of the target protein antagonist, the target protein antagonist is an antibody comprising an immunoglobulin light chain or variable region thereof comprising three light chain CDRs and an immunoglobulin heavy chain or variable region thereof comprising three heavy chain CDRs, the three light chain CDRs comprise, consist essentially of, or consist of the sequences set forth in SEQ ID NOS: 12, 14, and 16, respectively, and the three heavy chain CDRs comprise, consist essentially of, or consist of the sequences set forth in SEQ ID NOS: 4, 6, and 8, respectively.

In some such combinations, the target protein is IL2RG, and the mutation comprises a mutation encoded by nucleotides within exon 2 and/or exon 3 of the IL2RG gene at the IL2RG genomic locus. In some such combinations, the target protein is IL2RG, and the mutation comprises a mutation or substitution within a region from position T127 to position N150 and/or within a region from position L87 to position D97. In some such combinations, the target protein is IL2RG, and the mutation comprises a mutation or substitution at position M145, position W90, position K92, position N93, position D95, position D97, position T127, position R139, position R140, position Q141, position T143, and/or position K147. In some such combinations, the target protein is IL2RG, and the mutation comprises a mutation or substitution at position M145, optionally wherein the substitution is a M145K substitution, a M145D substitution, a M145E substitution, a M145P substitution, a M145W substitution, or an M145Y substitution. In some such combinations, the target protein is IL2RG, and the mutation comprises a M145K substitution. In some such combinations, the target protein is IL2RG, and the mutation comprises a mutation or substitution at position W90, optionally wherein the substitution is a W90V substitution, a W90R substitution, a W90Q substitution, a W90L substitution, a W90K substitution, a W90E substitution, or a W90D substitution, optionally wherein the mutation comprises a W90Q substitution. In some such combinations, the target protein is IL2RG, and the mutation comprises a mutation or substitution at position M145 and a mutation or substitution at position W90.

In some such combinations, the target protein antagonist is an antigen-binding protein. In some such combinations, the antigen-binding protein is an antibody or an antigen-binding fragment thereof. In some such combinations, the target protein is IL2RG, the antigen-binding protein comprises an immunoglobulin light chain or variable region thereof comprising three light chain CDRs and an immunoglobulin heavy chain or variable region thereof comprising three heavy chain CDRs, the three light chain CDRs comprise, consist essentially of, or consist of sequences at least 90% identical to the sequences set forth in SEQ ID NOS: 12, 14, and 16, respectively, and the three heavy chain CDRs comprise, consist essentially of, or consist of sequences at least 90% identical to the sequences set forth in SEQ ID NOS: 4, 6, and 8, respectively. In some such combinations, the three light chain CDRs comprise, consist essentially of, or consist of the sequences set forth in SEQ ID NOS: 12, 14, and 16, respectively, and the three heavy chain CDRs comprise, consist essentially of, or consist of the sequences set forth in SEQ ID NOS: 4, 6, and 8, respectively. In some such combinations, the target protein is IL2RG, the antigen-binding protein comprises an immunoglobulin light chain variable region that comprises, consists essentially of, or consists of a sequence at least 90% identical to the sequence set forth in SEQ ID NO: 10, and the antigen-binding protein comprises an immunoglobulin heavy chain variable region that comprises, consists essentially of, or consists of a sequence at least 90% identical to the sequence set forth in SEQ ID NO: 2. In some such combinations, the target protein is IL2RG, the immunoglobulin light chain variable region comprises, consists essentially of, or consists of the sequence set forth in SEQ ID NO: 10, and the immunoglobulin heavy chain variable region comprises, consists essentially of, or consists of the sequence set forth in SEQ ID NO: 2. In some such combinations, the target protein is IL2RG, the antigen-binding protein comprises an immunoglobulin light chain that comprises, consists essentially of, or consists of the sequence set forth in SEQ ID NO: 20, and the antigen-binding protein comprises an immunoglobulin heavy chain that comprises, consists essentially of, or consists of the sequence set forth in SEQ ID NO: 18.

In some such combinations, the donor cells are hematopoietic cells. In some such combinations, the donor cells are immune cells. In some such combinations, the donor cells are lymphocytes or lymphoid progenitor cells. In some such combinations, the donor cells are T cells. In some such combinations, the donor cells are tumor infiltrating lymphocytes (TILs). In some such combinations, the donor cells are B cells. In some such combinations, the donor cells are NK cells. In some such combinations, the donor cells are hematopoietic stem cells or hematopoietic stem and progenitor cells. In some such combinations, the donor cells are derived from induced pluripotent stem cells or are derived from hematopoietic stem cells or hematopoietic stem and progenitor cells.

In some such combinations, the subject is a mammal or a non-human mammal, and the donor cells are mammalian cells or non-human mammalian cells. In some such combinations, the subject is a human, and the donor cells are human cells.

In some such combinations, the donor cells comprise or express a therapeutic molecule. In some such combinations, the therapeutic molecule does not target the target protein.

In some such combinations, the donor cells comprise or express an immunoglobulin. In some such combinations, the immunoglobulin does not target the target protein. In some such combinations, the donor cells comprise a chimeric antigen receptor (CAR) or an exogenous T cell receptor (TCR). In some such combinations, the CAR or the exogenous TCR does not target the target protein.

In some such combinations, the donor cells are autologous. In some such combinations, the donor cells are allogeneic or syngeneic.

In some such combinations, the subject has a disease or disorder, and the combination is for treating the disease or disorder. In some such combinations, the subject has cancer. In some such combinations, the cancer is a solid tumor cancer. In some such combinations, the cancer is a hematologic cancer. In some such combinations, the subject has a hematopoietic malignancy, and the combination medicament is for treating the hematopoietic malignancy in the subject. In some such combinations, the subject has defective immune cells or a genetic deficiency in hematopoiesis. In some such combinations, the genetic deficiency in hematopoiesis is sickle cell disease or severe combined immunodeficiency (SCID). In some such combinations, the therapeutic molecule targets diseased cells.

In another aspect, provided are isolated cells or populations of cells modified to express a first isoform of interleukin-2 receptor subunit gamma (IL2RG) that is different from a second isoform of IL2RG. In some such cells or populations, the first isoform of IL2RG is genetically engineered to comprise a mutation to provide an altered epitope, the altered epitope is a binding region of an IL2RG antagonist (e.g., an antigen-binding protein) such that the IL2RG antagonist exhibits reduced or abolished ability to bind and/or inhibit the first isoform of IL2RG (e.g., compared to its ability to bind and/or inhibit the second isoform of IL2RG), and the first isoform of IL2RG retains binding to its endogenous ligands. In some such cells or populations, the mutation is an artificial mutation. In some such cells or populations, both the first isoform of IL2RG and the second isoform of IL2RG retain the ability to bind to an endogenous ligand, optionally wherein the IL2RG antagonist (e.g., antigen-binding protein) blocks the binding of the endogenous ligand to the second isoform of IL2RG but not the first isoform of IL2RG.

In some such cells or populations, the first isoform and the second isoform are functionally indistinguishable but immunologically distinguishable. In some such cells or populations, the cell or cells express both the first isoform of IL2RG and the second isoform of IL2RG. In some such cells or populations, the cell or cells express only the first isoform of IL2RG.

In some such cells or populations, the first isoform of IL2RG is expressed from an expression vector in the cell or cells. In some such cells or populations, a genomic locus has been edited to express the first isoform of IL2RG in the cell or cells. In some such cells or populations, the genomic locus is an endogenous IL2RG genomic locus. In some such cells or populations, the genomic locus is not an endogenous IL2RG genomic locus.

In some such cells or populations, the altered epitope is in a binding region of the IL2RG antagonist, the IL2RG antagonist is an antibody comprising an immunoglobulin light chain or variable region thereof comprising three light chain CDRs and an immunoglobulin heavy chain or variable region thereof comprising three heavy chain CDRs, the three light chain CDRs comprise, consist essentially of, or consist of the sequences set forth in SEQ ID NOS: 12, 14, and 16, respectively, and the three heavy chain CDRs comprise, consist essentially of, or consist of the sequences set forth in SEQ ID NOS: 4, 6, and 8, respectively.

In some such cells or populations, the mutation comprises a mutation encoded by nucleotides within exon 2 and/or exon 3 of the IL2RG gene at the IL2RG genomic locus. In some such cells or populations, the mutation comprises a mutation or substitution within a region from position T127 to position N150 and/or within a region from position L87 to position D97. In some such cells or populations, the mutation comprises a mutation or substitution at position M145, position W90, position K92, position N93, position D95, position D97, position T127, position R139, position R140, position Q141, position T143, and/or position K147. In some such cells or populations, the mutation comprises a mutation or substitution at position M145, optionally wherein the substitution is a M145K substitution, a M145D substitution, a M145E substitution, a M145P substitution, a M145W substitution, or an M145Y substitution. In some such cells or populations, the mutation comprises a M145K substitution. In some such cells or populations, the mutation comprises a mutation or substitution at position W90, optionally wherein the substitution is a W90V substitution, a W90R substitution, a W90Q substitution, a W90L substitution, a W90K substitution, a W90E substitution, or a W90D substitution, optionally wherein the mutation comprises a W90Q substitution. In some such cells or populations, the mutation comprises a mutation or substitution at position M145 and a mutation or substitution at position W90.

In some such cells or populations, the first isoform and the second isoform are immunologically distinguishable by the IL2RG antagonist, wherein the IL2RG antagonist specifically binds to the second isoform of IL2RG but does not specifically bind to the first isoform of IL2RG. In some such cells or populations, the IL2RG antagonist is an antigen-binding protein. In some such cells or populations, the antigen-binding protein is an antibody or an antigen-binding fragment thereof. In some such cells or populations, the antigen-binding protein comprises an immunoglobulin light chain or variable region thereof comprising three light chain CDRs and an immunoglobulin heavy chain or variable region thereof comprising three heavy chain CDRs, the three light chain CDRs comprise, consist essentially of, or consist of sequences at least 90% identical to the sequences set forth in SEQ ID NOS: 12, 14, and 16, respectively, and the three heavy chain CDRs comprise, consist essentially of, or consist of sequences at least 90% identical to the sequences set forth in SEQ ID NOS: 4, 6, and 8, respectively. In some such cells or populations, the three light chain CDRs comprise, consist essentially of, or consist of the sequences set forth in SEQ ID NOS: 12, 14, and 16, respectively, and the three heavy chain CDRs comprise, consist essentially of, or consist of the sequences set forth in SEQ ID NOS: 4, 6, and 8, respectively. In some such cells or populations, the antigen-binding protein comprises an immunoglobulin light chain variable region that comprises, consists essentially of, or consists of a sequence at least 90% identical to the sequence set forth in SEQ ID NO: 10, and the antigen-binding protein comprises an immunoglobulin heavy chain variable region that comprises, consists essentially of, or consists of a sequence at least 90% identical to the sequence set forth in SEQ ID NO: 2. In some such cells or populations, the immunoglobulin light chain variable region comprises, consists essentially of, or consists of the sequence set forth in SEQ ID NO: 10, and the immunoglobulin heavy chain variable region comprises, consists essentially of, or consists of the sequence set forth in SEQ ID NO: 2. In some such cells or populations, the antigen-binding protein comprises an immunoglobulin light chain that comprises, consists essentially of, or consists of the sequence set forth in SEQ ID NO: 20, and the antigen-binding protein comprises an immunoglobulin heavy chain that comprises, consists essentially of, or consists of the sequence set forth in SEQ ID NO: 18.

In some such cells or populations, the cell or cells are hematopoietic cell(s). In some such cells or populations, the cell or cells are immune cell(s). In some such cells or populations, the cell or cells are lymphocytes or lymphoid progenitor cell(s). In some such cells or populations, the cell or cells are T cell(s). In some such cells or populations, the cell or cells are tumor infiltrating lymphocyte(s) (TILs). In some such cells or populations, the cell or cells are B cell(s). In some such cells or populations, the cell or cells are NK cell(s). In some such cells or populations, the cell or cells are hematopoietic stem cell(s) or hematopoietic stem and progenitor cell(s). In some such cells or populations, the cell or cells are induced pluripotent stem cell(s). In some such cells or populations, the cell or cells are mammalian cell(s) or non-human mammalian cell(s). In some such cells or populations, the cell or cells are human cell(s).

In some such cells or populations, the cell or cells comprise or express a therapeutic molecule. In some such cells or populations, the therapeutic molecule does not target IL2RG. In some such cells or populations, the cell or cells comprise or express an immunoglobulin. In some such cells or populations, the immunoglobulin does not target IL2RG. In some such cells or populations, the cell or cells comprise a chimeric antigen receptor (CAR) or an exogenous T cell receptor (TCR). In some such cells or populations, the CAR or the exogenous TCR does not target IL2RG.

In some such cells or populations, the cell or cells are isolated from a subject. In some such cells or populations, the cell or cells are for use in treatment of a subject having cells expressing the second isoform of IL2RG.

In another aspect, provided are methods of making any of the above isolated cells or populations of cells. Some such methods comprise modifying a cell or population of cells to express the first isoform of IL2RG. In some such methods, the modifying comprises introducing an expression vector encoding the first isoform of IL2RG. In some such methods, the modifying comprises editing a genomic locus to express the first isoform of IL2RG. In some such methods, the genomic locus is an endogenous IL2RG genomic locus. In some such methods, the genomic locus is not an endogenous IL2RG genomic locus. In some such methods, the editing comprises introducing into the cells: (1) a nuclease agent or one or more nucleic acids encoding the nuclease agent, wherein the nuclease agent targets a nuclease target sequence in the genomic locus; and (2) an exogenous donor nucleic acid, wherein the nuclease agent cleaves the genomic locus and the exogenous donor nucleic acid is inserted into the genomic locus or recombines with the genomic locus to generate the donor cells that express the first isoform of IL2RG. In some such methods, the nuclease agent comprises: (a) a zinc finger nuclease (ZFN); (b) a transcription activator-like effector nuclease (TALEN); or (c) (i) a Cas protein; and (ii) a guide RNA, wherein the guide RNA comprises a DNA-targeting segment that targets a guide RNA target sequence that is the nuclease target sequence, and wherein the guide RNA binds to the Cas protein and targets the Cas protein to the guide RNA target sequence. In some such methods, the nuclease agent comprises the Cas protein and the guide RNA, optionally wherein the target protein is IL2RG, and the DNA-targeting segment comprises the sequence set forth in any one of SEQ ID NOS: 76-87 or the guide RNA target sequence comprises the sequence set forth in any one of SEQ ID NOS: 64-75, or optionally wherein the target protein is IL2RG, and the DNA-targeting segment comprises the sequence set forth in any one of SEQ ID NOS: 136-153 or the guide RNA target sequence comprises the sequence set forth in any one of SEQ ID NOS: 118-135. In some such methods, the Cas protein is a Cas9 protein. In some such methods, the exogenous donor nucleic acid comprises homology arms. In some such methods, the exogenous donor nucleic acid is a single-stranded oligodeoxynucleotide (ssODN), optionally wherein the target protein is IL2RG, and the ssODN comprises the nucleic acid sequence set forth in any one of SEQ ID NOS: 88-117 or optionally wherein the target protein is IL2RG, and the ssODN comprises the nucleic acid sequence set forth in any one of SEQ ID NOS: 154-173.

In some such methods, the target protein is IL2RG, and (I) the DNA-targeting segment comprises the sequence set forth in SEQ ID NO: 77 or the guide RNA target sequence comprises the sequence set forth in SEQ ID NO: 65, and the ssODN comprises the nucleic acid sequence set forth in any one of SEQ ID NOS: 88-97; (II) the DNA-targeting segment comprises the sequence set forth in SEQ ID NO: 83 or the guide RNA target sequence comprises the sequence set forth in SEQ ID NO: 71, and the ssODN comprises the nucleic acid sequence set forth in any one of SEQ ID NOS: 98-10⁷; (III) the DNA-targeting segment comprises the sequence set forth in SEQ ID NO: 86 or the guide RNA target sequence comprises the sequence set forth in SEQ ID NO: 74, and the ssODN comprises the nucleic acid sequence set forth in any one of SEQ ID NOS: 108-117; (IV) the DNA-targeting segment comprises the sequence set forth in SEQ ID NO: 137 or the guide RNA target sequence comprises the sequence set forth in SEQ ID NO: 119, and the ssODN comprises the nucleic acid sequence set forth in any one of SEQ ID NOS: 154-163; or (V) the DNA-targeting segment comprises the sequence set forth in SEQ ID NO: 138 or the guide RNA target sequence comprises the sequence set forth in SEQ ID NO: 120, and the ssODN comprises the nucleic acid sequence set forth in any one of SEQ ID NOS: 164-173.

In another aspect, provided are genetically engineered human interleukin-2 receptor subunit gamma (IL2RG) proteins. In some such genetically engineered IL2RG proteins, the protein comprises an artificial mutation to provide an altered epitope, the altered epitope is a binding region of an IL2RG antagonist (e.g., an antigen-binding protein) such that the IL2RG antagonist exhibits reduced or abolished ability to bind and/or inhibit the genetically engineered IL2RG protein (e.g., compared to its ability to bind and/or inhibit a wild type human IL2RG protein), and the genetically engineered IL2RG protein retains binding to its endogenous ligands. In some such genetically engineered IL2RG proteins, the genetically engineered IL2RG protein is functionally indistinguishable but immunologically distinguishable from a native IL2RG protein.

In some such genetically engineered IL2RG proteins, the altered epitope is in a binding region of the IL2RG antagonist, the IL2RG antagonist is an antibody comprising an immunoglobulin light chain or variable region thereof comprising three light chain CDRs and an immunoglobulin heavy chain or variable region thereof comprising three heavy chain CDRs, the three light chain CDRs comprise, consist essentially of, or consist of the sequences set forth in SEQ ID NOS: 12, 14, and 16, respectively, and the three heavy chain CDRs comprise, consist essentially of, or consist of the sequences set forth in SEQ ID NOS: 4, 6, and 8, respectively.

In some such genetically engineered IL2RG proteins, the mutation comprises a mutation encoded by nucleotides within exon 2 and/or exon 3 of the IL2RG gene at the IL2RG genomic locus. In some such genetically engineered IL2RG proteins, the mutation comprises a mutation or substitution within a region from position T127 to position N150 and/or within a region from position L87 to position D97. In some such genetically engineered IL2RG proteins, the artificial mutation comprises a mutation or substitution at position M145, position W90, position K92, position N93, position D95, position D97, position T127, position R139, position R140, position Q141, position T143, and/or position K147. In some such genetically engineered IL2RG proteins, the artificial mutation comprises a mutation or substitution at position M145, optionally wherein the substitution is a M145K substitution, a M145D substitution, a M145E substitution, a M145P substitution, a M145W substitution, or an M145Y substitution. In some such genetically engineered IL2RG proteins, the artificial mutation comprises a M145K substitution. In some such genetically engineered IL2RG proteins, the mutation comprises a mutation or substitution at position W90, optionally wherein the substitution is a W90V substitution, a W90R substitution, a W90Q substitution, a W90L substitution, a W90K substitution, a W90E substitution, or a W90D substitution, optionally wherein the mutation comprises a W90Q substitution. In some such genetically engineered IL2RG proteins, the mutation comprises a mutation or substitution at position M145 and a mutation or substitution at position W90.

In some such genetically engineered IL2RG proteins, the genetically engineered IL2RG protein and the native IL2RG protein are functionally indistinguishable but immunologically distinguishable by the IL2RG antagonist. In some such genetically engineered IL2RG proteins, the IL2RG antagonist is an antigen-binding protein. In some such genetically engineered IL2RG proteins, the antigen-binding protein is an antibody or an antigen-binding fragment thereof. In some such genetically engineered IL2RG proteins, the antigen-binding protein comprises an immunoglobulin light chain or variable region thereof comprising three light chain CDRs and an immunoglobulin heavy chain or variable region thereof comprising three heavy chain CDRs, the three light chain CDRs comprise, consist essentially of, or consist of sequences at least 90% identical to the sequences set forth in SEQ ID NOS: 12, 14, and 16, respectively, and the three heavy chain CDRs comprise, consist essentially of, or consist of sequences at least 90% identical to the sequences set forth in SEQ ID NOS: 4, 6, and 8, respectively. In some such genetically engineered IL2RG proteins, the three light chain CDRs comprise, consist essentially of, or consist of the sequences set forth in SEQ ID NOS: 12, 14, and 16, respectively, and the three heavy chain CDRs comprise, consist essentially of, or consist of the sequences set forth in SEQ ID NOS: 4, 6, and 8, respectively. In some such genetically engineered IL2RG proteins, the antigen-binding protein comprises an immunoglobulin light chain variable region that comprises, consists essentially of, or consists of a sequence at least 90% identical to the sequence set forth in SEQ ID NO: 10, and the antigen-binding protein comprises an immunoglobulin heavy chain or variable region that comprises, consists essentially of, or consists of a sequence at least 90% identical to the sequence set forth in SEQ ID NO: 2. In some such genetically engineered IL2RG proteins, the immunoglobulin light chain variable region comprises, consists essentially of, or consists of the sequence set forth in SEQ ID NO: 10, and the immunoglobulin heavy chain variable region comprises, consists essentially of, or consists of the sequence set forth in SEQ ID NO: 2. In some such genetically engineered IL2RG proteins, the antigen-binding protein comprises an immunoglobulin light chain that comprises, consists essentially of, or consists of the sequence set forth in SEQ ID NO: 20, and the antigen-binding protein comprises an immunoglobulin heavy chain that comprises, consists essentially of, or consists of the sequence set forth in SEQ ID NO: 18.

In another aspect, provided are nucleic acids encoding any of the above genetically engineered IL2RG proteins. Some such nucleic acids are expression vectors encoding the genetically engineered human IL2RG protein.

Methods for in vivo selective depletion of non-edited cells in a subject thereof are provided. Such methods can comprise providing cells edited to express a first isoform of a target protein (e.g., interleukin-2 receptor subunit gamma (IL2RG)), administering the edited cells to the subject, and then selectively depleting non-edited cells in the subject based on their expression of a second isoform of the target protein. Such methods can comprise providing cells edited to express a first isoform of a target protein (e.g., interleukin-2 receptor subunit gamma (IL2RG)) that is functionally indistinguishable but immunologically distinguishable from a second isoform of the target protein, administering the edited cells to the subject, and then selectively depleting non-edited cells in the subject based on their expression of the second isoform of the target protein. Also provided are combinations for administration to a subject in need thereof, wherein the combination comprises (1) a population of cells edited to express a first isoform of a target protein (e.g., IL2RG) and (2) an antagonist (e.g., anti-IL2RG antigen-binding protein) that specifically binds to a second isoform of the target protein but does not specifically bind to the first isoform of the target protein. Also provided are isolated cells or populations of cells modified to express a first isoform of interleukin-2 receptor subunit gamma (IL2RG) that is different from a second isoform of IL2RG. Also provided are isolated cells or populations of cells in which a genomic locus has been edited to express a first isoform of interleukin-2 receptor subunit gamma (IL2RG) that is different from a second isoform of IL2RG. Also provided are isolated cells or populations of cells in which an IL2RG genomic locus has been edited to express a first isoform of interleukin-2 receptor subunit gamma (IL2RG) that is different from a second isoform of IL2RG, wherein the first isoform and the second isoform are functionally indistinguishable but immunologically distinguishable. Also provided are methods of making the isolated cells or populations of cells. Also provided are genetically engineered interleukin-2 receptor subunit gamma (IL2RG) proteins and nucleic acids encoding the proteins.

In one aspect, provided are methods for in vivo selective depletion of non-edited cells in a subject in need thereof. Some such methods comprise: (a) providing edited cells in which an IL2RG genomic locus has been edited to express a first isoform of interleukin-2 receptor subunit gamma (IL2RG) that is different from a second isoform of IL2RG, wherein the first isoform and the second isoform are functionally indistinguishable but immunologically distinguishable, and wherein the second isoform is expressed in non-edited cells of the subject; (b) administering the edited cells to the subject, and (c) selectively depleting non-edited cells in the subject based on their expression of the second isoform of IL2RG. In one aspect, provided are methods for in vivo selective depletion of non-edited cells and repopulation with edited cells in a subject in need thereof. Some such methods comprise: (a) providing edited cells that have been modified to express a first isoform of interleukin-2 receptor subunit gamma (IL2RG) that is different from a second isoform of IL2RG, and wherein the second isoform is expressed in non-edited cells of the subject; (b) administering the edited cells to the subject, and (c) selectively depleting non-edited cells in the subject based on their expression of the second isoform of IL2RG. In some such methods, the first isoform and the second isoform are functionally indistinguishable but immunologically distinguishable. In some such methods, the edited cells express both the first isoform of IL2RG and the second isoform of IL2RG. In some such methods, the edited cells express only the first isoform of IL2RG.

In some such methods, the first isoform of IL2RG is a genetically engineered isoform of IL2RG. In some such methods, the first isoform of IL2RG is genetically engineered to comprise a mutation to provide an altered epitope, optionally wherein the mutation is an artificial mutation.

In some such methods, the altered epitope is in a binding region of an antibody comprising an immunoglobulin light chain or variable region thereof comprising three light chain CDRs and an immunoglobulin heavy chain or variable region thereof comprising three heavy chain CDRs, wherein the three light chain CDRs comprise, consist essentially of, or consist of the sequences set forth in SEQ ID NOS: 12, 14, and 16, respectively, and wherein the three heavy chain CDRs comprise, consist essentially of, or consist of the sequences set forth in SEQ ID NOS: 4, 6, and 8, respectively.

In some such methods, the mutation comprises a mutation encoded by nucleotides within exon 2 and/or exon 3 of the IL2RG gene at the IL2RG genomic locus. In some such methods, the mutation comprises a mutation or substitution within a region from position T127 to position N150 and/or within a region from position L87 to position D97. In some such methods, the mutation comprises a mutation or substitution at position M145, position W90, position K92, position N93, position D95, position D97, position T127, position R139, position R140, position Q141, position T143, or position K147. In some such methods, the mutation comprises a mutation or substitution at position M145, optionally wherein the substitution is a M145K substitution, a M145D substitution, a M145E substitution, a M145P substitution, a M145W substitution, or an M145Y substitution. In some such methods, the mutation comprises a M145K substitution. In some such methods, the mutation comprises a mutation or substitution at position W90, optionally wherein the substitution is a W90V substitution, a W90R substitution, a W90Q substitution, a W90L substitution, a W90K substitution, a W90E substitution, or a W90D substitution. In some such methods, the mutation comprises a mutation or substitution at position M145 and a mutation or substitution at position W90.

In some such methods, the selective depletion in step (c) comprises administering an IL2RG antagonist to the subject, wherein the IL2RG antagonist specifically binds to the second isoform of IL2RG but does not specifically bind to the first isoform of IL2RG. In some such methods, the IL2RG antagonist is an antigen-binding protein. In some such methods, the antigen-binding protein is an antibody or an antigen-binding fragment thereof. In some such methods, the antigen-binding protein comprises an immunoglobulin light chain or variable region thereof comprising three light chain CDRs and an immunoglobulin heavy chain or variable region thereof comprising three heavy chain CDRs, wherein the three light chain CDRs comprise, consist essentially of, or consist of sequences at least 90% identical to the sequences set forth in SEQ ID NOS: 12, 14, and 16, respectively, and wherein the three heavy chain CDRs comprise, consist essentially of, or consist of sequences at least 90% identical to the sequences set forth in SEQ ID NOS: 4, 6, and 8, respectively. In some such methods, the three light chain CDRs comprise, consist essentially of, or consist of the sequences set forth in SEQ ID NOS: 12, 14, and 16, respectively, and the three heavy chain CDRs comprise, consist essentially of, or consist of the sequences set forth in SEQ ID NOS: 4, 6, and 8, respectively. In some such methods, the immunoglobulin light chain or variable region thereof comprises, consists essentially of, or consists of a sequence at least 90% identical to the sequence set forth in SEQ ID NO: 10, and the immunoglobulin heavy chain or variable region thereof comprises, consists essentially of, or consists of a sequence at least 90% identical to the sequence set forth in SEQ ID NO: 2. In some such methods, the immunoglobulin light chain or variable region thereof comprises, consists essentially of, or consists of the sequence set forth in SEQ ID NO: 10, and the immunoglobulin heavy chain or variable region thereof comprises, consists essentially of, or consists of the sequence set forth in SEQ ID NO: 2.

In some such methods, the edited cells are hematopoietic cells. In some such methods, the edited cells are lymphocytes or lymphoid progenitor cells. In some such methods, the edited cells are T cells. In some such methods, the edited cells are tumor infiltrating lymphocytes (TILs). In some such methods, the edited cells are B cells. In some such methods, the edited cells are NK cells. In some such methods, the edited cells are hematopoietic stem and progenitor cells. In some such methods, the edited cells are derived from induced pluripotent stem cells. In some such methods, the edited cells are derived from hematopoietic stem cells or hematopoietic stem and progenitor cells. In some such methods, the subject is a mammal or a non-human mammal, and the edited cells are mammalian cells or non-human mammalian cells. In some such methods, the subject is a human, and the edited cells are human cells. In some such methods, the edited cells comprise or express a therapeutic molecule. In some such methods, the edited cells comprise or express an immunoglobulin. In some such methods, the edited cells comprise a chimeric antigen receptor (CAR) or an exogenous T cell receptor (TCR). In some such methods, the edited cells are autologous. In some such methods, the edited cells are allogeneic or syngeneic.

In some such methods, the subject has a hematopoietic malignancy, and the method is for treating the hematopoietic malignancy in the subject. In some such methods, the subject has cancer. In some such methods, the cancer is a hematologic cancer. In some such methods, the subject has defective immune cells or a genetic deficiency in hematopoiesis. In some such methods, the genetic deficiency in hematopoiesis is sickle cell disease or severe combined immunodeficiency (SCID).

In some such methods, steps (b) and (c) occur simultaneously. In some such methods, step (b) occurs prior to step (c). In some such methods, step (b) occurs subsequent to step (c).

Some such methods further comprise generating the edited cells by modifying a population of cells to express the first isoform of IL2RG prior to step (a). In some such methods, the population of cells is a population of induced pluripotent stem cells, and the method further comprises differentiating the edited induced pluripotent stem cells prior to step (a) into the edited cells that are administered in step (a), optionally wherein the induced pluripotent stem cells are differentiated into hematopoietic cells, lymphocytes or lymphoid progenitor cells, T cells, B cells, NK cells, hematopoietic stem cells, or hematopoietic stem and progenitor cells. In some such methods, the population of cells is a population of hematopoietic stem cells or hematopoietic stem and progenitor cells, and the method further comprises differentiating the edited hematopoietic stem cells or hematopoietic stem and progenitor cells prior to step (a) into the edited cells that are administered in step (a), optionally wherein the hematopoietic stem cells or hematopoietic stem and progenitor cells are differentiated into differentiated hematopoietic cells, lymphocytes or lymphoid progenitor cells, T cells, B cells, or NK cells.

In some such methods, a genomic locus has been edited to express the first isoform of IL2RG in the edited cells. In some such methods, the genomic locus is an IL2RG genomic locus. In some such methods, the genomic locus is not an IL2RG genomic locus. In some such methods, the method further comprises generating the edited cells by editing the genomic locus in a population of cells to express the first isoform of IL2RG prior to step (a). In some such methods, the method further comprises generating the edited cells by editing the IL2RG genomic locus in a population of cells to express the first isoform of IL2RG prior to step (a). In some such methods, the population of cells is a population of induced pluripotent stem cells, the IL2RG locus is edited in induced pluripotent stem cells to generate edited induced pluripotent stem cells that express the first isoform of IL2RG, and the method further comprises differentiating the edited induced pluripotent stem cells prior to step (a) into the edited cells that are administered in step (a), optionally wherein the induced pluripotent stem cells are differentiated into hematopoictic cells, lymphocytes or lymphoid progenitor cells, T cells, B cells, NK cells, hematopoictic stem cells, or hematopoictic stem and progenitor cells. In some such methods, the population of cells is a population of hematopoietic stem cells or hematopoietic stem and progenitor cells, the IL2RG locus is edited in hematopoietic stem cells or hematopoietic stem and progenitor cells to generate edited hematopoietic stem cells or hematopoietic stem and progenitor cells that express the first isoform of IL2RG, and the method further comprises differentiating the edited hematopoietic stem cells or hematopoietic stem and progenitor cells prior to step (a) into the edited cells that are administered in step (a), optionally wherein the hematopoietic stem cells or hematopoietic stem and progenitor cells are differentiated into differentiated hematopoictic cells, lymphocytes or lymphoid progenitor cells, T cells, B cells, or NK cells.

In some such methods, the editing comprises introducing into the population of cells: (1) a nuclease agent or one or more nucleic acids encoding the nuclease agent, wherein the nuclease agent targets a nuclease target sequence in the genomic locus; and (2) an exogenous donor nucleic acid, wherein the nuclease agent cleaves the genomic locus and the exogenous donor nucleic acid is inserted into the genomic locus or recombines with the genomic locus to generate the edited cells that express the first isoform of IL2RG. In some such methods, the editing comprises introducing into the population of cells: (1) a nuclease agent or one or more nucleic acids encoding the nuclease agent, wherein the nuclease agent targets a nuclease target sequence in the IL2RG genomic locus; and (2) an exogenous donor nucleic acid, wherein the nuclease agent cleaves the IL2RG genomic locus and the exogenous donor nucleic acid is inserted into the IL2RG genomic locus or recombines with the IL2RG genomic locus to generate the edited cells that express the first isoform of IL2RG. In some such methods, the nuclease agent comprises: (a) a zinc finger nuclease (ZFN); (b) a transcription activator-like effector nuclease (TALEN); or (c) (i) a Cas protein; and (ii) a guide RNA, wherein the guide RNA comprises a DNA-targeting segment that targets a guide RNA target sequence that is the nuclease target sequence, and wherein the guide RNA binds to the Cas protein and targets the Cas protein to the guide RNA target sequence. In some such methods, the nuclease agent comprises the Cas protein and the guide RNA, optionally wherein the DNA-targeting segment comprises the sequence set forth in any one of SEQ ID NOS: 76-87 or optionally wherein the guide RNA target sequence comprises the sequence set forth in any one of SEQ ID NOS: 64-75. In some such methods, the Cas protein is a Cas9 protein. In some such methods, the exogenous donor nucleic acid comprises homology arms. In some such methods, the exogenous donor nucleic acid is a single-stranded oligodeoxynucleotide (ssODN), optionally wherein the ssODN comprises the nucleic acid sequence set forth in any one of SEQ ID NOS: 88-117.

In some such methods, the method further comprises isolating the population of cells from the subject or from a different subject prior to modifying the population of cells. In some such methods, the method further comprises isolating the population of cells from the subject or from a different subject prior to editing the IL2RG genomic locus.

In another aspect, provided is a combination or a combination medicament for administration to a subject in need thereof. In some such combinations, the combination comprises: (a) a population of cells modified to express a first isoform of interleukin-2 receptor subunit gamma (IL2RG) that is different from a second isoform of IL2RG; and (b) an IL2RG antagonist that specifically binds to the second isoform of IL2RG but does not specifically bind to the first isoform of IL2RG. In some such combinations, the first isoform and the second isoform are functionally indistinguishable but immunologically distinguishable. In some such combinations, a genomic locus has been edited to express the first isoform of IL2RG in the population of cells. In some such combinations, the genomic locus is an IL2RG genomic locus. In some such combinations, wherein the genomic locus is not an IL2RG genomic locus. In some such combinations, the combination comprises: (a) a population of cells in which an IL2RG genomic locus has been edited to express a first isoform of interleukin-2 receptor subunit gamma (IL2RG) that is different from a second isoform of IL2RG, wherein the first isoform and the second isoform are functionally indistinguishable but immunologically distinguishable; and (b) an IL2RG antagonist that specifically binds to the second isoform of IL2RG but does not specifically bind to the first isoform of IL2RG. In some such combinations, the cells express both the first isoform of IL2RG and the second isoform of IL2RG. In some such combinations, the cells express only the first isoform of IL2RG.

In some such combinations, the first isoform of IL2RG is a genetically engineered isoform of IL2RG. In some such combinations, the first isoform of IL2RG is genetically engineered to comprise a mutation to provide an altered epitope, optionally wherein the mutation is an artificial mutation. In some such combinations, the altered epitope is in a binding region of an antibody comprising an immunoglobulin light chain or variable region thereof comprising three light chain CDRs and an immunoglobulin heavy chain or variable region thereof comprising three heavy chain CDRs, wherein the three light chain CDRs comprise, consist essentially of, or consist of the sequences set forth in SEQ ID NOS: 12, 14, and 16, respectively, and wherein the three heavy chain CDRs comprise, consist essentially of, or consist of the sequences set forth in SEQ ID NOS: 4, 6, and 8, respectively.

In some such combinations, the mutation comprises a mutation encoded by nucleotides within exon 2 and/or exon 3 of the IL2RG gene at the IL2RG genomic locus. In some such combinations, the mutation comprises a mutation or substitution within a region from position T127 to position N150 and/or within a region from position L87 to position D97. In some such combinations, the mutation comprises a mutation or substitution at position M145, position W90, position K92, position N93, position D95, position D97, position T127, position R139, position R140, position Q141, position T143, or position K147. In some such combinations, the mutation comprises a mutation or substitution at position M145, optionally wherein the substitution is a M145K substitution, a M145D substitution, a M145E substitution, a M145P substitution, a M145W substitution, or an M145Y substitution. In some such combinations, the mutation comprises a M145K substitution. In some such combinations, the mutation comprises a mutation or substitution at position W90, optionally wherein the substitution is a W90V substitution, a W90R substitution, a W90Q substitution, a W90L substitution, a W90K substitution, a W90E substitution, or a W90D substitution. In some such combinations, the mutation comprises a mutation or substitution at position M145 and a mutation or substitution at position W90.

In some such combinations, the IL2RG antagonist is an antigen-binding protein. In some such combinations, the antigen-binding protein is an antibody or an antigen-binding fragment thereof. In some such combinations, the antigen-binding protein comprises an immunoglobulin light chain or variable region thereof comprising three light chain CDRs and an immunoglobulin heavy chain or variable region thereof comprising three heavy chain CDRs, wherein the three light chain CDRs comprise, consist essentially of, or consist of sequences at least 90% identical to the sequences set forth in SEQ ID NOS: 12, 14, and 16, respectively, and wherein the three heavy chain CDRs comprise, consist essentially of, or consist of sequences at least 90% identical to the sequences set forth in SEQ ID NOS: 4, 6, and 8, respectively. In some such combinations, the three light chain CDRs comprise, consist essentially of, or consist of the sequences set forth in SEQ ID NOS: 12, 14, and 16, respectively, and the three heavy chain CDRs comprise, consist essentially of, or consist of the sequences set forth in SEQ ID NOS: 4, 6, and 8, respectively. In some such combinations, the immunoglobulin light chain or variable region thereof comprises, consists essentially of, or consists of a sequence at least 90% identical to the sequence set forth in SEQ ID NO: 10, and the immunoglobulin heavy chain or variable region thereof comprises, consists essentially of, or consists of a sequence at least 90% identical to the sequence set forth in SEQ ID NO: 2. In some such combinations, the immunoglobulin light chain or variable region thereof comprises, consists essentially of, or consists of the sequence set forth in SEQ ID NO: 10, and the immunoglobulin heavy chain or variable region thereof comprises, consists essentially of, or consists of the sequence set forth in SEQ ID NO: 2.

In some such combinations, the cells are hematopoietic cells. In some such combinations, the cells are lymphocytes or lymphoid progenitor cells. In some such combinations, the cells are T cells. In some such combinations, the cells are tumor infiltrating lymphocytes (TILs). In some such combinations, the cells are B cells. In some such combinations, the cells are NK cells. In some such combinations, the cells are hematopoictic stem cells or hematopoietic stem and progenitor cells. In some such combinations, the cells are derived from induced pluripotent stem cells. In some such combinations, the cells are derived from hematopoietic stem cells or hematopoictic stem and progenitor cells. In some such combinations, the subject is a mammal or a non-human mammal, and the cells are mammalian cells or non-human mammalian cells. In some such combinations, the subject is a human, and the cells are human cells. In some such combinations, the cells comprise or express a therapeutic molecule. In some such combinations, the cells comprise or express an immunoglobulin. In some such combinations, the cells comprise a chimeric antigen receptor (CAR) or an exogenous T cell receptor (TCR). In some such combinations, the cells are autologous. In some such combinations, the cells are allogeneic or syngeneic.

In some such combinations, the subject has a hematopoietic malignancy, and the combination medicament is for treating the hematopoietic malignancy in the subject. In some such combinations, the subject has cancer. In some such combinations, the cancer is a hematologic cancer. In some such combinations, the subject has defective immune cells or a genetic deficiency in hematopoiesis. In some such combinations, the genetic deficiency in hematopoiesis is sickle cell disease or severe combined immunodeficiency (SCID).

In another aspect, provided are isolated cells or populations of cells modified to express a first isoform of interleukin-2 receptor subunit gamma (IL2RG) that is different from a second isoform of IL2RG. In some such cells or populations of cells, the first isoform and the second isoform are functionally indistinguishable but immunologically distinguishable. In some such cells or populations of cells, a genomic locus has been edited to express the first isoform of IL2RG in the cell or cells. In some such cells or populations of cells, the genomic locus is an IL2RG genomic locus. In some such cells or populations of cells, the genomic locus is not an IL2RG genomic locus. In another aspect, provided are isolated cells or populations of cells in which an IL2RG genomic locus has been edited to express a first isoform of interleukin-2 receptor subunit gamma (IL2RG) that is different from a second isoform of IL2RG, wherein the first isoform and the second isoform are functionally indistinguishable but immunologically distinguishable. In some such cells or populations of cells, the cell or cells express both the first isoform of IL2RG and the second isoform of IL2RG. In some such cells or populations of cells, the cell or cells express only the first isoform of IL2RG.

In some such cells or populations of cells, the first isoform of IL2RG is a genetically engineered isoform of IL2RG. In some such cells or populations of cells, the first isoform of IL2RG is genetically engineered to comprise a mutation to provide an altered epitope, optionally wherein the mutation is an artificial mutation.

In some such cells or populations of cells, the cell or cells further comprise an exogenous donor nucleic acid comprising the artificial mutation and a nuclease agent or one or more nucleic acids encoding the nuclease agent, wherein the nuclease agent targets a nuclease target sequence in the genomic locus. In some such cells or populations of cells, the cell or cells further comprise an exogenous donor nucleic acid comprising the artificial mutation and a nuclease agent or one or more nucleic acids encoding the nuclease agent, wherein the nuclease agent targets a nuclease target sequence in the IL2RG genomic locus. In some such cells or populations of cells, the nuclease agent comprises: (a) a zinc finger nuclease (ZFN); (b) a transcription activator-like effector nuclease (TALEN); or (c) (i) a Cas protein; and (ii) a guide RNA, wherein the guide RNA comprises a DNA-targeting segment that targets a guide RNA target sequence that is the nuclease target sequence, and wherein the guide RNA binds to the Cas protein and targets the Cas protein to the guide RNA target sequence. In some such cells or populations of cells, the nuclease agent comprises the Cas protein and the guide RNA, optionally wherein the DNA-targeting segment comprises the sequence set forth in any one of SEQ ID NOS: 76-87 or optionally wherein the guide RNA target sequence comprises the sequence set forth in any one of SEQ ID NOS: 64-75. In some such cells or populations of cells, the Cas protein is a Cas9 protein. In some such cells or populations of cells, the exogenous donor nucleic acid comprises homology arms. In some such cells or populations of cells, the exogenous donor nucleic acid is a single-stranded oligodeoxynucleotide (ssODN), optionally wherein the ssODN comprises the nucleic acid sequence set forth in any one of SEQ ID NOS: 88-117.

In some such cells or populations of cells, the altered epitope is in a binding region of an antibody comprising an immunoglobulin light chain or variable region thereof comprising three light chain CDRs and an immunoglobulin heavy chain or variable region thereof comprising three heavy chain CDRs, wherein the three light chain CDRs comprise, consist essentially of, or consist of the sequences set forth in SEQ ID NOS: 12, 14, and 16, respectively, and wherein the three heavy chain CDRs comprise, consist essentially of, or consist of the sequences set forth in SEQ ID NOS: 4, 6, and 8, respectively.

In some such cells or populations of cells, the mutation comprises a mutation encoded by nucleotides within exon 2 and/or exon 3 of the IL2RG gene at the IL2RG genomic locus. In some such cells or populations of cells, the mutation comprises a mutation or substitution within a region from position T127 to position N150 and/or within a region from position L87 to position D97. In some such cells or populations of cells, the mutation comprises a mutation or substitution at position M145, position W90, position K92, position N93, position D95, position D97, position T127, position R139, position R140, position Q141, position T143, or position K147. In some such cells or populations of cells, the mutation comprises a mutation or substitution at position M145, optionally wherein the substitution is a M145K substitution, a M145D substitution, a M145E substitution, a M145P substitution, a M145W substitution, or an M145Y substitution. In some such cells or populations of cells, the mutation comprises a M145K substitution. In some such cells or populations of cells, the mutation comprises a mutation or substitution at position W90, optionally wherein the substitution is a W90V substitution, a W90R substitution, a W90Q substitution, a W90L substitution, a W90K substitution, a W90E substitution, or a W90D substitution. In some such cells or populations of cells, the mutation comprises a mutation or substitution at position M145 and a mutation or substitution at position W90.

In some such cells or populations of cells, the first isoform and the second isoform are functionally indistinguishable but immunologically distinguishable by an IL2RG antagonist. In some such cells or populations of cells, the IL2RG antagonist is an antigen-binding protein. In some such cells or populations of cells, the antigen-binding protein is an antibody or an antigen-binding fragment thereof. In some such cells or populations of cells, the antigen-binding protein comprises an immunoglobulin light chain or variable region thereof comprising three light chain CDRs and an immunoglobulin heavy chain or variable region thereof comprising three heavy chain CDRs, wherein the three light chain CDRs comprise, consist essentially of, or consist of sequences at least 90% identical to the sequences set forth in SEQ ID NOS: 12, 14, and 16, respectively, and wherein the three heavy chain CDRs comprise, consist essentially of, or consist of sequences at least 90% identical to the sequences set forth in SEQ ID NOS: 4, 6, and 8, respectively. In some such cells or populations of cells, the three light chain CDRs comprise, consist essentially of, or consist of the sequences set forth in SEQ ID NOS: 12, 14, and 16, respectively, and the three heavy chain CDRs comprise, consist essentially of, or consist of the sequences set forth in SEQ ID NOS: 4, 6, and 8, respectively. In some such cells or populations of cells, the immunoglobulin light chain or variable region thereof comprises, consists essentially of, or consists of a sequence at least 90% identical to the sequence set forth in SEQ ID NO: 10, and the immunoglobulin heavy chain or variable region thereof comprises, consists essentially of, or consists of a sequence at least 90% identical to the sequence set forth in SEQ ID NO: 2. In some such cells or populations of cells, the immunoglobulin light chain or variable region thereof comprises, consists essentially of, or consists of the sequence set forth in SEQ ID NO: 10, and the immunoglobulin heavy chain or variable region thereof comprises, consists essentially of, or consists of the sequence set forth in SEQ ID NO: 2.

In some such cells or populations of cells, the cell or cells are hematopoietic cell(s). In some such cells or populations of cells, the cell or cells are lymphocytes or lymphoid progenitor cell(s). In some such cells or populations of cells, the cell or cells are T cell(s). In some such cells or populations of cells, the cell or cells are tumor infiltrating lymphocyte(s) (TILs). In some such cells or populations of cells, the cell or cells are B cell(s). In some such cells or populations of cells, the cell or cells are NK cell(s). In some such cells or populations of cells, the cell or cells are hematopoietic stem cell(s) or hematopoietic stem and progenitor cell(s). In some such cells or populations of cells, the cell or cells are induced pluripotent stem cell(s). In some such cells or populations of cells, the cell or cells are mammalian cell(s) or non-human mammalian cell(s). In some such cells or populations of cells, the cell or cells are human cell(s). In some such cells or populations of cells, the cell or cells comprise or express a therapeutic molecule. In some such cells or populations of cells, the cell or cells comprise or express an immunoglobulin. In some such cells or populations of cells, the cell or cells comprise a chimeric antigen receptor (CAR) or an exogenous T cell receptor (TCR). In some such cells or populations of cells, the cell or cells are isolated from a subject. In some such cells or populations of cells, the cell or cells are for use in treatment of a subject having cells expressing the second isoform of IL2RG. In some such cells or populations of cells, the cell or cells are isolated from the subject.

In another aspect, provided are methods of making any of the above isolated cells or populations of cells. In some such methods, the method comprises modifying a cell or population of cells to express the first isoform of IL2RG. In some such methods, the modifying comprises editing a genomic locus to express the first isoform of IL2RG. In some such methods, the genomic locus is an IL2RG genomic locus. In some such methods, the genomic locus is not an IL2RG genomic locus. In some such methods, the method comprises editing the IL2RG genomic locus to express the first isoform of IL2RG.

In some such methods, the editing comprises introducing into the cells: (1) a nuclease agent or one or more nucleic acids encoding the nuclease agent, wherein the nuclease agent targets a nuclease target sequence in the genomic locus; and (2) an exogenous donor nucleic acid, wherein the nuclease agent cleaves the genomic locus and the exogenous donor nucleic acid is inserted into the genomic locus or recombines with the genomic locus to generate the edited cells that express the first isoform of IL2RG. In some such methods, the editing comprises introducing into the cells: (1) a nuclease agent or one or more nucleic acids encoding the nuclease agent, wherein the nuclease agent targets a nuclease target sequence in the IL2RG genomic locus; and (2) an exogenous donor nucleic acid, wherein the nuclease agent cleaves the IL2RG genomic locus and the exogenous donor nucleic acid is inserted into the IL2RG genomic locus or recombines with the IL2RG genomic locus to generate the edited cells that express the first isoform of IL2RG. In some such methods, the nuclease agent comprises: (a) a zinc finger nuclease (ZFN); (b) a transcription activator-like effector nuclease (TALEN); or (c) (i) a Cas protein; and (ii) a guide RNA, wherein the guide RNA comprises a DNA-targeting segment that targets a guide RNA target sequence that is the nuclease target sequence, and wherein the guide RNA binds to the Cas protein and targets the Cas protein to the guide RNA target sequence. In some such methods, the nuclease agent comprises the Cas protein and the guide RNA, optionally wherein the DNA-targeting segment comprises the sequence set forth in any one of SEQ ID NOS: 76-87 or optionally wherein the guide RNA target sequence comprises the sequence set forth in any one of SEQ ID NOS: 64-75. In some such methods, the Cas protein is a Cas9 protein. In some such methods, the exogenous donor nucleic acid comprises homology arms. In some such methods, the exogenous donor nucleic acid is a single-stranded oligodeoxynucleotide (ssODN), optionally wherein the ssODN comprises the nucleic acid sequence set forth in any one of SEQ ID NOS: 88-117.

In another aspect, provided are genetically engineered interleukin-2 receptor subunit gamma (IL2RG) proteins comprising an artificial mutation to provide an altered epitope. In some such proteins, the genetically engineered IL2RG protein is functionally indistinguishable but immunologically distinguishable from a native IL2RG protein. In another aspect, provided are genetically engineered interleukin-2 receptor subunit gamma (IL2RG) proteins comprising an artificial mutation to provide an altered epitope, wherein the genetically engineered IL2RG protein is functionally indistinguishable but immunologically distinguishable from a native IL2RG protein. In some such proteins, the altered epitope is in a binding region of an antibody comprising an immunoglobulin light chain or variable region thereof comprising three light chain CDRs and an immunoglobulin heavy chain or variable region thereof comprising three heavy chain CDRs, wherein the three light chain CDRs comprise, consist essentially of, or consist of the sequences set forth in SEQ ID NOS: 12, 14, and 16, respectively, and wherein the three heavy chain CDRs comprise, consist essentially of, or consist of the sequences set forth in SEQ ID NOS: 4, 6, and 8, respectively. In some such proteins, the mutation comprises a mutation encoded by nucleotides within exon 2 and/or exon 3 of the IL2RG gene at the IL2RG genomic locus. In some such proteins, the mutation comprises a mutation or substitution within a region from position T127 to position N150 and/or within a region from position L87 to position D97. In some such proteins, the artificial mutation comprises a mutation or substitution at position M145, position W90, position K92, position N93, position D95, position D97, position T127, position R139, position R140, position Q141, position T143, or position K147. In some such proteins, the artificial mutation comprises a mutation or substitution at position M145, optionally wherein the substitution is a M145K substitution, a M145D substitution, a M145E substitution, a M145P substitution, a M145W substitution, or an M145Y substitution. In some such proteins, the artificial mutation comprises a M145K substitution. In some such proteins, the mutation comprises a mutation or substitution at position W90, optionally wherein the substitution is a W90V substitution, a W90R substitution, a W90Q substitution, a W90L substitution, a W90K substitution, a W90E substitution, or a W90D substitution. In some such proteins, the mutation comprises a mutation or substitution at position M145 and a mutation or substitution at position W90.

In some such proteins, the genetically engineered IL2RG protein and the native IL2RG protein are functionally indistinguishable but immunologically distinguishable by an IL2RG antagonist. In some such proteins, the IL2RG antagonist is an antigen-binding protein. In some such proteins, the antigen-binding protein is an antibody or an antigen-binding fragment thereof. In some such proteins, the antigen-binding protein comprises an immunoglobulin light chain or variable region thereof comprising three light chain CDRs and an immunoglobulin heavy chain or variable region thereof comprising three heavy chain CDRs, wherein the three light chain CDRs comprise, consist essentially of, or consist of sequences at least 90% identical to the sequences set forth in SEQ ID NOS: 12, 14, and 16, respectively, and wherein the three heavy chain CDRs comprise, consist essentially of, or consist of sequences at least 90% identical to the sequences set forth in SEQ ID NOS: 4, 6, and 8, respectively. In some such proteins, the three light chain CDRs comprise, consist essentially of, or consist of the sequences set forth in SEQ ID NOS: 12, 14, and 16, respectively, and the three heavy chain CDRs comprise, consist essentially of, or consist of the sequences set forth in SEQ ID NOS: 4, 6, and 8, respectively. In some such proteins, the immunoglobulin light chain or variable region thereof comprises, consists essentially of, or consists of a sequence at least 90% identical to the sequence set forth in SEQ ID NO: 10, and the immunoglobulin heavy chain or variable region thereof comprises, consists essentially of, or consists of a sequence at least 90% identical to the sequence set forth in SEQ ID NO: 2. In some such proteins, the immunoglobulin light chain or variable region thereof comprises, consists essentially of, or consists of the sequence set forth in SEQ ID NO: 10, and the immunoglobulin heavy chain or variable region thereof comprises, consists essentially of, or consists of the sequence set forth in SEQ ID NO: 2. In some such proteins, the IL2RG protein is a human IL2RG protein.

In another aspect, provided are nucleic acids encoding any of the above genetically engineered IL2RG proteins.

### BRIEF DESCRIPTION OF THE FIGURES

FIG. 1 shows a schematic of the antibody-resistant modified receptor (ARMoR) concept in which targeted host conditioning is combined with donor cell editing to enhance cell-based therapies and transplants. The concept is a two-tiered approach to afford grafted cells a competitive advantage: (1) targeted suppression of host cells (e.g., T cells, NK cells, B cells, lymphoid progenitors, hematologic cancer) with an antibody targeting IL2RG; and (2) ex vivo modification of grafted cells (IL2RG variant-antibody resistant modified receptor (ARMoR)) to enhance survival in host. The IL2RG variant retains cytokine signaling function but does not bind to the anti-IL2RG antibody.

FIGS. 2A-2B show that host T cell suppression with IL2RG blockade creates a niche for durable engraftment of donor REGN7257-resistant T cells in recipient Il2rg^{hu/hu} mice. FIG. 2A shows a schematic of the experimental protocol. REGN7257 or REGN1945 isotype control was injected by subcutaneous injection two times per week at 25 mg/kg starting at Day-25 (indicated by closed arrows). 20×10⁶ donor CD4+ and CD8+ T cells were injected at Day 0 (open arrow), and bone marrow, spleen, and blood samples were taken at Day 24 (open arrow). FIG. 2B shows anti-IL2RG conditioning facilitates uptake of “ARMoR′d” donor T cells, which persist and proliferate over the course of 3 weeks post-transfer. At the indicated time-points post-transfer, blood was collected from mice treated with REGN7257 or isotype control, and the proportions (top row, as a percentage of total T cells) or counts (bottom row, as #cells/μL blood) of donor T cells were quantified by flow cytometry. Data are represented as mean+/− SEM. Multiple T Tests were run per week of blood collection between REGN1945-treated group (n=5 mice) and REGN7257-treated group (n=6 mice) using the two-stage step-up (Benjamini, Krieger, and Yekutieli) multiple comparison test with False Discovery Rate (FDR) set at 1%. Statistical significance was recorded for adjusted p values as such: *<0.05, **<0.005, ***<0.001, ****<0.0001.

FIGS. 3A-3C show that host T cell suppression with IL2RG blockade enhances MC38-OVA tumor infiltration of antigen-specific, REGN7257-resistant T cells in Il2rg^{hu/hu} mice. FIG. 3A shows a schematic of the experimental protocol. REGN7257 or REGN1945 isotype control was injected by subcutaneous injection two times per week at 25 mg/kg starting at Day-25 (indicated by closed arrows). MC38 tumor cells expressing the ovalbumin (OVA) antigen were implanted at Day 0 (open arrow), 0.1-1×10⁶ donor CD8+ T cells from OT-I mice were injected at Day 3 (open arrow), and bone marrow, spleen, blood, tumor-draining lymph node, and tumor samples were taken at Day 24 (open arrow). FIGS. 3B-3C show that donor OT-I CD8+ T cells dominate within tumor-infiltrating T cells in REGN7257-conditioned 112rg^{hu/hu} mice. Three weeks after implantation, tumor-infiltrating leukocyte populations were quantified by flow cytometry. FIG. 3B shows representative plots quantifying total CD4+ and CD8+ T cells (left column) and OVA-specific T cells identified by pMHC tetramer staining (right column) in mice receiving indicated treatments. FIG. 3C shows proportions (top row) and counts (bottom row, #cells per mg tumor) of the indicated cell populations for all mice in the study. Data are represented as mean+/− SEM. Ordinary one-way ANOVA was used to compare the following groups: REGN1945-treated, no OT-I transfer (n=5 mice), REGN1945-treated, 1E6 OT-I cells transferred (n=3 mice), REGN7257-treated, no OT-I transfer (n=6 mice), REGN7257-treated, 1E5 OT-I cells transferred (n=3 mice), and REGN7257-treated, 1E6 OT-I cells transferred (n=6 mice). Tukey's multiple comparison test was performed, and statistical significance was recorded for adjusted p values as such: *<0.05, **<0.005, ***<0.001, <0.0001.

FIGS. 4A-4C show that REGN7257 conditioning dramatically enhances “ARMoR′d” T cell infiltration in lymph nodes draining B16.F10.9-OVA tumors. FIG. 4A shows a schematic of the experimental protocol. REGN7257 or REGN1945 isotype control was injected by subcutaneous injection two times per week at 25 mg/kg starting at Day-25 (indicated by closed arrows). B16.F10.9 tumor cells expressing the ovalbumin (OVA) antigen were implanted at Day 0 (open arrow), 1×10⁶ donor CD8+ T cells from OT-I mice were injected at Day 3 (open arrow), and samples were taken at Day 24 (open arrow). FIGS. 4B-4C show that REGN7257-conditioned mice showed an enhanced infiltration of tumor-specific OT-I T cells into the tumor draining lymph node. Three weeks after implantation, leukocyte populations were quantified in tumor-draining lymph nodes (LNs) by flow cytometry. FIG. 4B shows representative plots quantifying total CD4+ and CD8+ T cells (left column) and OVA-specific T cells identified by pMHC staining (right column) in mice receiving indicated treatments. FIG. 4C shows proportions (top row) and counts (bottom row, #cells per LN) of the indicated cell populations for all mice in the study. Data are represented as mean+/− SEM. Ordinary one-way ANOVA was used to compare the following groups: REGN1945-treated, no OT-I transfer (n=7 mice), REGN1945-treated, 1E6 OT-I cells transferred (n=7 mice), REGN7257-treated, no OT-I transfer (n=5 mice), and REGN7257-treated, 1E6 OT-I cells transferred (n=7 mice). Tukey's multiple comparison test was performed, and statistical significance was recorded for adjusted p values as such: *<0.05, **<0.005, ***<0.001, ****<0.0001.

FIGS. 5A-5D show that REGN7257 conditioning dramatically enhances the ability of adoptively transferred “ARMoR′d” T cells to regress established B16.F10.9-OVA tumors concurrent with enhanced tumor infiltration. FIG. 5A shows a schematic of the experimental protocol. REGN7257 or REGN1945 isotype control was injected by subcutaneous injection two times per week at 25 mg/kg starting at Day-25 (indicated by closed arrows). B16.F10.9 tumor cells expressing the ovalbumin (OVA) antigen were implanted at Day 0 (open arrow), 1×10⁶ donor CD8+ T cells from OT-I mice were injected at Days 13, 18, and 24 (open arrows), and samples were later taken for analysis. FIG. 5B shows that REGN7257-conditioned mice receiving OT-I T cell infusions starting 13 days post tumor implantation show enhanced tumor regression, as measured by tumor volume, compared to isotype (REGN1945)-conditioned mice. FIG. 5C shows representative flow cytometry plots (left) and quantified summaries (right) of CD8+ T cells infiltrating tumor-draining lymph nodes (LNs). Infiltration of OVA-specific OT-I T cells, identified by pMHC tetramer staining, was enhanced in REGN7257-conditioned mice. FIG. 5D shows quantification of tumor-infiltrating OVA-specific T cells as a proportion of total live cells from dissected tumors. Infiltration of OVA-specific OT-I T cells, identified by pMHC tetramer staining, was enhanced in REGN7257-conditioned mice. Data are represented as mean+/− SEM. Ordinary one-way ANOVA was used to compare the following groups: REGN1945-treated, no OT-I transfer (n=8 mice), REGN1945-treated, 1E6 OT-I cells transferred (n=8 mice), REGN7257-treated, no OT-I transfer (n=6 mice), and REGN7257-treated, 1E6 OT-I cells transferred (n=7 mice). Tukey's multiple comparison test was performed, and statistical significance was recorded for adjusted p values as such: *<0.05, **<0.005, ***<0.001, <0.0001.

FIGS. 6A-6E show REGN7257 binding region 1 and generation of variants. FIG. 6A shows comparison of human and mouse IL2RG sequences in binding region 1, and generation of mutants. FIG. 6B shows quantification of expression and REGN7257 binding of IL2RG (myc-tagged) variants. Constructs were transfected into 293T cells, and cells were stained with anti-myc (left column) or REGN7257 (right column). FIG. 6C shows REGN 7257 binding to YT cells expressing IL2RG variants. YT cells were transduced with lentiviral vectors encoding the indicated variants and stained with REGN7257 at concentrations indicated on the x-axis. Binding was quantified by flow cytometry (y-axis, gMFI=geometric mean fluorescence intensity). FIGS. 6D-6E show screening of additional substitutions at M145.

FIG. 6D shows anti-myc binding to myc-tagged IL2RG variants with indicated amino acid substitutions at M145, expressed in 293T cells. FIG. 6E shows REGN7257 binding to myc-tagged IL2RG variants with indicated amino acid substitutions at M145, expressed in 293T cells. Binding was quantified by flow cytometry (y-axis, gMFI=geometric mean fluorescence intensity).

FIGS. 7A-7F show that IL2RG variants with mutations in REGN7257 binding region 1 signal efficiently in response to y-chain cytokines and evade signaling blockade by REGN7257. FIG. 7A shows expression of endogenous IL2RG in YT cells engineered with a STAT5-responsive luciferase response (YT.STAT5.luc, top row), compared to YT.STAT5.luc cells in which endogenous IL2RG was knocked out and the indicated IL2RG variants re-introduced by lentiviral transduction (remaining rows). FIG. 7B shows luciferase activity in YT.STAT5.luc cells expressing the indicated IL2RG variants in response to a titration of human IL2 (triangles), or at a constant IL2 concentration (3.3e-11 M) in the presence of REGN7257 (squares) or isotype (circles). FIG. 7C shows luciferase activity in YT.STAT5.luc cells expressing the indicated IL2RG variants in response to a titration of human IL15 (triangles), or at a constant IL15 concentration (3.9e-11 M) in the presence of REGN7257 (squares) or isotype (circles). FIG. 7D shows luciferase activity in YT.STAT5.luc.hIL7Ra cells expressing the indicated IL2RG variants in response to a titration of human IL7 (triangles), or at a constant IL7 concentration (2.9e-11 M) in the presence of REGN7257 (squares) or isotype (circles). FIG. 7E shows luciferase activity in YT.STAT5.luc cells expressing the indicated IL2RG variants in response to a titration of human IL21 (triangles), or at a constant IL21 concentration (3.2e-11 M) in the presence of REGN7257 (squares) or isotype (circles). FIG. 7F shows luciferase activity in Ramos.STAT3.luc cells expressing the indicated IL2RG variants in response to a titration of human IL4 (triangles), or at a constant IL4 concentration (3.6e-11 M) in the presence of REGN7257 (squares) or isotype (circles).

FIG. 8 shows that REGN7257 suppresses the growth of primary human T cells in vitro. T cells isolated from peripheral blood were activated and grown in the presence of REGN7257 (squares) or isotype (circles) at low (20 U/mL, left plot) or high (100 U/mL) levels of human IL2.

FIGS. 9A-B show that shows IL2RG variants with mutations in REGN7257 binding region 1 support IL2-dependent growth of primary T cells and confer resistance to REGN7257 growth suppression. T cells isolated from human peripheral blood were activated and transduced with lentiviral vectors co-expressing IL2RG variants and green fluorescent protein (GFP). Cells were then cultured in the presence of human IL2 along with REGN7257 or isotype, and the cells expressing the indicated IL2RG variants were quantified over time based on GFP expression. FIG. 9A shows that REGN7257 treatment (bottom plots) increased the proportion of GFP+ cells expressing REGN7257-resistant variants but not REGN7257-susceptible variants. Treatment with isotype (top plots) did not alter the proportion of GFP+ cells in any cases. Therefore, cells expressing IL2RG variants resistant to REGN7257 evaded growth suppression and had growth advantage compared with non-transduced T cells. FIG. 9B shows comparisons of IL2-dependent growth kinetics of primary T cells expressing transduced IL2RG variants (GFP+, squares) compared to non-transduced cells expressing only endogenous IL2RG (GFP-, circles). In the presence of REGN7257, GFP+ cells expressing REGN7257-resistant variants (bottom row) but not REGN7257-susceptible variants (top row) showed similar expansion as non-transduced cells. Therefore, cells expressing IL2RG variants with mutations in REGN7257 binding region 1 that disrupt REGN7257 binding evade growth suppression and support normal IL2-dependent growth of human T cells.

FIGS. 10A-10C show screening and optimization of sgRNAs targeting REGN7257 binding region 1 encoded within exon 3 of IL2RG in human T cells. FIG. 10A shows a schematic for editing exon 3 of the human IL2RG gene locus. FIG. 10B shows the editing rate (indel formation) of 12 sgRNAs targeting exon 3. Three sgRNAs (sgRNAs 2, 8, and 11) showed the highest indel rates and were each tested further in the presence of ssODN donor templates. FIG. 10C shows the editing rate (introduction of M145K mutation) when sgRNAs 2, 8, and 11 were co-transfected with 10 different ssODN designs (D1-D10) each, with various length, symmetry, and sense/antisense orientation with respect to sgRNAs cut sites.

FIGS. 11A-11C show expression and REGN7257 binding to IL2RG variants with mutations in REGN7257 binding region 2. FIG. 11A shows comparison of human and mouse IL2RG sequences in binding region 2 (top) and expression and REGN7257 binding of IL2RG (myc-tagged) W90 variants measured by flow cytometry (bottom). Constructs were transfected into 293T cells, and cells were stained with anti-myc (left column) or REGN7257 (right column). FIG. 11B shows quantification of expression of transfected myc-tagged IL2RG variants by flow cytometry (gMFI=geometric mean fluorescence intensity). FIG. 11C shows quantification of REGN7257 binding to transfected IL2RG variants (gMFI=geometric mean fluorescence intensity).

FIGS. 12A-12E show that IL2RG variants with mutations in REGN7257 binding region 2 signal efficiently in response to y-chain cytokines and evade signaling blockade by REGN7257. FIG. 12A shows luciferase activity in YT.STAT5.luc cells expressing the indicated IL2RG variants in response to a titration of human IL2 (triangles), or at a constant IL2 concentration (3.3e-11 M) in the presence of REGN7257 (squares) or isotype (circles). FIG. 12B shows luciferase activity in YT.STAT5.luc cells expressing the indicated IL2RG variants in response to a titration of human IL15 (triangles), or at a constant IL15 concentration (3.9e-11 M) in the presence of REGN7257 (squares) or isotype (circles). FIG. 12C shows luciferase activity in YT.STAT5.luc.hIL7Ra cells expressing the indicated IL2RG variants in response to a titration of human IL7 (triangles), or at a constant IL7 concentration (2.9e-11 M) in the presence of REGN7257 (squares) or isotype (circles). FIG. 12D shows luciferase activity in YT.STAT5.luc cells expressing the indicated IL2RG variants in response to a titration of human IL21 (triangles), or at a constant IL21 concentration (3.2e-11 M) in the presence of REGN7257 (squares) or isotype (circles). FIG. 12E shows luciferase activity in Ramos.STAT3.luc cells expressing the indicated IL2RG variants in response to a titration of human IL4 (triangles), or at a constant IL4 concentration (3.6e-11 M) in the presence of REGN7257 (squares) or isotype (circles).

FIG. 13 shows that IL2RG variants with mutations in REGN7257 binding region 2 support IL2-dependent growth of primary T cells and confer resistance to REGN7257 growth suppression. T cells isolated from human peripheral blood were activated and transduced with lentiviral vectors co-expressing IL2RG variants and green fluorescent protein (GFP). Cells were then cultured in the presence of human IL2 along with REGN7257 or isotype, and the cells expressing the indicated IL2RG variants were quantified over time based on GFP expression. Kinetic growth curves of primary T cells expressing transduced IL2RG variants (GFP+, squares) compared to non-transduced cells expressing only endogenous IL2RG (GFP-, circles). In the presence of REGN7257, GFP+ cells expressing REGN7257-resistant variants (bottom row) but not REGN7257-susceptible variants (top row) showed similar expansion as non-transduced cells. Therefore, cells expressing IL2RG variants with mutations in REGN7257 binding region 2 that disrupt REGN7257 binding evade growth suppression and support normal IL2-dependent growth of human T cells.

FIGS. 14A-14C show screening and optimization of sgRNAs targeting REGN7257 binding region 2 encoded within exon 2 of IL2RG in human T cells. FIG. 14A shows a schematic for editing exon 2 of the human IL2RG gene locus. FIG. 14B shows the editing rate (indel formation) of 18 sgRNAs targeting exon 2. Two sgRNAs that target near the codon encoding W90 (sgRNAs 14 and 15) and showed high indel rates were each tested further in the presence of ssODN donor templates. FIG. 14C shows the editing rate (introduction of W90Q mutation) when sgRNAs 14 and 15 were co-transfected with 10 different ssODN designs (D1-D10) each, with various length, symmetry, and sense/antisense orientation with respect to sgRNAs cut sites.

FIGS. 15A-15D show generation of INK^{ARMoR} with targeted editing of the endogenous IL2RG locus to introduce resistance to REGN725. FIG. 15A shows the schematic design of generation of INK^{ARMoR} through knock-in of ARMoR (IL2RG M145K) in induced pluripotent stem cells (iPSCs) and differentiating iPSC^{ARMoR} to iPSC-derived NK (INK) cells.

FIG. 15B shows iPSC^{ARMoR} have similar NK differentiation potential as iPSC^{WT}. iPSC^{WT} and iPSC^{ARMoR} cells were differentiated to iNK cells in vitro, and the resulting proportion of CD45+CD56+ iNK cells was determined by flow cytometry. FIG. 15C shows that iNK^{ARMoR} cells have reduced REGN7257 binding compared to iNKWT cells, measured by flow cytometry. FIG. 15D shows that iNK ARMoR and iNKWT cells bind equivalently to a commercial anti-IL2RG that recognizes a distant epitope from REGN7257.

FIG. 16 shows iNKAR.MOR cells escape suppression of IL2 and IL15-dependent growth by REGN7257. Differentiated iNK ARMoR and iNKWT cells were grown with IL2 (left graphs) or IL15 (right graphs) in the presence of REGN7257, isotype (REGN1945), or no antibody. Cells counts were determined at the indicated time points and are reported normalized to cell counts at day 0 (DO).

FIGS. 17A-17B show human iNK^{ARMoR} cells engrafted in immunodeficient mice are resistant to suppression by REGN7257. FIG. 17A shows a schematic of the experimental protocol. FIG. 17B shows that iNK^{ARMoR} have a competitive advantage against iNKWT in vivo in the presence of REGN7257. INK ARMoR and iNKWT cells were administered to at a 1:1 ratio to irradiated host mice treated with REGN7257 or isotype control. At the indicated time points post-transfer, iNK cells (as a proportion of blood leukocytes) were quantified in peripheral blood by flow cytometry. Student t tests were performed, and statistical significance was recorded for p values as such: **<0.01.

### DEFINITIONS

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

Proteins are said to have an “N-terminus” (amino-terminus) and a “C-terminus” (carboxy-terminus or carboxyl-terminus). The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “expression vector” or “expression construct” or “expression cassette” refers to a recombinant nucleic acid containing a desired coding sequence operably linked to appropriate nucleic acid sequences necessary for the expression of the operably linked coding sequence in a particular host cell or organism. Nucleic acid sequences necessary for expression in prokaryotes usually include a promoter, an operator (optional), and a ribosome binding site, as well as other sequences. Eukaryotic cells are generally known to utilize promoters, enhancers, and termination and polyadenylation signals, although some elements may be deleted and other elements added without sacrificing the necessary expression.

A “promoter” is a regulatory region of DNA usually comprising a TATA box capable of directing RNA polymerase II to initiate RNA synthesis at the appropriate transcription initiation site for a particular polynucleotide sequence. In general, a “promoter” or “promoter sequence” is a DNA regulatory region capable of binding an RNA polymerase in a cell (e.g., directly or through other promoter-bound proteins or substances) and initiating transcription of a coding sequence. A promoter may be operably linked to other expression control sequences, including enhancer and repressor sequences and/or with a polynucleotide of the invention. Promoters which may be used to control gene expression include, but are not limited to, cytomegalovirus (CMV) promoter (U.S. Pat. Nos. 5,385,839 and 5,168,062, each of which is herein incorporated by reference in its entirety for all purposes), the SV40 early promoter region (Benoist et al. (1981) Nature 290:304-310, herein incorporated by reference in its entirety for all purposes), the promoter contained in the 3′ long terminal repeat of Rous sarcoma virus (Yamamoto et al. (1980) Cell 22:787-797, herein incorporated by reference in its entirety for all purposes), the herpes thymidine kinase promoter (Wagner et al. (1981) Proc. Natl. Acad. Sci. U.S.A. 78:1441-1445, herein incorporated by reference in its entirety for all purposes), the regulatory sequences of the metallothionein gene (Brinster et al. (1982) Nature 296:39-42, herein incorporated by reference in its entirety for all purposes); prokaryotic expression vectors such as the beta-lactamase promoter (VIlla-Komaroff et al. (1978) Proc. Natl. Acad. Sci. U.S.A. 75:3727-3731, herein incorporated by reference in its entirety for all purposes), or the tac promoter (DeBoer et al. (1983) Proc. Natl. Acad. Sci. U.S.A. 80:21-25; see also “Useful proteins from recombinant bacteria” in Scientific American (1980) 242:74-94, each of which is herein incorporated by reference in its entirety for all purposes); and promoter elements from yeast or other fungi such as the Gal4 promoter, the ADC (alcohol dehydrogenase) promoter, PGK (phosphoglycerol kinase) promoter or the alkaline phosphatase promoter.

In some embodiments, a promoter may additionally comprise other regions which influence the transcription initiation rate. The promoter sequences modulate transcription of an operably linked polynucleotide. A promoter can be active in one or more of the cell types (e.g., but not limited to, a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., but not limited to, a developmentally regulated promoter), or a spatially restricted promoter (e.g., but not limited to, a cell-specific or tissue-specific promoter).

“Operable linkage” or being “operably linked” includes juxtaposition of two or more components (e.g., but not limited to, a promoter and another sequence element) such that both components function normally and allow the possibility that at least one of the components can mediate a function that is exerted upon at least one of the other components. As a non-limiting example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., but not limited to, a regulatory sequence can act at a distance to control transcription of the coding sequence). A polynucleotide encoding a polypeptide is “operably linked” to a promoter or other expression control sequence when, in a cell or other expression system, the sequence directs RNA polymerase mediated transcription of the coding sequence into RNA, preferably mRNA, which then may be RNA spliced (if it contains introns) and, optionally, translated into a protein encoded by the coding sequence.

The term “isolated” with respect to proteins, nucleic acids, and cells includes proteins, nucleic acids, and cells that are relatively purified with respect to other cellular or organism components that may normally be present in situ, up to and including a substantially pure preparation of the protein, nucleic acid, or cell. In some embodiments, the term “isolated” may include proteins and nucleic acids that have no naturally occurring counterpart or proteins or nucleic acids that have been chemically synthesized and are thus substantially uncontaminated by other proteins or nucleic acids. The term “isolated” may include proteins, nucleic acids, or cells that have been separated or purified from most other cellular components or organism components with which they are naturally accompanied (e.g., but not limited to, other cellular proteins, nucleic acids, or cellular or extracellular components). “Isolated” antigen-binding proteins (e.g., antibodies or antigen-binding fragments thereof), polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. Generally, the term “isolated” is not intended to refer to a complete absence of such biological molecules (e.g., minor or insignificant amounts of impurity may remain) or to an absence of water, buffers, or salts or to components of a pharmaceutical formulation that includes the antigen-binding proteins (e.g., antibodies or antigen-binding fragments).

“Codon optimization” takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. As a non-limiting example, a nucleic acid encoding a protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Res. 28 (1): 292, herein incorporated by reference in its entirety for all purposes. Computer algorithms for codon optimization of a particular sequence for expression in a particular host are also available (see, e.g., Gene Forge).

The term “locus” refers to a specific location of a gene (or significant sequence), DNA sequence, polypeptide-encoding sequence, or position on a chromosome of the genome of an organism. As a non-limiting example, an “IL2RG locus” may refer to the specific location of an IL2RG gene, IL2RG DNA sequence, IL2RG-protein-encoding sequence, or IL2RG position on a chromosome of the genome of an organism that has been identified as to where such a sequence resides. An “IL2RG locus” may comprise a regulatory element of an IL2RG gene, including, as a non-limiting example, an enhancer, a promoter, 5′ and/or 3′ untranslated region (UTR), or a combination thereof.

The term “gene” refers to DNA sequences in a chromosome that may contain, if naturally present, at least one coding and at least one non-coding region. The DNA sequence in a chromosome that codes for a product (e.g., but not limited to, an RNA product and/or a polypeptide product) can include the coding region interrupted with non-coding introns and sequence located adjacent to the coding region on both the 5′ and 3′ ends such that the gene corresponds to the full-length mRNA (including the 5′ and 3′ untranslated sequences). Additionally, other non-coding sequences including regulatory sequences (e.g., but not limited to, promoters, enhancers, and transcription factor binding sites), polyadenylation signals, internal ribosome entry sites, silencers, insulating sequence, and matrix attachment regions may be present in a gene. These sequences may be close to the coding region of the gene (e.g., but not limited to, within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

The term “wild type” includes entities having a structure (e.g., but not limited to, nucleotide sequence or amino acid sequence sequence) as found in a normal (as contrasted with mutant, diseased, altered, or so forth) state or context. Wild type genes and polypeptides often exist in multiple different forms (e.g., alleles).

The term “variant” refers to a nucleotide sequence differing from the sequence most prevalent in a population (e.g., but not limited to, by one nucleotide) or a protein sequence different from the sequence most prevalent in a population (e.g., but not limited to, by one amino acid).

The term “fragment,” when referring to a protein, means a protein that is shorter or has fewer amino acids than the full-length protein. The term “fragment,” when referring to a nucleic acid, means a nucleic acid that is shorter or has fewer nucleotides than the full-length nucleic acid. Non-limiting examples of a protein fragment can include an N-terminal fragment (i.e., removal of a portion of the C-terminal end of the protein), a C-terminal fragment (i.e., removal of a portion of the N-terminal end of the protein), or an internal fragment (i.e., removal of a portion of an internal portion of the protein).

“Sequence identity” or “identity” in the context of two polynucleotides or polypeptide sequences refers to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., but not limited to, charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, as a non-limiting example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “in vitro” includes artificial environments and processes or reactions that occur within an artificial environment (e.g., but not limited to, a test tube or an isolated cell or cell line). The term “in vivo” includes natural environments (e.g., but not limited to, an organism or body or a cell or tissue within an organism or body) and to processes or reactions that occur within a natural environment. The term “ex vivo” includes cells that have been removed from the body of an individual and processes or reactions that occur within such cells.

“Optional” or “optionally” means that the subsequently described event or circumstance may or may not occur and that the description includes instances in which the event or circumstance occurs and instances in which the event or circumstance does not.

Designation of a range of values includes all integers within or defining the range, and all subranges defined by integers within the range.

Unless otherwise apparent from the context, the term “about” encompasses values ±5 of a stated value.

The term “and/or” refers to and encompasses any and all possible combinations of one or more of the associated listed items, as well as the lack of combinations when interpreted in the alternative (“or”).

The term “or” refers to any one member of a particular list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

### DETAILED DESCRIPTION

### I. Overview

Immune cell therapies hold enormous promise for many human diseases. One of the oldest examples is bone marrow transplantation, in which a recipient's entire immune system can be replaced with an autologous or allogeneic bone marrow graft. This procedure allows the correction of congenital hematopoietic deficiencies, and also the re-population of the immune system following treatments to eradicate hematologic malignancies. Newer examples include immune cells engineered with antigen receptors to target tumors (e.g., CAR-T, eTCR, CAR-NK, and CAR-macrophage). In all these cases, patients must undergo “conditioning” regimens prior to cell transplant, which can serve to “make room” in the host immune niche to support donor cell uptake, and in some cases to suppress host versus graft immune responses that can lead to graft rejection.

Conditioning regimens range in intensity from partially to fully myeloablative, the latter necessary when pathogenic host immune cells must be fully eradicated (e.g., for hematologic malignances). Regardless, the current standards of care for host conditioning have major drawbacks. First, conditioning agents are toxins (e.g., DNA damagers) that are not specific for the desired target cells, and thus carry harmful and even life-threatening risks for patients. Moreover, conditioning agents are as toxic to donor cells as to host, and so must be discontinued prior to transplant to avoid inhibition of life-saving cellular therapies. These challenges usually limit the application of cell therapies to dire instances when no treatment alternatives remain.

Lymphosuppressive agents have numerous potential applications to host conditioning for transplant and adoptive cell therapies: (1) preventing rejection of allogeneic grafts through T and NK cell suppression (e.g., bone marrow transplant; gene corrective cell therapies); (2) non-genotoxic clearance of immune niche space for engineered cell therapies (e.g., CAR-T, TCR-T, Treg, NK, B cell, progenitor cells); (3) elimination of endogenous cytokine “sinks,” making essential factors more available to grafter cells; and (4) immune suppression post-transplant that is gentler and less toxic than standard of care agents.

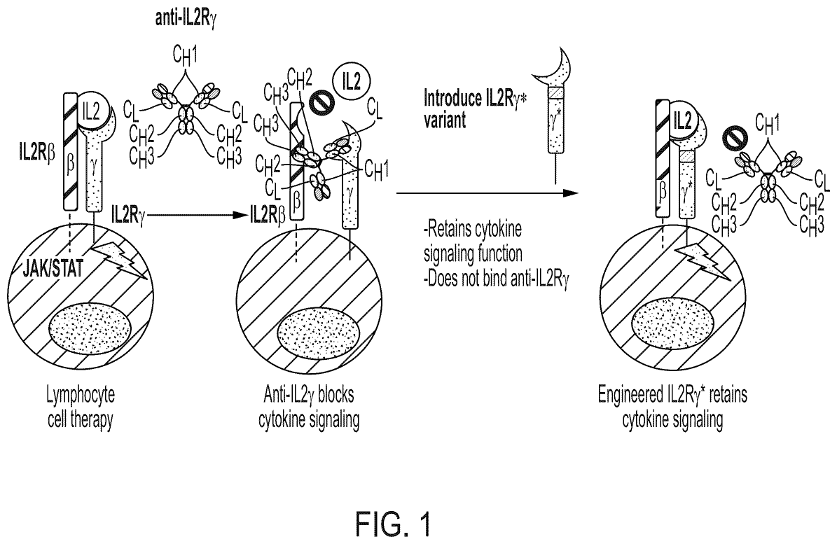

An obstacle to use of lymphosuppressive agents as conditioning therapies is the susceptibility of grafted cells, in addition to the target host cells, to their effects. Provided herein is a strategy to address these challenges through (1) the development of targeted conditioning regimens leveraging, e.g., antibodies that specifically target the desired host cells—as monotherapies, combinations, bispecific antibodies, antibody drug conjugates (ADCs), or scFv-engineered CAR-T, and (2) modification of donor cells to render them resistant to, e.g., these antibody-based conditioning agents. Collectively this comprises the antibody-resistant modified receptor (ARMoR) concept. The underlying idea is to engineer minimal changes to immune cell receptors in grafted donor cells that will abolish binding by suppressive antibody agents. A schematic of this strategy is shown in FIG. 1, using the non-limiting example of an engineered interleukin 2 receptor subunit gamma (IL2RG) variant that is resistant to anti-IL2RG mediated blockade of cytokine signals essential for lymphocyte function. The goal is to introduce into a cell therapy product a fully functional form of the receptor that is not recognized by the conditioning agent. This can be achieved with alterations to the antibody recognition site that abolish binding but preserve receptor function. As a result, host cells remain susceptible to the conditioning agent, but engineered grafted cells are resistant and thus gain a competitive advantage in re-populating the host. The fundamental aim is to afford grafted cellular therapies a competitive advantage in the host patient, by applying selective pressure that specifically targets host cells while sparing donor-derived cell therapies.

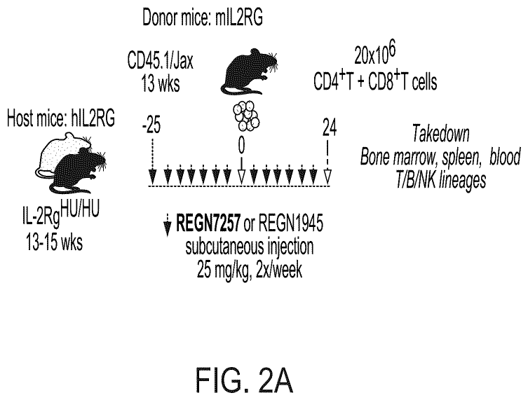

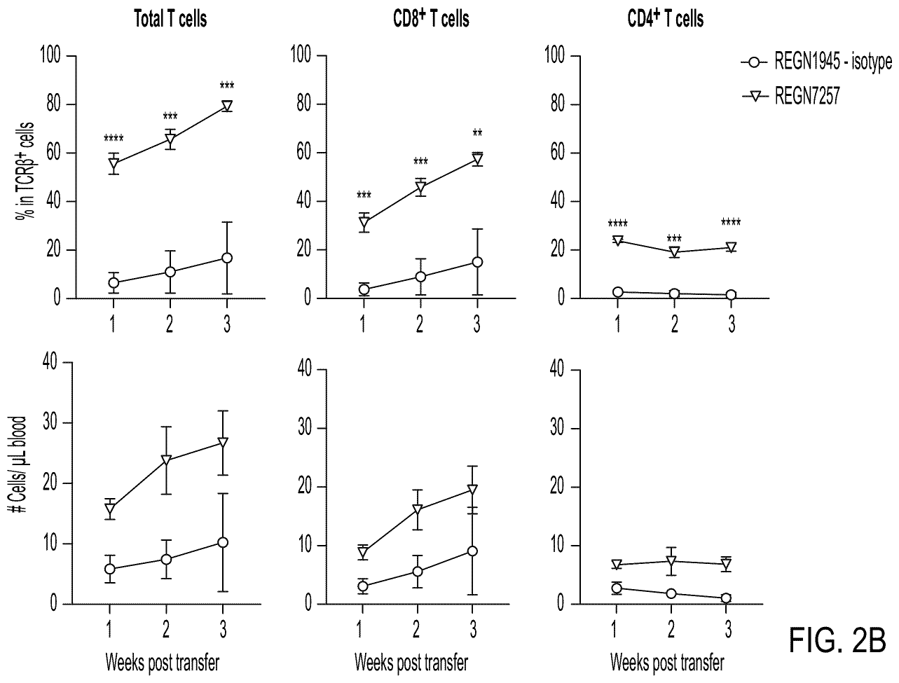

Methods for improving engraftment of donor cells in a subject thereof are provided. Such methods can comprise providing donor cells that express (e.g., that have been modified to express) a first isoform of a target protein (e.g., interleukin-2 receptor subunit gamma (IL2RG)), administering the donor cells to the subject, and then selectively depleting host cells in the subject based on their expression of a second isoform of the target protein, thereby improving engraftment of donor cells in the subject. For example, such methods can comprise providing donor cells that express (e.g., that have been modified to express) a first isoform of a target protein (e.g., interleukin-2 receptor subunit gamma (IL2RG)), administering the donor cells to the subject, and then selectively inhibiting host cells in the subject based on their expression of a second isoform of the target protein, thereby improving engraftment of donor cells in the subject. Alternatively, such methods can comprise providing donor cells that express (e.g., that have been modified to express) a first isoform of a target protein (e.g., interleukin-2 receptor subunit gamma (IL2RG)), administering the donor cells to the subject, and then selectively ablating host cells in the subject based on their expression of a second isoform of the target protein, thereby improving engraftment of donor cells in the subject. The donor cells can express only the first isoform, or they can express both the first and second isoforms of the target protein. The first isoform can be functionally indistinguishable but immunologically distinguishable from the second isoform of the target protein. In some embodiments, the target protein is a protein that is expressed on the cell surface of hematopoietic cells, such as lymphocytes. In some embodiments, the target protein is a receptor, such as a cytokine receptor or a chemokine receptor (e.g., in some embodiments, the receptor is a cytokine receptor). In some embodiments, the target protein is a cytokine receptor sub-unit of an interleukin-2 (IL-2) receptor, an IL-4 receptor, an IL-7 receptor, an IL-9 receptor, an IL-15 receptor, or an IL-21 receptor. For example, the target protein can be IL2RG. In some embodiments, the selective inhibition of host cells can be based on their expression of only the second isoform of the target protein and their lack of expression of the first isoform of the target protein. Alternatively, the selective inhibition of host cells can be based on their expression of the second isoform regardless of their expression of the first isoform of the target protein. Selective inhibition of host cells does not comprise ablation (i.e., killing) of host cells by a mechanism extrinsic to a cell, such as via an active killing mechanism. Selective inhibition of host cells does not comprise ablation (i.e., killing) of host cells by an active killing mechanism. An active killing mechanism means the agent directly kills the host cells by cytotoxic mechanisms (e.g., antibody-drug conjugate (ADC), antibody radioconjugate (ARC), CAR-T, or other engineered cytotoxicity) or recruits host cytotoxic effector mechanisms (e.g., complement-dependent cytotoxicity (CDC), antibody-dependent cellular cytotoxicity (ADCC), antibody-dependent cellular phagocytosis (ADCP)), as opposed blocking a cellular function (e.g. growth or cytokine signaling, chemotactic tissue homing, cell-cell adhesion; e.g., as with selective inhibition) without engaging extrinsic cytotoxic effectors. Such host cytotoxic effector mechanisms are well known. See, e.g., Yu et al. (2020) J. Hematol. Oncol. 13 (1): 45 and Gogesch et al. (2021) Int. J. Mol. Sci. 22 (16): 8947, each of which is herein incorporated by reference in its entirety for all purposes. As a novel conditioning strategy, selective inhibition of host cells without cytotoxic ablation has potential to improve the safety and efficacy of cell therapy and transplant treatments. Non-ablative conditioning may avoid undesired and harmful effects of ablative agents, including direct killing of non-target (e.g., non-hematopoietic) cells expressing drug target antigens, indirect toxicities to target-adjacent tissues, and prolonged immune suppression in the post-transplant period. Selective blockade or suppression of essential host cell factors can enhance the expansion, persistence, and trafficking of resistant donor cells by affording favorable competition for limiting host factors (e.g., cytokine, chemokines) and immune niche space, without harsh and potentially toxic ablative agents. See, e.g., FIGS. 2A-5D. In some embodiments, the selective inhibition of host cells can comprise: (1) blocking growth of the host cells to provide a competitive growth advantage to the donor cells; (2) blocking localization or trafficking of the host cells to provide a competitive homing advantage to the donor cells; (3) blocking a cell-cell interaction or adhesion of the host cells to provide a competitive tissue infiltration advantage to the donor cells; or (4) blocking immune cell activation in the host cells to provide a competitive advantage to the donor cells. For example, in some embodiments, the selective inhibition of host cells comprises blocking growth (i.e., proliferation) and/or blocking immune cell activation. For example, in some embodiments, the selective inhibition of host cells comprises blocking growth (i.e., proliferation) and blocking immune cell activation. For example, in some embodiments, the selective inhibition of host cells comprises blocking growth (i.e., proliferation). For example, in some embodiments, the selective inhibition comprises blocking immune cell activation. Also provided are combinations for administration to a subject in need thereof, wherein the combination comprises (1) a population of donor cells that express (e.g., that have been modified to express) a first isoform of a target protein (e.g., IL2RG) and (2) an agent (e.g., antagonist, such as an antigen-binding protein) that specifically binds to the second isoform of the target protein but does not specifically bind to the first isoform of the target protein.

Methods for in vivo selective depletion of non-edited cells in a subject thereof are provided. Methods for in vivo selective depletion of non-edited cells and repopulation with edited cells in a subject thereof are provided. Such methods can comprise providing cells edited to express a first isoform of a target protein (e.g., interleukin-2 receptor subunit gamma (IL2RG)), administering the edited cells to the subject, and then selectively depleting non-edited cells in the subject based on their expression of a second isoform of the target protein. Such methods can comprise providing cells edited to express a first isoform of a target protein (e.g., interleukin-2 receptor subunit gamma (IL2RG)) that is functionally indistinguishable but immunologically distinguishable from a second isoform of the target protein, administering the edited cells to the subject, and then selectively depleting non-edited cells in the subject based on their expression of the second isoform of the target protein. For example, the selective depletion of non-edited cells can be based on their expression of only the second isoform of the target protein and their lack of expression of the first isoform of the target protein. Alternatively, they can be depleted based on their expression of the second isoform regardless of their expression of the first isoform of the target protein. The first isoform can be functionally indistinguishable but immunologically distinguishable from a second isoform of the target protein. The edited cells can express only the first isoform, or they can express both the first and second isoforms of the target protein. Also provided are combinations for administration to a subject in need thereof, wherein the combination comprises (1) a population of cells edited to express a first isoform of a target protein (e.g., IL2RG) and (2) an agent (e.g., antagonist, such as an anti-IL2RG antigen-binding protein) that specifically binds to a second isoform of the target protein but does not specifically bind to the first isoform of the target protein.

Isolated cells or populations of cells are also provided modified to express a first isoform of a target protein that is different from a second isoform of the target protein. The cells can express only the first isoform, or they can express both the first and second isoforms. In some embodiments, the target protein is a protein that is expressed on the cell surface of hematopoietic cells, such as lymphocytes. In some embodiments, the target protein is a receptor, such as a cytokine receptor or a chemokine receptor (e.g., in some embodiments, the receptor is a cytokine receptor). In some embodiments, the target protein is a cytokine receptor sub-unit of an interleukin-2 (IL-2) receptor, an IL-4 receptor, an IL-7 receptor, an IL-9 receptor, an IL-15 receptor, or an IL-21 receptor. For example, the target protein can be IL2RG. The first isoform can be functionally indistinguishable but immunologically distinguishable from the second isoform. The cells can express only the first isoform, or they can express both the first and second isoforms. Isolated cells or populations of cells are also provided in which a genomic locus has been edited to express the first isoform of the target protein that is different from the second isoform. Methods of making such cells are also provided.

Isolated cells or populations of cells are also provided that are edited (i.e., modified) to express a first isoform of IL2RG that is different from a second isoform of IL2RG. The first isoform can be functionally indistinguishable but immunologically distinguishable from a second isoform of the IL2RG. The edited cells can express only the first isoform, or they can express both the first and second isoforms of the IL2RG. Isolated cells or populations of cells are also provided in which a genomic locus has been edited to express a first isoform of IL2RG that is different from a second isoform of IL2RG. The first isoform can be functionally indistinguishable but immunologically distinguishable from a second isoform of the IL2RG. The edited cells can express only the first isoform, or they can express both the first and second isoforms of the IL2RG. Isolated cells or populations of cells are also provided in which an IL2RG genomic locus has been edited to express a first isoform of IL2RG that is different from a second isoform of IL2RG, wherein the first isoform and the second isoform are functionally indistinguishable but immunologically distinguishable. Methods of making such cells are also provided, and engineered IL2RG proteins and nucleic acids encoding engineered IL2RG proteins are also provided.

In some embodiments, the cells (e.g., donor cells or edited cells) in the compositions and methods comprise or express a therapeutic molecule, such as a therapeutic protein or enzyme, an immunoglobulin (e.g., antibody or antigen-binding fragment thereof), a chimeric antigen receptor (CAR) (e.g., CAR-T cells, CAR-NK cells), or an exogenous T cell receptor (TCR). In some embodiments, the therapeutic molecule, immunoglobulin, CAR, or exogenous TCR does not target the target protein (e.g., IL2RG). For example, the donor cells or edited cells can be engineered to express a therapeutic molecule with therapeutic activity against any disease, such as any type of cancer (e.g., not dependent on whether the target protein is related to the disease or cancer), including disease or cancers that are unrelated to the target protein (e.g., the target protein discussed above is not what is being targeted to treat the disease or cancer, but the compositions and methods disclosed herein can provide a competitive advantage to the cells comprising or expressing the therapeutic molecule). For example, the disease or cancer can be a disease or cancer that is not associated with the target protein (e.g., the target protein does not cause the disease or cancer, and/or expression of the target protein is not correlated with the disease or cancer). In some such embodiments, the therapeutic molecule may target diseased cells and/or an antigen expressed on the diseased cells (e.g., a tumor-associated antigen).

### II. Methods for Improving Engraftment of Donor Cells in a Subject

In some embodiments of the present invention, methods for improving engraftment of donor cells in a subject are provided. Such methods can comprise providing donor cells that express (e.g., that have been modified to express) a first isoform of a target protein that is different from a second isoform of the target protein, wherein the second isoform is expressed in host cells of the subject. The target protein can be, for example, a protein expressed on the cell surface of hematopoietic cells. The first isoform can be functionally indistinguishable but immunologically distinguishable from a second isoform of the target protein. In some embodiments, the donor cells express only the first isoform of the target protein. In other embodiments, the donor cells express both the first and second isoforms of the target protein. Such methods can comprise providing donor cells in which a target genomic locus has been edited to express the first isoform of a target protein. The donor cells can then be administered to the subject, and host cells in the subject can be selectively depleted based on their expression of the second isoform of the target protein, thereby improving engraftment of donor cells in the subject. For example, the selective depletion of host cells can be based on their expression of only the second isoform of the target protein and their lack of expression of the first isoform of the target protein. Alternatively, the selective depletion of host cells can be based on their expression of the second isoform regardless of their expression of the first isoform of the target protein. For example, host cells in the subject can be selectively inhibited based on their expression of the second isoform of the target protein, thereby improving engraftment of donor cells in the subject. For example, the selective inhibition of host cells can be based on their expression of only the second isoform of the target protein and their lack of expression of the first isoform of the target protein. Alternatively, the selective inhibition of host cells can be based on their expression of the second isoform regardless of their expression of the first isoform of the target protein. Alternatively, host cells in the subject can be selectively ablated based on their expression of the second isoform of the target protein, thereby improving engraftment of donor cells in the subject. For example, the selective ablation of host cells can be based on their expression of only the second isoform of the target protein and their lack of expression of the first isoform of the target protein. Alternatively, the selective depletion of host cells can be based on their expression of the second isoform regardless of their expression of the first isoform of the target protein.

In some embodiments of the present invention, methods for selective depletion of non-edited cells in a subject are provided. In some embodiments of the present invention, methods for selective depletion of non-edited cells and repopulation with edited cells in a subject are provided. Such methods can comprise providing edited cells that have been modified to express a first isoform of a target protein that is different from a second isoform of the target protein, wherein the second isoform is expressed in non-edited cells of the subject. The first isoform can be functionally indistinguishable but immunologically distinguishable from a second isoform of the target protein. In some embodiments, the edited cells express only the first isoform of the target protein. In other embodiments, the edited cells express both the first and second isoforms of the target protein. Such methods can comprise providing edited cells in which a target genomic locus has been edited to express a first isoform of a target protein that is different from a second isoform of the target protein, wherein the second isoform is expressed in non-edited cells of the subject. The first isoform can be functionally indistinguishable but immunologically distinguishable from a second isoform of the target protein. In some embodiments, the edited cells express only the first isoform of the target protein. In other embodiments, the edited cells express both the first and second isoforms of the target protein. Such methods can comprise providing edited cells in which a target genomic locus has been edited to express a first isoform of a target protein that is different from a second isoform of the target protein, wherein the first isoform and the second isoform are functionally indistinguishable but immunologically distinguishable, and wherein the second isoform is expressed in non-edited cells of the subject. The edited cells can then be administered to the subject, and non-edited cells in the subject can be selectively depleted based on their expression of the second isoform of the target protein. For example, the selective depletion of non-edited cells can be based on their expression of only the second isoform of the target protein and their lack of expression of the first isoform of the target protein. Alternatively, they can be depleted based on their expression of the second isoform regardless of their expression of the first isoform of the target protein.

The donor cells or edited cells can be any suitable cells. Likewise, the host cells or non-edited cells can be any suitable cells. In some embodiments, the cells are hematopoietic cells. The term hematopoietic cell refers to a cell originated from a hematopoietic stem cell or a hematopoictic progenitor cell and/or originated from an erythroid, lymphoid, or myeloid lineage. In some embodiments, the cells are immune cells. The term immune cell refers to any cell derived from a hematopoietic stem cell that plays a role in the immune response. Immune cells include, without limitation, lymphocytes, such as T cells and B cells, antigen-presenting cells (APC), dendritic cells, monocytes, macrophages, natural killer (NK) cells, mast cells, basophils, eosinophils, or neutrophils, as well as any progenitors of such cells. In some embodiments, the cells are lymphocytes or lymphoid progenitor cells. In some embodiments, the cells are T cells (e.g., CD4+ T cells, CD8+ T cells, memory T cells, regulatory T cells, gamma delta T cells, mucosal-associated invariant T cells (MAIT), tumor infiltrating lymphocytes (TILs), or any combination thereof). In some embodiments, the cells are TILs. In some embodiments, the cells are B cells. In some embodiments, the cells are natural killer (NK) cells. In some embodiments, the cells are innate lymphoid cells. In some embodiments, the cells are dendritic cells. In some embodiments, the cells are hematopoietic stem cells (HSCs) or hematopoietic stem and progenitor cells (HSPCs) or descendants thereof. HSCs are capable of giving rise to both myeloid and lymphoid progenitor cells that further give rise to myeloid cells (e.g., monocytes, macrophages, neutrophils, basophils, dendritic cells, erythrocytes, platelets, etc.) and lymphoid cells (e.g., T cells, B cells, NK cells), respectively. In some embodiments, the cells are derived from induced pluripotent stem cells (e.g., NK cells derived from induced pluripotent stem cells). In some embodiments, the cells are derived from HSCs or HSPCs.

In some embodiments, the cells (e.g., donor cells or edited cells) comprise a genetic modification (insertion of a transgene, correction of a mutation, deletion or inactivation of a gene (e.g., insertion of premature stop codon or insertion of regulatory repressor sequence), or a change in an epigenetic modification important for expression of a gene) correcting or counteracting a disease-related gene defect present in a subject. In some embodiments, the cells (e.g., donor cells or edited cells) comprise a transgene. In some embodiments, the cells (e.g., donor cells or edited cells) comprise or express a therapeutic molecule, such as a therapeutic protein or enzyme, an immunoglobulin (e.g., antibody or antigen-binding fragment thereof), a chimeric antigen receptor (CAR) (e.g., CAR-T cells, CAR-NK cells), or an exogenous T cell receptor (TCR). In some embodiments, the cells (e.g., donor cells or edited cells) comprise a bicistronic nucleic acid construct encoding the therapeutic molecule and the first isoform of the target protein. See, e.g., Yeku et al. (2017) Sci. Rep. 7 (1): 10541 and Rafiq et al. (2018) Nat. Biotechnol. 36 (9): 847-856, each of which is herein incorporated by reference in its entirety for all purposes, for examples of bicistronic constructs for expressing CARs and another molecule. For example, the bicistronic construct can encode both a therapeutic protein (e.g., a CAR) and the first isoform (e.g., a modified isoform) of the target protein (e.g., IL2RG). In one embodiment, the bicistronic construct encodes a therapeutic protein (e.g., a CAR) and a modified isoform of IL2RG. In some embodiments, the therapeutic molecule, immunoglobulin, CAR, or exogenous TCR does not target the target protein (e.g., IL2RG). In some embodiments, the cells (e.g., donor cells or edited cells) comprise or express an immunoglobulin, a CAR, or an exogenous TCR. In some embodiments, the cells (e.g., donor cells or edited cells) comprise a CAR or an exogenous TCR. For example, the donor cells or edited cells can be engineered to express a therapeutic molecule with therapeutic activity against any disease, such as any type of cancer (e.g., not dependent on whether the target protein is related to the disease or cancer), including disease or cancers that are unrelated to the target protein (e.g., the target protein discussed above is not what is being targeted to treat the disease or cancer, but the compositions and methods disclosed herein can provide a competitive advantage to the cells comprising or expressing the therapeutic molecule). In some embodiments, the therapeutic molecule targets diseased cells and/or an antigen expressed on the diseased cells (e.g., a tumor-associated antigen). For example, the disease or cancer can be a disease or cancer that is not associated with the target protein (e.g., the target protein does not cause the disease or cancer, and/or expression of the target protein is not correlated with the disease or cancer). Exemplary types of cancers and tumors that can be treated are described elsewhere herein.

In some embodiments, the donor cells are autologous (i.e., from the subject). In some embodiments, the donor cells are allogeneic (i.e., not from the subject) or syngeneic (i.e., genetically identical, or sufficiently identical and immunologically compatible as to allow for transplantation). In some embodiments, the cells are mammalian cells or non-human mammalian cells (e.g., mouse or rat cells or non-human primate cells) (e.g., the subject is a mammal or a non-human mammal, and the donor cells are mammalian cells or non-human mammalian cells). In some embodiments, the cells are human cells (e.g., the subject is a human, and the donor cells are human cells).

Any suitable target protein can be used. In some embodiments, the target protein is a cell surface protein, such as a receptor. For example, the target protein can be a cell surface protein (e.g., a receptor, such as a cytokine receptor or a chemokine receptor) that is expressed on the cell surface of hematopoietic cells, such as lymphocytes. In some embodiments, the cell surface protein is selected from CD1a, CD1b, CD1c, CD1e, CD1e, CD2, CD3, CD3d, CD3c, CD3g, CD4, CD5, CD6, CD7, CD8a, CD8b, CD9, CD10, CD11a, CD11b, CD11c, CD11d, CDw12, CD13, CD14, CD15, CD15u, CD15s, CD15su, CD16, CD16b, CD17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49c, CD49f, CD50, CD51, CD52, CD53, CD54, CD55, CD56, CD57, CD58, CD59, CD60a, CD60b, CD60c, CD61, CD62E, CD62L, CD62P, CD63, CD64, CD65, CD65s, CD66a, CD66b, CD66c, CD66d, CD66c, CD66f, CD68, CD69, CD70, CD71, CD72, CD73, CD74, CD75, CD75s, CD77, CD79a, CD79b, CD80, CD81, CD82, CD83, CD84, CD85a, CD85d, CD85j, CD85k, CD86, CD87, CD88, CD89, CD90, CD91, CD92, CD93, CD94, CD95, CD96, CD97, CD98, CD99, CD99R, CD100, CD10¹, CD102, CD103, CD104, CD105, CD10⁶, CD10⁷a, CD10⁷b, CD108, CD109, CD110, CD111, CD112, CD113, CD114, CD115, CD116, CD117, CD118, CD119, CD120a, CD120b, CD121a, CD121b, CD122, CD123, CD124, CD125, CD126, CD127, CD129, CD130, CD131, CD132, CD133, CD134, CD135, CD136, CD137, CD138, CD139, CD140a, CD140b, CD141, CD142, CD143, CD144, CDw145, CD146, CD147, CD148, CDw149, CD150, CD151, CD152, CD153, CD154, CD155, CD156a, CD156b, CD156c, CD157, CD158c, CD158i, CD158k, CD159a, CD159c, CD160, CD161, CD162, CD163, CD164, CD165, CD166, CD167a, CD167b, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197, CD198/CDw198, CD199/CDw199, CD200, CD201, CD202b, CD203c, CD204, CD205, CD206, CD207, CD208, CD209, CD210/CD210A, CD210B/CDw210b, CD212, CD213a1, CD213a2, CD215, CD217a, CD218a, CD218b, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240CE, CD240DCE, CD240D, CD241, CD242, CD243, CD244, CD245, CD246, CD247, CD248, CD249, CD252, CD253, CD254, CD256, CD262, CD263, CD264, CD265, CD266, CD267, CD268, CD269 (BCMA), CD270, CD271, CD272, CD273, CD274, CD275, CD276, CD277, CD278, CD279, CD280, CD281, CD282, CD283, CD284, CD286, CD289, CD290, CD292, CDw293, CD294, CD295, CD296, CD297, CD298, CD299, CD300a, CD300c, CD300c, CD301, CD302, CD303, CD304, CD305, CD306, CD307a, CD307b, CD307c, CD307d, CD307c, CD308, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD360, CD361, CD362, CD363, CD364, CD365, CD366, CD367, CD368, CD369, CD370, CD371, ACKR2, ACKR4, CCR10, CCRL2, CNTFR, CX3CR1, CXCR7, CXCR8, EDA2R, EDAR, EPOR, FLT1, FLT4, GHR, GPR75, IFNAR1, IFNAR2, IFNGR2, IL11RA, IL12RB2, IL17RB, IL17RC, IL17RD, IL17RE, IL1RAP, IL1RAPL, IL1RL1, IL1RL2, IL20RA, IL20RB, IL22RA1, IL22RA2, IL23R, IL27RA, IL28RA, IL31RA, IL3RB, OSMR, PRLR, RELL, RELT, SIGIRR, TNFRSF11B, TNFRSF19, TNFRSF22, TNFRSF23, TNFRSF25, TNFRSF26, TNFRSF3, TNFRSF6B, TSLPR, XCR1, an immunoglobulin light chain (lambda or kappa), an HLA protein (HLA refers to “human leukocyte antigen” and includes HLA-A, HLA.B, HLA-C, HLA-E, HLA-F, HLA-G, HLA-DM, HLA-DO, HLA-DP, HLA-DQ and HLA-DR), and β2-microglobulin. In some embodiments, the cell surface protein is selected from CD1a, CD1b, CD1c, CD1e, CD1e, CD2, CD3, CD3d, CD3c, CD3g, CD4, CD5, CD6, CD7, CD8a, CD8b, CD9, CD10, CD11a, CD11b, CD11c, CD11d, CDw12, CD13, CD14, CD15, CD15u, CD15s, CD15su, CD16, CD16b, CD17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49c, CD49f, CD50, CD51, CD52, CD53, CD54, CD55, CD56, CD57, CD58, CD59, CD60a, CD60b, CD60c, CD61, CD62E, CD62L, CD62P, CD63, CD64, CD65, CD65s, CD66a, CD66b, CD66c, CD66d, CD66c, CD66f, CD68, CD69, CD70, CD71, CD72, CD73, CD74, CD75, CD75s, CD77, CD79a, CD79b, CD80, CD81, CD82, CD83, CD84, CD85a, CD85d, CD85j, CD85k, CD86, CD87, CD88, CD89, CD90, CD91, CD92, CD93, CD94, CD95, CD96, CD97, CD98, CD99, CD99R, CD100, CD10¹, CD102, CD103, CD104, CD105, CD10⁶, CD10⁷a, CD10⁷b, CD108, CD109, CD110, CD111, CD112, CD113, CD114, CD115, CD116, CD117, CD118, CD119, CD120a, CD120b, CD121a, CD121b, CD122, CD123, CD124, CD125, CD126, CD127, CD129, CD130, CD131, CD132, CD133, CD134, CD135, CD136, CD137, CD138, CD139, CD140a, CD140b, CD141, CD142, CD143, CD144, CDw145, CD146, CD147, CD148, CDw149, CD150, CD151, CD152, CD153, CD154, CD155, CD156a, CD156b, CD156c, CD157, CD158c, CD158i, CD158k, CD159a, CD159c, CD160, CD161, CD162, CD163, CD164, CD165, CD166, CD167a, CD167b, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197, CDw198, CD199, CD200, CD201, CD202b, CD203c, CD204, CD205, CD206, CD207, CD208, CD209, CD210, CDw210b, CD212, CD213a1, CD213a2, CD215, CD217a, CD218a, CD218b, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240CE, CD240DCE, CD240D, CD241, CD242, CD243, CD244, CD245, CD246, CD247, CD248, CD249, CD252, CD253, CD254, CD256, CD266, CD267, CD268, CD269 (BCMA), CD270, CD271, CD272, CD273, CD274, CD275, CD276, CD277, CD278, CD279, CD280, CD281, CD282, CD283, CD284, CD286, CD289, CD290, CD292, CDw293, CD294, CD295, CD296, CD297, CD298, CD299, CD300a, CD300c, CD300c, CD301, CD302, CD303, CD304, CD305, CD306, CD307a, CD307b, CD307c, CD307d, CD307c, CD308, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD360, CD361, CD362, CD363, CD364, CD365, CD366, CD367, CD368, CD369, CD370, CD371, an immunoglobulin light chain (lambda or kappa), an HLA protein (HLA refers to “human leukocyte antigen” and includes HLA-A, HLA.B, HLA-C, HLA-E, HLA-F, HLA-G, HLA-DM, HLA-DO, HLA-DP, HLA-DQ and HLA-DR), and β2-microglobulin.

In some embodiments, the target protein can be a lineage-specific cell surface protein. In some embodiments, the target protein can be chosen from CD19; CD123; CD22; CD30; CD171; CS-1 (also referred to as CD2 subset 1, CRACC, SLAMF7, CD319, and 19A24); C-type lectin-like molecule-1 (CLECLi); CD33; epidermal growth factor receptor variant III (EGFRvIII); ganglioside G2 (CD2); ganglioside GD3 (aNeu5Ac(2-8) aNeu5Ac(2-3)bDGalp(1-4)bDGlep (1-1)Cer); TNF receptor family member B cell maturation (BCMA), Tn antigen ((Tn Ag) or (GalNAca-Ser/Thr)); prostate-specific membrane antigen (PSMA); Receptor tyrosine kinase-like orphan receptor 1 (ROR1); Fms-Like tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis (Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor I receptor (IGF-I receptor), carbonic anhydrase IX (CAIX), Proteasome (Prosome, Macropain) Subunit, Beta Type 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNcu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEMI/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY—BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex; locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1), ETS translocation-variant gene 6, located on chromosome 12p (ETV6-AML); sperm protein 17 (SPA17); X Antigen Family, member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-AP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B 1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES 1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxy esterase; heat shock protein 70-2 mutated (mut hsp70-2); CD79a; CD79b; CD72; Leukocyte-associated immunoglobulin-like receptor 1 (LAIR 1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2), lymphocyte antigen 75 (LY75); Glypican-3 (GPC3); Fc receptor-like 5 (FCRL5); and immunoglobulin lambda-like polypeptide 1 (IGLL1).

In some embodiments, the target protein is a protein expressed on the cell surface of hematopoietic cells. In some embodiments, the target protein is a receptor expressed on the cell surface of hematopoietic cells. For example, in some embodiments the target protein can be a receptor expressed on the cell surface of hematopoietic cells selected from ACKR2, ACKR4, CCR10, CCRL2, CD25, CD27, CD30, CD40, CD95, CD110, CD114, CD115, CD116, CD117, CD118, CD119, CD120a, CD120b, CD121a, CD121b, CD122, CD123, CD124, CD125, CD126, CD127, CD129, CD130, CD132, CD134, CD135, CD136, CD137, CD140a, CD140b, CD181, CD182, CD183, CD184, CD185, CD186, CD191, CD192, CD193, CD194, CD195, CD196, CD197, CD198/CDw198, CD199/CDw199, CD202b, CD210A, CD210B, CD212, CD213A1, CD213A2, CD215, CD217, CD218a, CD218b, CD220, CD221, CD234, CD246, CD261, CD262, CD263, CD264, CD265, CD266, CD267, CD268, CD269, CD270, CD271, CD295, CD309, CD331, CD332, CD333, CD334, CD357, CD358, CD360, CNTFR, CX3CR1, CXCR7, CXCR8, EDA2R, EDAR, EPOR, FLT1, FLT4, GHR, GPR75, IFNAR1, IFNAR2, IFNGR2, IL11RA, IL12RB2, IL17RB, IL17RC, IL17RD, IL17RE, IL1RAP, IL1RAPL, IL1RL1, IL1RL2, IL20RA, IL20RB, IL22RA1, IL22RA2, IL23R, IL27RA, IL28RA, IL31RA, IL3RB, OSMR, PRLR, RELL, RELT, SIGIRR, TNFRSF11B, TNFRSF19, TNFRSF22, TNFRSF23, TNFRSF25, TNFRSF26, TNFRSF3, TNFRSF6B, TSLPR, and XCR1.

In some embodiments, the target protein is a cytokine receptor or a chemokine receptor (e.g., expressed on the cell surface of hematopoietic cells). For example, in some embodiments the target protein can be a cytokine receptor or chemokine receptor expressed on the cell surface of hematopoictic cells selected from CD25, CD30, CD95, CD110, CD114, CD115, CD116, CD117, CD118, CD119, CD120a, CD120b, CD121a, CD121b, CD122, CD123, CD124, CD125, CD126, CD127, CD129, CD130, CD132, CD135, CD136, CD140a, CD140b, CD202b, CD210A, CD210B, CD212, CD213A1, CD213A2, CD215, CD217, CD218a, CD218b, CD220, CD221, CD246, CD261, CD262, CD263, CD264, CD265, CD266, CD267, CD268, CD269, CD270, CD295, CD309, CD331, CD332, CD333, CD334, CD360, EPOR, FLT1, FLT4, IL11RA, IL12RB2, IL17RB, IL17RC, IL17RD, IL17RE, IL1RAP, IL1RAPL, IL1RL1, IL1RL2, IL20RA, IL20RB, IL22RA1, IL22RA2, IL23R, IL27RA, IL28RA, IL31RA, IL3RB, OSMR, RELT, SIGIRR, TNFRSF25, TNFRSF3, TNFRSF6B, TSLPR, ACKR2, CCRL2, CCR10, CD181, CD182, CD183, CD184, CD185, CD186, CD191, CD192, CD193, CD194, CD195, CD196, CD197, CD198/CDw198, CD199/CDw199, CD234, CXCR7, CXCR8, CX3CR1, GPR75, and XCR1.

In some embodiments, the target protein is a cytokine receptor (e.g., expressed on the cell surface of hematopoietic cells). For example, in some embodiments the target protein can be a cytokine receptor expressed on the cell surface of hematopoietic cells selected from CD25, CD30, CD95, CD110, CD114, CD115, CD116, CD117, CD118, CD119, CD120a, CD120b, CD121a, CD121b, CD122, CD123, CD124, CD125, CD126, CD127, CD129, CD130, CD132, CD135, CD136, CD140a, CD140b, CD202b, CD210A, CD210B, CD212, CD213A1, CD213A2, CD215, CD217, CD218a, CD218b, CD220, CD221, CD246, CD261, CD262, CD263, CD264, CD265, CD266, CD267, CD268, CD269, CD270, CD295, CD309, CD331, CD332, CD333, CD334, CD360, EPOR, FLT1, FLT4, IL11RA, IL12RB2, IL17RB, IL17RC, IL17RD, IL17RE, IL1RAP, IL1RAPL, IL1RL1, IL1RL2, IL20RA, IL20RB, IL22RA1, IL22RA2, IL23R, IL27RA, IL28RA, IL31RA, IL3RB, OSMR, RELT, SIGIRR, TNFRSF25, TNFRSF3, TNFRSF6B, and TSLPR.

In some embodiments, the target protein is a chemokine receptor (e.g., expressed on the cell surface of hematopoietic cells). For example, in some embodiments the target protein can be a chemokine receptor expressed on the cell surface of hematopoietic cells selected from ACKR2, CCRL2, CCR10, CD181, CD182, CD183, CD184, CD185, CD186, CD191, CD192, CD193, CD194, CD195, CD196, CD197, CD198/CDw198, CD199/CDw199, CD234, CXCR7, CXCR8, CX3CR1, GPR75, and XCR1.

In some embodiments, the target protein is a cytokine receptor or a chemokine receptor expressed on the cell surface of lymphocytes. For example, in some embodiments the target protein can be selected from CD25, CD30, CD95, CD117, CD118, CD120a, CD120b, CD121a, CD121b, CD122, CD124, CD125, CD126, CD127, CD129, CD130, CD132, CD210A, CD210B, CD212, CD215, CD217, CD267, CD268, CD269, CD360, IL12RB2, IL23R, IL27RA, SIGIRR, TNFRSF25, CCRL2, CCR10, CD183, CD184, CD185, CD186, CD191, CD194, CD195, CD196, CD197, CXCR7, CX3CR1, and XCR1.

In some embodiments, the target protein is a cytokine receptor expressed on the cell surface of lymphocytes. For example, in some embodiments the target protein can be selected from CD25, CD30, CD95, CD117, CD118, CD120a, CD120b, CD121a, CD121b, CD122, CD124, CD125, CD126, CD127, CD129, CD130, CD132, CD210A, CD210B, CD212, CD215, CD217, CD267, CD268, CD269, CD360, IL12RB2, IL23R, IL27RA, SIGIRR, and TNFRSF25.

In some embodiments, the target protein is a chemokine receptor expressed on the cell surface of lymphocytes. For example, in some embodiments the target protein can be selected from CCRL2, CCR10, CD183, CD184, CD185, CD186, CD191, CD194, CD195, CD196, CD197, CXCR7, CX3CR1, and XCR1.

In some embodiments, the target protein is a cytokine receptor sub-unit of an interleukin-2 (IL-2) receptor, an IL-4 receptor, an IL-7 receptor, an IL-9 receptor, an IL-15 receptor, or an IL-21 receptor. For example, in some embodiments the target protein can be selected from CD25, CD122, CD124, CD127, CD129, CD132 (IL2RG), CD215, and CD360.

In some embodiments of the present invention, the target protein is interleukin-2 receptor subunit gamma (also known as IL2RG, cytokine receptor common subunit gamma, IL-2 receptor subunit gamma, IL-2R subunit gamma, IL-2RG, IL2Rγ, IL-2Rγ and CD132). The IL2RG blocking antibody REGN7257 efficiently suppresses lymphocytes in vivo. See, e.g., WO 2020/160242 A1 and US 2020/0247894 A1, each of which is herein incorporated by reference in its entirety for all purposes. IL2RG is a subunit which is common to several interleukin receptors including IL-2R, IL-4R, IL-7R, IL-9R, IL-15R and IL-21R. The common cytokine receptor gamma chain was first identified as the third chain of the interleukin-2 (IL-2) receptor complex and named IL2RG. The same subunit was identified as part of several other cytokine receptors complexes: IL-4, IL-7, IL-9, IL-15, and IL-21, and therefore may be referred to as γc (common cytokine receptor gamma chain). The γc is involved in the signal transduction of these cytokine receptors as well as ligand binding. Human IL2RG is assigned UniProt Accession No. P31785. The canonical isoform of human IL2RG is assigned UniProt Accession No. P31785-1 and NCBI Accession No. NP_000197.1 and is set forth in SEQ ID NO: 21. An engineered M145K variant of human IL2RG is set forth in SEQ ID NO: 22. An exemplary messenger RNA encoding the canonical isoform of human IL2RG is assigned NCBI Accession No. NM_000206.3 and is set forth in SEQ ID NO: 23. The coding sequence for the canonical isoform of human IL2RG is assigned CCDS ID CCDS14406.1 and is set forth in SEQ ID NO: 24. The gene encoding human interleukin-2 receptor subunit gamma is called IL2RG, is on chromosome X, and is assigned NCBI GeneID 3561. It is at location Xq13.1 (assembly: GRCh38.p14 (GCF_000001405.40); location: NC_000023.11 (71107404 . . . 71111577, complement)).

The expression “functionally indistinguishable” refers to a first and a second isoform that are equally capable of performing the same function within a cell without significant impairment. For example, the function can be binding to an endogenous ligand and/or activating downstream signaling pathways within a cell. In other words, the first and the second isoform are functionally largely indistinguishable. In certain embodiments, a slight functional impairment can be acceptable. The function that is largely indistinguishable can be, for example, binding to an endogenous ligand and/or activating downstream signaling pathways. In some embodiments, the function can be binding to the endogenous ligand and activating downstream signaling pathways. The expression “immunologically distinguishable” refers to a first and a second isoform of a protein that can be distinguished by an antigen-binding protein specifically binding to either the first or the second isoform but not the other, such as the antigen-binding protein specifically binding only to the second (unmodified) isoform of the target protein. In other words, the antigen-binding proteins are able to discriminate between the two isoforms by specifically binding only one isoform, but not the other one. In a specific embodiment, the endogenous ligand binds (or endogenous ligands bind) both the first and second isoforms (e.g., equally, or with only slight impairment), but an engineered antigen-binding protein such as an antibody is able to discriminate between the two isoforms by specifically binding only one isoform, but not the other one (e.g., specifically binding only to the second but not the first isoform).

In some embodiments, the second isoform of the target protein refers to the form that is present in the subject. In some embodiments, the second isoform of the target protein refers to the wild type form or native form of the target protein (i.e., the form that usually occurs in nature), and the first isoform refers to an isoform obtained by introducing a mutation in the nucleic acid sequence encoding the second isoform. The native form of a protein refers to a protein that is encoded by a nucleic acid sequence within the genome of the cell and that has not been inserted or mutated by genetic manipulation (i.e., a native protein is a protein that is not a transgenic protein or a genetically engineered protein).

The mutation in the first isoform can be any type of mutation and any size mutation. In some embodiments, the mutation comprises an insertion, a deletion and/or a substitution of 1, 2, 3, 4, 5, 6, 7, 8, 9, 10, 11, 12, 13, 14, 15, 16, 17, 18, 19, or 20 amino acids (e.g., 1-20 amino acids, 1-5 amino acids, 1-3 amino acids, or 1 amino acid). In some embodiments, the mutation comprises a substitution (e.g., comprises a substitution of 1 amino acid). In some embodiments, the mutation consists essentially of a substitution (e.g., consists essentially of a substitution of 1 amino acid). In some embodiments, the mutation consists of a substitution (e.g., consists of a substitution of 1 amino acid). The mutation can be at any site in the target protein. For example, if the target protein is a cell surface protein, the mutation can in some embodiments be in the extracellular domain of the target protein. In some embodiments, the site of the mutation can be a site that is non-conserved between different mammalian species. In some embodiments, the mutation does not result in a secondary structure change in the surface protein. In some embodiments, the mutation is within the epitope targeted by an antigen-binding protein or is at a site that is accessible to ligand binding. In some embodiments, the mutation is not located at a site involved in a predicted or experimentally established or confirmed protein-protein interaction of the surface protein. In some embodiments, the mutation does not result in deleting or introducing a disulfide bond inter- or intramolecular interaction or a hydrophobic stacking. In some embodiments, the mutation does not result in deleting or introducing a posttranslational protein modification site, such as a glycosylation site. In some embodiments, the mutation is located at a site that has a unique topology compared to other mammalian proteins according to crystal structure analysis or computer-aided structure prediction. In instances where an antibody or antigen-binding protein reactive against a target protein already exists, the information regarding the epitope of the target protein that is recognized by the antibody or antigen-binding protein can be used to select the site of the mutation.

In some embodiments, the first isoform of the target protein is a genetically engineered isoform of the target protein. For example, the first isoform of the target protein can be genetically engineered to comprise a mutation (e.g., an artificial mutation that is not naturally occurring) to provide an altered epitope. The altered epitope can be, for example, in the binding region of an antigen-binding protein such as an antibody. In some embodiments, the target protein is IL2RG (e.g., human IL2RG), and the altered epitope is in a binding region of the REGN7257 anti-IL2RG antibody described elsewhere herein. REGN7257 binding region 1 is encoded by exon 3 of IL2RG (e.g., human IL2RG) and includes T127, F128, V129, V130, Q131, L132, Q133, D134, P135, R136, E137, P138, R139, R140, Q141, A142, T143, Q144, M145, L146, K147, L148, Q149, and N150. REGN7257 binding region 2 is encoded by exons 2 and 3 of IL2RG (e.g., human IL2RG) and includes L87, H88, Y89 (exon 2), W90 (codon is split between exons 2 and 3), Y91, K92, N93, S94, D95, N96, and D97 (exon 3). In some embodiments, the mutation can comprise a mutation (e.g., a substitution) encoded by nucleotides within exon 2 of the IL2RG gene (e.g., human IL2RG gene). In some embodiments, the mutation can comprise a mutation (e.g., a substitution) encoded by nucleotides within exon 3 of the IL2RG gene (e.g., human IL2RG gene). In some embodiments, the mutation can comprise a mutation (e.g., a substitution) encoded by nucleotides within exons 2 and 3 of the IL2RG gene (e.g., human IL2RG gene). In some embodiments, the mutation can comprise a mutation (e.g., a substitution) within the region from position T127 to position N150 of IL2RG (e.g., human IL2RG). In some embodiments, the mutation can comprise a mutation (e.g., a substitution) within the region from position L87 to position D97 of IL2RG (e.g., human IL2RG). In some embodiments, the mutation can comprise a mutation (e.g., a substitution) within the region from position T127 to position N150 of IL2RG (e.g., human IL2RG) and within the region from position L87 to position D97 of IL2RG (e.g., human IL2RG). In some embodiments, the mutation can comprise a mutation (e.g., a substitution) within REGN7257 binding region 1 (SEQ ID NO: 25). In some embodiments, the mutation can comprise a mutation (e.g., a substitution) within REGN7257 binding region 2 (SEQ ID NO: 62). In some embodiments, the mutation can comprise a mutation (e.g., a substitution) within REGN7257 binding region 1 (SEQ ID NO: 25) and within REGN7257 binding region 2 (SEQ ID NO: 62). In some embodiments, the mutation can comprise a mutation (e.g., a substitution) at one or more of the following positions: T127, F128, V129, V130, Q131, L132, Q133, D134, P135, R136, E137, P138, R139, R140, Q141, A142, T143, Q144, M145, L146, K147, L148, Q149, and N150. In some embodiments, the mutation can comprise a mutation (e.g., a substitution) at one or more of the following positions: L87, H88, Y89, W90, Y91, K92, N93, S94, D95, N96, and D97. In some embodiments, the mutation can comprise a mutation (e.g., a substitution) at one or more of the following positions: T127, F128, V129, V130, Q131, L132, Q133, D134, P135, R136, E137, P138, R139, R140, Q141, A142, T143, Q144, M145, L146, K147, L148, Q149, N150, L87, H88, Y89, W90, Y91, K92, N93, S94, D95, N96, and D97. For example, the mutation can comprise a mutation (e.g., a substitution) at position M145, position W90, position K92, position N93, position D95, position D97, position T127, position R139, position R140, position Q141, position T143, position K147, or any combination thereof of IL2RG (e.g., human IL2RG). A mutation at a position within IL2RG encompasses mutations (e.g., substitutions) including only the residue at that position or mutations (e.g., substitutions) including the residue at that position as well as other residues at other positions. The nomenclature of the amino acid position for the mutations or residue disclosed herein refer to the position of the mutation or residue in the canonical isoform of human IL2RG set forth in SEQ ID NO: 21. In some embodiments, the mutation comprises a mutation (e.g., a substitution) at position M145. Examples of suitable M145 substitutions include a M145K substitution, a M145D substitution, a M145E substitution, a M145P substitution, a M145W substitution, or an M145Y substitution. In some embodiments, the mutation comprises a M145K substitution. In some embodiments, the mutation comprises a mutation (e.g., a substitution) at position W90. Examples of suitable W90 substitutions include a W90V substitution, a W90R substitution, a W90Q substitution, a W90L substitution, a W90K substitution, a W90E substitution, or a W90D substitution. In some embodiments, the mutation comprises a W90Q substitution. In some embodiments, the mutation comprises a mutation (e.g., a substitution) at positions M145 and W90.

The donor cells or edited cells can be administered to the subject by any suitable means. The term administering refers to administration of a composition (e.g., the donor cells or edited cells) to a subject or system (e.g., but not limited to, to a cell, organ, tissue, organism, or relevant component or set of components thereof). The route of administration may vary depending, for example, on the subject or system to which the composition is being administered, the nature of the composition, the purpose of the administration, and so forth. The term “administration” or “administering” is intended to include routes of introducing the donor cells or edited cells to a subject to perform their intended function. In some embodiments, non-limiting examples of routes of administration which can be used include, e.g., injection (subcutaneous, intravenous, parenterally, intraperitoneally, intrathecal), such as intravenous injection. In some embodiments of the present invention, the donor cells or edited cells are administered by intravenous injection. Administration may involve intermittent dosing or continuous dosing (e.g., but not limited to, perfusion) for at least a selected period of time. The donor cells or edited cells can be administered alone, or in conjunction with either another agent (e.g., but not limited to, an agent for selective inhibition or selective depletion of host cells or non-edited cells in the subject) or with a pharmaceutically acceptable carrier, or both. The donor cells or edited cells can be administered prior to the administration of the other agent, simultaneously with the agent, or after the administration of the agent.

The host cells or non-edited cells in the subject can be selectively inhibited or selectively depleted based on their expression of the second isoform of the target protein by any suitable means. For example, they can be depleted or inhibited based on their expression of only the second isoform of the target protein and their lack of expression of the first isoform of the target protein. Alternatively, they can be depleted or inhibited based on their expression of the second isoform regardless of their expression of the first isoform of the target protein. The selective inhibition or selective depletion of the host cells or non-edited cells can occur before administration of the donor cells or edited cells, simultaneously with the administration of the donor cells or edited cells, or after administration of the donor cells or edited cells. Selective depletion refers to selectively reducing the total number or concentration of cells expressing a certain isoform of the target protein. Selective depletion of cells expressing a second isoform can correspond to enrichment of cells expressing the first isoform.

In some embodiments, selective depletion refers to selective ablation of host cells. Selective ablation of host cells refers to ablation (i.e., killing) of host cells by an active killing mechanism. An active killing mechanism means the agent directly kills the host cells by cytotoxic mechanisms (e.g., antibody-drug conjugate (ADC), antibody radioconjugate (ARC), CAR-T, or other engineered cytotoxicity) or recruits host cytotoxic effector mechanisms (e.g., complement-dependent cytotoxicity (CDC), antibody-dependent cellular cytotoxicity (ADCC), antibody-dependent cellular phagocytosis (ADCP)), as opposed blocking a cellular function (e.g. growth or cytokine signaling, chemotactic tissue homing, cell-cell adhesion; e.g., as with selective inhibition) without engaging extrinsic cytotoxic effectors. In some embodiments, the selective depletion of host cells or non-edited cells comprises ablating the host cells or non-edited cells via an active killing mechanism such as complement-dependent cytotoxicity (CDC), antibody-dependent cellular cytotoxicity (ADCC), antibody-dependent cellular phagocytosis (ADCP), antibody-drug conjugate (ADC), CAR-T, or other engineered cytotoxicity.

In other embodiments, selective depletion refers to selective inhibition of host cells. In contrast to selective ablation, selective inhibition of host cells does not comprise ablation (i.e., killing) of host cells by an active killing mechanism. That is, selective inhibition of host cells does not comprise cytotoxic ablation of host cells. In some embodiments, the selective inhibition of host cells or non-edited cells does not comprise ablation or killing of host cells or non-edited cells, even indirectly. In some embodiments, the selective inhibition or selective depletion of host cells or non-edited cells comprises: (1) blocking growth of the host cells or non-edited cells (e.g., blocking proliferation or immune cell activation) to provide a competitive growth advantage to the donor cells or edited cells; (2) blocking localization or trafficking of the host cells or non-edited cells to provide a competitive homing advantage to the donor cells or edited cells; (3) blocking a cell-cell interaction or adhesion of the host cells or non-edited cells to provide a competitive tissue infiltration advantage to the donor cells or edited cells; or (4) blocking immune cell activation in the host cells or non-edited cells to provide a competitive advantage to the donor cells or edited cells. For example, in some embodiments, the selective inhibition or selective depletion of host cells or non-edited cells comprises blocking growth (i.e., proliferation) and/or blocking immune cell activation. For example, in some embodiments, the selective inhibition or selective depletion of host cells or non-edited cells comprises blocking growth (i.e., proliferation) and blocking immune cell activation. For example, in some embodiments, the selective inhibition or selective depletion of host cells or non-edited cells comprises blocking growth (i.e., proliferation). For example, in some embodiments, the selective inhibition or selective depletion of host cells or non-edited cells comprises blocking immune cell activation. As a novel conditioning strategy, selective inhibition of host cells without cytotoxic ablation has potential to improve the safety and efficacy of cell therapy and transplant treatments. Non-ablative conditioning may avoid undesired and harmful effects of ablative agents, including direct killing of non-target (e.g., non-hematopoietic) cells expressing drug target antigens, indirect toxicities to target-adjacent tissues, and prolonged immune suppression in the post-transplant period. Selective blockade or suppression of essential host cell factors can enhance the expansion, persistence, and trafficking of resistant donor cells by affording favorable competition for limiting host factors (e.g., cytokine, chemokines) and immune niche space, without harsh and potentially toxic ablative agents. See, e.g., FIGS. 2A-5D.

In some embodiments of the present invention, the selective inhibition or selective depletion of the host cells or non-edited cells in the subject can comprise administering an agent (e.g., an antagonist, an antigen-binding protein, or a population of cells (i.e., immune effector cells such as chimeric antigen receptor T cells (CAR-T)) expressing an antigen-binding protein)) that specifically binds to the second isoform of the target protein but does not specifically bind to the first isoform of the target protein. For example, the agent can be an antagonist that blocks interaction between an endogenous ligand and the second isoform of the target protein but does not block interaction between the endogenous ligand and the first isoform of the target protein. In some embodiments of the present invention, the selective inhibition or selective depletion of the host cells or non-edited cells in the subject can comprise administering an antagonist (e.g., an antigen-binding protein or a population of cells (i.e., immune effector cells such as chimeric antigen receptor T cells (CAR-T)) expressing an antigen-binding protein) that specifically binds to the second isoform of the target protein but does not specifically bind to the first isoform of the target protein. In some embodiments of the present invention, the selective inhibition or selective depletion of the host cells or non-edited cells in the subject can comprise administering an antigen-binding protein (e.g., an isolated antigen-binding protein) or one or more nucleic acids encoding the antigen-binding protein, such as an antibody (e.g., human antibody, monoclonal antibody, and/or recombinant antibody) or an antigen-binding fragment thereof that specifically binds to the second isoform of the target protein (or an antigenic fragment thereof (e.g., the extracellular domain)) but does not specifically bind to the first isoform of the target protein. In some embodiments of the present invention, the selective inhibition or selective depletion of the host cells or non-edited cells in the subject can comprise administering a population of cells (i.e., immune effector cells) expressing an antigen-binding protein (e.g., T cells expressing a chimeric antigen receptor or an exogenous T cell receptor) that specifically binds to the second isoform of the target protein but does not specifically bind to the first isoform of the target protein. Immune effector cells are cells that are capable of effecting or enhancing an immune response. In some embodiments (i.e., for selective ablation), selective depletion (e.g., selective ablation) can be achieved by cytotoxic mechanisms (e.g., antibody-drug conjugate (ADC), antibody radioconjugate (ARC), CAR-T, or other engineered cytotoxicity). In some embodiments (i.e., for selective ablation), selective depletion (e.g., selective ablation) can be achieved by recruiting host cytotoxic effector mechanisms (e.g., complement-dependent cytotoxicity (CDC), antibody-dependent cellular cytotoxicity (ADCC), antibody-dependent cellular phagocytosis (ADCP)), as opposed blocking a cellular function (e.g. growth or cytokine signaling, chemotactic tissue homing, cell-cell adhesion; e.g., as with selective inhibition) without engaging extrinsic cytotoxic effectors. In some embodiments (i.e., for selective ablation), the antigen-binding protein is coupled to a toxin, thereby forming an immunotoxin. In some embodiments, the antigen-binding protein is not coupled to a toxin. In some embodiments, the antigen-binding protein is a bispecific antigen-binding protein that can simultaneously bind to two different antigens. In some embodiments, selective depletion (e.g., selective ablation) can be achieved by complement-dependent cytotoxicity (CDC), by antibody-dependent cellular cytotoxicity (ADCC), antibody-dependent cellular phagocytosis (ADCP), or by an antibody-drug conjugate (ADC). In some embodiments, selective inhibition or selective depletion is not achieved by cytotoxic mechanisms or by recruiting host cytotoxic effector mechanisms. In some embodiments, selective inhibition or selective depletion is achieved by blocking a cellular function (e.g. growth or cytokine signaling, chemotactic tissue homing, cell-cell adhesion) without engaging extrinsic cytotoxic effectors. In some embodiments, selective inhibition or selective depletion is not achieved by complement-dependent cytotoxicity (CDC), by antibody-dependent cellular cytotoxicity (ADCC), antibody-dependent cellular phagocytosis (ADCP), or by an antibody-drug conjugate (ADC). Toxins or drugs compatible for use in antibody-drug conjugate are well known in the art. See, e.g., Peters et al. (2015) Biosci. Rep. 35 (4): e00225, Beck et al. (2017) Nature Reviews Drug Discovery 16:315-337; Marin-Acevedo et al. (2018) J. Hematol. Oncol. 11:8; Elgundi et al. (2017) Advanced Drug Delivery Reviews 122:2-19, each of which is herein incorporated by reference in its entirety for all purposes. In some embodiments, the antibody-drug conjugate may further comprise a linker (e.g., a peptide linker, such as a cleavable linker) attaching the antigen-binding protein (e.g., antibody) and drug molecule. Selective inhibition or selective depletion can also be effected by administration of an antigen-binding protein that is not coupled to an effector compound such as a drug or a toxin. In some embodiments, selective inhibition or selective depletion is achieved by blocking binding by an endogenous ligand.

In methods in which an agent for selective inhibition or selective depletion of host cells or non-edited cells is administered, the agent can in some embodiments be administered simultaneously with the donor cells or edited cells. In some embodiments, the donor cells or edited cells are administered after the agent. For example, in some embodiments, the donor cells or edited cells are administered within 1 day after the agent, or at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 3 months, at least about 4 months, at least about 5 months, at least about 6 months or more after the agent. In some embodiments, the donor cells or edited cells are administered before the agent. For example, in some embodiments, the donor cells or edited cells are administered within 1 day before the agent, or at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 3 months, at least about 4 months, at least about 5 months, at least about 6 months or more before the agent.

In some embodiments, the donor cells or edited cells are administered in multiple administrations (e.g., doses). In some embodiments, the donor cells or edited cells are administered to the subject once. In some embodiments, the donor cells or edited cells are administered to the subject more than once (e.g., at least 2, at least 3, at least 4, at least 5 or more times). In some embodiments, the donor cells or edited cells are administered to the subject at a regular interval (e.g., every 6 months). In methods in which an agent for selective inhibition or selective depletion of host cells or non-edited cells is administered, it can, in some embodiments, be administered in multiple administrations (e.g., doses). In some embodiments, the agent is administered to the subject once. In some embodiments, the agent is administered to the subject more than once (e.g., at least 2, at least 3, at least 4, at least 5 or more times). In some embodiments, the agent is administered to the subject at a regular interval (e.g., every 6 months).

In some embodiments, the agent is administered to the subject prior to administration of the donor cells or edited cells and after administration of the donor cells or edited cells. In some embodiments, the agent is administered to the subject prior to administration of the donor cells or edited cells and after administration of the donor cells or edited cells, and the donor cells or edited cells are administered to the subject once. In some embodiments, for example, the agent is administered to the subject about 1 to about 2 weeks prior to administration of the donor cells or edited cells and is administered to the subject about 1 to about 2 weeks after administration of the donor cells or edited cells (e.g., to give the donor cells or edited cells a competitive advantage).

In some embodiments, the agent is administered to the subject in multiple administrations (e.g., at least 2, at least 3, at least 4, at least 5 or more times) prior to administration of the donor cells or edited cells. In some embodiments, the agent is administered to the subject in multiple administrations (e.g., at least 2, at least 3, at least 4, at least 5 or more times) after administration of the donor cells or edited cells. In some embodiments, the agent is administered to the subject in multiple administrations (e.g., at least 2, at least 3, at least 4, at least 5 or more times) prior to administration of the donor cells or edited cells and in multiple administrations (e.g., at least 2, at least 3, at least 4, at least 5 or more times) after administration of the donor cells or edited cells.

In some embodiments, about 10⁶ to 10¹¹ donor cells or edited cells are administered. In some embodiments it may be desirable to administer fewer than 10⁶ cells to the subject. In some embodiments, it may be desirable to administer more than 10¹¹ cells to the subject. In some embodiments, one or more doses of cells includes about 10⁶ cells to about 10¹¹ cells, about 10⁷ cells to about 10¹⁰ cells, about 10⁸ cells to about 10⁹ cells, about 10⁶ cells to about 10⁸ cells, about 10⁷ cells to about 10⁹ cells, about 10⁷ cells to about 10¹⁰ cells, about 10⁷ cells to about 10¹¹ cells, about 10⁸ cells to about 10¹⁰ cells, about 10⁸ cells to about 10¹¹ cells, about 10⁸ cells to about 10¹⁰ cells, about 10⁹ cells to about 10¹¹ cells, or about 10¹⁰ cells to about 10¹¹ cells. In some embodiments, one or more doses of cells includes about 10⁶ to 10⁷ cells per kg.

An “antagonist” includes molecules that inhibit an activity of the target protein to any detectable degree. For example, an antagonist of IL2RG includes molecules that inhibit an activity of IL2RG (e.g., binding of a hybrid receptor comprising IL2RG complexed with a cytokine-specific receptor subunit from binding to a cytokine such as IL-2, IL-4, IL-7, IL-9, IL-15 and/or IL-21) to any detectable degree.

In some embodiments, the agent for selective inhibition or selective depletion of host cells or non-edited cells is an antigen-binding protein.

The term “specifically binds” or “binds specifically” refers to those antigen-binding proteins (e.g., antibodies or antigen-binding fragments thereof) having a binding affinity to an antigen, such as IL2RG protein, expressed as K_{D}, of at least about 10⁻⁷ M (e.g., 10⁻⁸ M, 10⁻⁹ M, 10⁻¹⁰ M, 10⁻¹¹ M or 10⁻¹² M), as measured by real-time, label free bio-layer interferometry assay, for example, at 25° C. or 37° C., e.g., an Octet® HTX biosensor, or by surface plasmon resonance, e.g., BIACORE™, or by solution-affinity ELISA. In some embodiments, the antigen-binding proteins used herein specifically bind to IL2RG protein or human IL2RG protein (e.g., wild type or native IL2RG protein, such as wild type or native human IL2RG protein). “Anti-IL2RG” refers to an antigen-binding protein (or another molecule), for example an antibody or antigen-binding fragment thereof, that binds specifically to IL2RG.

An antigen is a molecule, such as a peptide (e.g., IL2RG or a fragment thereof (an antigenic fragment)), to which, for example, an antibody binds. The specific region on an antigen that an antibody recognizes and binds to is called the epitope.

The term “epitope” refers to an antigenic determinant (e.g., on IL2RG) that interacts with a specific antigen-binding site of an antigen-binding protein, e.g., a variable region of an antibody molecule, known as a paratope. A single antigen may have more than one epitope. Thus, different antigen-binding proteins (e.g., antibodies) may bind to different areas on an antigen and may have different biological effects. The term “epitope” may also refer to a site on an antigen to which B and/or T cells respond and/or to a region of an antigen that is bound by an antibody. Epitopes may be defined as structural or functional. Functional epitopes are generally a subset of the structural epitopes and have those residues that directly contribute to the affinity of the interaction. Epitopes may be linear or conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. Epitopes to which the antigen-binding proteins used in the present invention bind may be included in fragments of IL2RG, e.g., human IL2RG, for example the ectodomain, domain 1 or domain 2 thereof.

Methods for determining the epitope of an antigen-binding protein, e.g., antibody or fragment or polypeptide, include alanine scanning mutational analysis, peptide blot analysis (Reineke (2004) Methods Mol. Biol. 248:443-63, herein incorporated by reference in its entirety for all purposes), peptide cleavage analysis, crystallographic studies and NMR analysis. In addition, methods such as epitope excision, epitope extraction and chemical modification of antigens can be employed (Tomer (2000) Prot. Sci. 9:487-496, herein incorporated by reference in its entirety for all purposes). Another method that can be used to identify the amino acids within a polypeptide with which an antigen-binding protein (e.g., antibody or fragment or polypeptide) interacts is hydrogen/deuterium exchange detected by mass spectrometry. See, e.g., Ehring (1999) Analytical Biochemistry 267:252-259; Engen and Smith (2001) Anal. Chem. 73: 256A-265A, each of which is herein incorporated by reference in its entirety for all purposes.

The term “antibody,” as used herein, refers to immunoglobulin molecules comprising four polypeptide chains, two heavy chains (HCs) and two light chains (LCs) inter-connected by disulfide bonds (i.e., “full antibody molecules”) (e.g., IgG)—for example REGN7257 (also called H4H12889P). In some embodiments, each antibody heavy chain (HC) comprises a heavy chain variable region (“HCVR” or “V_{H}”) (e.g., SEQ ID NO: 2 or a variant thereof) and a heavy chain constant region (including domains C_{H}1, C_{H}2 and C_{H}3); and each antibody light chain (LC) comprises a light chain variable region (“LCVR” or “V_{L}”) (e.g., SEQ ID NO: 10 or a variant thereof) and a light chain constant region (C_{L}). The V_{H} and V_{L}, regions can be further subdivided into regions of hypervariability, termed complementarity determining regions (CDR), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. In certain embodiments, the FRs of the antibody (or antigen binding fragment thereof) are identical to the human germline sequences or are naturally or artificially modified.

Typically, the variable domains of both the heavy and light immunoglobulin chains comprise three hypervariable regions, also called complementarity determining regions (CDRs), located within relatively conserved framework regions (FR). In general, from N-terminal to C-terminal, both light and heavy chains variable domains comprise FR1, CDR1, FR2, CDR2, FR3, CDR3 and FR4. In some embodiments, the assignment of amino acids to each domain is in accordance with the definitions of Sequences of Proteins of Immunological Interest, Kabat et al.; National Institutes of Health, Bethesda, Md.; 5th ed.; NIH Publ. No. 91-3242 (1991); Kabat (1978) Adv. Prot. Chem. 32:1-75; Kabat et al., (1977) J. Biol. Chem. 252:6609-6616; Chothia et al., (1987) J. Mol. Biol. 196:901-917 or Chothia et al., (1989) Nature 342:878-883, each of which is herein incorporated by reference in its entirety for all purposes. Thus, antigen-binding proteins in some embodiments include antibodies and antigen-binding fragments including the CDRs of a V_{H} and the CDRs of a V_{L}, which V_{H} and V_{L} comprise amino acid sequences as set forth herein (or a variant thereof), wherein the CDRs are as defined according to Kabat and/or Chothia.

The terms “antigen-binding portion” or “antigen-binding fragment” of an antibody or antigen-binding protein, and the like, as used herein, include any naturally occurring, enzymatically obtainable, synthetic, or genetically engineered polypeptide or glycoprotein that specifically binds an antigen to form a complex. Non-limiting examples of antigen-binding fragments include: (i) Fab fragments; (ii) F(ab′) 2 fragments; (iii) Fd fragments (heavy chain portion of a Fab fragment cleaved with papain); (iv) Fv fragments (a V_{H} or V_{L}); and (v) single-chain Fv (scFv) molecules; consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies and small modular immunopharmaceuticals (SMIPs), are also encompassed within the expression “antigen-binding fragment,” as used herein. In some embodiments, the antigen-binding fragment comprises three or more CDRs of H4H12889P (e.g., CDR-H1, CDR-H2 and CDR-H3; or CDR-L1, CDR-L2 and CDR-L3).

In some embodiments, the antigen-binding protein is a “neutralizing” or “antagonist” anti-target protein antigen-binding protein (e.g., antibody or antigen-binding fragment), including molecules that inhibit an activity of the target protein (e.g., inhibiting binding of a receptor to one of its ligands) to any detectable degree.

In some embodiments, the antigen-binding proteins can comprise monoclonal antigen-binding proteins, e.g., antibodies and antigen-binding fragments thereof, as well as monoclonal compositions comprising a plurality of isolated monoclonal antigen-binding proteins. The term “monoclonal antibody” or “mAb,” as used herein, refers to a member of a population of substantially homogenous antibodies, i.e., the antibody molecules comprising the population are identical in amino acid sequence except for possible naturally occurring mutations that may be present in minor amounts. A “plurality” of such monoclonal antibodies and fragments in a composition refers to a concentration of identical (in amino acid sequence except for possible naturally occurring mutations that may be present in minor amounts) antibodies and fragments which is above that which would normally occur in nature, e.g., in the blood of a host organism such as a mouse or a human.

In some embodiments, the antigen-binding protein, e.g., antibody or antigen-binding fragment comprises a heavy chain constant domain, e.g., of the type IgA (e.g., IgA1 or IgA2), IgD, IgE, IgG (e.g., IgG1, IgG2, IgG3 and IgG4 (e.g., comprising a S228P and/or S108P mutation)) or IgM. In some embodiments, the antigen-binding protein, e.g., antibody or antigen-binding fragment, comprises a light chain constant domain, e.g., of the type kappa or lambda. In some embodiments, the antigen-binding protein includes antigen-binding proteins comprising the variable domains set forth herein (e.g., H4H12889P) which are linked to a heavy and/or light chain constant domain, e.g., as set forth above.

In some embodiments, the antigen-binding protein is a human antigen-binding protein (e.g., antibodies or antigen-binding fragments thereof such as H4H12889P)). The term “human” antigen-binding protein, such as an antibody or antigen-binding fragment, as used herein, includes antibodies and fragments having variable and constant regions derived from human germline immunoglobulin sequences whether in a human cell or grafted into a non-human cell, e.g., a mouse cell. See, e.g., U.S. Pat. Nos. 8,502,018, 6,596,541, or U.S. Pat. No. 5,789,215, each of which is herein incorporated by reference in its entirety for all purposes. The human antibodies and antigen-binding fragments may, in some embodiments, include amino acid residues not encoded by human germline immunoglobulin sequences (e.g., having mutations introduced by random or site-specific mutagenesis in vitro or by somatic mutation in vivo), for example in the CDRs and, in particular, CDR3. However, the term “human antibody,” as used herein, is not intended to include mAbs in which CDR sequences derived from the germline of another mammalian species (e.g., mouse) have been grafted onto human FR sequences. The term includes antibodies recombinantly produced in a non-human mammal or in cells of a non-human mammal. The term is not intended to include antibodies isolated from or generated in a human subject.

In some embodiments, the antigen-binding protein is a chimeric antigen-binding protein (e.g., chimeric antibodies comprising the variable domains which are set forth herein (e.g., from H4H12889P)). As used herein, a “chimeric antibody” is an antibody having the variable domain from a first antibody and the constant domain from a second antibody, where the first and second antibodies are from different species. See, e.g., U.S. Pat. No. 4,816,567; and Morrison et al., (1984) Proc. Natl. Acad. Sci. U.S.A. 81:6851-6855, each of which is herein incorporated by reference in its entirety for all purposes.

In some embodiments, the antigen-binding protein is a recombinant antigen-binding protein (e.g., recombinant antigen-binding proteins as set forth herein (e.g., H4H12889P)). The term “recombinant” antigen-binding proteins, such as antibodies or antigen-binding fragments thereof, refers to such molecules created, expressed, isolated or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a host cell (e.g., Chinese hamster ovary (CHO) cell) or cellular expression system or isolated from a recombinant combinatorial human antibody library.

In some embodiments, the antigen-binding protein is an antigen-binding fragment of an antibody (e.g., an antigen-binding fragment of an antigen-binding protein set forth herein, for example, H4H12889P). An antigen-binding fragment of an antibody will, in some embodiments, comprise at least one variable domain. The variable domain may be of any size or amino acid composition and will generally comprise at least one (e.g., 3) CDR(s), which is adjacent to or in frame with one or more framework sequences. In antigen-binding fragments having a V_{H} domain associated with a V_{L} domain, the V_{H} and V_{L}, domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} and/or V_{L} domain which are bound non-covalently.

In certain embodiments, the antigen-binding fragment of an antibody may contain at least one variable domain covalently linked to at least one constant domain. Non-limiting, exemplary configurations of variable and constant domains that may be found within an antigen-binding fragment of an antibody include: (i) V_{H}-C_{H}1; (ii) V_{H}-C_{H}2; OD V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-CHI; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60, or more) amino acids, which result in a flexible or semi-flexible linkage between adjacent variable and/or constant domains in a single polypeptide molecule. Moreover, an antigen-binding fragment of an antibody may comprise a homo-dimer or hetero-dimer (or other multimer) of any of the variable and constant domain configurations listed above in noncovalent association with one another and/or with one or more monomeric V_{H} or V_{L} domain (e.g., by disulfide bond(s)).

The antigen-binding proteins (e.g., antibodies and antigen-binding fragments) may be monospecific or multi-specific (e.g., bispecific), such as monospecific as well as multispecific (e.g., bispecific) antigen-binding fragments comprising one or more variable domains from an antigen-binding protein that is specifically set forth herein (e.g., H4H12889P).

In some embodiments, the antigen-binding protein is an antigen-binding protein, such as an antibody (e.g., human antibody, monoclonal antibody, or recombinant antibody) or an antigen-binding fragment thereof, that specifically binds to IL2RG protein or an antigenic fragment thereof (e.g., the extracellular domain of IL2RG or human IL2RG). See, e.g., WO 2020/160242 A1 and US 2020/0247894 A1, each of which is herein incorporated by reference in its entirety for all purposes. For example, the antigen-binding protein can comprise any polypeptide that includes an amino acid sequence set forth in SEQ ID NO: 18 and/or 20 or a variant thereof. Optionally, the antigen-binding protein comprises one or more other polypeptides, e.g., a human Fc (e.g., a human IgG such as an IgG1 or IgG4 (e.g., comprising a S108P mutation)). Antigen-binding proteins that bind to the same epitope on IL2RG as or compete for binding to IL2RG with any of the antigen-binding proteins set forth herein (e.g., H4H12889P or REGN7257), can also be used.

In some embodiments, the antigen-binding protein (e.g., an antibody or antigen-binding fragment thereof) includes an immunoglobulin heavy chain that comprises a V_{H} (e.g., an HC) including the combination of heavy chain CDRs (CDR-H1, CDR-H2 and CDR-H3) set forth in SEQ ID NOS: 4, 6, and 8, respectively, and/or an immunoglobulin light chain that comprises a V_{L} (e.g., a LC) including the combination of light chain CDRs (CDR-L1, CDR-L2 and CDR-L3) set forth in SEQ ID NOS: 12, 14, and 16, respectively.

In some embodiments, the antigen-binding protein (e.g., an antibody or antigen-binding fragment thereof) includes an immunoglobulin heavy and light chain that comprises a V_{H} (e.g., an HC) and a V_{L} (e.g., a LC), respectively, including the combination of heavy and light chain CDRs (CDR-H1, CDR-H2 and CDR-H3; and CDR-L1, CDR-L2 and CDR-L3) set forth in SEQ ID NOS: 4, 6, 8, 12, 14, and 16, respectively.

In some embodiments, the antigen-binding protein (e.g., an antibody or antigen-binding fragment thereof) comprises polypeptide pairs that comprise the following V_{H} and V_{L} amino acid sequences: SEQ ID NOS: 2 and 10.

In some embodiments, the antigen-binding protein (e.g., an antibody or antigen-binding fragment thereof) comprises the following amino acid sequence pairs encoding HC and LC: SEQ ID NOS: 18 and 20.

In some embodiments, the antigen-binding protein (e.g., an antibody or antigen-binding fragment thereof) comprises immunoglobulin V_{H}s and V_{L}s, or HCs and LCs, which comprise a variant amino acid sequence having 70% or more (e.g., 80%, 85%, 90%, 95%, 97% or 99%) overall amino acid sequence identity or similarity to the amino acid sequences of the corresponding V_{H}s, V_{L}s, HCs or LCs specifically set forth herein, but wherein the CDR-L1, CDR-L2, CDR-L3, CDR-H1, CDR-H2 and CDR-H3 of such immunoglobulins are not variants and comprise the amino acid sequences set forth herein. Thus, in such embodiments, the CDRs within variant antigen-binding proteins are not, themselves, variants.

In some embodiments, the antigen-binding protein (e.g., an antibody or antigen-binding fragment thereof) binds to the same epitope as H4H12889P. In some embodiments, the antigen-binding protein (e.g., an antibody or antigen-binding fragment thereof) competes for binding to IL2RG with H4H12889P. The term “competes” as used herein, refers to an antigen-binding protein (e.g., antibody or antigen-binding fragment thereof) that binds to an antigen (e.g., IL2RG) and inhibits or blocks the binding of another antigen-binding protein (e.g., antibody or antigen-binding fragment thereof) to the antigen. Unless otherwise stated, the term also includes competition between two antigen-binding proteins e.g., antibodies, in both orientations, i.e., a first antibody that binds antigen and blocks binding by a second antibody and vice versa. Thus, in some embodiments, competition occurs in one such orientation. In certain embodiments, the first antigen-binding protein (e.g., antibody) and second antigen-binding protein (e.g., antibody) may bind to the same epitope. Alternatively, the first and second antigen-binding proteins (e.g., antibodies) may bind to different, but, for example, overlapping or non-overlapping epitopes, wherein binding of one inhibits or blocks the binding of the second antibody, e.g., via steric hindrance. Competition between antigen-binding proteins (e.g., antibodies) may be measured by methods known in the art, for example, by a real-time, label-free bio-layer interferometry assay. Also, binding competition between anti-IL2RG antigen-binding proteins (e.g., monoclonal antibodies (mAbs)) can be determined using a real time, label-free bio-layer interferometry assay on an Octet RED384 biosensor (Pall ForteBio Corp.).

In some embodiments, the antigen-binding protein is a variant of H4H12889P. Typically, an antibody or antigen-binding fragment which is modified in some way retains the ability to specifically bind to IL2RG, e.g., retains at least 10% of its IL2RG binding activity (when compared to the parental antibody) when that activity is expressed on a molar basis. Preferably, an antibody or antigen-binding fragment retains at least 20%, 50%, 70%, 80%, 90%, 95% or 100% or more of the IL2RG binding affinity as the parental antibody. It is also intended that an antibody or antigen-binding fragment may include conservative or non-conservative amino acid substitutions (referred to as “conservative variants” or “function conserved variants” of the antibody) that do not substantially alter its biologic activity.

A “variant” of such a polypeptide, such as an immunoglobulin chain (e.g., an H4H12889P V_{H}, V_{L}, HC or LC or CDR thereof comprising the amino acid sequence specifically set forth herein), refers to a polypeptide comprising an amino acid sequence that is at least about 70-99.9% (e.g., at least 70, 72, 74, 75, 76, 79, 80, 81, 82, 83, 84, 85, 86, 87, 88, 89, 90, 91, 92, 93, 94, 95, 96, 97, 98, 99, 99.5, or 99.9%) identical or similar to a referenced amino acid sequence that is set forth herein (e.g., any of SEQ ID NOS: 2, 4, 6, 8, 10, 12, 14, 16, 18, or 20); when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment).

Moreover, a variant of a polypeptide may include a polypeptide such as an immunoglobulin chain (e.g., an H4H12889P V_{H}, V_{L}, HC or LC or CDR thereof) which may include the amino acid sequence of the reference polypeptide whose amino acid sequence is specifically set forth herein but for one or more (e.g., 1, 2, 3, 4, 5, 6, 7, 8, 9 or 10) mutations, e.g., one or more missense mutations (e.g., conservative substitutions), non-sense mutations, deletions, or insertions. For example, anti-IL2RG antigen-binding proteins which include an immunoglobulin light chain (or V_{L}) variant comprising the amino acid sequence set forth in SEQ ID NO: 10 but having one or more of such mutations and/or an immunoglobulin heavy chain (or V_{H}) variant comprising the amino acid sequence set forth in SEQ ID NO: 2 but having one or more of such mutations may be used in some embodiments. In some embodiments, an anti-IL2RG antigen-binding protein includes an immunoglobulin light chain variant comprising CDR-L1, CDR-L2 and CDR-L3 wherein one or more (e.g., 1 or 2 or 3) of such CDRs has one or more of such mutations (e.g., conservative substitutions) and/or an immunoglobulin heavy chain variant comprising CDR-H1, CDR-H2 and CDR-H3 wherein one or more (e.g., 1 or 2 or 3) of such CDRs has one or more of such mutations (e.g., conservative substitutions).

The following references relate to BLAST algorithms often used for sequence analysis: BLAST ALGORITHMS: Altschul et al. (2005) FEBS J. 272 (20): 5101-5109; Altschul et al. (1990) J. Mol. Biol. 215:403-410; Gish et al. (1993) Nature Genet. 3:266-272; Madden et al. (1996) Meth. Enzymol. 266:131-141; Altschul et al. (1997) Nucleic Acids Res. 25:3389-3402; Zhang et al. (1997) Genome Res. 7:649-656; Wootton et al. (1993) Comput. Chem. 17:149-163; Hancock et al. (1994) Comput. Appl. Biosci. 10:67-70; ALIGNMENT SCORING SYSTEMS: Dayhoff et al. “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” In Atlas of Protein Sequence and Structure, (1978) vol. 5, suppl. 3.” M. O. Dayhoff (ed.), pp. 353-358, Natl. Biomed. Res. Found., Washington, D.C.; Altschul (1991) J. Mol. Biol. 219:555-565; States et al. (1991) Methods 3:66-70; Henikoff et al. (1992) Proc. Natl. Acad. Sci. U.S.A. 89:10915-10919; Altschul et al. (1993) J. Mol. Evol. 36:290-300; ALIGNMENT STATISTICS: Karlin et al. (1990) Proc. Natl. Acad. Sci. U.S.A. 87:2264-2268; Karlin et al. (1993) Proc. Natl. Acad. Sci. U.S.A. 90:5873-5877; Dembo et al. (1994) Ann. Prob. 22:2022-2039; and Altschul, “Evaluating the statistical significance of multiple distinct local alignments.” in Theoretical and Computational Methods in Genome Research (S. Suhai, ed.), (1997) pp. 1-14, Plenum, N.Y., each of which is herein incorporated by reference in its entirety for all purposes.

A “conservatively modified variant” or a “conservative substitution,” e.g., of an immunoglobulin chain set forth herein, refers to a variant wherein there is one or more substitutions of amino acids in a polypeptide with other amino acids having similar characteristics (e.g., charge, side-chain size, hydrophobicity/hydrophilicity, backbone conformation and rigidity, etc.). Such changes can frequently be made without significantly disrupting the biological activity of the antibody or fragment. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.), herein incorporated by reference in its entirety for all purposes). In addition, substitutions of structurally or functionally similar amino acids are less likely to significantly disrupt biological activity. Anti-IL2RG antigen-binding proteins comprising such conservatively modified variant immunoglobulin chains may be used in some embodiments.

Examples of groups of amino acids that have side chains with similar chemical properties include: (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains: cysteine and methionine. Alternatively, a conservative replacement is any change having a positive value in the PAM250 log-likelihood matrix disclosed in Gonnet et al. (1992) Science 256:1443-45, herein incorporated by reference in its entirety for all purposes.

“H4H12889P” (also called REGN7257), unless otherwise stated, refers to an anti-IL2RG antigen-binding protein (e.g., antibodies and antigen-binding fragments thereof (including multispecific antigen-binding proteins)) comprising an immunoglobulin heavy chain or variable region thereof (V_{H}) comprising the amino acid sequence specifically set forth herein for H4H12889P (e.g., SEQ ID NO: 2 (or a variant thereof)) and/or an immunoglobulin light chain or variable region thereof (V_{L}) comprising the amino acid sequence specifically set forth herein for H4H12889P (e.g., SEQ ID NO: 10 (or a variant thereof)), respectively; and/or that comprise a heavy chain or V_{H} that comprises the CDRs thereof (CDR-H1 (or a variant thereof), CDR-H2 (or a variant thereof) and CDR-H3 (or a variant thereof)) and/or a light chain or V_{L} that comprises the CDRs thereof (CDR-L1 (or a variant thereof), CDR-L2 (or a variant thereof) and CDR-L3 (or a variant thereof)). In some embodiments, the V_{H} is linked to an IgG constant heavy chain domain, for example, human IgG constant heavy chain domain (e.g., IgG1 or IgG4 (e.g., comprising the S228P and/or S108P mutation)) and/or the V_{L}, is linked to a light chain constant domain, for example a human light chain constant domain (e.g., lambda or kappa constant light chain domain). In some embodiments, polynucleotides encoding one or more of any such immunoglobulin chains (e.g., V_{H}, V_{L}, HC and/or LC) are provided.

In some embodiments, the antigen-binding protein (e.g., antibodies and antigen-binding fragments thereof (e.g., H4H12889P)) comprises immunoglobulin chains including the amino acid sequences specifically set forth herein (and variants thereof) as well as cellular and in vitro post-translational modifications to the antibody or fragment. For example, the antigen-binding proteins include antibodies and antigen-binding fragments thereof that specifically bind to IL2RG comprising heavy and/or light chain amino acid sequences set forth herein as well as antibodies and fragments wherein one or more asparagine, serine and/or threonine residues is glycosylated, one or more asparagine residues is deamidated, one or more residues (e.g., Met, Trp and/or His) is oxidized, the N-terminal glutamine is pyroglutamate (pyroE) and/or the C-terminal lysine or other amino acid is missing.

In some embodiments, nucleic acid(s) encoding antigen-binding proteins are provided. A nucleic acid encoding an antigen-binding protein comprises deoxyribonucleic acid (DNA) or ribonucleic acid (RNA). Such nucleic acids can be DNA, RNA, or hybrids or derivatives of either DNA or RNA. Optionally, in some embodiments, the nucleic acid can be codon-optimized for efficient translation into protein in a particular cell or organism. As a non-limiting example, the nucleic acid can be modified to substitute codons having a higher frequency of usage in a human cell, a mammalian cell, a rodent cell, a mouse cell, a rat cell, or any other host cell of interest, as compared to the naturally occurring polynucleotide sequence. Any portion or fragment of a nucleic acid molecule can be produced by: (1) isolating the molecule from its natural milieu; (2) using recombinant DNA technology (e.g., but not limited to, PCR amplification or cloning); or (3) using chemical synthesis methods. Nucleic acids can comprise modifications for improved stability or reduced immunogenicity. Non-limiting examples of modifications include: (1) alteration or replacement of one or both of the non-linking phosphate oxygens and/or of one or more of the linking phosphate oxygens in the phosphodiester backbone linkage; (2) alteration or replacement of a constituent of a ribose sugar such as alteration or replacement of the 2′ hydroxyl on the ribose sugar; (3) replacement of the phosphate moiety with dephospho linkers; (4) modification or replacement of a naturally occurring nucleobase; (5) replacement or modification of a ribose-phosphate backbone; (6) modification of the 3′ end or 5′ end of the oligonucleotide (e.g., but not limited to, removal, modification or replacement of a terminal phosphate group or conjugation of a moiety); and (7) modification of the sugar.

Such nucleic acids can include any polynucleotide, for example, encoding an immunoglobulin V_{H}, V_{L}, CDR-H, CDR-L, HC, or LC of H4H12889P; optionally, which is operably linked to a promoter or other expression control sequence. For example, such nucleic acids can include any polynucleotide (e.g., DNA) that includes a nucleotide sequence set forth in SEQ ID NO: 1, 3, 5, 7, 9, 11, 13, 15, 17, or 19. In some embodiments, a polynucleotide of interest is fused to a secretion signal sequence.

In some embodiments, the nucleic acids can be in the form of an expression construct as defined elsewhere herein. As a non-limiting example, the nucleic acids can include regulatory regions that control expression of the nucleic acid molecule (e.g., but not limited to, transcription or translation control regions), full-length or partial coding regions, and combinations thereof. As a non-limiting example, the nucleic acids can be operably linked to a promoter active in a cell or organism of interest. Promoters that can be used in such expression constructs include promoters active, for example, in one or more of a eukaryotic cell, such as a mammalian cell (e.g., a non-human mammalian cell or a human cell), such as a rodent cell (e.g., but not limited to, a mouse cell, or a rat cell). Such promoters can be, for example, conditional promoters, inducible promoters, constitutive promoters, or tissue-specific promoters.

In general, a “promoter” or “promoter sequence” is a DNA regulatory region capable of binding an RNA polymerase in a cell (e.g., directly or through other promoter-bound proteins or substances) and initiating transcription of a coding sequence. A promoter may be operably linked to other expression control sequences, including enhancer and repressor sequences and/or with a polynucleotide encoding an antigen-binding protein (e.g., an antibody or antigen binding fragment thereof). Promoters which may be used to control gene expression include, but are not limited to, cytomegalovirus (CMV) promoter (U.S. Pat. Nos. 5,385,839 and 5,168,062, each of which is herein incorporated by reference in its entirety for all purposes), the SV40 early promoter region (Benoist et al. (1981) Nature 290:304-310, herein incorporated by reference in its entirety for all purposes), the promoter contained in the 3′ long terminal repeat of Rous sarcoma virus (Yamamoto et al. (1980) Cell 22:787-797, herein incorporated by reference in its entirety for all purposes), the herpes thymidine kinase promoter (Wagner et al. (1981) Proc. Natl. Acad. Sci. U.S.A. 78:1441-1445, herein incorporated by reference in its entirety for all purposes), the regulatory sequences of the metallothionein gene (Brinster et al. (1982) Nature 296:39-42, herein incorporated by reference in its entirety for all purposes); prokaryotic expression vectors such as the beta-lactamase promoter (VIlla-Komaroff et al. (1978) Proc. Natl. Acad. Sci. U.S.A. 75:3727-3731, herein incorporated by reference in its entirety for all purposes), or the tac promoter (DeBoer et al. (1983) Proc. Natl. Acad. Sci. U.S.A. 80:21-25; see also “Useful proteins from recombinant bacteria” in Scientific American (1980) 242:74-94, each of which is herein incorporated by reference in its entirety for all purposes); and promoter elements from yeast or other fungi such as the Gal4 promoter, the ADC (alcohol dehydrogenase) promoter, PGK (phosphoglycerol kinase) promoter or the alkaline phosphatase promoter.

A polynucleotide encoding a polypeptide is “operably linked” to a promoter or other expression control sequence when, in a cell or other expression system, the sequence directs RNA polymerase mediated transcription of the coding sequence into RNA, preferably mRNA, which then may be RNA spliced (if it contains introns) and, optionally, translated into a protein encoded by the coding sequence.

In some embodiments, the nucleic acid(s) comprise the following polynucleotide pair encoding a V_{H} and V_{L}: SEQ ID NO: 1 and SEQ ID NO: 9. In some embodiments, the nucleic acid(s) comprise the following polynucleotide set which encode a CDR-H1, CDR-H2, CDR-H3, CDR-L1, CDR-L2 and CDR-L3: SEQ ID NOS: 3, 5, 7, 11, 13, and 15. In some embodiments, the nucleic acid(s) comprise the following polynucleotide pair encoding a HC and LC: SEQ ID NOS: 17 and 19. In some embodiments, the nucleic acid(s) include polynucleotides encoding immunoglobulin polypeptide chains which are variants of those whose nucleotide sequence is specifically set forth herein. A “variant” of such a polynucleotide or nucleic acids refers to a polynucleotide or nucleic acid comprising a nucleotide sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical to a referenced nucleotide sequence that is set forth herein; when the comparison is performed by a BLAST algorithm wherein the parameters of the algorithm are selected to give the largest match between the respective sequences over the entire length of the respective reference sequences (e.g., expect threshold: 10; word size: 28; max matches in a query range: 0; match/mismatch scores: 1, −2; gap costs: linear). In some embodiments, a variant of a nucleotide sequence specifically set forth herein comprises one or more (e.g., 1, 2, 3, 4, 5, 6, 7, 8, 9, 10, 11 or 12) point mutations, insertions (e.g., in frame insertions) or deletions (e.g., in frame deletions) of one or more nucleotides. Such mutations may, in some embodiments, be missense or nonsense mutations. In some embodiments, such a variant polynucleotide encodes an immunoglobulin polypeptide chain which can be incorporated into an anti-IL2RG antigen-binding protein, i.e., such that the protein retains specific binding to IL2RG.

In some embodiments, the antigen-binding protein is an anti-IL2RG antibody or antigen-binding fragment thereof. In some embodiments, the antigen-binding protein comprises an immunoglobulin light chain or variable region thereof comprising three light chain CDRs and an immunoglobulin heavy chain or variable region thereof comprising three heavy chain CDRs, wherein the three light chain CDRs comprise sequences at least 90% identical to the sequences set forth in SEQ ID NOS: 12, 14, and 16, respectively, and wherein the three heavy chain CDRs comprise sequences at least 90% identical to the sequences set forth in SEQ ID NOS: 4, 6, and 8, respectively. In some embodiments, the antigen-binding protein comprises an immunoglobulin light chain or variable region thereof comprising three light chain CDRs and an immunoglobulin heavy chain or variable region thereof comprising three heavy chain CDRs, wherein the three light chain CDRs consist essentially of sequences at least 90% identical to the sequences set forth in SEQ ID NOS: 12, 14, and 16, respectively, and wherein the three heavy chain CDRs consist essentially of sequences at least 90% identical to the sequences set forth in SEQ ID NOS: 4, 6, and 8, respectively. In some embodiments, the antigen-binding protein comprises an immunoglobulin light chain or variable region thereof comprising three light chain CDRs and an immunoglobulin heavy chain or variable region thereof comprising three heavy chain CDRs, wherein the three light chain CDRs consist of sequences at least 90% identical to the sequences set forth in SEQ ID NOS: 12, 14, and 16, respectively, and wherein the three heavy chain CDRs consist of sequences at least 90% identical to the sequences set forth in SEQ ID NOS: 4, 6, and 8, respectively.

In some embodiments, the three light chain CDRs comprise the sequences set forth in SEQ ID NOS: 12, 14, and 16, respectively, and the three heavy chain CDRs comprise the sequences set forth in SEQ ID NOS: 4, 6, and 8, respectively. In some embodiments, the three light chain CDRs consist essentially of the sequences set forth in SEQ ID NOS: 12, 14, and 16, respectively, and the three heavy chain CDRs consist essentially of the sequences set forth in SEQ ID NOS: 4, 6, and 8, respectively. In some embodiments, the three light chain CDRs consist of the sequences set forth in SEQ ID NOS: 12, 14, and 16, respectively, and the three heavy chain CDRs consist of the sequences set forth in SEQ ID NOS: 4, 6, and 8, respectively.

In some embodiments, the immunoglobulin light chain or variable region thereof comprises a sequence at least 90% identical to the sequence set forth in SEQ ID NO: 10, and the immunoglobulin heavy chain or variable region thereof comprises a sequence at least 90% identical to the sequence set forth in SEQ ID NO: 2. In some embodiments, the immunoglobulin light chain or variable region thereof consists essentially of a sequence at least 90% identical to the sequence set forth in SEQ ID NO: 10, and the immunoglobulin heavy chain or variable region thereof consists essentially of a sequence at least 90% identical to the sequence set forth in SEQ ID NO: 2. In some embodiments, the immunoglobulin light chain or variable region thereof consists of a sequence at least 90% identical to the sequence set forth in SEQ ID NO: 10, and the immunoglobulin heavy chain or variable region thereof consists of a sequence at least 90% identical to the sequence set forth in SEQ ID NO: 2.

In some embodiments, the immunoglobulin light chain or variable region thereof comprises the sequence set forth in SEQ ID NO: 10, and the immunoglobulin heavy chain or variable region thereof comprises the sequence set forth in SEQ ID NO: 2. In some embodiments, the immunoglobulin light chain or variable region thereof consists essentially of the sequence set forth in SEQ ID NO: 10, and the immunoglobulin heavy chain or variable region thereof consists essentially of the sequence set forth in SEQ ID NO: 2. In some embodiments, the immunoglobulin light chain or variable region thereof consists of the sequence set forth in SEQ ID NO: 10, and the immunoglobulin heavy chain or variable region thereof consists of the sequence set forth in SEQ ID NO: 2.

In some embodiments, the immunoglobulin light chain comprises a sequence at least 90% identical to the sequence set forth in SEQ ID NO: 20, and the immunoglobulin heavy chain comprises a sequence at least 90% identical to the sequence set forth in SEQ ID NO: 18. In some embodiments, the immunoglobulin light chain consists essentially of a sequence at least 90% identical to the sequence set forth in SEQ ID NO: 20, and the immunoglobulin heavy chain consists essentially of a sequence at least 90% identical to the sequence set forth in SEQ ID NO: 18. In some embodiments, the immunoglobulin light chain consists of a sequence at least 90% identical to the sequence set forth in SEQ ID NO: 20, and the immunoglobulin heavy chain consists of a sequence at least 90% identical to the sequence set forth in SEQ ID NO: 18.

In some embodiments, the immunoglobulin light chain comprises the sequence set forth in SEQ ID NO: 20, and the immunoglobulin heavy chain comprises the sequence set forth in SEQ ID NO: 18. In some embodiments, the immunoglobulin light chain consists essentially of the sequence set forth in SEQ ID NO: 20, and the immunoglobulin heavy chain consists essentially of the sequence set forth in SEQ ID NO: 18. In some embodiments, the immunoglobulin light chain consists of the sequence set forth in SEQ ID NO: 20, and the immunoglobulin heavy chain consists of the sequence set forth in SEQ ID NO: 18.

The agent for selective inhibition or selective depletion of host cells or non-edited cells can be administered to the subject by any suitable means. The term administering refers to administration of a composition to a subject or system (e.g., but not limited to, to a cell, organ, tissue, organism, or relevant component or set of components thereof). The route of administration may vary depending, for example, on the subject or system to which the composition is being administered, the nature of the composition, the purpose of the administration, and so forth. The term “administration” or “administering” is intended to include routes of introducing the agent to a subject to perform its intended function. In some embodiments, non-limiting examples of routes of administration which can be used include injection (subcutaneous, intravenous, parenterally, intraperitoneally, intrathecal), oral, inhalation, rectal and transdermal. As non-limiting examples, administration to a subject (e.g., but not limited to, to a human or a rodent) may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and/or vitreal. Agents can be administered in tablets or capsule form (e.g., but not limited to, by injection, inhalation, eye lotion, ointment, suppository, and so forth), topically by lotion or ointment, or rectally by suppositories. Administration can be in a bolus or can be by continuous infusion. Administration may involve intermittent dosing or continuous dosing (e.g., but not limited to, perfusion) for at least a selected period of time. Depending on the route of administration, the agent can be coated with or disposed in a selected material to protect it from natural conditions which may detrimentally affect its ability to perform its intended function. The agent can be administered alone, or in conjunction with either another agent (e.g., but not limited to, the donor cells or edited cells described herein) or with a pharmaceutically acceptable carrier, or both. The agent can be administered prior to the administration of the other agent, simultaneously with the agent, or after the administration of the agent. Furthermore, the agent can also be administered in a proform which is converted into its active metabolite, or more active metabolite in vivo.

In some embodiments of the present invention, a subject can include, for example, any type of animal or mammal. Mammals include, for example, humans, non-human mammals, non-human primates, monkeys, apes, cats, dogs, horses, bulls, deer, bison, sheep, rabbits, rodents (e.g., but not limited to, mice, rats, hamsters, and guinea pigs), and livestock (e.g., but not limited to, bovine species such as cows and steer; ovine species such as sheep and goats; and porcine species such as pigs and boars). Birds include, for example, chickens, turkeys, ostrich, geese, and ducks. Domesticated animals and agricultural animals are also included. The term “non-human mammal” excludes humans. Particular non-limiting examples of non-human mammals include rodents, such as mice and rats. In some embodiments of the present invention, the subject is a human.

In some embodiments of the present invention, any of the methods for improving engraftment of donor cells or for selective inhibition or selective depletion of host cells or non-edited cells in a subject described herein can further comprise generating the donor cells or edited cells by modifying a population of cells to express the first isoform of the target protein (e.g., IL2RG protein). Suitable methods and reagents for generating donor cells or edited cells are described in more detail elsewhere herein. In some embodiments of the present invention, any of the methods for improving engraftment of donor cells or for selective inhibition or selective depletion of host cells or non-edited cells in a subject described herein can further comprise isolating a population of cells from the subject (or from a different subject) prior to modifying the population of cells. In some embodiments of the present invention, any of the methods for improving engraftment of donor cells or for selective inhibition or selective depletion of host cells or non-edited cells in a subject described herein can further comprise generating the donor cells or edited cells by editing a target genomic locus (e.g., IL2RG locus) in a population of cells to express the first isoform of the target protein (e.g., IL2RG protein). Suitable methods and reagents for editing a target genomic locus are described in more detail elsewhere herein. In some embodiments, the genomic locus is an endogenous genomic locus encoding the target protein (e.g., the target protein is IL2RG, and the genomic locus is an IL2RG genomic locus). In some embodiments, the genomic locus is not an endogenous genomic locus encoding the target protein (e.g., the target protein is IL2RG, and the genomic locus is not an IL2RG genomic locus). In some embodiments of the present invention, any of the methods for improving engraftment of donor cells or for selective inhibition or selective depletion of host cells or non-edited cells in a subject described herein can further comprise isolating a population of cells from the subject (or from a different subject) prior to editing the target genomic locus in the population of cells. The isolated cells can be, for example, any suitable cells. In some embodiments, the cells are immune cells. In some embodiments, the cells are hematopoietic cells. In some embodiments, the cells are lymphocytes or lymphoid progenitor cells. In some embodiments, the cells are T cells (e.g., CD4+ T cells, CD8+ T cells, memory T cells, regulatory T cells, gamma delta T cells, mucosal-associated invariant T cells (MAIT), tumor infiltrating lymphocytes (TILs), or any combination thereof). In some embodiments, the cells are TILs. In some embodiments, the cells are B cells. In some embodiments, the cells are natural killer (NK) cells. In some embodiments, the cells are innate lymphoid cells. In some embodiments, the cells are dendritic cells. In some embodiments, the cells are hematopoietic stem cells (HSCs) or hematopoietic stem and progenitor cells (HSPCs) or descendants thereof. HSCs are capable of giving rise to both myeloid and lymphoid progenitor cells that further give rise to myeloid cells (e.g., monocytes, macrophages, neutrophils, basophils, dendritic cells, erythrocytes, platelets, etc.) and lymphoid cells (e.g., T cells, B cells, NK cells), respectively. In some embodiments, the cells are autologous (i.e., from the subject). In some embodiments, the cells are allogeneic (i.e., not from the subject) or syngeneic (i.e., genetically identical, or sufficiently identical and immunologically compatible as to allow for transplantation). In some embodiments, the cells are mammalian cells or non-human mammalian cells (e.g., mouse or rat cells or non-human primate cells) (e.g., the subject is a mammal or a non-human mammal, and the cells are mammalian cells or non-human mammalian cells). In some embodiments, the cells are human cells (e.g., the subject is a human, and the cells are human cells.

In some embodiments of the present invention, any of the methods for improving engraftment of donor cells or for selective inhibition or selective depletion of host cells or non-edited cells in a subject described herein can further comprise generating the donor cells or edited cells by modifying a population of induced pluripotent stem cells (e.g., human induced pluripotent stem cells (iPSCs)) to express the first isoform of the target protein (e.g., IL2RG protein) and then differentiating the induced pluripotent cells into a different cell type prior to administration to the subject. In some embodiments of the present invention, any of the methods for improving engraftment of donor cells or for selective inhibition or selective depletion of host cells or non-edited cells in a subject described herein can further comprise generating the donor cells or edited cells by editing a target genomic locus (e.g., IL2RG locus) in a population of induced pluripotent stem cells (e.g., human induced pluripotent stem cells (iPSCs)) to express the first isoform of the target protein (e.g., IL2RG protein) and then differentiating the induced pluripotent cells into a different cell type prior to administration to the subject. For example, the induced pluripotent stem cells can be differentiated into any suitable cells. In some embodiments, the cells are immune cells. In some embodiments, the cells are hematopoietic cells. In some embodiments, the cells are lymphocytes or lymphoid progenitor cells. In some embodiments, the cells are T cells (e.g., CD4+ T cells, CD8+ T cells, memory T cells, regulatory T cells, gamma delta T cells, mucosal-associated invariant T cells (MAIT), tumor infiltrating lymphocytes (TILs), or any combination thereof). In some embodiments, the cells are TILs. In some embodiments, the cells are B cells. In some embodiments, the cells are natural killer (NK) cells. In some embodiments, the cells are innate lymphoid cells. In some embodiments, the cells are dendritic cells. In some embodiments, the cells are hematopoietic stem cells (HSCs) or hematopoietic stem and progenitor cells (HSPCs) or descendants thereof. HSC refers to the true stem cells that give rise to all blood and immune lineages. HPSCs include HSCs but also more differentiated progenitors that give rise to more restricted lineages. For instance, some HSPCs might only be able to develop to myeloid lineages, or lymphoid, or erythroid, and so forth. HSCs are capable of giving rise to both myeloid and lymphoid progenitor cells that further give rise to myeloid cells (e.g., monocytes, macrophages, neutrophils, basophils, dendritic cells, erythrocytes, platelets, etc.) and lymphoid cells (e.g., T cells, B cells, NK cells), respectively. In some embodiments, the donor cells or edited cells are autologous (i.e., from the subject). In some embodiments, the donor cells or edited cells are allogeneic (i.e., not from the subject) or syngeneic (i.e., genetically identical, or sufficiently identical and immunologically compatible as to allow for transplantation). In some embodiments, the cells are mammalian cells or non-human mammalian cells (e.g., mouse or rat cells or non-human primate cells) (e.g., the subject is a mammal or a non-human mammal, and the cells are mammalian cells or non-human mammalian cells). In some embodiments, the cells are human cells (e.g., the subject is a human, and the cells are human cells.

In some embodiments of the present invention, any of the methods for improving engraftment of donor cells or for selective inhibition or selective depletion of host cells or non-edited cells in a subject described herein can further comprise generating the donor cells or edited cells by modifying a population of hematopoietic stem cells (HSCs) or hematopoietic stem and progenitor cells (HSPCs) (e.g., human HSCs or HSPCs) to express the first isoform of the target protein (e.g., IL2RG protein) and then differentiating the HSCs or HSPCs into a different cell type prior to administration to the subject. In some embodiments of the present invention, any of the methods for improving engraftment of donor cells or for selective inhibition or selective depletion of host cells or non-edited cells in a subject described herein can further comprise generating the donor cells or edited cells by editing a target genomic locus (e.g., IL2RG locus) in a population of hematopoietic stem cells (HSCs) or hematopoietic stem and progenitor cells (HSPCs) (e.g., human HSCs or HSPCs) to express the first isoform of the target protein (e.g., IL2RG protein) and then differentiating the HSCs or HSPCs into a different cell type prior to administration to the subject. For example, the HSCs or HSPCs can be differentiated into any suitable cells. In some embodiments, the cells are immune cells. In some embodiments, the cells are hematopoietic cells. In some embodiments, the cells are lymphocytes or lymphoid progenitor cells. In some embodiments, the cells are T cells (e.g., CD4+ T cells, CD8+ T cells, memory T cells, regulatory T cells, gamma delta T cells, mucosal-associated invariant T cells (MAIT), tumor infiltrating lymphocytes (TILs), or any combination thereof). In some embodiments, the cells are TILs. In some embodiments, the cells are B cells. In some embodiments, the cells are natural killer (NK) cells. In some embodiments, the cells are innate lymphoid cells. In some embodiments, the cells are dendritic cells. In some embodiments, the donor cells or edited cells are autologous (i.e., from the subject). In some embodiments, the donor cells or edited cells are allogeneic (i.e., not from the subject) or syngeneic (i.e., genetically identical, or sufficiently identical and immunologically compatible as to allow for transplantation). In some embodiments, the cells are mammalian cells or non-human mammalian cells (e.g., mouse or rat cells or non-human primate cells) (e.g., the subject is a mammal or a non-human mammal, and the cells are mammalian cells or non-human mammalian cells). In some embodiments, the cells are human cells (e.g., the subject is a human, and the cells are human cells.

### III. Methods for Generating Donor Cells or Edited Cells

In some embodiments of the present invention, any of the methods for improving engraftment of donor cells or for selective inhibition or selective depletion of host cells or non-edited cells in a subject described herein can further comprise generating the donor cells or edited cells. The donor cells or edited cells can be generated by modifying a population of cells to express the first isoform of the target protein (e.g., IL2RG protein). In some embodiments of the present invention, any of the methods for improving engraftment of donor cells or for selective inhibition or selective depletion of host cells or non-edited cells in a subject described herein can further comprise generating the donor cells or edited cells by editing a target genomic locus (e.g., IL2RG locus) in a population of cells to express the first isoform of the target protein (e.g., IL2RG protein). The donor cells or edited cells can express only the first isoform, or they can express both the first and second isoforms of the target protein (e.g., being modified to express the first isoform of the target protein but retaining expression of the second isoform of the target protein).

In some embodiments, generating donor cells or edited cells can comprise introducing an expression vector into a population of cells, wherein the expression vector expresses the first isoform of the target protein (e.g., IL2RG protein). Such expression vectors can comprise the entire coding sequence for the first isoform of the target protein (e.g., IL2RG), operably linked to a promoter suitable for driving expression in the donor cells or edited cells. Any suitable promoter can be used. In one example, a promoter specific for or active in hematopoietic cells or a subset of hematopoietic cells can be used. In another example, a constitutive promoter can be used. Examples of such promoters include human cytomegalovirus (hCMV), chicken beta-actin/CMV enhancer (CAG), and elongation factor-1 alpha (EF1alpha). As another example, an inducible promoter can be used. In some embodiments, the expression vector can be a bicistronic expression vector encoding the therapeutic molecule and the first isoform of the target protein (e.g., IL2RG) and a therapeutic molecule (e.g., a CAR) as described elsewhere herein. See, e.g., Yeku et al. (2017) Sci. Rep. 7 (1): 10541 and Rafiq et al. (2018) Nat. Biotechnol. 36 (9): 847-856, each of which is herein incorporated by reference in its entirety for all purposes, for examples of bicistronic constructs expressing CARs and another molecule. Any suitable vector can be used. For example, the vector can be a viral vector, such as a lentiviral vector or an adeno-associated virus (AAV) vector. In some embodiments, a lentiviral vector is used. In some embodiments, an AAV vector is used, such as an AAV vector with a serotype for expression in hematopoietic cells (e.g., AAV6). Optionally, the endogenous locus encoding the second isoform of the target protein can also be modified (e.g., disrupted) so that the first isoform is expressed but the second isoform is not. As one example, the endogenous locus can be modified to comprise an insertion, a deletion, or one or more point mutations in the endogenous locus (e.g., IL2RG locus) resulting in loss of expression of functional target protein (e.g., IL2RG). Such loci can comprise a deletion or disruption of all of the endogenous coding sequence or can comprise a deletion or disruption of a fragment of (i.e., a part of or portion of) the endogenous locus. In one example, a 5′ fragment of the coding sequence can be deleted or disrupted (e.g., including the start codon). As one example, the endogenous locus can be modified such that the start codon of the endogenous locus has been deleted or has been disrupted or mutated such that the start codon is no longer functional. For example, the start codon can be disrupted by a deletion or insertion within the start codon. Alternatively, the start codon can be mutated by, for example, by a substitution of one or more nucleotides. In another example, a 3′ fragment of the endogenous locus can be deleted or disrupted (e.g., including the stop codon). In another example, an internal fragment of the endogenous locus can be deleted or disrupted. In another example, all of the coding sequence in the endogenous locus can be deleted or disrupted. Alternatively, the endogenous locus can remain unmodified, and both the first and second isoforms are expressed.

In some embodiments, generating donor cells or edited cells can comprise editing a genomic locus in a population of cells to express the first isoform of the target protein (e.g., IL2RG protein). The genomic locus can be the endogenous locus encoding the target protein, it can be a safe harbor locus, or it can be a random genomic locus targeted by random integration. Safe harbor loci include chromosomal loci where transgenes or other exogenous nucleic acid inserts can be stably and reliably expressed in all tissues of interest without overtly altering cell behavior or phenotype (i.e., without any deleterious effects on the host cell). See, e.g., Sadelain et al. (2012) Nat. Rev. Cancer 12:51-58, herein incorporated by reference in its entirety for all purposes. For example, the safe harbor locus can be one in which expression of the inserted gene sequence is not perturbed by any read-through expression from neighboring genes. For example, safe harbor loci can include chromosomal loci where exogenous DNA can integrate and function in a predictable manner without adversely affecting endogenous gene structure or expression. Safe harbor loci can include extragenic regions or intragenic regions such as, for example, loci within genes that are non-essential, dispensable, or able to be disrupted without overt phenotypic consequences. Such safe harbor loci can offer an open chromatin configuration in all tissues and can be ubiquitously expressed during embryonic development and in adults. See, e.g., Zambrowicz et al. (1997) Proc. Natl. Acad. Sci. U.S.A. 94:3789-3794, herein incorporated by reference in its entirety for all purposes. In addition, the safe harbor loci can be targeted with high efficiency, and safe harbor loci can be disrupted with no overt phenotype. Examples of safe harbor loci include albumin, CCR5, HPRT, AAVS1, and Rosa26. See, e.g., U.S. Pat. Nos. 7,888,121; 7,972,854; 7,914,796; 7,951,925; 8,110,379; 8,409,861; 8,586,526; and US Patent Publication Nos. 2003/0232410; 2005/0208489; 2005/0026157; 2006/0063231; 2008/0159996; 2010/00218264; 2012/0017290; 2011/0265198; 2013/0137104; 2013/0122591; 2013/0177983; 2013/0177960; and 2013/0122591, each of which is herein incorporated by reference in its entirety for all purposes. In some embodiments, the genomic locus is an endogenous genomic locus encoding the target protein (e.g., the target protein is IL2RG, and the genomic locus is an IL2RG genomic locus). In some embodiments, the genomic locus is not an endogenous genomic locus encoding the target protein (e.g., the target protein is IL2RG, and the genomic locus is not an IL2RG genomic locus). The coding sequence for the first isoform of the target protein (e.g., IL2RG) can be operably linked to a promoter suitable for driving expression in the donor cells or edited cells. Optionally, the endogenous locus encoding the second isoform of the target protein can also be modified (e.g., disrupted) so that the first isoform is expressed but the second isoform is not. As one example, the endogenous locus can be modified to comprise an insertion, a deletion, or one or more point mutations in the endogenous locus (e.g., IL2RG locus) resulting in loss of expression of functional target protein (e.g., IL2RG). Such loci can comprise a deletion or disruption of all of the endogenous coding sequence or can comprise a deletion or disruption of a fragment of (i.e., a part of or portion of) the endogenous locus. In one example, a 5′ fragment of the coding sequence can be deleted or disrupted (e.g., including the start codon). As one example, the endogenous locus can be modified such that the start codon of the endogenous locus has been deleted or has been disrupted or mutated such that the start codon is no longer functional. For example, the start codon can be disrupted by a deletion or insertion within the start codon. Alternatively, the start codon can be mutated by, for example, by a substitution of one or more nucleotides. In another example, a 3′ fragment of the endogenous locus can be deleted or disrupted (e.g., including the stop codon). In another example, an internal fragment of the endogenous locus can be deleted or disrupted. In another example, all of the coding sequence in the endogenous locus can be deleted or disrupted. Alternatively, the endogenous locus can remain unmodified, and both the first and second isoforms are expressed.

In some embodiments, generated donor cells or edited cells can comprise editing a target genomic locus (e.g., IL2RG locus) in a population of cells to express the first isoform of the target protein (e.g., IL2RG protein).

In some embodiments, the editing can comprise introducing into the population of cells (1) a nuclease agent or one or more nucleic acids encoding the nuclease agent, wherein the nuclease agent targets a nuclease target sequence in the target genomic locus and (2) an exogenous donor nucleic acid. The nuclease can cleave the target genomic locus, and the exogenous donor nucleic acid can be inserted into the target genomic locus or can recombine with the target genomic locus to generate the donor cells or edited cells that express the first isoform of the target protein. However, the skilled person is aware that alternative methods can also be used. For example, in some embodiments, the isoform switch can be effected using base editors. See, e.g., Komor et al. (2016) Nature 533 (7603): 420-424, herein incorporated by reference in its entirety for all purposes. This approach allows editing of the desired amino acid without the need for a double-stranded DNA break.

Any suitable nuclease agent can be used. In some embodiments, for example, the methods can utilize nuclease agents such as Clustered Regularly Interspersed Short Palindromic Repeats (CRISPR)/CRISPR-associated (Cas) systems, zinc finger nuclease (ZFN) systems, or Transcription Activator-Like Effector Nuclease (TALEN) systems or components of such systems to modify a target genomic locus (e.g., an IL2RG gene such as a human IL2RG gene). Generally, the nuclease agents involve the use of engineered cleavage systems to induce a double strand break or a nick (i.e., a single strand break) in a nuclease target site. Cleavage or nicking can occur through the use of specific nucleases such as engineered ZFNs, TALENs, or CRISPR/Cas systems with an engineered guide RNA to guide specific cleavage or nicking of the nuclease target site. Any nuclease agent that induces a nick or double-strand break at a desired target sequence can be used in the methods and compositions disclosed herein. The nuclease agent can be used to create a targeted genetic modification in the IL2RG gene (e.g., human IL2RG gene). For example, the targeted genetic modification in some embodiments can comprise a targeted genetic modification in exon 3 of IL2RG (e.g., exon 3 of human IL2RG). In some embodiments, the targeted genetic modification is in exon 3 of human IL2RG. For example, the targeted genetic modification in some embodiments can comprise a targeted genetic modification in exon 2 of IL2RG (e.g., exon 2 of human IL2RG). In some embodiments, the targeted genetic modification is in exon 2 of human IL2RG. For example, the targeted genetic modification in some embodiments can comprise a targeted genetic modification in exons 2 and 3 of IL2RG (e.g., exons 2 and 3 of human IL2RG). In some embodiments, the targeted genetic modification is in exons 2 and 3 of human IL2RG.

In some embodiments, the nuclease agent is a CRISPR/Cas system. In some embodiments, the nuclease agent comprises one or more ZFNs. In some embodiments, the nuclease agent comprises one or more TALENs. CRISPR/Cas systems include transcripts and other elements involved in the expression of, or directing the activity of, Cas genes. A CRISPR/Cas system can be, for example, a type I, a type II, a type III system, or a type V system (e.g., subtype V-A or subtype V-B). The methods and compositions disclosed herein can employ CRISPR/Cas systems by utilizing CRISPR complexes (comprising a guide RNA (gRNA) complexed with a Cas protein) for site-directed binding or cleavage of nucleic acids. A CRISPR/Cas system targeting a target genomic locus comprises a Cas protein (or a nucleic acid encoding the Cas protein) and one or more guide RNAs (or DNAs encoding the one or more guide RNAs), with each of the one or more guide RNAs targeting a different guide RNA target sequence in the target genomic locus.

CRISPR/Cas systems used in the compositions and methods disclosed herein can be non-naturally occurring. A non-naturally occurring system includes anything indicating the involvement of the hand of man, such as one or more components of the system being altered or mutated from their naturally occurring state, being at least substantially free from at least one other component with which they are naturally associated in nature, or being associated with at least one other component with which they are not naturally associated. For example, some CRISPR/Cas systems employ non-naturally occurring CRISPR complexes comprising a gRNA and a Cas protein that do not naturally occur together, employ a Cas protein that does not occur naturally, or employ a gRNA that does not occur naturally.

The nuclease agents and CRISPR/Cas systems described in the compositions and methods disclosed herein target a nuclease target sequence (e.g., a guide RNA target sequence) in a target genomic locus encoding a target protein. In some embodiments, the nuclease target sequence is in an IL2RG gene. In some embodiments, the nuclease target sequence is in a human IL2RG gene. In some embodiments, the nuclease target sequence is in exon 3 of an IL2RG gene. In some embodiments, the nuclease target sequence is in exon 3 of a human IL2RG gene. In some embodiments, the nuclease target sequence is in exon 2 of an IL2RG gene. In some embodiments, the nuclease target sequence is in exon 2 of a human IL2RG gene. In some embodiments, the nuclease target sequence is in exons 2 and 3 of an IL2RG gene. In some embodiments, the nuclease target sequence is in exons 2 and 3 of a human IL2RG gene.

Cas Proteins. Cas proteins generally comprise at least one RNA recognition or binding domain that can interact with guide RNAs. Cas proteins can also comprise nuclease domains (e.g., DNase domains or RNase domains), DNA-binding domains, helicase domains, protein-protein interaction domains, dimerization domains, and other domains. Some such domains (e.g., DNase domains) can be from a native Cas protein. Other such domains can be added to make a modified Cas protein. A nuclease domain possesses catalytic activity for nucleic acid cleavage, which includes the breakage of the covalent bonds of a nucleic acid molecule. Cleavage can produce blunt ends or staggered ends, and it can be single-stranded or double-stranded. For example, a wild type Cas9 protein will typically create a blunt cleavage product. Alternatively, a wild type Cpf1 protein (e.g., FnCpf1) can result in a cleavage product with a 5-nucleotide 5′ overhang, with the cleavage occurring after the 18th base pair from the PAM sequence on the non-targeted strand and after the 23rd base on the targeted strand. A Cas protein can have full cleavage activity to create a double-strand break at a target genomic locus (e.g., a double-strand break with blunt ends), or it can be a nickase that creates a single-strand break at a target genomic locus.

Examples of Cas proteins include Cas1, Cas1B, Cas2, Cas3, Cas4, Cas5, Cas5c (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Csel (CasA), Csc2 (CasB), Csc3 (CasE), Csc4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

An exemplary Cas protein is a Cas9 protein or a protein derived from a Cas9 protein. Cas9 proteins are from a type II CRISPR/Cas system and typically share four key motifs with a conserved architecture. Motifs 1, 2, and 4 are RuvC-like motifs, and motif 3 is an HNH motif. Exemplary Cas9 proteins are from Streptococcus pyogenes, Streptococcus thermophilus, Streptococcus sp., Staphylococcus aureus, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Neisseria meningitidis, or Campylobacter jejuni. Additional examples of the Cas9 family members are described in WO 2014/131833, herein incorporated by reference in its entirety for all purposes. Cas9 from S. pyogenes (SpCas9) (e.g., assigned UniProt accession number Q99ZW2) is an exemplary Cas9 protein. An exemplary SpCas9 protein sequence is set forth in SEQ ID NO: 41 (encoded by the DNA sequence set forth in SEQ ID NO: 42). Smaller Cas9 proteins (e.g., Cas9 proteins whose coding sequences are compatible with the maximum AAV packaging capacity when combined with a guide RNA coding sequence and regulatory elements for the Cas9 and guide RNA, such as SaCas9 and CjCas9 and Nme2Cas9) are other exemplary Cas9 proteins. For example, Cas9 from S. aureus (SaCas9) (e.g., assigned UniProt accession number J7RUA5) is another exemplary Cas9 protein. Likewise, Cas9 from Campylobacter jejuni (CjCas9) (e.g., assigned UniProt accession number QOP897) is another exemplary Cas9 protein. See, e.g., Kim et al. (2017) Nat. Commun. 8:14500, herein incorporated by reference in its entirety for all purposes. SaCas9 is smaller than SpCas9, and CjCas9 is smaller than both SaCas9 and SpCas9. Cas9 from Neisseria meningitidis (Nme2Cas9) is another exemplary Cas9 protein. See, e.g., Edraki et al. (2019) Mol. Cell 73 (4): 714-726, herein incorporated by reference in its entirety for all purposes. Cas9 proteins from Streptococcus thermophilus (e.g., Streptococcus thermophilus LMD-9 Cas9 encoded by the CRISPR1 locus (St1Cas9) or Streptococcus thermophilus Cas9 from the CRISPR3 locus (St3Cas9)) are other exemplary Cas9 proteins. Cas9 from Francisella novicida (FnCas9) or the RHA Francisella novicida Cas9 variant that recognizes an alternative PAM (E1369R/E1449H/R1556A substitutions) are other exemplary Cas9 proteins. These and other exemplary Cas9 proteins are reviewed, e.g., in Cebrian-Serrano and Davies (2017) Mamm. Genome 28 (7): 247-261, herein incorporated by reference in its entirety for all purposes. Examples of Cas9 coding sequences, Cas9 mRNAs, and Cas9 protein sequences are provided in WO 2013/176772, WO 2014/065596, WO 2016/106121, WO 2019/067910, WO 2020/082042, US 2020/0270617, WO 2020/082041, US 2020/0268906, WO 2020/082046, and US 2020/0289628, each of which is herein incorporated by reference in its entirety for all purposes. Specific examples of ORFs and Cas9 amino acid sequences are provided in Table 30 at paragraph WO 2019/067910, and specific examples of Cas9 mRNAs and ORFs are provided in paragraphs [0214]-[0234] of WO 2019/067910. See also WO 2020/082046 A2 (pp. 84-85) and Table 24 in WO 2020/069296, each of which is herein incorporated by reference in its entirety for all purposes.

Another example of a Cas protein is a Cpf1 (CRISPR from Prevotella and Francisella 1; Cas12a) protein. Cpf1 is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in Cas9 proteins, and the RuvC-like domain is contiguous in the Cpf1 sequence, in contrast to Cas9 where it contains long inserts including the HNH domain. See, e.g., Zetsche et al. (2015) Cell 163 (3): 759-771, herein incorporated by reference in its entirety for all purposes. Exemplary Cpf1 proteins are from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. Cpf1 from Francisella novicida U112 (FnCpf1; assigned UniProt accession number A0Q7Q2) is an exemplary Cpf1 protein.

Another example of a Cas protein is CasX (Cas12e). CasX is an RNA-guided DNA endonuclease that generates a staggered double-strand break in DNA. CasX is less than 1000 amino acids in size. Exemplary CasX proteins are from Deltaproteobacteria (DpbCasX or DpbCas12e) and Planctomycetes (PlmCasX or PlmCas12e). Like Cpf1, CasX uses a single RuvC active site for DNA cleavage. See, e.g., Liu et al. (2019) Nature 566 (7743): 218-223, herein incorporated by reference in its entirety for all purposes.

Another example of a Cas protein is CasΦ (CasPhi or Cas12j), which is uniquely found in bacteriophages. CasΦ is less than 1000 amino acids in size (e.g., 700-800 amino acids). CasΦ cleavage generates staggered 5′ overhangs. A single RuvC active site in CasΦ is capable of crRNA processing and DNA cutting. See, e.g., Pausch et al. (2020) Science 369 (6501): 333-337, herein incorporated by reference in its entirety for all purposes.

Cas proteins can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins. Active variants or fragments with respect to catalytic activity can comprise at least 80%, 85%, 90%, 91%, 92%, 93%, 94%, 95%, 96%, 97%, 98%, 99% or more sequence identity to the wild type or modified Cas protein or a portion thereof, wherein the active variants retain the ability to cut at a desired cleavage site and hence retain nick-inducing or double-strand-break-inducing activity. Assays for nick-inducing or double-strand-break-inducing activity are known and generally measure the overall activity and specificity of the Cas protein on DNA substrates containing the cleavage site.

One example of a modified Cas protein is the modified SpCas9-HF1 protein, which is a high-fidelity variant of Streptococcus pyogenes Cas9 harboring alterations (N497A/R661A/Q695A/Q926A) designed to reduce non-specific DNA contacts. See, e.g., Kleinstiver et al. (2016) Nature 529 (7587): 490-495, herein incorporated by reference in its entirety for all purposes. Another example of a modified Cas protein is the modified eSpCas9 variant (K848A/K1003A/R10⁶⁰A) designed to reduce off-target effects. See, e.g., Slaymaker et al. (2016) Science 351 (6268): 84-88, herein incorporated by reference in its entirety for all purposes. Other SpCas9 variants include K855A and K810A/K1003A/R10⁶⁰A. These and other modified Cas proteins are reviewed, e.g., in Cebrian-Serrano and Davies (2017) Mamm. Genome 28 (7): 247-261, herein incorporated by reference in its entirety for all purposes. Another example of a modified Cas9 protein is xCas9, which is a SpCas9 variant that can recognize an expanded range of PAM sequences. See, e.g., Hu et al. (2018) Nature 556:57-63, herein incorporated by reference in its entirety for all purposes.

Cas proteins can be modified to increase or decrease one or more of nucleic acid binding affinity, nucleic acid binding specificity, and enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of or a property of the Cas protein.

Cas proteins can comprise at least one nuclease domain, such as a DNase domain. For example, a wild type Cpf1 protein generally comprises a RuvC-like domain that cleaves both strands of target DNA, perhaps in a dimeric configuration. Likewise, CasX and Cas generally comprise a single RuvC-like domain that cleaves both strands of a target DNA. Cas proteins can also comprise at least two nuclease domains, such as DNase domains. For example, a wild type Cas9 protein generally comprises a RuvC-like nuclease domain and an HNH-like nuclease domain. The RuvC and HNH domains can each cut a different strand of double-stranded DNA to make a double-stranded break in the DNA. See, e.g., Jinek et al. (2012) Science 337 (6096): 816-821, herein incorporated by reference in its entirety for all purposes.

One C or more of the nuclease domains can be deleted or mutated so that they are no longer functional or have reduced nuclease activity. For example, if one of the nuclease domains is deleted or mutated in a Cas9 protein, the resulting Cas9 protein can be referred to as a nickase and can generate a single-strand break within a double-stranded target DNA but not a double-strand break (i.e., it can cleave the complementary strand or the non-complementary strand, but not both). If none of the nuclease domains is deleted or mutated in a Cas9 protein, the Cas9 protein will retain double-strand-break-inducing activity. An example of a mutation that converts Cas9 into a nickase is a D10A (aspartate to alanine at position 10 of Cas9) mutation in the RuvC domain of Cas9 from S. pyogenes. Likewise, H939A (histidine to alanine at amino acid position 839), H840A (histidine to alanine at amino acid position 840), or N863A (asparagine to alanine at amino acid position N863) in the HNH domain of Cas9 from S. pyogenes can convert the Cas9 into a nickase. Other examples of mutations that convert Cas9 into a nickase include the corresponding mutations to Cas9 from S. thermophilus. See, e.g., Sapranauskas et al. (2011) Nucleic Acids Res. 39 (21): 9275-9282 and WO 2013/141680, each of which is herein incorporated by reference in its entirety for all purposes. Such mutations can be generated using methods such as site-directed mutagenesis, PCR-mediated mutagenesis, or total gene synthesis. Examples of other mutations creating nickases can be found, for example, in WO 2013/176772 and WO 2013/142578, each of which is herein incorporated by reference in its entirety for all purposes.

Examples of inactivating mutations in the catalytic domains of xCas9 are the same as those described above for SpCas9. Examples of inactivating mutations in the catalytic domains of Staphylococcus aureus Cas9 proteins are also known. For example, the Staphylococcus aureus Cas9 enzyme (SaCas9) may comprise a substitution at position N580 (e.g., N580A substitution) or a substitution at position D10 (e.g., D10A substitution) to generate a Cas nickase. See, e.g., WO 2016/106236, herein incorporated by reference in its entirety for all purposes. Examples of inactivating mutations in the catalytic domains of Nme2Cas9 are also known (e.g., D16A or H588A). Examples of inactivating mutations in the catalytic domains of St1Cas9 are also known (e.g., D9A, D598A, H599A, or N622A). Examples of inactivating mutations in the catalytic domains of St3Cas9 are also known (e.g., D10A or N870A). Examples of inactivating mutations in the catalytic domains of CjCas9 are also known (e.g., combination of D8A or H559A). Examples of inactivating mutations in the catalytic domains of FnCas9 and RHA FnCas9 are also known (e.g., N995A).

Examples of inactivating mutations in the catalytic domains of Cpf1 proteins are also known. With reference to Cpf1 proteins from Francisella novicida U112 (FnCpf1), Acidaminococcus sp. BV3L6 (AsCpf1), Lachnospiraceae bacterium ND2006 (LbCpf1), and Moraxella bovoculi 237 (MbCpf1 Cpf1), such mutations can include mutations at positions 908, 993, or 1263 of AsCpf1 or corresponding positions in Cpf1 orthologs, or positions 832, 925, 947, or 1180 of LbCpf1 or corresponding positions in Cpf1 orthologs. Such mutations can include, for example one or more of mutations D908A, E993A, and D1263A of AsCpf1 or corresponding mutations in Cpf1 orthologs, or D832A, E925A, D947A, and D1180A of LbCpf1 or corresponding mutations in Cpf1 orthologs. See, e.g., US 2016/0208243, herein incorporated by reference in its entirety for all purposes. Examples of inactivating mutations in the catalytic domains of CasX proteins are also known. With reference to CasX proteins from Deltaproteobacteria, D672A, E769A, and D935A (individually or in combination) or corresponding positions in other CasX orthologs are inactivating. See, e.g., Liu et al. (2019) Nature 566 (7743): 218-223, herein incorporated by reference in its entirety for all purposes. Examples of inactivating mutations in the catalytic domains of CasΦ proteins are also known. For example, D371A and D394A, alone or in combination, are inactivating mutations. See, e.g., Pausch et al. (2020) Science 369 (6501): 333-337, herein incorporated by reference in its entirety for all purposes.

Cas proteins can also be operably linked to heterologous polypeptides as fusion proteins. For example, a Cas protein can be fused to a cleavage domain. See WO 2014/089290, herein incorporated by reference in its entirety for all purposesCas proteins can also be fused to a heterologous polypeptide providing increased or decreased stability. The fused domain or heterologous polypeptide can be located at the N-terminus, the C-terminus, or internally within the Cas protein.

As one example, a Cas protein can be fused to one or more heterologous polypeptides that provide for subcellular localization. Such heterologous polypeptides can include, for example, one or more nuclear localization signals (NLS) such as the monopartite SV40 NLS and/or a bipartite alpha-importin NLS for targeting to the nucleus, a mitochondrial localization signal for targeting to the mitochondria, an ER retention signal, and the like. See, e.g., Lange et al. (2007) J. Biol. Chem. 282 (8): 5101-5105, herein incorporated by reference in its entirety for all purposes. Such subcellular localization signals can be located at the N-terminus, the C-terminus, or anywhere within the Cas protein. An NLS can comprise a stretch of basic amino acids and can be a monopartite sequence or a bipartite sequence. Optionally, a Cas protein can comprise two or more NLSs, including an NLS (e.g., an alpha-importin NLS or a monopartite NLS) at the N-terminus and an NLS (e.g., an SV40 NLS or a bipartite NLS) at the C-terminus. A Cas protein can also comprise two or more NLSs at the N-terminus and/or two or more NLSs at the C-terminus.

A Cas protein may, for example, be fused with 1-10 NLSs (e.g., fused with 1-5 NLSs or fused with one NLS. Where one NLS is used, the NLS may be linked at the N-terminus or the C-terminus of the Cas protein sequence. It may also be inserted within the Cas protein sequence. Alternatively, the Cas protein may be fused with more than one NLS. For example, the Cas protein may be fused with 2, 3, 4, or 5 NLSs. In a specific example, the Cas protein may be fused with two NLSs. In certain circumstances, the two NLSs may be the same (e.g., two SV40 NLSs) or different. For example, the Cas protein can be fused to two SV40 NLS sequences linked at the carboxy terminus. Alternatively, the Cas protein may be fused with two NLSs, one linked at the N-terminus and one at the C-terminus. In other examples, the Cas protein may be fused with 3 NLSs or with no NLS. The NLS may be a monopartite sequence, such as, e.g., the SV40 NLS, PKKKRKV (SEQ ID NO: 43) or PKKKRRV (SEQ ID NO: 44). The NLS may be a bipartite sequence, such as the NLS of nucleoplasmin, KRPAATKKAGQAKKKK (SEQ ID NO: 45). In a specific example, a single PKKKRKV (SEQ ID NO: 43) NLS may be linked at the C-terminus of the Cas protein. One or more linkers are optionally included at the fusion site.

Cas proteins can also be operably linked to a cell-penetrating domain or protein transduction domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. See, e.g., WO 2014/089290 and WO 2013/176772, each of which is herein incorporated by reference in its entirety for all purposes. The cell-penetrating domain can be located at the N-terminus, the C-terminus, or anywhere within the Cas protein.

Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Optionally, the nucleic acid encoding the Cas protein can be codon optimized for efficient translation into protein in a particular cell or organism. For example, the nucleic acid encoding the Cas protein can be modified to substitute codons having a higher frequency of usage in a bacterial cell, a yeast cell, a human cell, a non-human cell, a mammalian cell, a rodent cell, a mouse cell, a rat cell, or any other host cell of interest, as compared to the naturally occurring polynucleotide sequence. When a nucleic acid encoding the Cas protein is introduced into the cell, the Cas protein can be transiently, conditionally, or constitutively expressed in the cell.

Nucleic acids encoding Cas proteins can be stably integrated in the genome of a cell and operably linked to a promoter active in the cell. Alternatively, nucleic acids encoding Cas proteins can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding a gRNA. Alternatively, it can be in a vector or plasmid that is separate from the vector comprising the DNA encoding the gRNA. Promoters that can be used in an expression construct include promoters active, for example, in a human cell or a human hematopoietic cell. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allow for the generation of compact expression cassettes to facilitate delivery. In certain embodiments, promotors are accepted by regulatory authorities for use in humans. In certain embodiments, promotors drive expression in hematopoietic cells.

Different promoters can be used to drive Cas expression or Cas9 expression. In some methods, small promoters are used so that the Cas or Cas9 coding sequence can fit into an AAV construct. For example, Cas or Cas9 and one or more gRNAs (e.g., 1 gRNA or 2 gRNAs or 3 gRNAs or 4 gRNAs) can be delivered via LNP-mediated delivery (e.g., in the form of RNA) or adeno-associated virus (AAV)-mediated delivery (e.g., AAV8-mediated delivery). For example, the nuclease agent can be CRISPR/Cas9, and a Cas9 mRNA and a gRNA (e.g., targeting an IL2RG gene (e.g., a human IL2RG gene)) can be delivered via LNP-mediated delivery or AAV-mediated delivery. The Cas or Cas9 and the gRNA(s) can be delivered in a single AAV or via two separate AAVs. For example, a first AAV can carry a Cas or Cas9 expression cassette, and a second AAV can carry a gRNA expression cassette. Similarly, a first AAV can carry a Cas or Cas9 expression cassette, and a second AAV can carry two or more gRNA expression cassettes. Alternatively, a single AAV can carry a Cas or Cas9 expression cassette (e.g., Cas or Cas9 coding sequence operably linked to a promoter) and a gRNA expression cassette (e.g., gRNA coding sequence operably linked to a promoter). Similarly, a single AAV can carry a Cas or Cas9 expression cassette (e.g., Cas or Cas9 coding sequence operably linked to a promoter) and two or more gRNA expression cassettes (e.g., gRNA coding sequences operably linked to promoters). Different promoters can be used to drive expression of the gRNA, such as a U6 promoter or the small tRNA Gln. Likewise, different promoters can be used to drive Cas9 expression. For example, small promoters are used so that the Cas9 coding sequence can fit into an AAV construct. Similarly, small Cas9 proteins (e.g., SaCas9 or CjCas9 are used to maximize the AAV packaging capacity).

Cas proteins provided as mRNAs can be modified for improved stability and/or immunogenicity properties. The modifications may be made to one or more nucleosides within the mRNA. mRNA encoding Cas proteins can also be capped. Cas mRNAs can further comprise a poly-adenylated (poly-A or poly(A) or poly-adenine) tail. For example, a Cas mRNA can include a modification to one or more nucleosides within the mRNA, the Cas mRNA can be capped, and the Cas mRNA can comprise a poly(A) tail.

Guide RNAs. A “guide RNA” or “gRNA” is an RNA molecule that binds to a Cas protein (e.g., Cas9 protein) and targets the Cas protein to a specific location within a target DNA. Guide RNAs can comprise two segments: a “DNA-targeting segment” (also called “guide sequence”) and a “protein-binding segment.” “Segment” includes a section or region of a molecule, such as a contiguous stretch of nucleotides in an RNA. Some gRNAs, such as those for Cas9, can comprise two separate RNA molecules: an “activator-RNA” (e.g., tracrRNA) and a “targeter-RNA” (e.g., CRISPR RNA or crRNA). Other gRNAs are a single RNA molecule (single RNA polynucleotide), which can also be called a “single-molecule gRNA,” a “single-guide RNA,” or an “sgRNA.” See, e.g., WO 2013/176772, WO 2014/065596, WO 2014/089290, WO 2014/093622, WO 2014/099750, WO 2013/142578, and WO 2014/131833, each of which is herein incorporated by reference in its entirety for all purposes. A guide RNA can refer to either a CRISPR RNA (crRNA) or the combination of a crRNA and a trans-activating CRISPR RNA (tracrRNA). The crRNA and tracrRNA can be associated as a single RNA molecule (single guide RNA or sgRNA) or in two separate RNA molecules (dual guide RNA or dgRNA). For Cas9, for example, a single-guide RNA can comprise a crRNA fused to a tracrRNA (e.g., via a linker). For Cpf1 and CasΦ, for example, only a crRNA is needed to achieve binding to a target sequence. The terms “guide RNA” and “gRNA” include both double-molecule (i.e., modular) gRNAs and single-molecule gRNAs. In some of the methods and compositions disclosed herein, a gRNA is a S. pyogenes Cas9 gRNA or an equivalent thereof. In some of the methods and compositions disclosed herein, a gRNA is a S. aureus Cas9 gRNA or an equivalent thereof.

An exemplary two-molecule gRNA comprises a crRNA-like (“CRISPR RNA” or “targeter-RNA” or “crRNA” or “crRNA repeat”) molecule and a corresponding tracrRNA-like (“trans-activating CRISPR RNA” or “activator-RNA” or “tracrRNA”) molecule. A crRNA comprises both the DNA-targeting segment (single-stranded) of the gRNA and a stretch of nucleotides that forms one half of the dsRNA duplex of the protein-binding segment of the gRNA. An example of a crRNA tail (e.g., for use with S. pyogenes Cas9), located downstream (3′) of the DNA-targeting segment, comprises, consists essentially of, or consists of GUUUUAGAGCUAUGCU (SEQ ID NO: 46) or GUUUUAGAGCUAUGCUGUUUUG (SEQ ID NO: 47). Any of the DNA-targeting segments disclosed herein can be joined to the 5′ end of SEQ ID NO: 46 or 47 to form a crRNA.

A corresponding tracrRNA (activator-RNA) comprises a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. Examples of tracrRNA sequences (e.g., for use with S. pyogenes Cas9) comprise, consist essentially of, or consist of any one of

(SEQ ID NO: 48)
AGCAUAGCAAGUUAAAAUAAGGCUAGUCCGUUAUCAACUUGAAAAAGUG
GCACCGAGUCGGUGCUUU,
(SEQ ID NO: 49)
AAACAGCAUAGCAAGUUAAAAUAAGGCUAGUCCGUUAUCAACUUGAAAA
AGUGGCACCGAGUCGGUGCUUUU,
or
(SEQ ID NO: 50)
GUUGGAACCAUUCAAAACAGCAUAGCAAGUUAAAAUAAGGCUAGUCCGU
UAUCAACUUGAAAAAGUGGCACCGAGUCGGUGC.

In systems in which both a crRNA and a tracrRNA are needed, the crRNA and the corresponding tracrRNA hybridize to form a gRNA. In systems in which only a crRNA is needed, the crRNA can be the gRNA. The crRNA additionally provides the single-stranded DNA-targeting segment that hybridizes to the complementary strand of a target DNA. If used for modification within a cell, the exact sequence of a given crRNA or tracrRNA molecule can be designed to be specific to the species in which the RNA molecules will be used. See, e.g., Mali et al. (2013) Science 339 (6121): 823-826; Jinek et al. (2012) Science 337 (6096): 816-821; Hwang et al. (2013) Nat. Biotechnol. 31 (3): 227-229; Jiang et al. (2013) Nat. Biotechnol. 31 (3): 233-239; and Cong et al. (2013) Science 339 (6121): 819-823, each of which is herein incorporated by reference in its entirety for all purposes.

The DNA-targeting segment (crRNA) of a given gRNA comprises a nucleotide sequence that is complementary to a sequence on the complementary strand of the target DNA, as described in more detail below. The DNA-targeting segment of a gRNA interacts with the target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the CRISPR/Cas system and organism but often contain a targeting segment of between 21 to 72 nucleotides length, flanked by two direct repeats (DR) of a length of between 21 to 46 nucleotides (see, e.g., WO 2014/131833, herein incorporated by reference in its entirety for all purposes). In the case of S. pyogenes, the DRs are 36 nucleotides long and the targeting segment is 30 nucleotides long. The 3′ located DR is complementary to and hybridizes with the corresponding tracrRNA, which in turn binds to the Cas protein.

The DNA-targeting segment can have, for example, a length of at least about 12, at least about 15, at least about 17, at least about 18, at least about 19, at least about 20, at least about 25, at least about 30, at least about 35, or at least about 40 nucleotides. Such DNA-targeting segments can have, for example, a length from about 12 to about 100, from about 12 to about 80, from about 12 to about 50, from about 12 to about 40, from about 12 to about 30, from about 12 to about 25, or from about 12 to about 20 nucleotides. For example, the DNA targeting segment can be from about 15 to about 25 nucleotides (e.g., from about 17 to about 20 nucleotides, or about 17, 18, 19, or 20 nucleotides). See, e.g., US 2016/0024523, herein incorporated by reference in its entirety for all purposes. For Cas9 from S. pyogenes, a typical DNA-targeting segment is between 16 and 20 nucleotides in length or between 17 and 20 nucleotides in length. For Cas9 from S. aureus, a typical DNA-targeting segment is between 21 and 23 nucleotides in length. For Cpf1, a typical DNA-targeting segment is at least 16 nucleotides in length or at least 18 nucleotides in length.

In one example, the DNA-targeting segment can be about 20 nucleotides in length. However, shorter and longer sequences can also be used for the targeting segment (e.g., 15-25 nucleotides in length, such as 15, 16, 17, 18, 19, 20, 21, 22, 23, 24, or 25 nucleotides in length). The degree of identity between the DNA-targeting segment and the corresponding guide RNA target sequence (or degree of complementarity between the DNA-targeting segment and the other strand of the guide RNA target sequence) can be, for example, about 75%, about 80%, about 85%, about 90%, about 95%, or 100%. The DNA-targeting segment and the corresponding guide RNA target sequence can contain one or more mismatches. For example, the DNA-targeting segment of the guide RNA and the corresponding guide RNA target sequence can contain 1-4, 1-3, 1-2, 1, 2, 3, or 4 mismatches (e.g., where the total length of the guide RNA target sequence is at least 17, at least 18, at least 19, or at least 20 or more nucleotides). For example, the DNA-targeting segment of the guide RNA and the corresponding guide RNA target sequence can contain 1-4, 1-3, 1-2, 1, 2, 3, or 4 mismatches where the total length of the guide RNA target sequence 20 nucleotides.

As one example, a guide RNA targeting human IL2RG (e.g., human IL2RG exon 3, such as to modify position M145 in human IL2RG) can comprise a DNA-targeting segment (i.e., guide sequence) comprising, consisting essentially of, or consisting of the sequence (DNA-targeting segment) set forth in any one of SEQ ID NOS: 76-87. Alternatively, a guide RNA targeting human IL2RG (e.g., human IL2RG exon 3) can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 76-87. Alternatively, a guide RNA targeting human IL2RG (e.g., human IL2RG exon 3) can comprise a DNA-targeting segment that is at least 75%, at least 80%, at least 85%, at least 90%, or at least 95% identical to the sequence (DNA-targeting segment) set forth in any one of SEQ ID NOS: 76-87. Alternatively, a guide RNA targeting human IL2RG (e.g., human IL2RG exon 3) can comprise a DNA-targeting segment that is at least 90% or at least 95% identical to the sequence (DNA-targeting segment) set forth in any one of SEQ ID NOS: 76-87. Alternatively, a guide RNA targeting human IL2RG (e.g., human IL2RG exon 3) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 76-87. Alternatively, a guide RNA targeting human IL2RG (e.g., human IL2RG exon 3) can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 76-87. Alternatively, a guide RNA targeting human IL2RG (e.g., human IL2RG exon 3) can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 76-87. Alternatively, a guide RNA targeting human IL2RG (e.g., human IL2RG exon 3) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 76-87. In some cases, two or more guide RNAs targeting the target genomic locus (e.g., IL2RG or human IL2RG) are used.

As one example, a guide RNA targeting human IL2RG (e.g., human IL2RG exon 3, such as to modify position M145 in human IL2RG) can comprise a DNA-targeting segment (i.e., guide sequence) comprising, consisting essentially of, or consisting of the sequence (DNA-targeting segment) set forth in any one of SEQ ID NOS: 77, 83, and 86. Alternatively, a guide RNA targeting human IL2RG (e.g., human IL2RG exon 3) can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 77, 83, and 86. Alternatively, a guide RNA targeting human IL2RG (e.g., human IL2RG exon 3) can comprise a DNA-targeting segment that is at least 75%, at least 80%, at least 85%, at least 90%, or at least 95% identical to the sequence (DNA-targeting segment) set forth in any one of SEQ ID NOS: 77, 83, and 86. Alternatively, a guide RNA targeting human IL2RG (e.g., human IL2RG exon 3) can comprise a DNA-targeting segment that is at least 90% or at least 95% identical to the sequence (DNA-targeting segment) set forth in any one of SEQ ID NOS: 77, 83, and 86. Alternatively, a guide RNA targeting human IL2RG (e.g., human IL2RG exon 3) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 77, 83, and 86. Alternatively, a guide RNA targeting human IL2RG (e.g., human IL2RG exon 3) can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 77, 83, and 86. Alternatively, a guide RNA targeting human IL2RG (e.g., human IL2RG exon 3) can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 77, 83, and 86. Alternatively, a guide RNA targeting human IL2RG (e.g., human IL2RG exon 3) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 77, 83, and 86. In some cases, two or more guide RNAs targeting the target genomic locus (e.g., IL2RG or human IL2RG) are used.

As one example, a guide RNA targeting human IL2RG (e.g., human IL2RG exon 3, such as to modify position M145 in human IL2RG) can comprise a DNA-targeting segment (i.e., guide sequence) comprising, consisting essentially of, or consisting of the sequence (DNA-targeting segment) set forth in SEQ ID NO: 77. Alternatively, a guide RNA targeting human IL2RG (e.g., human IL2RG exon 3) can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 77. Alternatively, a guide RNA targeting human IL2RG (e.g., human IL2RG exon 3) can comprise a DNA-targeting segment that is at least 75%, at least 80%, at least 85%, at least 90%, or at least 95% identical to the sequence (DNA-targeting segment) set forth in SEQ ID NO: 77. Alternatively, a guide RNA targeting human IL2RG (e.g., human IL2RG exon 3) can comprise a DNA-targeting segment that is at least 90% or at least 95% identical to the sequence (DNA-targeting segment) set forth in SEQ ID NO: 77. Alternatively, a guide RNA targeting human IL2RG (e.g., human IL2RG exon 3) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 77. Alternatively, a guide RNA targeting human IL2RG (e.g., human IL2RG exon 3) can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 77. Alternatively, a guide RNA targeting human IL2RG (e.g., human IL2RG exon 3) can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 77. Alternatively, a guide RNA targeting human IL2RG (e.g., human IL2RG exon 3) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 77. In some cases, two or more guide RNAs targeting the target genomic locus (e.g., IL2RG or human IL2RG) are used. In some embodiments, such guide RNAs are used together with an ssODN comprising, consisting essentially of, or consisting of the sequence set forth in any one of SEQ ID NOS: 88-97.

As one example, a guide RNA targeting human IL2RG (e.g., human IL2RG exon 3, such as to modify position M145 in human IL2RG) can comprise a DNA-targeting segment (i.e., guide sequence) comprising, consisting essentially of, or consisting of the sequence (DNA-targeting segment) set forth in SEQ ID NO: 83. Alternatively, a guide RNA targeting human IL2RG (e.g., human IL2RG exon 3) can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 83. Alternatively, a guide RNA targeting human IL2RG (e.g., human IL2RG exon 3) can comprise a DNA-targeting segment that is at least 75%, at least 80%, at least 85%, at least 90%, or at least 95% identical to the sequence (DNA-targeting segment) set forth in SEQ ID NO: 83. Alternatively, a guide RNA targeting human IL2RG (e.g., human IL2RG exon 3) can comprise a DNA-targeting segment that is at least 90% or at least 95% identical to the sequence (DNA-targeting segment) set forth in SEQ ID NO: 83. Alternatively, a guide RNA targeting human IL2RG (e.g., human IL2RG exon 3) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 83. Alternatively, a guide RNA targeting human IL2RG (e.g., human IL2RG exon 3) can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 83. Alternatively, a guide RNA targeting human IL2RG (e.g., human IL2RG exon 3) can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 83. Alternatively, a guide RNA targeting human IL2RG (e.g., human IL2RG exon 3) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 83. In some cases, two or more guide RNAs targeting the target genomic locus (e.g., IL2RG or human IL2RG) are used. In some embodiments, such guide RNAs are used together with an ssODN comprising, consisting essentially of, or consisting of the sequence set forth in any one of SEQ ID NOS: 98-10⁷.

As one example, a guide RNA targeting human IL2RG (e.g., human IL2RG exon 3, such as to modify position M145 in human IL2RG) can comprise a DNA-targeting segment (i.e., guide sequence) comprising, consisting essentially of, or consisting of the sequence (DNA-targeting segment) set forth in SEQ ID NO: 86. Alternatively, a guide RNA targeting human IL2RG (e.g., human IL2RG exon 3) can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 86. Alternatively, a guide RNA targeting human IL2RG (e.g., human IL2RG exon 3) can comprise a DNA-targeting segment that is at least 75%, at least 80%, at least 85%, at least 90%, or at least 95% identical to the sequence (DNA-targeting segment) set forth in SEQ ID NO: 86. Alternatively, a guide RNA targeting human IL2RG (e.g., human IL2RG exon 3) can comprise a DNA-targeting segment that is at least 90% or at least 95% identical to the sequence (DNA-targeting segment) set forth in SEQ ID NO: 86. Alternatively, a guide RNA targeting human IL2RG (e.g., human IL2RG exon 3) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 86. Alternatively, a guide RNA targeting human IL2RG (e.g., human IL2RG exon 3) can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 86. Alternatively, a guide RNA targeting human IL2RG (e.g., human IL2RG exon 3) can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 86. Alternatively, a guide RNA targeting human IL2RG (e.g., human IL2RG exon 3) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 86. In some cases, two or more guide RNAs targeting the target genomic locus (e.g., IL2RG or human IL2RG) are used. In some embodiments, such guide RNAs are used together with an ssODN comprising, consisting essentially of, or consisting of the sequence set forth in any one of SEQ ID NOS: 108-117.

As one example, a guide RNA targeting human IL2RG (e.g., human IL2RG exon 2, exon 3, or exons 2 and 3 (e.g., exon 2), such as to modify position W90 in human IL2RG) can comprise a DNA-targeting segment (i.e., guide sequence) comprising, consisting essentially of, or consisting of the sequence (DNA-targeting segment) set forth in any one of SEQ ID NOS: 136-153. Alternatively, a guide RNA targeting human IL2RG can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 136-153. Alternatively, a guide RNA targeting human IL2RG can comprise a DNA-targeting segment that is at least 75%, at least 80%, at least 85%, at least 90%, or at least 95% identical to the sequence (DNA-targeting segment) set forth in any one of SEQ ID NOS: 136-153. Alternatively, a guide RNA targeting human IL2RG can comprise a DNA-targeting segment that is at least 90% or at least 95% identical to the sequence (DNA-targeting segment) set forth in any one of SEQ ID NOS: 136-153. Alternatively, a guide RNA targeting human IL2RG can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 136-153. Alternatively, a guide RNA targeting human IL2RG can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 136-153. Alternatively, a guide RNA targeting human IL2RG can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 136-153. Alternatively, a guide RNA targeting human IL2RG can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 136-153. In some cases, two or more guide RNAs targeting the target genomic locus (e.g., IL2RG or human IL2RG) are used.

As one example, a guide RNA targeting human IL2RG (e.g., human IL2RG exon 2, exon 3, or exons 2 and 3 (e.g., exon 2), such as to modify position W90 in human IL2RG) can comprise a DNA-targeting segment (i.e., guide sequence) comprising, consisting essentially of, or consisting of the sequence (DNA-targeting segment) set forth in any one of SEQ ID NOS: 137 and 138. Alternatively, a guide RNA targeting human IL2RG can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 137 and 138. Alternatively, a guide RNA targeting human IL2RG can comprise a DNA-targeting segment that is at least 75%, at least 80%, at least 85%, at least 90%, or at least 95% identical to the sequence (DNA-targeting segment) set forth in any one of SEQ ID NOS: 137 and 138. Alternatively, a guide RNA targeting human IL2RG can comprise a DNA-targeting segment that is at least 90% or at least 95% identical to the sequence (DNA-targeting segment) set forth in any one of SEQ ID NOS: 137 and 138. Alternatively, a guide RNA targeting human IL2RG can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 137 and 138. Alternatively, a guide RNA targeting human IL2RG can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 137 and 138. Alternatively, a guide RNA targeting human IL2RG can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 137 and 138. Alternatively, a guide RNA targeting human IL2RG can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 137 and 138. In some cases, two or more guide RNAs targeting the target genomic locus (e.g., IL2RG or human IL2RG) are used.

As one example, a guide RNA targeting human IL2RG (e.g., human IL2RG exon 2, exon 3, or exons 2 and 3 (e.g., exon 2), such as to modify position W90 in human IL2RG) can comprise a DNA-targeting segment (i.e., guide sequence) comprising, consisting essentially of, or consisting of the sequence (DNA-targeting segment) set forth in SEQ ID NO: 137. Alternatively, a guide RNA targeting human IL2RG can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 137. Alternatively, a guide RNA targeting human IL2RG can comprise a DNA-targeting segment that is at least 75%, at least 80%, at least 85%, at least 90%, or at least 95% identical to the sequence (DNA-targeting segment) set forth in SEQ ID NO: 137. Alternatively, a guide RNA targeting human IL2RG can comprise a DNA-targeting segment that is at least 90% or at least 95% identical to the sequence (DNA-targeting segment) set forth in SEQ ID NO: 137. Alternatively, a guide RNA targeting human IL2RG can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 137. Alternatively, a guide RNA targeting human IL2RG can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 137. Alternatively, a guide RNA targeting human IL2RG can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 137. Alternatively, a guide RNA targeting human IL2RG can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 137. In some cases, two or more guide RNAs targeting the target genomic locus (e.g., IL2RG or human IL2RG) are used. In some embodiments, such guide RNAs are used together with an ssODN comprising, consisting essentially of, or consisting of the sequence set forth in any one of SEQ ID NOS: 154-163.

As one example, a guide RNA targeting human IL2RG (e.g., human IL2RG exon 2, exon 3, or exons 2 and 3 (e.g., exon 2), such as to modify position W90 in human IL2RG) can comprise a DNA-targeting segment (i.e., guide sequence) comprising, consisting essentially of, or consisting of the sequence (DNA-targeting segment) set forth in SEQ ID NO: 138. Alternatively, a guide RNA targeting human IL2RG can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 138. Alternatively, a guide RNA targeting human IL2RG can comprise a DNA-targeting segment that is at least 75%, at least 80%, at least 85%, at least 90%, or at least 95% identical to the sequence (DNA-targeting segment) set forth in SEQ ID NO: 138. Alternatively, a guide RNA targeting human IL2RG can comprise a DNA-targeting segment that is at least 90% or at least 95% identical to the sequence (DNA-targeting segment) set forth in SEQ ID NO: 138. Alternatively, a guide RNA targeting human IL2RG can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 138. Alternatively, a guide RNA targeting human IL2RG can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 138. Alternatively, a guide RNA targeting human IL2RG can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 138. Alternatively, a guide RNA targeting human IL2RG can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 138. In some cases, two or more guide RNAs targeting the target genomic locus (e.g., IL2RG or human IL2RG) are used. In some embodiments, such guide RNAs are used together with an ssODN comprising, consisting essentially of, or consisting of the sequence set forth in any one of SEQ ID NOS: 164-173.

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise, consist essentially of, or consist of all or a portion of a wild type tracrRNA sequence (e.g., about or more than about 20, 26, 32, 45, 48, 54, 63, 67, 85, or more nucleotides of a wild type tracrRNA sequence). Examples of wild type tracrRNA sequences from S. pyogenes include 171-nucleotide, 89-nucleotide, 75-nucleotide, and 65-nucleotide versions. See, e.g., Deltcheva et al. (2011) Nature 471 (7340): 602-607; WO 2014/093661, each of which is herein incorporated by reference in its entirety for all purposes. Examples of tracrRNAs within single-guide RNAs (sgRNAs) include the tracrRNA segments found within +48, +54, +67, and +85 versions of sgRNAs, where “+n” indicates that up to the +n nucleotide of wild type tracrRNA is included in the sgRNA. See U.S. Pat. No. 8,697,359, herein incorporated by reference in its entirety for all purposes.

The percent complementarity between the DNA-targeting segment of the guide RNA and the complementary strand of the target DNA can be at least 60% (e.g., at least 65%, at least 70%, at least 75%, at least 80%, at least 85%, at least 90%, at least 95%, or 100%). The percent complementarity between the DNA-targeting segment and the complementary strand of the target DNA can be at least 60% over about 20 contiguous nucleotides. As an example, the percent complementarity between the DNA-targeting segment and the complementary strand of the target DNA can be 100% over the 14 contiguous nucleotides at the 5′ end of the complementary strand of the target DNA and as low as 0% over the remainder. In such a case, the DNA-targeting segment can be considered to be 14 nucleotides in length. As another example, the percent complementarity between the DNA-targeting segment and the complementary strand of the target DNA can be 100% over the seven contiguous nucleotides at the 5′ end of the complementary strand of the target DNA and as low as 0% over the remainder. In such a case, the DNA-targeting segment can be considered to be 7 nucleotides in length. In some guide RNAs, at least 17 nucleotides within the DNA-targeting segment are complementary to the complementary strand of the target DNA. For example, the DNA-targeting segment can be 20 nucleotides in length and can comprise 1, 2, or 3 mismatches with the complementary strand of the target DNA. In one example, the mismatches are not adjacent to the region of the complementary strand corresponding to the protospacer adjacent motif (PAM) sequence (i.e., the reverse complement of the PAM sequence) (e.g., the mismatches are in the 5′ end of the DNA-targeting segment of the guide RNA, or the mismatches are at least 2, 3, 4, 5, 6, 7, 8, 9, 10, 11, 12, 13, 14, 15, 16, 17, 18, or 19 base pairs away from the region of the complementary strand corresponding to the PAM sequence).

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, and the gRNA directs the bound Cas protein to a specific nucleotide sequence within target DNA via the DNA-targeting segment.

Single-guide RNAs can comprise a DNA-targeting segment and a scaffold sequence (i.e., the protein-binding or Cas-binding sequence of the guide RNA). For example, such guide RNAs can have a 5′ DNA-targeting segment joined to a 3′ scaffold sequence. Exemplary scaffold sequences (e.g., for use with S. pyogenes Cas9) comprise, consist essentially of, or consist of:

(version 1; SEQ ID NO: 51)
GUUUUAGAGCUAGAAAUAGCAAGUUAAAAUAAGGCUAGUCCGUUAUCAA
CUUGAAAAAGUGGCACCGAGUCGGUGCU;
(version 2; SEQ ID NO: 52)
GUUGGAACCAUUCAAAACAGCAUAGCAAGUUAAAAUAAGGCUAGUCCGU
UAUCAACUUGAAAAAGUGGCACCGAGUCGGUGC;
(version 3; SEQ ID NO: 53)
GUUUUAGAGCUAGAAAUAGCAAGUUAAAAUAAGGCUAGUCCGUUAUCAA
CUUGAAAAAGUGGCACCGAGUCGGUGC;
and
(version 4; SEQ ID NO: 54)
GUUUAAGAGCUAUGCUGGAAACAGCAUAGCAAGUUUAAAUAAGGCUAGU
CCGUUAUCAACUUGAAAAAGUGGCACCGAGUCGGUGC;
(version 5; SEQ ID NO: 55)
GUUUUAGAGCUAGAAAUAGCAAGUUAAAAUAAGGCUAGUCCGUUAUCAA
CUUGAAAAAGUGGCACCGAGUCGGUGCUUUUUUU;
(version 6; SEQ ID NO: 56)
GUUUUAGAGCUAGAAAUAGCAAGUUAAAAUAAGGCUAGUCCGUUAUCAA
CUUGAAAAAGUGGCACCGAGUCGGUGCUUUU;
(version 7; SEQ ID NO: 57)
GUUUAAGAGCUAUGCUGGAAACAGCAUAGCAAGUUUAAAUAAGGCUAGU
CCGUUAUCAACUUGAAAAAGUGGCACCGAGUCGGUGCUUUUUU;
or
(version 8; SEQ ID NO: 58)
GUUUUAGAGCUAGAAAUAGCAAGUUAAAAUAAGGCUAGUCCGUUAUCAA
CUUGGCACCGAGUCGGUGC.

In some guide sgRNAs, the four terminal U residues of version 6 are not present. In some sgRNAs, only 1, 2, or 3 of the four terminal U residues of version 6 are present. Guide RNAs targeting any of the guide RNA target sequences disclosed herein can include, for example, a DNA-targeting segment on the 5′ end of the guide RNA fused to any of the exemplary guide RNA scaffold sequences on the 3′ end of the guide RNA. That is, any of the DNA-targeting segments disclosed herein can be joined to the 5′ end of any one of the above scaffold sequences to form a single guide RNA (chimeric guide RNA).

Guide RNAs can include modifications or sequences that provide for additional desirable features (e.g., modified or regulated stability; subcellular targeting; tracking with a fluorescent label; a binding site for a protein or protein complex; and the like). That is, guide RNAs can include one or more modified nucleosides or nucleotides, or one or more non-naturally and/or naturally occurring components or configurations that are used instead of or in addition to the canonical A, G, C, and U residues. Examples of such modifications include, for example, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin); a modification or sequence that targets the RNA to a subcellular location (e.g., nucleus); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof. Other examples of modifications include engineered stem loop duplex structures, engineered bulge regions, engineered hairpins 3′ of the stem loop duplex structure, or any combination thereof. See, e.g., US 2015/0376586, herein incorporated by reference in its entirety for all purposes. A bulge can be an unpaired region of nucleotides within the duplex made up of the crRNA-like region and the minimum tracrRNA-like region. A bulge can comprise, on one side of the duplex, an unpaired 5′-XXXY-3′ where X is any purine and Y can be a nucleotide that can form a wobble pair with a nucleotide on the opposite strand, and an unpaired nucleotide region on the other side of the duplex.

Guide RNAs can comprise modified nucleosides and modified nucleotides including, for example, one or more of the following: (1) alteration or replacement of one or both of the non-linking phosphate oxygens and/or of one or more of the linking phosphate oxygens in the phosphodiester backbone linkage (an exemplary backbone modification); (2) alteration or replacement of a constituent of the ribose sugar such as alteration or replacement of the 2′ hydroxyl on the ribose sugar (an exemplary sugar modification); (3) replacement (e.g., wholesale replacement) of the phosphate moiety with dephospho linkers (an exemplary backbone modification); (4) modification or replacement of a naturally occurring nucleobase, including with a non-canonical nucleobase (an exemplary base modification); (5) replacement or modification of the ribose-phosphate backbone (an exemplary backbone modification); (6) modification of the 3′ end or 5′ end of the oligonucleotide (e.g., removal, modification or replacement of a terminal phosphate group or conjugation of a moiety, cap, or linker (such 3′ or 5′ cap modifications may comprise a sugar and/or backbone modification); and (7) modification or replacement of the sugar (an exemplary sugar modification). Other possible guide RNA modifications include modifications of or replacement of uracils or poly-uracil tracts. See, e.g., WO 2015/048577 and US 2016/0237455, each of which is herein incorporated by reference in its entirety for all purposes. Similar modifications can be made to Cas-encoding nucleic acids, such as Cas mRNAs. For example, Cas mRNAs can be modified by depletion of uridine using synonymous codons.

Chemical modifications such as those listed above can be combined to provide modified gRNAs and/or mRNAs comprising residues (nucleosides and nucleotides) that can have two, three, four, or more modifications. For example, a modified residue can have a modified sugar and a modified nucleobase. In one example, every base of a gRNA is modified (e.g., all bases have a modified phosphate group, such as a phosphorothioate group). For example, all or substantially all of the phosphate groups of a gRNA can be replaced with phosphorothioate groups. Alternatively or additionally, a modified gRNA can comprise at least one modified residue at or near the 5′ end. Alternatively or additionally, a modified gRNA can comprise at least one modified residue at or near the 3′ end.

Some gRNAs comprise one, two, three or more modified residues. For example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the positions in a modified gRNA can be modified nucleosides or nucleotides.

Unmodified nucleic acids can be prone to degradation. Exogenous nucleic acids can also induce an innate immune response. Modifications can help introduce stability and reduce immunogenicity. Some gRNAs described herein can contain one or more modified nucleosides or nucleotides to introduce stability toward intracellular or serum-based nucleases. Some modified gRNAs described herein can exhibit a reduced innate immune response when introduced into a population of cells.

In a dual guide RNA, each of the crRNA and the tracrRNA can contain modifications. Such modifications may be at one or both ends of the crRNA and/or tracrRNA. In a sgRNA, one or more residues at one or both ends of the sgRNA may be chemically modified, and/or internal nucleosides may be modified, and/or the entire sgRNA may be chemically modified. Some gRNAs comprise a 5′ end modification. Some gRNAs comprise a 3′ end modification. Some gRNAs comprise a 5′ end modification and a 3′ end modification.

The guide RNAs disclosed herein can comprise one of the modification patterns disclosed in WO 2018/107028 A1, herein incorporated by reference in its entirety for all purposes. The guide RNAs disclosed herein can also comprise one of the structures/modification patterns disclosed in US 2017/0114334, herein incorporated by reference in its entirety for all purposes. The guide RNAs disclosed herein can also comprise one of the structures/modification patterns disclosed in WO 2017/136794, WO 2017/004279, US 2018/0187186, or US 2019/0048338, each of which is herein incorporated by reference in its entirety for all purposes.

As one example, any of the guide RNAs described herein can comprise at least one modification. In one example, the at least one modification comprises a 2′-O-methyl (2′-O-Mc) modified nucleotide, a phosphorothioate (PS) bond between nucleotides, a 2′-fluoro (2′-F) modified nucleotide, or a combination thereof. For example, the at least one modification can comprise a 2′-O-methyl (2′-O-Me) modified nucleotide. Alternatively or additionally, the at least one modification can comprise a phosphorothioate (PS) bond between nucleotides. Alternatively or additionally, the at least one modification can comprise a 2′-fluoro (2′-F) modified nucleotide. In one example, a guide RNA described herein comprises one or more 2′-O-methyl (2′-O-Me) modified nucleotides and one or more phosphorothioate (PS) bonds between nucleotides.

Guide RNAs can be provided in any form. For example, the gRNA can be provided in the form of RNA, either as two molecules (separate crRNA and tracrRNA) or as one molecule (sgRNA), and optionally in the form of a complex with a Cas protein. The gRNA can also be provided in the form of DNA encoding the gRNA. The DNA encoding the gRNA can encode a single RNA molecule (sgRNA) or separate RNA molecules (e.g., separate crRNA and tracrRNA). In the latter case, the DNA encoding the gRNA can be provided as one DNA molecule or as separate DNA molecules encoding the crRNA and tracrRNA, respectively.

When a gRNA is provided in the form of DNA, the gRNA can be transiently, conditionally, or constitutively expressed in the cell. DNAs encoding gRNAs can be stably integrated into the genome of the cell and operably linked to a promoter active in the cell. Alternatively, DNAs encoding gRNAs can be operably linked to a promoter in an expression construct. For example, the DNA encoding the gRNA can be in a vector comprising a heterologous nucleic acid, such as a nucleic acid encoding a Cas protein. Alternatively, it can be in a vector or a plasmid that is separate from the vector comprising the nucleic acid encoding the Cas protein. Promoters that can be used in such expression constructs include promoters active, for example, in a human cell or a human hematopoietic cell. Such promoters can be, for example, conditional promoters, inducible promoters, constitutive promoters, or tissue-specific promoters. Such promoters can also be, for example, bidirectional promoters. Specific examples of suitable promoters include an RNA polymerase III promoter, such as a human U6 promoter, a rat U6 polymerase III promoter, or a mouse U6 polymerase III promoter.

Alternatively, gRNAs can be prepared by various other methods. For example, gRNAs can be prepared by in vitro transcription using, for example, T7 RNA polymerase (see, e.g., WO 2014/089290 and WO 2014/065596, each of which is herein incorporated by reference in its entirety for all purposes). Guide RNAs can also be a synthetically produced molecule prepared by chemical synthesis.

Guide RNAs (or nucleic acids encoding guide RNAs) can be in compositions comprising one or more guide RNAs (e.g., 1, 2, 3, 4, or more guide RNAs) and a carrier increasing the stability of the guide RNA (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules. Such compositions can further comprise a Cas protein, such as a Cas9 protein, or a nucleic acid encoding a Cas protein.

Guide RNA Target Sequences. Target DNAs for guide RNAs include nucleic acid sequences present in a DNA to which a DNA-targeting segment of a gRNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual, 3rd Ed. (Sambrook et al., Harbor Laboratory Press 2001), herein incorporated by reference in its entirety for all purposes). The strand of the target DNA that is complementary to and hybridizes with the gRNA can be called the “complementary strand,” and the strand of the target DNA that is complementary to the “complementary strand” (and is therefore not complementary to the Cas protein or gRNA) can be called “noncomplementary strand” or “template strand.”

The target DNA includes both the sequence on the complementary strand to which the guide RNA hybridizes and the corresponding sequence on the non-complementary strand (e.g., adjacent to the protospacer adjacent motif (PAM)). The term “guide RNA target sequence” as used herein refers specifically to the sequence on the non-complementary strand corresponding to (i.e., the reverse complement of) the sequence to which the guide RNA hybridizes on the complementary strand. That is, the guide RNA target sequence refers to the sequence on the non-complementary strand adjacent to the PAM (e.g., upstream or 5′ of the PAM in the case of Cas9). A guide RNA target sequence is equivalent to the DNA-targeting segment of a guide RNA, but with thymines instead of uracils. As one example, a guide RNA target sequence for an SpCas9 enzyme can refer to the sequence upstream of the 5′-NGG-3′ PAM on the non-complementary strand. A guide RNA is designed to have complementarity to the complementary strand of a target DNA, where hybridization between the DNA-targeting segment of the guide RNA and the complementary strand of the target DNA promotes the formation of a CRISPR complex. Full complementarity is not necessarily required, provided that there is sufficient complementarity to cause hybridization and promote formation of a CRISPR complex. If a guide RNA is referred to herein as targeting a guide RNA target sequence, what is meant is that the guide RNA hybridizes to the complementary strand sequence of the target DNA that is the reverse complement of the guide RNA target sequence on the non-complementary strand.

A target DNA or guide RNA target sequence can comprise any polynucleotide, and can be located, for example, in the nucleus or cytoplasm of a cell or within an organelle of a cell. A target DNA or guide RNA target sequence can be any nucleic acid sequence endogenous or exogenous to a cell. The guide RNA target sequence can be a sequence coding a gene product (e.g., a protein) or a non-coding sequence (e.g., a regulatory sequence) or can include both.

Site-specific binding and cleavage of a target DNA by a Cas protein can occur at locations determined by both (i) base-pairing complementarity between the guide RNA and the complementary strand of the target DNA and (ii) a short motif, called the protospacer adjacent motif (PAM), in the non-complementary strand of the target DNA. The PAM can flank the guide RNA target sequence. Optionally, the guide RNA target sequence can be flanked on the 3′ end by the PAM (e.g., for Cas9). Alternatively, the guide RNA target sequence can be flanked on the 5′ end by the PAM (e.g., for Cpf1). For example, the cleavage site of Cas proteins can be about 1 to about 10 or about 2 to about 5 base pairs (e.g., 3 base pairs) upstream or downstream of the PAM sequence (e.g., within the guide RNA target sequence). In the case of SpCas9, the PAM sequence (i.e., on the non-complementary strand) can be 5′-N₁GG-3′, where N₁ is any DNA nucleotide, and where the PAM is immediately 3′ of the guide RNA target sequence on the non-complementary strand of the target DNA. As such, the sequence corresponding to the PAM on the complementary strand (i.e., the reverse complement) would be 5′-CCN₂-3′, where N₂ is any DNA nucleotide and is immediately 5′ of the sequence to which the DNA-targeting segment of the guide RNA hybridizes on the complementary strand of the target DNA. In some such cases, N₁ and N₂ can be complementary and the N₁-N₂ base pair can be any base pair (e.g., N₁═C and N₂=G; N₁=G and N₂=C; N₁=A and N₂=T; or N₁=T, and N₂=A). In the case of Cas9 from S. aureus, the PAM can be NNGRRT or NNGRR, where N can A, G, C, or T, and R can be G or A. In the case of Cas9 from C. jejuni, the PAM can be, for example, NNNNACAC or NNNNRYAC, where N can be A, G, C, or T, and R can be G or A. In some cases (e.g., for FnCpf1), the PAM sequence can be upstream of the 5′ end and have the sequence 5′-TTN-3′. In the case of DpbCasX, the PAM can have the sequence 5′-TTCN-3′. In the case of CasΦ, the PAM can have the sequence 5′-TBN-3′, where B is G, T, or C.

An example of a guide RNA target sequence is a 20-nucleotide DNA sequence immediately preceding an NGG motif recognized by an SpCas9 protein. For example, two examples of guide RNA target sequences plus PAMs are GN₁₉NGG (SEQ ID NO: 59) or N₂₀NGG (SEQ ID NO: 60). See, e.g., WO 2014/165825, herein incorporated by reference in its entirety for all purposes. The guanine at the 5′ end can facilitate transcription by RNA polymerase in cells. Other examples of guide RNA target sequences plus PAMs can include two guanine nucleotides at the 5′ end (e.g., GGN₂₀NGG; SEQ ID NO: 61) to facilitate efficient transcription by T7 polymerase in vitro. See, e.g., WO 2014/065596, herein incorporated by reference in its entirety for all purposes. Other guide RNA target sequences plus PAMs can have between 4-22 nucleotides in length of SEQ ID NOS: 59-61, including the 5′ G or GG and the 3′ GG or NGG. Yet other guide RNA target sequences plus PAMs can have between 14 and 20 nucleotides in length of SEQ ID NOS: 59-61.

Formation of a CRISPR complex hybridized to a target DNA can result in cleavage of one or both strands of the target DNA within or near the region corresponding to the guide RNA target sequence (i.e., the guide RNA target sequence on the non-complementary strand of the target DNA and the reverse complement on the complementary strand to which the guide RNA hybridizes). For example, the cleavage site can be within the guide RNA target sequence (e.g., at a defined location relative to the PAM sequence). The “cleavage site” includes the position of a target DNA at which a Cas protein produces a single-strand break or a double-strand break. The cleavage site can be on only one strand (e.g., when a nickase is used) or on both strands of a double-stranded DNA. Cleavage sites can be at the same position on both strands (producing blunt ends; e.g., Cas9)) or can be at different sites on each strand (producing staggered ends (i.e., overhangs); e.g., Cpf1). Staggered ends can be produced, for example, by using two Cas proteins, each of which produces a single-strand break at a different cleavage site on a different strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the guide RNA target sequence or cleavage site of the nickase on the first strand is separated from the guide RNA target sequence or cleavage site of the nickase on the second strand by at least 2, 3, 4, 5, 6, 7, 8, 9, 10, 15, 20, 25, 30, 40, 50, 75,100,250, 500, or 1,000 base pairs.

The guide RNA target sequence can also be selected to minimize off-target modification or avoid off-target effects (e.g., by avoiding two or fewer mismatches to off-target genomic sequences).

As one example, a guide RNA targeting human IL2RG (e.g., exon 3 of human IL2RG, such as to modify position M145 of human IL2RG) can target the guide RNA target sequence set forth in any one of SEQ ID NOS: 64-75. As another example, a guide RNA targeting human IL2RG (e.g., exon 3 of human IL2RG) can target at least 17, at least 18, at least 19, or at least 20 contiguous nucleotides of the guide RNA target sequence set forth in any one of SEQ ID NOS: 64-75.

As one example, a guide RNA targeting human IL2RG (e.g., exon 3 of human IL2RG, such as to modify position M145 of human IL2RG) can target the guide RNA target sequence set forth in any one of SEQ ID NOS: 65, 71, and 74. As another example, a guide RNA targeting human IL2RG (e.g., exon 3 of human IL2RG) can target at least 17, at least 18, at least 19, or at least 20 contiguous nucleotides of the guide RNA target sequence set forth in any one of SEQ ID NOS: 65, 71, and 74.

As one example, a guide RNA targeting human IL2RG (e.g., exon 3 of human IL2RG, such as to modify position M145 of human IL2RG) can target the guide RNA target sequence set forth in SEQ ID NO: 65. As another example, a guide RNA targeting human IL2RG (e.g., exon 3 of human IL2RG) can target at least 17, at least 18, at least 19, or at least 20 contiguous nucleotides of the guide RNA target sequence set forth in SEQ ID NO: 65. In some embodiments, such guide RNAs are used together with an ssODN comprising, consisting essentially of, or consisting of the sequence set forth in any one of SEQ ID NOS: 88-97.

As one example, a guide RNA targeting human IL2RG (e.g., exon 3 of human IL2RG, such as to modify position M145 of human IL2RG) can target the guide RNA target sequence set forth in SEQ ID NO: 71. As another example, a guide RNA targeting human IL2RG (e.g., exon 3 of human IL2RG) can target at least 17, at least 18, at least 19, or at least 20 contiguous nucleotides of the guide RNA target sequence set forth in SEQ ID NO: 71. In some embodiments, such guide RNAs are used together with an ssODN comprising, consisting essentially of, or consisting of the sequence set forth in any one of SEQ ID NOS: 98-107.

As one example, a guide RNA targeting human IL2RG (e.g., exon 3 of human IL2RG, such as to modify position M145 of human IL2RG) can target the guide RNA target sequence set forth in SEQ ID NO: 74. As another example, a guide RNA targeting human IL2RG (e.g., exon 3 of human IL2RG) can target at least 17, at least 18, at least 19, or at least 20 contiguous nucleotides of the guide RNA target sequence set forth in SEQ ID NO: 74. In some embodiments, such guide RNAs are used together with an ssODN comprising, consisting essentially of, or consisting of the sequence set forth in any one of SEQ ID NOS: 108-117.

As one example, a guide RNA targeting human IL2RG (e.g., human IL2RG exon 2, exon 3, or exons 2 and 3 (e.g., exon 2), such as to modify position W90 of human IL2RG) can target the guide RNA target sequence set forth in any one of SEQ ID NOS: 118-135. As another example, a guide RNA targeting human IL2RG can target at least 17, at least 18, at least 19, or at least 20 contiguous nucleotides of the guide RNA target sequence set forth in any one of SEQ ID NOS: 118-135.

As one example, a guide RNA targeting human IL2RG (e.g., human IL2RG exon 2, exon 3, or exons 2 and 3 (e.g., exon 2), such as to modify position W90 of human IL2RG) can target the guide RNA target sequence set forth in any one of SEQ ID NOS: 119 and 120. As another example, a guide RNA targeting human IL2RG can target at least 17, at least 18, at least 19, or at least 20 contiguous nucleotides of the guide RNA target sequence set forth in any one of SEQ ID NOS: 119 and 120.

As one example, a guide RNA targeting human IL2RG (e.g., human IL2RG exon 2, exon 3, or exons 2 and 3 (e.g., exon 2), such as to modify position W90 of human IL2RG) can target the guide RNA target sequence set forth in SEQ ID NO: 119. As another example, a guide RNA targeting human IL2RG (e.g., exon 3 of human IL2RG) can target at least 17, at least 18, at least 19, or at least 20 contiguous nucleotides of the guide RNA target sequence set forth in SEQ ID NO: 119. In some embodiments, such guide RNAs are used together with an ssODN comprising, consisting essentially of, or consisting of the sequence set forth in any one of SEQ ID NOS: 154-163.

As one example, a guide RNA targeting human IL2RG (e.g., human IL2RG exon 2, exon 3, or exons 2 and 3 (e.g., exon 2), such as to modify position W90 of human IL2RG) can target the guide RNA target sequence set forth in SEQ ID NO: 120. As another example, a guide RNA targeting human IL2RG can target at least 17, at least 18, at least 19, or at least 20 contiguous nucleotides of the guide RNA target sequence set forth in SEQ ID NO: 120. In some embodiments, such guide RNAs are used together with an ssODN comprising, consisting essentially of, or consisting of the sequence set forth in any one of SEQ ID NOS: 164-173.

Exogenous Donor Nucleic Acids. Any suitable exogenous donor nucleic acid can be used in the methods disclosed herein. In some embodiments, the exogenous donor nucleic acid comprises the entire coding sequence for the first isoform of the target protein (e.g., IL2RG). In some embodiments, the coding sequence for the first isoform of the target protein (e.g., IL2RG) is operably linked to a promoter suitable for driving expression in the donor cells or edited cells. Alternatively, the coding sequence for the first isoform of the target protein (e.g., IL2RG) is not operably linked to a promoter in the exogenous donor nucleic acid but will be operably linked to an endogenous promoter at the target genomic locus once the exogenous donor nucleic acid recombines with or is integrated into the target genomic locus. In other embodiments, the exogenous donor nucleic acid does not comprise the entire coding sequence for the first isoform of the target protein (e.g., IL2RG). For example, the exogenous donor nucleic acid may comprise a portion of the coding sequence for the first isoform of the target protein (e.g., IL2RG), wherein the portion comprises the mutation that distinguishes the first isoform from the second isoform. For example, the exogenous donor nucleic acids can comprise a mutation to modify the target genomic locus encoding the target protein so that it encodes the first isoform of the target protein. In some embodiments, the exogenous donor nucleic acid recombines with the target genomic locus via non-homologous end joining (NHEJ)-mediated ligation or through a homology-directed repair event. Optionally, repair with the exogenous donor nucleic acid removes or disrupts the nuclease target sequence so that alleles that have been targeted cannot be re-targeted by the nuclease agent.

The exogenous donor nucleic acid can target any sequence in the target genomic locus (e.g., IL2RG gene, human IL2RG gene). In some embodiments, the exogenous donor nucleic acid targets exon 3 of an IL2RG gene (e.g., exon 3 of a human IL2RG gene). In some embodiments, the exogenous donor nucleic acid targets exon 2 of an IL2RG gene (e.g., exon 2 of a human IL2RG gene). In some embodiments, the exogenous donor nucleic acid targets exons 2 and 3 of an IL2RG gene (e.g., exons 2 and 3 of a human IL2RG gene). Some exogenous donor nucleic acids comprise homology arms. Other exogenous donor nucleic acids do not comprise homology arms. The exogenous donor nucleic acids can be capable of insertion into a target genomic locus (e.g., IL2RG) by homology-directed repair, and/or they can be capable of insertion into a target genomic locus (e.g., IL2RG) by non-homologous end joining.

Exogenous donor nucleic acids can comprise deoxyribonucleic acid (DNA) or ribonucleic acid (RNA), they can be single-stranded or double-stranded, and they can be in linear or circular form. For example, an exogenous donor nucleic acid can be a single-stranded oligodeoxynucleotide (ssODN). See, e.g., Yoshimi et al. (2016) Nat. Commun. 7:10431, herein incorporated by reference in its entirety for all purposes. Exogenous donor nucleic acids can be naked nucleic acids or can be delivered by viruses, such as AAV. In a specific example, the exogenous donor nucleic acid can be delivered via AAV and can be capable of insertion into a target genomic locus by non-homologous end joining (e.g., the exogenous donor nucleic acid can be one that does not comprise homology arms).

An exemplary exogenous donor nucleic acid is between about 50 nucleotides to about 5 kb in length, is between about 50 nucleotides to about 3 kb in length, or is between about 50 to about 1,000 nucleotides in length. Other exemplary exogenous donor nucleic acids are between about 40 to about 200 nucleotides in length. For example, an exogenous donor nucleic acid can be between about 50-60, 60-70, 70-80, 80-90, 90-100, 100-110, 110-120, 120-130, 130-140, 140-150, 150-160, 160-170, 170-180, 180-190, or 190-200 nucleotides in length. Alternatively, an exogenous donor nucleic acid can be between about 50-100, 100-200, 200-300, 300-400, 400-500, 500-600, 600-700, 700-800, 800-900, or 900-1000 nucleotides in length. Alternatively, an exogenous donor nucleic acid can be between about 1-1.5, 1.5-2, 2-2.5, 2.5-3, 3-3.5, 3.5-4, 4-4.5, or 4.5-5 kb in length. Alternatively, an exogenous donor nucleic acid can be, for example, no more than 5 kb, 4.5 kb, 4 kb, 3.5 kb, 3 kb, 2.5 kb, 2 kb, 1.5 kb, 1 kb, 900 nucleotides, 800 nucleotides, 700 nucleotides, 600 nucleotides, 500 nucleotides, 400 nucleotides, 300 nucleotides, 200 nucleotides, 100 nucleotides, or 50 nucleotides in length. Exogenous donor nucleic acids (e.g., targeting vectors) can also be longer.

In one example, an exogenous donor nucleic acid is an ssODN that is between about 80 nucleotides and about 200 nucleotides in length. In another example, an exogenous donor nucleic acid is an ssODN that is between about 80 nucleotides and about 3 kb in length. Such an ssODN can have homology arms, for example, that are each between about 40 nucleotides and about 60 nucleotides in length. Such an ssODN can also have homology arms, for example, that are each between about 30 nucleotides and 100 nucleotides in length. The homology arms can be symmetrical (e.g., each 40 nucleotides or each 60 nucleotides in length), or they can be asymmetrical (e.g., one homology arm that is 36 nucleotides in length, and one homology arm that is 91 nucleotides in length).

In one example, an exogenous donor nucleic acid is an ssODN that is between about 30 nucleotides and about 200 nucleotides in length. In another example, the exogenous donor nucleic acid is an ssODN that is between about 30 nucleotides and about 120 nucleotides in length. In another example, the exogenous donor nucleic acid is an ssODN that is between about 30 nucleotides and about 90 nucleotides in length. In another example, the exogenous donor nucleic acid is an ssODN that is between about 30 nucleotides and about 60 nucleotides in length. In another example, the exogenous donor nucleic acid is an ssODN that is between about 60 nucleotides and about 120 nucleotides in length. In another example, the exogenous donor nucleic acid is an ssODN that is between about 90 nucleotides and about 120 nucleotides in length. In another example, the exogenous donor nucleic acid is an ssODN that is between about 60 nucleotides and about 90 nucleotides in length. In another example, the exogenous donor nucleic acid is an ssODN that is between about 30 nucleotides and about 130 nucleotides in length. In another example, the exogenous donor nucleic acid is an ssODN that is between about 30 nucleotides and about 140 nucleotides in length. In another example, the exogenous donor nucleic acid is an ssODN that is between about 30 nucleotides and about 150 nucleotides in length. In another example, the exogenous donor nucleic acid is an ssODN that is between about 30 nucleotides and about 160 nucleotides in length. In another example, the exogenous donor nucleic acid is an ssODN that is between about 30 nucleotides and about 170 nucleotides in length. In another example, the exogenous donor nucleic acid is an ssODN that is between about 30 nucleotides and about 180 nucleotides in length. In another example, the exogenous donor nucleic acid is an ssODN that is between about 30 nucleotides and about 190 nucleotides in length. In another example, the exogenous donor nucleic acid is an ssODN that is between about 30 nucleotides and about 200 nucleotides in length. In another example, the exogenous donor nucleic acid is an ssODN that is between about 60 nucleotides and about 130 nucleotides in length. In another example, the exogenous donor nucleic acid is an ssODN that is between about 60 nucleotides and about 140 nucleotides in length. In another example, the exogenous donor nucleic acid is an ssODN that is between about 60 nucleotides and about 150 nucleotides in length. In another example, the exogenous donor nucleic acid is an ssODN that is between about 60 nucleotides and about 160 nucleotides in length. In another example, the exogenous donor nucleic acid is an ssODN that is between about 60 nucleotides and about 170 nucleotides in length. In another example, the exogenous donor nucleic acid is an ssODN that is between about 60 nucleotides and about 180 nucleotides in length. In another example, the exogenous donor nucleic acid is an ssODN that is between about 60 nucleotides and about 190 nucleotides in length. In another example, the exogenous donor nucleic acid is an ssODN that is between about 60 nucleotides and about 200 nucleotides in length. In another example, the exogenous donor nucleic acid is an ssODN that is between about 90 nucleotides and about 130 nucleotides in length. In another example, the exogenous donor nucleic acid is an ssODN that is between about 90 nucleotides and about 140 nucleotides in length. In another example, the exogenous donor nucleic acid is an ssODN that is between about 90 nucleotides and about 150 nucleotides in length. In another example, the exogenous donor nucleic acid is an ssODN that is between about 90 nucleotides and about 160 nucleotides in length. In another example, the exogenous donor nucleic acid is an ssODN that is between about 90 nucleotides and about 170 nucleotides in length. In another example, the exogenous donor nucleic acid is an ssODN that is between about 90 nucleotides and about 180 nucleotides in length. In another example, the exogenous donor nucleic acid is an ssODN that is between about 90 nucleotides and about 190 nucleotides in length. In another example, the exogenous donor nucleic acid is an ssODN that is between about 90 nucleotides and about 200 nucleotides in length. In another example, the exogenous donor nucleic acid is an ssODN that is between about 50 and about 100 nucleotides in length. In another example, the exogenous donor nucleic acid is an ssODN that is between about 40 nucleotides and about 110 nucleotides in length.

In one example, the exogenous donor nucleic acid is an ssODN that is about 120 nucleotides in length. In another example, the exogenous donor nucleic acid is an ssODN that is about 90 nucleotides in length. In another example, the exogenous donor nucleic acid is an ssODN that is about 60 nucleotides in length. In another example, the exogenous donor nucleic acid is an ssODN that is about 30 nucleotides in length. In another example, the exogenous donor nucleic acid is an ssODN that is between about 110 nucleotides and about 130 nucleotides in length. In another example, the exogenous donor nucleic acid is an ssODN that is between about 100 nucleotides and about 140 nucleotides in length. In another example, the exogenous donor nucleic acid is an ssODN that is between about 90 nucleotides and about 150 nucleotides in length. In another example, the exogenous donor nucleic acid is an ssODN that is between about 80 nucleotides and about 160 nucleotides in length. In another example, the exogenous donor nucleic acid is an ssODN that is between about 70 nucleotides and about 170 nucleotides in length. In another example, the exogenous donor nucleic acid is an ssODN that is between about 60 nucleotides and about 180 nucleotides in length. In another example, the exogenous donor nucleic acid is an ssODN that is between about 80 nucleotides and about 100 nucleotides in length. In another example, the exogenous donor nucleic acid is an ssODN that is between about 70 nucleotides and about 110 nucleotides in length. In another example, the exogenous donor nucleic acid is an ssODN that is between about 60 nucleotides and about 120 nucleotides in length. In another example, the exogenous donor nucleic acid is an ssODN that is between about 50 nucleotides and about 130 nucleotides in length. In another example, the exogenous donor nucleic acid is an ssODN that is between about 40 nucleotides and about 140 nucleotides in length. In another example, the exogenous donor nucleic acid is an ssODN that is between about 30 nucleotides and about 150 nucleotides in length. In another example, the exogenous donor nucleic acid is an ssODN that is between about 50 nucleotides and about 70 nucleotides in length. In another example, the exogenous donor nucleic acid is an ssODN that is between about 40 nucleotides and about 80 nucleotides in length. In another example, the exogenous donor nucleic acid is an ssODN that is between about 30 nucleotides and about 90 nucleotides in length. In another example, the exogenous donor nucleic acid is an ssODN that is between about 20 nucleotides and about 100 nucleotides in length. In another example, the exogenous donor nucleic acid is an ssODN that is between about 20 nucleotides and about 40 nucleotides in length.

In some embodiments, the exogenous donor nucleic acid is an ssODN that comprises the sequence set forth in any one of SEQ ID NOS: 88-117 (e.g., to generate an M145K modification in human IL2RG). In some embodiments, the exogenous donor nucleic acid is an ssODN that consists essentially of the sequence set forth in any one of SEQ ID NOS: 88-117. In some embodiments, the exogenous donor nucleic acid is an ssODN that consists of the sequence set forth in any one of SEQ ID NOS: 88-117.

In some embodiments, the exogenous donor nucleic acid is an ssODN that comprises the sequence set forth in any one of SEQ ID NOS: 88-97 (e.g., to generate an M145K modification in human IL2RG). In some embodiments, the exogenous donor nucleic acid is an ssODN that consists essentially of the sequence set forth in any one of SEQ ID NOS: 88-97. In some embodiments, the exogenous donor nucleic acid is an ssODN that consists of the sequence set forth in any one of SEQ ID NOS: 88-97.

In some embodiments, the exogenous donor nucleic acid is an ssODN that comprises the sequence set forth in any one of SEQ ID NOS: 98-10⁷ (e.g., to generate an M145K modification in human IL2RG). In some embodiments, the exogenous donor nucleic acid is an ssODN that consists essentially of the sequence set forth in any one of SEQ ID NOS: 98-10⁷. In some embodiments, the exogenous donor nucleic acid is an ssODN that consists of the sequence set forth in any one of SEQ ID NOS: 98-10⁷.

In some embodiments, the exogenous donor nucleic acid is an ssODN that comprises the sequence set forth in any one of SEQ ID NOS: 108-117 (e.g., to generate an M145K modification in human IL2RG). In some embodiments, the exogenous donor nucleic acid is an ssODN that consists essentially of the sequence set forth in any one of SEQ ID NOS: 108-117. In some embodiments, the exogenous donor nucleic acid is an ssODN that consists of the sequence set forth in any one of SEQ ID NOS: 108-117.

In some embodiments, the exogenous donor nucleic acid is an ssODN that comprises the sequence set forth in any one of SEQ ID NOS: 154-173 (e.g., to generate an W90Q modification in human IL2RG). In some embodiments, the exogenous donor nucleic acid is an ssODN that consists essentially of the sequence set forth in any one of SEQ ID NOS: 154-173. In some embodiments, the exogenous donor nucleic acid is an ssODN that consists of the sequence set forth in any one of SEQ ID NOS: 154-173.

In some embodiments, the exogenous donor nucleic acid is an ssODN that comprises the sequence set forth in any one of SEQ ID NOS: 154-163 (e.g., to generate an W90Q modification in human IL2RG). In some embodiments, the exogenous donor nucleic acid is an ssODN that consists essentially of the sequence set forth in any one of SEQ ID NOS: 154-163. In some embodiments, the exogenous donor nucleic acid is an ssODN that consists of the sequence set forth in any one of SEQ ID NOS: 154-163.

In some embodiments, the exogenous donor nucleic acid is an ssODN that comprises the sequence set forth in any one of SEQ ID NOS: 164-173 (e.g., to generate an W90Q modification in human IL2RG). In some embodiments, the exogenous donor nucleic acid is an ssODN that consists essentially of the sequence set forth in any one of SEQ ID NOS: 164-173. In some embodiments, the exogenous donor nucleic acid is an ssODN that consists of the sequence set forth in any one of SEQ ID NOS: 164-173.

Exogenous donor nucleic acids can include modifications or sequences that provide for additional desirable features (e.g., modified or regulated stability; tracking or detecting with a fluorescent label; a binding site for a protein or protein complex; and so forth). Exogenous donor nucleic acids can comprise one or more fluorescent labels, purification tags, epitope tags, or a combination thereof. For example, an exogenous donor nucleic acid can comprise one or more fluorescent labels (e.g., fluorescent proteins or other fluorophores or dyes), such as at least 1, at least 2, at least 3, at least 4, or at least 5 fluorescent labels. Exemplary fluorescent labels include fluorophores such as fluorescein (e.g., 6-carboxyfluorescein (6-FAM)), Texas Red, HEX, Cy3, Cy5, Cy5.5, Pacific Blue, 5-(and-6)-carboxytetramethylrhodamine (TAMRA), and Cy7. A wide range of fluorescent dyes are available commercially for labeling oligonucleotides (e.g., from Integrated DNA Technologies). Such fluorescent labels (e.g., internal fluorescent labels) can be used, for example, to detect an exogenous donor nucleic acid that has been directly integrated into a cleaved target nucleic acid having protruding ends compatible with the ends of the exogenous donor nucleic acid. The label or tag can be at the 5′ end, the 3′ end, or internally within the exogenous donor nucleic acid. For example, an exogenous donor nucleic acid can be conjugated at 5′ end with the IR700 fluorophore from Integrated DNA Technologies (5′IRDYE®700).

Exogenous donor nucleic acids can also comprise nucleic acid inserts including segments of DNA to be integrated in the target genomic locus (i.e., to modify the target genomic locus such that it encodes the first isoform of the target protein). Integration of a nucleic acid insert in the target genomic locus can result in addition of a nucleic acid sequence of interest to the target genomic locus, deletion of a nucleic acid sequence of interest in the target genomic locus, or replacement of a nucleic acid sequence of interest in the target genomic locus (i.e., deletion and insertion; or substitution). Some exogenous donor nucleic acids are designed for insertion of a nucleic acid insert in the target genomic locus without any corresponding deletion in the target genomic locus. Other exogenous donor nucleic acids are designed to delete a nucleic acid sequence of interest in the target genomic locus without any corresponding insertion of a nucleic acid insert. Yet other exogenous donor nucleic acids are designed to delete a nucleic acid sequence of interest in the target genomic locus and replace it with a nucleic acid insert (e.g., a substitution).

The nucleic acid insert or the corresponding nucleic acid in the target genomic locus being deleted and/or replaced can be various lengths. An exemplary nucleic acid insert or corresponding nucleic acid in the target genomic locus being deleted and/or replaced is between about 1 nucleotide to about 5 kb in length or is between about 1 nucleotide to about 1,000 nucleotides in length. For example, a nucleic acid insert or a corresponding nucleic acid in the target genomic locus being deleted and/or replaced can be between about 1-10, 10⁻²⁰, 20-30, 30-40, 40-50, 50-60, 60-70, 70-80, 80-90, 90-100, 100-110, 110-120, 120-130, 130-140, 140-150, 150-160, 160-170, 170-180, 180-190, or 190-120 nucleotides in length. Likewise, a nucleic acid insert or a corresponding nucleic acid in the target genomic locus being deleted and/or replaced can be between 1-100, 100-200, 200-300, 300-400, 400-500, 500-600, 600-700, 700-800, 800-900, or 900-1000 nucleotides in length. Likewise, a nucleic acid insert or a corresponding nucleic acid in the target genomic locus being deleted and/or replaced can be between about 1-1.5, 1.5-2, 2-2.5, 2.5-3, 3-3.5, 3.5-4, 4-4.5, or 4.5-5 kb in length or longer.

The nucleic acid insert can comprise a sequence that is homologous or orthologous to all or part of sequence targeted for replacement. For example, the nucleic acid insert can comprise a sequence that comprises one or more point mutations (e.g., 1, 2, 3, 4, 5, or more) compared with a sequence targeted for replacement in the target genomic locus. Optionally, such point mutations can result in a conservative amino acid substitution (e.g., substitution of aspartic acid [Asp, D] with glutamic acid [Glu, E]) in the encoded polypeptide.

Donor Nucleic Acids for Non-Homologous-End-Joining-Mediated Insertion. Some exogenous donor nucleic acids are capable of insertion into a target genomic locus by non-homologous end joining. In some cases, such exogenous donor nucleic acids do not comprise homology arms. For example, such exogenous donor nucleic acids can be inserted into a blunt end double-strand break following cleavage with a nuclease agent. In a specific example, the exogenous donor nucleic acid can be delivered via AAV and can be capable of insertion into a target genomic locus by non-homologous end joining (e.g., the exogenous donor nucleic acid can be one that does not comprise homology arms). In a specific example, the exogenous donor nucleic acid can be inserted via homology-independent targeted integration. For example, the insert sequence in the exogenous donor nucleic acid to be inserted into a target genomic locus can be flanked on each side by a target site for a nuclease agent (e.g., the same target site as in the target genomic locus, and the same nuclease agent being used to cleave the target site in the target genomic locus). The nuclease agent can then cleave the target sites flanking the insert sequence. In a specific example, the exogenous donor nucleic acid is delivered AAV-mediated delivery, and cleavage of the target sites flanking the insert sequence can remove the inverted terminal repeats (ITRs) of the AAV. In some methods, the target site in the target genomic locus (e.g., a gRNA target sequence including the flanking protospacer adjacent motif) is no longer present if the insert sequence is inserted into the target genomic locus in the correct orientation but it is reformed if the insert sequence is inserted into the target genomic locus in the opposite orientation. This can help ensure that the insert sequence is inserted in the correct orientation for expression.

Other exogenous donor nucleic acids have short single-stranded regions at the 5′ end and/or the 3′ end that are complementary to one or more overhangs created by nuclease-mediated cleavage in the target genomic locus. For example, some exogenous donor nucleic acids have short single-stranded regions at the 5′ end and/or the 3′ end that are complementary to one or more overhangs created by nuclease-mediated cleavage at 5′ and/or 3′ target sequences in the target genomic locus. Some such exogenous donor nucleic acids have a complementary region only at the 5′ end or only at the 3′ end. For example, some such exogenous donor nucleic acids have a complementary region only at the 5′ end complementary to an overhang created at a 5′ target sequence in the target genomic locus or only at the 3′ end complementary to an overhang created at a 3′ target sequence in the target genomic locus. Other such exogenous donor nucleic acids have complementary regions at both the 5′ and 3′ ends. For example, other such exogenous donor nucleic acids have complementary regions at both the 5′ and 3′ ends e.g., complementary to first and second overhangs, respectively, generated by nuclease-mediated cleavage in the target genomic locus. For example, if the exogenous donor nucleic acid is double-stranded, the single-stranded complementary regions can extend from the 5′ end of the top strand of the donor nucleic acid and the 5′ end of the bottom strand of the donor nucleic acid, creating 5′ overhangs on each end. Alternatively, the single-stranded complementary region can extend from the 3′ end of the top strand of the donor nucleic acid and from the 3′ end of the bottom strand of the template, creating 3′ overhangs.

The complementary regions can be of any length sufficient to promote ligation between the exogenous donor nucleic acid and the target nucleic acid. Exemplary complementary regions are between about 1 to about 5 nucleotides in length, between about 1 to about 25 nucleotides in length, or between about 5 to about 150 nucleotides in length. For example, a complementary region can be at least about 1, 2, 3, 4, 5, 6, 7, 8, 9, 10, 11, 12, 13, 14, 15, 16, 17, 18, 19, 20, 21, 22, 23, 24, or 25 nucleotides in length. Alternatively, the complementary region can be about 5-10, 10⁻²⁰, 20-30, 30-40, 40-50, 50-60, 60-70, 70-80, 80-90, 90-100, 100-110, 110-120, 120-130, 130-140, or 140-150 nucleotides in length, or longer.

Such complementary regions can be complementary to overhangs created by two pairs of nickases. Two double-strand breaks with staggered ends can be created by using first and second nickases that cleave opposite strands of DNA to create a first double-strand break, and third and fourth nickases that cleave opposite strands of DNA to create a second double-strand break. For example, a Cas protein can be used to nick first, second, third, and fourth guide RNA target sequences corresponding with first, second, third, and fourth guide RNAs. The first and second guide RNA target sequences can be positioned to create a first cleavage site such that the nicks created by the first and second nickases on the first and second strands of DNA create a double-strand break (i.e., the first cleavage site comprises the nicks within the first and second guide RNA target sequences). Likewise, the third and fourth guide RNA target sequences can be positioned to create a second cleavage site such that the nicks created by the third and fourth nickases on the first and second strands of DNA create a double-strand break (i.e., the second cleavage site comprises the nicks within the third and fourth guide RNA target sequences). Optionally, the nicks within the first and second guide RNA target sequences and/or the third and fourth guide RNA target sequences can be off-set nicks that create overhangs. The offset window can be, for example, at least about 5 bp, 10 bp, 20 bp, 30 bp, 40 bp, 50 bp, 60 bp, 70 bp, 80 bp, 90 bp, 100 bp or more. See Ran et al. (2013) Cell 154:1380-1389; Mali et al. (2013) Nat. Biotech. 31:833-838; and Shen et al. (2014) Nat. Methods 11:399-404, each of which is herein incorporated by reference in its entirety for all purposes. In such cases, a double-stranded exogenous donor nucleic acid can be designed with single-stranded complementary regions that are complementary to the overhangs created by the nicks within the first and second guide RNA target sequences and by the nicks within the third and fourth guide RNA target sequences. Such an exogenous donor nucleic acid can then be inserted by non-homologous-end-joining-mediated ligation.

Donor Nucleic Acids for Insertion by Homology-Directed Repair. Some exogenous donor nucleic acids comprise homology arms. If the exogenous donor nucleic acid also comprises a nucleic acid insert, the homology arms can flank the nucleic acid insert. For case of reference, the homology arms are referred to herein as 5′ and 3′ (i.e., upstream and downstream) homology arms. This terminology relates to the relative position of the homology arms to the nucleic acid insert within the exogenous donor nucleic acid. The 5′ and 3′ homology arms correspond to regions within the target genomic locus, which are referred to herein as “5′ target sequence” and “3′ target sequence,” respectively.

A homology arm and a 5′ target sequence or 3′ target sequence “correspond” or are “corresponding” to one another when the two regions share a sufficient level of sequence identity to one another to act as substrates for a homologous recombination reaction. The term “homology” includes DNA sequences that are either identical or share sequence identity to a corresponding sequence. The sequence identity between a given target sequence and the corresponding homology arm found in the exogenous donor nucleic acid can be any degree of sequence identity that allows for homologous recombination to occur. For example, the amount of sequence identity shared by the homology arm of the exogenous donor nucleic acid (or a fragment thereof) and the target sequence (or a fragment thereof) can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination. Moreover, a corresponding region of homology between the homology arm and the corresponding target sequence can be of any length that is sufficient to promote homologous recombination. Exemplary homology arms are between about 25 nucleotides to about 2.5 kb in length, are between about 25 nucleotides to about 1.5 kb in length, or are between about 25 to about 500 nucleotides in length. For example, a given homology arm (or each of the homology arms) and/or corresponding target sequence can comprise corresponding regions of homology that are between about 25-30, 30-40, 40-50, 50-60, 60-70, 70-80, 80-90, 90-100, 100-150, 150-200, 200-250, 250-300, 300-350, 350-400, 400-450, or 450-500 nucleotides in length, such that the homology arms have sufficient homology to undergo homologous recombination with the corresponding target sequences within the target nucleic acid. Alternatively, a given homology arm (or each homology arm) and/or corresponding target sequence can comprise corresponding regions of homology that are between about 0.5 kb to about 1 kb, about 1 kb to about 1.5 kb, about 1.5 kb to about 2 kb, or about 2 kb to about 2.5 kb in length. For example, the homology arms can each be about 750 nucleotides in length. The homology arms can be symmetrical (each about the same size in length), or they can be asymmetrical (one longer than the other).

When a nuclease agent is used in combination with an exogenous donor nucleic acid, the 5′ and 3′ target sequences are optionally located in sufficient proximity to the nuclease cleavage site (e.g., within sufficient proximity to the nuclease target sequence) so as to promote the occurrence of a homologous recombination event between the target sequences and the homology arms upon a single-strand break (nick) or double-strand break at the nuclease cleavage site. The term “nuclease cleavage site” includes a DNA sequence at which a nick or double-strand break is created by a nuclease agent (e.g., a Cas9 protein complexed with a guide RNA). The target sequences within the targeted locus that correspond to the 5′ and 3′ homology arms of the exogenous donor nucleic acid are “located in sufficient proximity” to a nuclease cleavage site if the distance is such as to promote the occurrence of a homologous recombination event between the 5′ and 3′ target sequences and the homology arms upon a single-strand break or double-strand break at the nuclease cleavage site. Thus, the target sequences corresponding to the 5′ and/or 3′ homology arms of the exogenous donor nucleic acid can be, for example, within at least 1 nucleotide of a given nuclease cleavage site or within at least 10 nucleotides to about 1,000 nucleotides of a given nuclease cleavage site. As an example, the nuclease cleavage site can be immediately adjacent to at least one or both of the target sequences.

The spatial relationship of the target sequences that correspond to the homology arms of the exogenous donor nucleic acid and the nuclease cleavage site can vary. For example, target sequences can be located 5′ to the nuclease cleavage site, target sequences can be located 3′ to the nuclease cleavage site, or the target sequences can flank the nuclease cleavage site.

Targeted Genetic Modifications. The mutation in the first isoform of the target protein that is generated by the editing or that is in the exogenous donor nucleic acid can be any type of mutation and any size mutation. In some embodiments, the mutation comprises an insertion, a deletion and/or a substitution of 1, 2, 3, 4, 5, 6, 7, 8, 9, 10, 11, 12, 13, 14, 15, 16, 17, 18, 19, or 20 amino acids (e.g., 1-20 amino acids, 1-5 amino acids, 1-3 amino acids, or 1 amino acid). In some embodiments, the mutation comprises a substitution (e.g., comprises a substitution of 1 amino acid). In some embodiments, the mutation consists essentially of a substitution (e.g., consists essentially of a substitution of 1 amino acid). In some embodiments, the mutation consists of a substitution (e.g., consists of a substitution of 1 amino acid). The mutation can be at any site in the target protein. For example, if the target protein is a cell surface protein, the mutation can be in the extracellular domain of the target protein. In some embodiments, the site of the mutation can be a site that is non-conserved between different mammalian species. In some embodiments, the mutation does not result in a secondary structure change in the surface protein. In some embodiments, the mutation is within the epitope targeted by an antigen-binding protein or is at a site that is accessible to ligand binding. In some embodiments, the mutation is not located at a site involved in a predicted or experimentally established or confirmed protein-protein interaction of the surface protein. In some embodiments, the mutation does not result in deleting or introducing a disulfide bond inter- or intramolecular interaction or a hydrophobic stacking. In some embodiments, the mutation does not result in deleting or introducing a posttranslational protein modification site, such as a glycosylation site. In some embodiments, the mutation is located at a site that has a unique topology compared to other mammalian proteins according to crystal structure analysis or computer-aided structure prediction. In instances where an antibody or antigen-binding protein reactive against a target protein already exists, the information regarding the epitope of the target protein that is recognized by the antibody or antigen-binding protein can be used to select the site of the mutation.

In some embodiments, the first isoform of the target protein is a genetically engineered isoform of the target protein. For example, the first isoform of the target protein can be genetically engineered to comprise a mutation (e.g., an artificial mutation that is not naturally occurring) to provide an altered epitope. The altered epitope can be, for example, in the binding region of an antigen-binding protein such as an antibody. In some embodiments, the target protein is IL2RG (e.g., human IL2RG), and the altered epitope is in a binding region of the REGN7257 anti-IL2RG antibody described elsewhere herein. REGN7257 binding region 1 is encoded by exon 3 of IL2RG (e.g., human IL2RG) and includes T127, F128, V129, V130, Q131, L132, Q133, D134, P135, R136, E137, P138, R139, R140, Q141, A142, T143, Q144, M145, L146, K147, L148, Q149, and N150. REGN7257 binding region 2 is encoded by exons 2 and 3 of IL2RG (e.g., human IL2RG) and includes L87, H88, Y89 (exon 2), W90 (codon is split between exons 2 and 3), Y91, K92, N93, S94, D95, N96, and D97 (exon 3). In some embodiments, the mutation can comprise a mutation (e.g., a substitution) encoded by nucleotides within exon 2 of the IL2RG gene (e.g., human IL2RG gene). In some embodiments, the mutation can comprise a mutation (e.g., a substitution) encoded by nucleotides within exon 3 of the IL2RG gene (e.g., human IL2RG gene). In some embodiments, the mutation can comprise a mutation (e.g., a substitution) encoded by nucleotides within exons 2 and 3 of the IL2RG gene (e.g., human IL2RG gene). In some embodiments, the mutation can comprise a mutation (e.g., a substitution) within the region from position T127 to position N150 of IL2RG (e.g., human IL2RG). In some embodiments, the mutation can comprise a mutation (e.g., a substitution) within the region from position L87 to position D97 of IL2RG (e.g., human IL2RG). In some embodiments, the mutation can comprise a mutation (e.g., a substitution) within the region from position T127 to position N150 of IL2RG (e.g., human IL2RG) and within the region from position L87 to position D97 of IL2RG (e.g., human IL2RG). In some embodiments, the mutation can comprise a mutation (e.g., a substitution) within REGN7257 binding region 1 (SEQ ID NO: 25). In some embodiments, the mutation can comprise a mutation (e.g., a substitution) within REGN7257 binding region 2 (SEQ ID NO: 62). In some embodiments, the mutation can comprise a mutation (e.g., a substitution) within REGN7257 binding region 1 (SEQ ID NO: 25) and within REGN7257 binding region 2 (SEQ ID NO: 62). In some embodiments, the mutation can comprise a mutation (e.g., a substitution) at one or more of the following positions: T127, F128, V129, V130, Q131, L132, Q133, D134, P135, R136, E137, P138, R139, R140, Q141, A142, T143, Q144, M145, L146, K147, L148, Q149, and N150. In some embodiments, the mutation can comprise a mutation (e.g., a substitution) at one or more of the following positions: L87, H88, Y89, W90, Y91, K92, N93, S94, D95, N96, and D97. In some embodiments, the mutation can comprise a mutation (e.g., a substitution) at one or more of the following positions: T127, F128, V129, V130, Q131, L132, Q133, D134, P135, R136, E137, P138, R139, R140, Q141, A142, T143, Q144, M145, L146, K147, L148, Q149, N150, L87, H88, Y89, W90, Y91, K92, N93, S94, D95, N96, and D97. For example, the mutation can comprise a mutation (e.g., a substitution) at position M145, position W90, position K92, position N93, position D95, position D97, position T127, position R139, position R140, position Q141, position T143, position K147, or a combination thereof. A mutation at a position within IL2RG encompasses mutations (e.g., substitutions) including only the residue at that position or mutations (e.g., substitutions) including the residue at that position as well as other residues at other positions. The nomenclature of the amino acid position for the mutations or residue disclosed herein refer to the position of the mutation or residue in the canonical isoform of human IL2RG set forth in SEQ ID NO: 21. In some embodiments, the mutation comprises a mutation (e.g., a substitution) at position M145. Examples of suitable M145 substitutions include a M145K substitution, a M145D substitution, a M145E substitution, a M145P substitution, a M145W substitution, or an M145Y substitution. In some embodiments, the mutation comprises a M145K substitution. In some embodiments, the mutation comprises a mutation (e.g., a substitution) at position W90. Examples of suitable W90 substitutions include a W90V substitution, a W90R substitution, a W90Q substitution, a W90L substitution, a W90K substitution, a W90E substitution, or a W90D substitution. In some embodiments, the mutation comprises a W90Q substitution. In some embodiments, the mutation comprises a mutation (e.g., a substitution) at positions M145 and W90.

### IV. Methods for Treatment in a Subject

In some embodiments of the present invention, any of the methods for improving engraftment of donor cells or for selective inhibition or selective depletion of host cells or non-edited cells in a subject described herein are methods for the treatment of a disease or disorder (e.g., any malignancy, such as any cancer) in a subject, and the methods can comprise administering a therapeutically effective amount of the donor cells or edited cells to the subject. In some embodiments of the present invention, any of the methods for improving engraftment of donor cells or for selective inhibition or selective depletion of host cells or non-edited cells in a subject described herein are methods for the treatment of a hematopoietic malignancy or a hematologic malignancy, and the methods can comprise administering a therapeutically effective amount of the donor cells or edited cells to the subject. In some embodiments, the methods can further comprise administering a therapeutically effective amount of the agent for selective inhibition or selective depletion of host cells or non-edited cells (e.g., antagonist or the antigen-binding protein or the population of immune effector cells).

In some embodiments, the administered cells comprise or express a therapeutic molecule, such as a therapeutic protein or enzyme, an immunoglobulin (e.g., antibody or antigen-binding fragment thereof), a chimeric antigen receptor (CAR), or an exogenous T cell receptor (TCR). In some embodiments, the therapeutic molecule, immunoglobulin, CAR, or exogenous TCR does not target the target protein (e.g., IL2RG). In some embodiments, the administered cells comprise or express an immunoglobulin, a CAR, or an exogenous TCR. In some embodiments, the administered cells comprise a CAR or an exogenous TCR.

As used herein, the terms “treat,” “treating,” and “treatment” mean to relieve or alleviate at least one symptom associated with the disease or disorder, or to slow or reverse the progression of the disease or disorder. Within the meaning of the present disclosure, the term “treat” also denotes to arrest, delay the onset (i.e., the period prior to clinical manifestation of a disease) and/or reduce the risk of developing or worsening a disease. For example, in connection with cancer, the term “treat” may mean eliminate or reduce the number or replication of cancer cells, and/or prevent, delay or inhibit metastasis, etc.

As used herein the term “effective amount” may be used interchangeably with the term “therapeutically effective amount” and refers to that quantity of an agent (e.g., antagonist or antigen-binding protein or population of immune effector cells), cell population (e.g., donor cells or edited cells), or pharmaceutical composition (e.g., a composition comprising an agent and/or donor cells or edited cells such as hematopoietic cells) that is sufficient to result in a desired activity upon administration to a subject in need thereof. Within the context of the present disclosure, the term “effective amount” refers to that quantity of a compound, cell population, or pharmaceutical composition that is sufficient to delay the manifestation, arrest the progression, relieve or alleviate at least one symptom of a disease or disorder (e.g., any malignancy, such as any cancer) treated by the methods of the present disclosure. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human. In some embodiments, the subject is a human patient having a hematopoietic malignancy or a hematologic malignancy. In some embodiments, the subject is a human patient having cancer (e.g., any type of cancer).

In some embodiments, a typical number of cells (e.g., immune cells or hematopoietic cells) administered to a mammal (e.g., a human) can be, for example, in the range of about 10⁶ to 10¹¹ cells. In some embodiments it may be desirable to administer fewer than 10⁶ cells to the subject. In some embodiments, it may be desirable to administer more than 10¹¹ cells to the subject. In some embodiments, one or more doses of cells includes about 10⁶ cells to about 10¹¹ cells, about 10⁷ cells to about 10¹⁰ cells, about 10⁸ cells to about 10⁹ cells, about 10⁶ cells to about 10⁸ cells, about 10⁷ cells to about 10⁹ cells, about 10⁷ cells to about 10¹⁰ cells, about 10⁷ cells to about 10¹¹ cells, about 10⁸ cells to about 10¹⁰ cells, about 10⁸ cells to about 10¹¹ cells, about 10⁸ cells to about 10¹⁰ cells, about 10⁸ cells to about 10¹¹ cells, or about 10¹⁰ cells to about 10¹¹ cells. In some embodiments, one or more doses of cells includes about 10⁶ to 10⁷ cells per kg.

The donor cells or edited cells and/or the agent for selective inhibition or selective depletion of host cells or non-edited cells may be administered in a pharmaceutically acceptable carrier or excipient as a pharmaceutical composition. See, e.g., Zhang et al. (2020) World J. Clin. Oncol. 11 (5): 275-282 and Atouf (2016) AAPS J. 18 (4): 844-848, each of which is herein incorporated by reference in its entirety for all purposes. The phrase “pharmaceutically acceptable,” as used in connection with compositions and/or cells described herein, refers to molecular entities and other ingredients of such compositions that are physiologically tolerable and do not typically produce untoward reactions when administered to a mammal (e.g., a human). In some embodiments, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions. Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover, herein incorporated by reference in its entirety for all purposes.

The agent for selective inhibition or selective depletion of host cells or non-edited cells can in some embodiments be administered simultaneously with the donor cells or edited cells. In some embodiments, the donor cells or edited cells are administered after agent. For example, in some embodiments, the donor cells or edited cells are administered within a day after the agent or at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 3 months, at least about 4 months, at least about 5 months, at least about 6 months or more after the agent. In some embodiments, the donor cells or edited cells are administered before the agent. For example, in some embodiments, the donor cells or edited cells are administered within a day before the agent or at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 3 months, at least about 4 months, at least about 5 months, at least about 6 months or more before the agent.

In some embodiments, the donor cells or edited cells are administered in multiple administrations (e.g., doses). In some embodiments, the donor cells or edited cells are administered to the subject once. In some embodiments, the donor cells or edited cells are administered to the subject more than once (e.g., at least 2, at least 3, at least 4, at least 5 or more times). In some embodiments, the donor cells or edited cells are administered to the subject at a regular interval (e.g., every 6 months). In some embodiments, the agent for selective inhibition or selective depletion of host cells or non-edited cells is administered in multiple administrations (e.g., doses). In some embodiments, the agent is administered to the subject once. In some embodiments, the agent is administered to the subject more than once (e.g., at least 2, at least 3, at least 4, at least 5 or more times). In some embodiments, the agent is administered to the subject at a regular interval (e.g., every 6 months).

In some embodiments, the agent is administered to the subject in multiple administrations (e.g., at least 2, at least 3, at least 4, at least 5 or more times) prior to administration of the donor cells or edited cells. In some embodiments, the agent is administered to the subject in multiple administrations (e.g., at least 2, at least 3, at least 4, at least 5 or more times) after administration of the donor cells or edited cells. In some embodiments, the agent is administered to the subject in multiple administrations (e.g., at least 2, at least 3, at least 4, at least 5 or more times) prior to administration of the donor cells or edited cells and in multiple administrations (e.g., at least 2, at least 3, at least 4, at least 5 or more times) after administration of the donor cells or edited cells.

In some embodiments, the subject has a disease, such as a cancer, and the methods are for treating the disease (e.g., the cancer). For example, the donor cells or edited cells can be engineered to express a therapeutic agent for treating that disease (e.g., if the disease is a cancer, the donor cells or edited cells can be engineered to express a CAR or exogenous TCR with anti-tumor reactivity). In some embodiments, the subject (e.g., human subject) has a hematopoietic malignancy. As used herein a hematopoietic malignancy refers to a malignant abnormality involving hematopoietic cells (e.g., blood cells, including progenitor and stem cells). Examples of hematopoietic malignancies include, without limitation, Hodgkin's lymphoma, non-Hodgkin's lymphoma, leukemia, and multiple myeloma. Exemplary leukemias include, without limitation, acute myeloid leukemia, acute lymphoid leukemia, chronic myelogenous leukemia, acute lymphoblastic leukemia or chronic lymphoblastic leukemia, and chronic lymphoid leukemia.

In some embodiments, the subject has a cancer, such as a solid tumor cancer or a liquid tumor cancer. In some embodiments, the subject has a solid tumor cancer. A solid tumor is a solid mass of cancer cells that grow in organ systems and can occur anywhere in the body, such as breast cancer. In contrast, liquid tumors are cancers that develop in the blood, bone marrow, or lymph nodes and includes leukemia, lymphoma, and myeloma. In some embodiments, the subject has a cancer, such as a hematologic cancer. Hematologic cancers are cancers that begin in blood-forming tissue, such as the bone marrow, or in the cells of the immune system. Examples of hematologic cancers include leukemia, lymphoma, and multiple myeloma. Hematologic cancers are also referred to blood cancer. In some embodiments, the subject has a hematopoietic disorder. In some embodiments, the subject has defective immune cells or a genetic deficiency in hematopoiesis, such as sickle cell disease or severe combined immunodeficiency (SCID). In some embodiments, the subject has a genetic hematopoietic disease (e.g., thalassemia). In some embodiments, the subject has a T-cell-mediated diseases, such as an IPEX-like syndrome, a CTLA-4-associated immune dysregulation, a hemophagocytic syndrome, ALPS syndrome, or a syndrome caused by heterozygous PTEN germline mutations. In some embodiments, the subject has an autoimmune disease. In some embodiments, the subject has graft-versus-host-disease. In some embodiments, the methods are for correction of congenital hematopoietic deficiencies.

In some embodiments of the present invention, any of the methods for improving engraftment of donor cells or for selective inhibition or selective depletion of host cells or non-edited cells in a subject described herein are methods for conditioning a subject's tissues (e.g., bone marrow) for engraftment or transplant. Such methods can be useful for treating such diseases without causing the toxicities that are observed in response to traditional conditioning therapies. In some embodiments of the present invention, any of the methods for improving engraftment of donor cells or for selective inhibition or selective depletion of host cells or non-edited cells in a subject described herein are methods for treating a subject defective or deficient in one or more cell types of the hematopoietic lineage. The methods can, in some embodiments, reconstitute the defective or deficient population of cells in vivo, thereby treating the pathology associated with the defect or depletion in the endogenous blood cell population. In some embodiments, the compositions and methods described herein can thus be used to treat a non-malignant hemoglobinopathy (e.g., a hemoglobinopathy selected from the group consisting of sickle cell anemia, thalassemia, Fanconi anemia, aplastic anemia, and Wiskott-Aldrich syndrome). In some embodiments, the compositions and methods described herein can be used to treat a malignancy or proliferative disorder, such as a cancer, such as hematologic cancer or a myeloproliferative disease. In the case of cancer treatment, in some embodiments the compositions and methods described herein may be administered to a patient so as to deplete a population of endogenous hematopoietic stem cells prior to hematopoietic stem cell transplantation therapy, in which case the transplanted cells can home to a niche created by the endogenous cell depletion step and establish productive hematopoiesis. This, in turn, can reconstitute a population of cells depleted during cancer cell eradication, such as during systemic chemotherapy.

In some embodiments, the donor cells or edited cells can be engineered to express a therapeutic agent for treating disease or the cancer (e.g., the donor cells or edited cells can be engineered to express a CAR or exogenous TCR with anti-tumor reactivity). For example, the donor cells or edited cells can be engineered to express a therapeutic molecule with therapeutic activity against any disease, such as any type of cancer (e.g., not dependent on whether the target protein is related to the disease or cancer), including disease or cancers that are unrelated to the target protein (e.g., the target protein discussed above is not what is being targeted to treat the disease or cancer, but the compositions and methods disclosed herein can provide a competitive advantage to the cells comprising or expressing the therapeutic molecule). For example, the disease or cancer can be a disease or cancer that is not associated with the target protein (e.g., the target protein does not cause the disease or cancer, and/or expression of the target protein is not correlated with the disease or cancer). In some embodiments, the therapeutic molecule may target the diseased cells and/or an antigen expressed on the diseased cells (e.g., a tumor-associated antigen).

Exemplary cancers that can be treated using the compositions and methods described herein include, without limitation, adenoid cystic carcinoma, adrenal gland cancer, anal cancer, basal cell carcinoma, bile duct cancer, bladder cancer, bone cancer, brain cancer, breast cancer, carcinoid tumor, cervical cancer, colorectal cancer, ductal carcinoma, endometrial cancer, esophageal cancer, gastric cancer, gastrointestinal stromal tumor-GIST, HER2-positive breast cancer, islet cell tumor, kidney cancer, laryngeal cancer, leukemia-acute lymphoblastic leukemia, leukemia-acute lymphocytic (ALL), leukemia-acute myeloid (AML), leukemia-adult, leukemia-childhood, leukemia-chronic lymphocytic (CLL), leukemia-chronic myeloid (CML), liver cancer, lobular carcinoma, lung cancer, lung cancer-small cell, lymphoma-Hodgkin's, lymphoma-non-Hodgkin's, malignant glioma, melanoma, meningioma, multiple myeloma, nasopharyngeal cancer, neuroendocrine cancer, oral cancer, osteosarcoma, ovarian cancer, pancreatic cancer, pancreatic neuroendocrine cancer, parathyroid cancer, penile cancer, peritoneal cancer, pituitary gland cancer, prostate cancer, renal cell carcinoma, retinoblastoma, salivary gland cancer, sarcoma, sarcoma-Kaposi, skin cancer, small intestine cancer, stomach cancer, testicular cancer, thymoma, thyroid cancer, uterine (endometrial) cancer, vaginal cancer, Wilms' tumor. Exemplary hematological cancers that can be treated using the compositions and methods described herein include, without limitation, acute myeloid leukemia, acute lymphoid leukemia, chronic myeloid leukemia, chronic lymphoid leukemia, multiple myeloma, diffuse large B-cell lymphoma, and non-Hodgkin's lymphoma, as well as other cancerous conditions, including neuroblastoma. Exemplary solid tumors that can be treated using the compositions and methods described herein include, without limitation, sarcomas and carcinomas. Sarcomas are tumors in a blood vessel, bone, fat tissue, ligament, lymph vessel, muscle or tendon. Carcinomas are tumors that form in epithelial cells. Epithelial cells are found in the skin, glands and the linings of organs.

### V. Combination Medicaments

In some embodiments of the present invention, combinations (e.g., combination medicaments) are provided for administration to a subject in need thereof. In some embodiments, such combinations comprise: (1) a population of donor cells or edited cells that express a first isoform of a target protein that is different from a second isoform of the target protein, wherein the second isoform is expressed in host cells or non-edited cells of the subject, and (2) an agent (e.g., an antagonist or an antigen-binding protein or a population of immune effector cells) that specifically binds to the second isoform of the target protein but does not specifically bind to the first isoform of the target protein. In some embodiments of the present invention, combinations (e.g., combination medicaments) are provided for administration to a subject in need thereof. In some embodiments, such combinations comprise: (1) a population of donor cells or edited cells modified to express a first isoform of a target protein that is different from a second isoform of the target protein, wherein the second isoform is expressed in host cells or non-edited cells of the subject, and (2) an agent (e.g., an antagonist or an antigen-binding protein or a population of immune effector cells) that specifically binds to the second isoform of the target protein but does not specifically bind to the first isoform of the target protein. In some embodiments, the first isoform and the second isoform are functionally indistinguishable but immunologically distinguishable. In some embodiments, such combinations comprise: (1) a population of donor cells or edited cells in which a target genomic locus has been edited to express a first isoform of a target protein that is different from a second isoform of the target protein, wherein the second isoform is expressed in host cells or non-edited cells of the subject, and (2) an agent (e.g., an antagonist or an antigen-binding protein or a population of immune effector cells) that specifically binds to the second isoform of the target protein but does not specifically bind to the first isoform of the target protein. In some embodiments, the first isoform and the second isoform are functionally indistinguishable but immunologically distinguishable. In some embodiments, such combinations comprise: (1) a population of donor cells or edited cells in which a target genomic locus has been edited to express a first isoform of a target protein that is different from a second isoform of the target protein, wherein the first isoform and the second isoform are functionally indistinguishable but immunologically distinguishable, and wherein the second isoform is expressed in host cells or non-edited cells of the subject, and (2) an agent (e.g., an antagonist or an antigen-binding protein or a population of immune effector cells) that specifically binds to the second isoform but does not specifically bind to the first isoform. In some embodiments, the donor cells or edited cells express only the first isoform of the target protein (e.g., IL2RG). In other embodiments, the donor cells or edited cells express both the first and second isoforms of the target protein (e.g., IL2RG).

The donor cells or edited cells can be any suitable cells as described above in the context of methods for improving engraftment of donor cells or for selective inhibition or selective depletion of host cells or non-edited cells in a subject. In some embodiments, the donor cells or edited cells comprise a genetic modification (insertion of a transgene, correction of a mutation, deletion or inactivation of a gene (e.g., insertion of premature stop codon or insertion of regulatory repressor sequence), or a change in an epigenetic modification important for expression of a gene) correcting or counteracting a disease-related gene defect present in a subject. In some embodiments, the donor cells or edited cells comprise a transgene. In some embodiments, the donor cells or edited cells comprise or express a therapeutic molecule, such as a therapeutic protein or enzyme, an immunoglobulin (e.g., antibody or antigen-binding fragment thereof), a chimeric antigen receptor (CAR) (e.g., CAR-T cells, CAR-NK cells), or an exogenous T cell receptor (TCR). In some embodiments, the donor cells or edited cells comprise a bicistronic nucleic acid construct encoding the therapeutic molecule and the first isoform of the target protein. See, e.g., Yeku et al. (2017) Sci. Rep. 7 (1): 10541 and Rafiq et al. (2018) Nat. Biotechnol. 36 (9): 847-856, each of which is herein incorporated by reference in its entirety for all purposes, for examples of bicistronic constructs expressing CARs and another molecule. For example, the bicistronic construct can encode both a therapeutic protein (e.g., a CAR) and the first isoform (e.g., a modified isoform) of the target protein (e.g., IL2RG). In one embodiment, the bicistronic construct encodes a therapeutic protein (e.g., a CAR) and a modified isoform of IL2RG. In some embodiments, the therapeutic molecule, immunoglobulin, CAR, or exogenous TCR does not target the target protein (e.g., IL2RG). In some embodiments, the donor cells or edited cells comprise or express an immunoglobulin, a CAR, or an exogenous TCR. In some embodiments, the donor cells or edited cells comprise a CAR or an exogenous TCR. In some embodiments, the donor cells or edited cells comprise a CAR or an exogenous TCR. For example, the donor cells or edited cells can be engineered to express a therapeutic molecule with therapeutic activity against any disease, such as any type of cancer (e.g., not dependent on whether the target protein is related to the disease or cancer), including disease or cancers that are unrelated to the target protein (e.g., the target protein discussed above is not what is being targeted to treat the disease or cancer, but the compositions and methods disclosed herein can provide a competitive advantage to the cells comprising or expressing the therapeutic molecule). For example, the disease or cancer can be a disease or cancer that is not associated with the target protein (e.g., the target protein does not cause the disease or cancer, and/or expression of the target protein is not correlated with the disease or cancer). Exemplary types of cancers and tumors that can be treated are described elsewhere herein. In some embodiments, the therapeutic molecule may target the diseased cells and/or an antigen expressed on the diseased cells (e.g., a tumor-associated antigen).

In some embodiments, the donor cells or edited cells are autologous (i.e., from the subject). In some embodiments, the donor cells or edited cells are allogeneic (i.e., not from the subject) or syngeneic (i.e., genetically identical, or sufficiently identical and immunologically compatible as to allow for transplantation). In some embodiments, the donor cells or edited cells are mammalian cells or non-human mammalian cells (e.g., mouse or rat cells or non-human primate cells) (e.g., the subject is a mammal or a non-human mammal, and the donor cells or edited cells are mammalian cells or non-human mammalian cells). In some embodiments, the donor cells or edited cells are human cells (e.g., the subject is a human, and the cells are human cells).

The target protein can be any suitable target protein as described above in the context of methods for improving engraftment of donor cells or for selective inhibition or selective depletion of host cells or non-edited cells in a subject. In some embodiments, the target protein is a protein that is expressed on the cell surface of hematopoietic cells, such as lymphocytes. In some embodiments, the target protein is a receptor, such as a cytokine receptor or a chemokine receptor. In some embodiments, the receptor is a cytokine receptor. In some embodiments, the target protein is a cytokine receptor sub-unit of an interleukin-2 (IL-2) receptor, an IL-4 receptor, an IL-7 receptor, an IL-9 receptor, an IL-15 receptor, or an IL-21 receptor. In some embodiments of the present invention, the target protein is interleukin-2 receptor subunit gamma (IL2RG).

In some embodiments, the second isoform of the target protein refers to the form that is present in the subject. In some embodiments, the second isoform of the target protein refers to the wild type form or native form of the target protein (i.e., the form that usually occurs in nature), and the first isoform refers to an isoform obtained by introducing a mutation in the nucleic acid sequence encoding the second isoform. The native form of a protein refers to a protein that is encoded by a nucleic acid sequence within the genome of the cell and that has not been inserted or mutated by genetic manipulation (i.e., a native protein is a protein that is not a transgenic protein or a genetically engineered protein).

In some embodiments, the mutation in the first isoform can be any type of mutation and any size mutation as described above in the context of methods for improving engraftment of donor cells or for selective inhibition or selective depletion of host cells or non-edited cells in a subject. In some embodiments, the first isoform of the target protein is a genetically engineered isoform of the target protein. For example, the first isoform of the target protein can be genetically engineered to comprise a mutation (e.g., an artificial mutation that is not naturally occurring) to provide an altered epitope. The altered epitope can be, for example, in the binding region of an antigen-binding protein such as an antibody. In some embodiments, the target protein is IL2RG (e.g., human IL2RG), and the altered epitope is in a binding region of the REGN7257 anti-IL2RG antibody described elsewhere herein. REGN7257 binding region 1 is encoded by exon 3 of IL2RG (e.g., human IL2RG) and includes T127, F128, V129, V130, Q131, L132, Q133, D134, P135, R136, E137, P138, R139, R140, Q141, A142, T143, Q144, M145, L146, K147, L148, Q149, and N150. REGN7257 binding region 2 is encoded by exons 2 and 3 of IL2RG (e.g., human IL2RG) and includes L87, H88, Y89 (exon 2), W90 (codon is split between exons 2 and 3), Y91, K92, N93, S94, D95, N96, and D97 (exon 3). In some embodiments, the mutation can comprise a mutation (e.g., a substitution) encoded by nucleotides within exon 2 of the IL2RG gene (e.g., human IL2RG gene). In some embodiments, the mutation can comprise a mutation (e.g., a substitution) encoded by nucleotides within exon 3 of the IL2RG gene (e.g., human IL2RG gene). In some embodiments, the mutation can comprise a mutation (e.g., a substitution) encoded by nucleotides within exons 2 and 3 of the IL2RG gene (e.g., human IL2RG gene). In some embodiments, the mutation can comprise a mutation (e.g., a substitution) within the region from position T127 to position N150 of IL2RG (e.g., human IL2RG). In some embodiments, the mutation can comprise a mutation (e.g., a substitution) within the region from position L87 to position D97 of IL2RG (e.g., human IL2RG). In some embodiments, the mutation can comprise a mutation (e.g., a substitution) within the region from position T127 to position N150 of IL2RG (e.g., human IL2RG) and within the region from position L87 to position D97 of IL2RG (e.g., human IL2RG). In some embodiments, the mutation can comprise a mutation (e.g., a substitution) within REGN7257 binding region 1 (SEQ ID NO: 25). In some embodiments, the mutation can comprise a mutation (e.g., a substitution) within REGN7257 binding region 2 (SEQ ID NO: 62). In some embodiments, the mutation can comprise a mutation (e.g., a substitution) within REGN7257 binding region 1 (SEQ ID NO: 25) and within REGN7257 binding region 2 (SEQ ID NO: 62). In some embodiments, the mutation can comprise a mutation (e.g., a substitution) at one or more of the following positions: T127, F128, V129, V130, Q131, L132, Q133, D134, P135, R136, E137, P138, R139, R140, Q141, A142, T143, Q144, M145, L146, K147, L148, Q149, and N150. In some embodiments, the mutation can comprise a mutation (e.g., a substitution) at one or more of the following positions: L87, H88, Y89, W90, Y91, K92, N93, S94, D95, N96, and D97. In some embodiments, the mutation can comprise a mutation (e.g., a substitution) at one or more of the following positions: T127, F128, V129, V130, Q131, L132, Q133, D134, P135, R136, E137, P138, R139, R140, Q141, A142, T143, Q144, M145, L146, K147, L148, Q149, N150, L87, H88, Y89, W90, Y91, K92, N93, S94, D95, N96, and D97. For example, the mutation can comprise a mutation (e.g., a substitution) at position M145, position W90, position K92, position N93, position D95, position D97, position T127, position R139, position R140, position Q141, position T143, position K147, or a combination thereof. A mutation at a position within IL2RG encompasses mutations (e.g., substitutions) including only the residue at that position or mutations (e.g., substitutions) including the residue at that position as well as other residues at other positions. The nomenclature of the amino acid position for the mutations or residue disclosed herein refer to the position of the mutation or residue in the canonical isoform of human IL2RG set forth in SEQ ID NO: 21. In some embodiments, the mutation comprises a mutation (e.g., a substitution) at position M145. Examples of suitable M145 substitutions include a M145K substitution, a M145D substitution, a M145E substitution, a M145P substitution, a M145W substitution, or an M145Y substitution. In some embodiments, the mutation comprises a M145K substitution. In some embodiments, the mutation comprises a mutation (e.g., a substitution) at position W90. Examples of suitable W90 substitutions include a W90V substitution, a W90R substitution, a W90Q substitution, a W90L substitution, a W90K substitution, a W90E substitution, or a W90D substitution. In some embodiments, the mutation comprises a W90Q substitution. In some embodiments, the mutation comprises a mutation (e.g., a substitution) at positions M145 and W90.

The agent for selective inhibition or selective depletion of cells expressing a first isoform can be any suitable agent as described above in the context of methods for improving engraftment of donor cells or for selective inhibition or selective depletion of host cells or non-edited cells in a subject. In some embodiments, the agent comprises an antagonist that specifically binds to the second isoform of the target protein but does not specifically bind to the first isoform of the target protein. In some embodiments, the agent comprises an antigen-binding protein that specifically binds to the second isoform of the target protein but does not specifically bind to the first isoform of the target protein. In some embodiments, the agent comprises a population of cells (i.e., immune effector cells such as chimeric antigen receptor T cells (CAR-T)) expressing an antigen-binding protein that specifically binds to the second isoform of the target protein but does not specifically bind to the first isoform of the target protein.

The agent can in some embodiments be for administration simultaneously with the donor cells or edited cells. In some embodiments, the donor cells or edited cells are for administration after the agent. For example, in some embodiments, the donor cells or edited cells are for administration within a day after the agent or at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 3 months, at least about 4 months, at least about 5 months, at least about 6 months or more after the agent. In some embodiments, the donor cells or edited cells are for administration before the agent. For example, in some embodiments, the donor cells or edited cells are for administration within a day before the agent or at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 3 months, at least about 4 months, at least about 5 months, at least about 6 months or more before the agent.

In some embodiments, the donor cells or edited cells are for administration in multiple administrations (e.g., doses). In some embodiments, the donor cells or edited cells are for administration to the subject once. In some embodiments, the donor cells or edited cells are for administration to the subject more than once (e.g., at least 2, at least 3, at least 4, at least 5 or more times). In some embodiments, the donor cells or edited cells are for administration to the subject at a regular interval (e.g., every 6 months). In some embodiments, the agent is for administration in multiple administrations (e.g., doses). In some embodiments, the agent is for administration to the subject once. In some embodiments, the agent is for administration to the subject more than once (e.g., at least 2, at least 3, at least 4, at least 5 or more times). In some embodiments, the agent is for administration to the subject at a regular interval (e.g., every 6 months).

In some embodiments, the agent is administered to the subject in multiple administrations (e.g., at least 2, at least 3, at least 4, at least 5 or more times) prior to administration of the donor cells or edited cells. In some embodiments, the agent is administered to the subject in multiple administrations (e.g., at least 2, at least 3, at least 4, at least 5 or more times) after administration of the donor cells or edited cells. In some embodiments, the agent is administered to the subject in multiple administrations (e.g., at least 2, at least 3, at least 4, at least 5 or more times) prior to administration of the donor cells or edited cells and in multiple administrations (e.g., at least 2, at least 3, at least 4, at least 5 or more times) after administration of the donor cells or edited cells.

In some embodiments, the agent comprises an antigen-binding protein as described above in the context of methods for improving engraftment of donor cells or for selective inhibition or selective depletion of host cells or non-edited cells in a subject (e.g., a method in which the agent blocks interaction of endogenous ligand with the target on the non-edited cells). In some embodiments, the agent comprises an anti-IL2RG antigen-binding protein as described above in the context of methods for improving engraftment of donor cells or for selective inhibition or selective depletion of host cells or non-edited cells in a subject. In some embodiments, the agent comprises REGN7257 (also called H4H12889P), or a variant thereof as described above in the context of methods for improving engraftment of donor cells or for selective inhibition or selective depletion of host cells or non-edited cells in a subject or an antigen-binding protein that binds to the same epitope as REGN7257. In some embodiments, the agent comprises one or more nucleic acids encoding an antigen-binding protein as described above in the context of methods for improving engraftment of donor cells or for selective inhibition or selective depletion of host cells or non-edited cells in a subject. In some embodiments, the donor cells or edited cells can be engineered to express a therapeutic molecule for cell therapy as described elsewhere herein with therapeutic activity against any disease, such as any type of cancer (e.g., not dependent on whether the target protein is related to the disease or cancer), including disease or cancers that are unrelated to the target protein (e.g., the target protein discussed above is not what is being targeted to treat the disease or cancer, but the compositions and methods disclosed herein can provide a competitive advantage to the cells comprising or expressing the therapeutic molecule). For example, the disease or cancer can be a disease or cancer that is not associated with the target protein (e.g., the target protein does not cause the disease or cancer, and/or expression of the target protein is not correlated with the disease or cancer). In some embodiments, the therapeutic molecule may target the diseased cells and/or an antigen expressed on the diseased cells (e.g., a tumor-associated antigen).

In some embodiments of the present invention, a subject can include, for example, any type of animal or mammal. Mammals include, for example, humans, non-human mammals, non-human primates, monkeys, apes, cats, dogs, horses, bulls, deer, bison, sheep, rabbits, rodents (e.g., but not limited to, mice, rats, hamsters, and guinea pigs), and livestock (e.g., but not limited to, bovine species such as cows and steer; ovine species such as sheep and goats; and porcine species such as pigs and boars). Birds include, for example, chickens, turkeys, ostrich, geese, and ducks. Domesticated animals and agricultural animals are also included. The term “non-human mammal” excludes humans. Particular non-limiting examples of non-human mammals include rodents, such as mice and rats. In some embodiments of the present invention, the subject is a human.

In some embodiments of the present invention, the combination (e.g., combination medicament) is used for any of the methods for the treatment as described in more detail above. In some embodiments of the present invention, the combination (e.g., combination medicament) is used for any of the methods for the treatment of a hematopoietic malignancy or a hematologic malignancy as described in more detail above. In some embodiments of the present invention, the combination (e.g., combination medicament) is used for any of the methods for the treatment of a cancer (e.g., any type of cancer) as described in more detail above.

In some embodiments, the subject has a disease, such as a cancer, and the methods are for treating the disease (e.g., the cancer). For example, the donor cells or edited cells can be engineered to express a therapeutic agent for treating that disease (e.g., if the disease is a cancer, the donor cells or edited cells can be engineered to express a CAR or exogenous TCR with anti-tumor reactivity). For example, the donor cells or edited cells can be engineered to express a therapeutic molecule with therapeutic activity against any disease, such as any type of cancer (e.g., not dependent on whether the target protein is related to the disease or cancer), including disease or cancers that are unrelated to the target protein (e.g., the target protein discussed above is not what is being targeted to treat the disease or cancer, but the compositions and methods disclosed herein can provide a competitive advantage to the cells comprising or expressing the therapeutic molecule). For example, the disease or cancer can be a disease or cancer that is not associated with the target protein (e.g., the target protein does not cause the disease or cancer, and/or expression of the target protein is not correlated with the disease or cancer). In some embodiments, the therapeutic molecule may target the diseased cells and/or an antigen expressed on the diseased cells (e.g., a tumor-associated antigen).

In some embodiments, the subject (e.g., human subject) has a hematopoietic malignancy. As used herein a hematopoietic malignancy refers to a malignant abnormality involving hematopoietic cells (e.g., blood cells, including progenitor and stem cells). Examples of hematopoietic malignancies include, without limitation, Hodgkin's lymphoma, non-Hodgkin's lymphoma, leukemia, and multiple myeloma. Exemplary leukemias include, without limitation, acute myeloid leukemia, acute lymphoid leukemia, chronic myelogenous leukemia, acute lymphoblastic leukemia or chronic lymphoblastic leukemia, and chronic lymphoid leukemia.

In some embodiments, the subject has a cancer, such as a solid tumor cancer or a liquid tumor cancer. In some embodiments, the subject has a solid tumor cancer. A solid tumor is a solid mass of cancer cells that grow in organ systems and can occur anywhere in the body, such as breast cancer. In contrast, liquid tumors are cancers that develop in the blood, bone marrow, or lymph nodes and includes leukemia, lymphoma, and myeloma. In some embodiments, the subject has a cancer, such as a hematologic cancer. Hematologic cancers are cancers that begin in blood-forming tissue, such as the bone marrow, or in the cells of the immune system. Examples of hematologic cancers include leukemia, lymphoma, and multiple myeloma.

Hematologic cancers are also referred to blood cancer. In some embodiments, the subject has a hematopoietic disorder. In some embodiments, the subject has defective immune cells or a genetic deficiency in hematopoiesis, such as sickle cell disease or severe combined immunodeficiency (SCID). In some embodiments, the subject has a genetic hematopoietic disease (e.g., thalassemia). In some embodiments, the subject has a T-cell-mediated diseases, such as an IPEX-like syndrome, a CTLA-4-associated immune dysregulation, a hemophagocytic syndrome, ALPS syndrome, or a syndrome caused by heterozygous PTEN germline mutations. In some embodiments, the subject has an autoimmune disease. In some embodiments, the subject has graft-versus-host-disease. In some embodiments, the methods are for correction of congenital hematopoietic deficiencies.

In some embodiments of the present invention, any of the methods for improving engraftment of donor cells or for selective inhibition or selective depletion of host cells or non-edited cells in a subject described herein are methods for conditioning a subject's tissues (e.g., bone marrow) for engraftment or transplant. Such methods can be useful for treating such diseases without causing the toxicities that are observed in response to traditional conditioning therapies. In some embodiments of the present invention, any of the methods for improving engraftment of donor cells or for selective inhibition or selective depletion of host cells or non-edited cells in a subject described herein are methods for treating a subject defective or deficient in one or more cell types of the hematopoietic lineage. The methods can, in some embodiments, reconstitute the defective or deficient population of cells in vivo, thereby treating the pathology associated with the defect or depletion in the endogenous blood cell population. In some embodiments, the compositions and methods described herein can thus be used to treat a non-malignant hemoglobinopathy (e.g., a hemoglobinopathy selected from the group consisting of sickle cell anemia, thalassemia, Fanconi anemia, aplastic anemia, and Wiskott-Aldrich syndrome). In some embodiments, the compositions and methods described herein can be used to treat a malignancy or proliferative disorder, such as a cancer, such as hematologic cancer or a myeloproliferative disease. In the case of cancer treatment, in some embodiments the compositions and methods described herein may be administered to a patient so as to deplete a population of endogenous hematopoietic stem cells prior to hematopoietic stem cell transplantation therapy, in which case the transplanted cells can home to a niche created by the endogenous cell depletion step and establish productive hematopoiesis. This, in turn, can reconstitute a population of cells depleted during cancer cell eradication, such as during systemic chemotherapy. In some embodiments, the donor cells or edited cells can be engineered to express a therapeutic agent for treating the cancer (e.g., the donor cells or edited cells can be engineered to express a CAR or exogenous TCR with anti-tumor reactivity). Exemplary cancers that can be treated using the compositions and methods described herein include, without limitation, adenoid cystic carcinoma, adrenal gland cancer, anal cancer, basal cell carcinoma, bile duct cancer, bladder cancer, bone cancer, brain cancer, breast cancer, carcinoid tumor, cervical cancer, colorectal cancer, ductal carcinoma, endometrial cancer, esophageal cancer, gastric cancer, gastrointestinal stromal tumor-GIST, HER2-positive breast cancer, islet cell tumor, kidney cancer, laryngeal cancer, leukemia-acute lymphoblastic leukemia, leukemia-acute lymphocytic (ALL), leukemia-acute myeloid (AML), leukemia-adult, leukemia-childhood, leukemia-chronic lymphocytic (CLL), leukemia-chronic myeloid (CML), liver cancer, lobular carcinoma, lung cancer, lung cancer-small cell, lymphoma-Hodgkin's, lymphoma-non-Hodgkin's, malignant glioma, melanoma, meningioma, multiple myeloma, nasopharyngeal cancer, neuroendocrine cancer, oral cancer, osteosarcoma, ovarian cancer, pancreatic cancer, pancreatic neuroendocrine cancer, parathyroid cancer, penile cancer, peritoneal cancer, pituitary gland cancer, prostate cancer, renal cell carcinoma, retinoblastoma, salivary gland cancer, sarcoma, sarcoma-Kaposi, skin cancer, small intestine cancer, stomach cancer, testicular cancer, thymoma, thyroid cancer, uterine (endometrial) cancer, vaginal cancer, Wilms' tumor. Exemplary hematological cancers that can be treated using the compositions and methods described herein include, without limitation, acute myeloid leukemia, acute lymphoid leukemia, chronic myeloid leukemia, chronic lymphoid leukemia, multiple myeloma, diffuse large B-cell lymphoma, and non-Hodgkin's lymphoma, as well as other cancerous conditions, including neuroblastoma. Exemplary solid tumors that can be treated using the compositions and methods described herein include, without limitation, sarcomas and carcinomas. Sarcomas are tumors in a blood vessel, bone, fat tissue, ligament, lymph vessel, muscle or tendon. Carcinomas are tumors that form in epithelial cells. Epithelial cells are found in the skin, glands and the linings of organs.

### VI. Engineered IL2RG Proteins and Cell(s) Comprising Modified IL2RG Proteins

In some embodiments of the present invention, genetically engineered interleukin-2 receptor subunit gamma (IL2RG) proteins are provided comprising an artificial mutation to provide an altered epitope. In some embodiments, the genetically engineered IL2RG protein is functionally indistinguishable but immunologically distinguishable from a native or wild type IL2RG protein.

The genetically engineered IL2RG protein can comprise any mutation as described above in the context of methods for improving engraftment of donor cells or for selective inhibition or selective depletion of host cells or non-edited cells in a subject. In some embodiments, the mutation in the genetically engineered IL2RG protein can be any type of mutation and any size mutation as described above in the context of methods for improving engraftment of donor cells or for selective inhibition or selective depletion of host cells or non-edited cells in a subject. The altered epitope can be, for example, in the binding region of an antigen-binding protein such as an antibody. In some embodiments, the altered epitope is in the binding region of a REGN7257 anti-IL2RG antibody described elsewhere herein. REGN7257 binding region 1 is encoded by exon 3 of IL2RG (e.g., human IL2RG) and includes T127, F128, V129, V130, Q131, L132, Q133, D134, P135, R136, E137, P138, R139, R140, Q141, A142, T143, Q144, M145, L146, K147, L148, Q149, and N150. REGN7257 binding region 2 is encoded by exons 2 and 3 of IL2RG (e.g., human IL2RG) and includes L87, H88, Y89 (exon 2), W90 (codon is split between exons 2 and 3), Y91, K92, N93, S94, D95, N96, and D97 (exon 3). In some embodiments, the mutation can comprise a mutation (e.g., a substitution) encoded by nucleotides within exon 2 of the IL2RG gene (e.g., human IL2RG gene). In some embodiments, the mutation can comprise a mutation (e.g., a substitution) encoded by nucleotides within exon 3 of the IL2RG gene (e.g., human IL2RG gene). In some embodiments, the mutation can comprise a mutation (e.g., a substitution) encoded by nucleotides within exons 2 and 3 of the IL2RG gene (e.g., human IL2RG gene). In some embodiments, the mutation can comprise a mutation (e.g., a substitution) within the region from position T127 to position N150 of IL2RG (e.g., human IL2RG). In some embodiments, the mutation can comprise a mutation (e.g., a substitution) within the region from position L87 to position D97 of IL2RG (e.g., human IL2RG). In some embodiments, the mutation can comprise a mutation (e.g., a substitution) within the region from position T127 to position N150 of IL2RG (e.g., human IL2RG) and within the region from position L87 to position D97 of IL2RG (e.g., human IL2RG). In some embodiments, the mutation can comprise a mutation (e.g., a substitution) within REGN7257 binding region 1 (SEQ ID NO: 25). In some embodiments, the mutation can comprise a mutation (e.g., a substitution) within REGN7257 binding region 2 (SEQ ID NO: 62). In some embodiments, the mutation can comprise a mutation (e.g., a substitution) within REGN7257 binding region 1 (SEQ ID NO: 25) and within REGN7257 binding region 2 (SEQ ID NO: 62). In some embodiments, the mutation can comprise a mutation (e.g., a substitution) at one or more of the following positions: T127, F128, V129, V130, Q131, L132, Q133, D134, P135, R136, E137, P138, R139, R140, Q141, A142, T143, Q144, M145, L146, K147, L148, Q149, and N150. In some embodiments, the mutation can comprise a mutation (e.g., a substitution) at one or more of the following positions: L87, H88, Y89, W90, Y91, K92, N93, S94, D95, N96, and D97. In some embodiments, the mutation can comprise a mutation (e.g., a substitution) at one or more of the following positions: T127, F128, V129, V130, Q131, L132, Q133, D134, P135, R136, E137, P138, R139, R140, Q141, A142, T143, Q144, M145, L146, K147, L148, Q149, N150, L87, H88, Y89, W90, Y91, K92, N93, S94, D95, N96, and D97.

In some embodiments, the mutation can comprise a mutation (e.g., a substitution) at position M145, position W90, position K92, position N93, position D95, position D97, position T127, position R139, position R140, position Q141, position T143, position K147, or any combination thereof. A mutation at a position within IL2RG encompasses mutations (e.g., substitutions) including only the residue at that position or mutations (e.g., substitutions) including the residue at that position as well as other residues at other positions. The nomenclature of the amino acid position for the mutations or residue disclosed herein refer to the position of the mutation or residue in the canonical isoform of human IL2RG set forth in SEQ ID NO: 21. In some embodiments, the mutation comprises a mutation (e.g., a substitution) at position M145. Examples of suitable M145 substitutions include a M145K substitution, a M145D substitution, a M145E substitution, a M145P substitution, a M145W substitution, or an M145Y substitution. In some embodiments, the mutation comprises a M145K substitution. In some embodiments, the mutation comprises a mutation (e.g., a substitution) at position W90. Examples of suitable W90 substitutions include a W90V substitution, a W90R substitution, a W90Q substitution, a W90L substitution, a W90K substitution, a W90E substitution, or a W90D substitution. In some embodiments, the mutation comprises a W90Q substitution. In some embodiments, the mutation comprises a mutation (e.g., a substitution) at positions M145 and W90.

In some embodiments of the present invention, nucleic acids encoding the genetically engineered IL2RG protein are also provided. In some embodiments, the nucleic acid comprises deoxyribonucleic acid (DNA) or ribonucleic acid (RNA). Such nucleic acids can be DNA, RNA, or hybrids or derivatives of either DNA or RNA. Optionally, in some embodiments, the nucleic acid can be codon-optimized for efficient translation into protein in a particular cell or organism. As a non-limiting example, the nucleic acid can be modified to substitute codons having a higher frequency of usage in a human cell, a mammalian cell, a rodent cell, a mouse cell, a rat cell, or any other host cell of interest, as compared to the naturally occurring polynucleotide sequence. Any portion or fragment of a nucleic acid molecule can be produced by: (1) isolating the molecule from its natural milieu; (2) using recombinant DNA technology (e.g., but not limited to, PCR amplification or cloning); or (3) using chemical synthesis methods. Nucleic acids can comprise modifications for improved stability or reduced immunogenicity. Non-limiting examples of modifications include: (1) alteration or replacement of one or both of the non-linking phosphate oxygens and/or of one or more of the linking phosphate oxygens in the phosphodiester backbone linkage; (2) alteration or replacement of a constituent of a ribose sugar such as alteration or replacement of the 2′ hydroxyl on the ribose sugar; (3) replacement of the phosphate moiety with dephospho linkers; (4) modification or replacement of a naturally occurring nucleobase; (5) replacement or modification of a ribose-phosphate backbone; (6) modification of the 3′ end or 5′ end of the oligonucleotide (e.g., but not limited to, removal, modification or replacement of a terminal phosphate group or conjugation of a moiety); and (7) modification of the sugar.

In some embodiments, the nucleic acids can be in the form of an expression construct as defined elsewhere herein. As a non-limiting example, the nucleic acids can include regulatory regions that control expression of the nucleic acid molecule (e.g., but not limited to, transcription or translation control regions), full-length or partial coding regions, and combinations thereof. As a non-limiting example, the nucleic acids can be operably linked to a promoter active in a cell or organism of interest. Promoters that can be used in such expression constructs include promoters active, for example, in one or more of a eukaryotic cell, such as a mammalian cell (e.g., a non-human mammalian cell or a human cell), such as a rodent cell (e.g., but not limited to, a mouse cell, or a rat cell). Such promoters can be, for example, conditional promoters, inducible promoters, constitutive promoters, or tissue-specific promoters.

In some embodiments of the present invention, cells or populations of cells comprising the genetically engineered IL2RG protein are also provided. In some embodiments, the genetically engineered IL2RG protein is the only form of IL2RG expressed by the cells. In other embodiments, the cells express both the genetically engineered IL2RG protein and the endogenous IL2RG protein. The cells can be any suitable cells as described above in the context of methods for improving engraftment of donor cells or for selective inhibition or selective depletion of host cells or non-edited cells in a subject. In some embodiments, the cells are immune cells. In some embodiments, the cells are hematopoietic cells. In some embodiments, the cells are lymphocytes or lymphoid progenitor cells. In some embodiments, the cells are T cells (e.g., CD4+ T cells, CD8+ T cells, memory T cells, regulatory T cells, gamma delta T cells, mucosal-associated invariant T cells (MAIT), tumor infiltrating lymphocytes (TILs), or any combination thereof). In some embodiments, the cells are TILs. In some embodiments, the cells are B cells. In some embodiments, the cells are natural killer (NK) cells. In some embodiments, the cells are innate lymphoid cells. In some embodiments, the cells are dendritic cells. In some embodiments, the cells are hematopoietic stem cells (HSCs) or hematopoietic stem and progenitor cells (HSPCs) or descendants thereof. HSCs are capable of giving rise to both myeloid and lymphoid progenitor cells that further give rise to myeloid cells (e.g., monocytes, macrophages, neutrophils, basophils, dendritic cells, erythrocytes, platelets, etc.) and lymphoid cells (e.g., T cells, B cells, NK cells), respectively. In some embodiments, the cells are induced pluripotent stem cells (e.g., human induced pluripotent stem cells). In some embodiments, the cells are derived from induced pluripotent stem cells (e.g., NK cells derived from induced pluripotent stem cells). In some embodiments, the cells are HSCs or HSPCs. In some embodiments, the cells are derived from HSCs or HSPCs.

In some embodiments, the cells comprise a genetic modification (insertion of a transgene, correction of a mutation, deletion or inactivation of a gene (e.g., insertion of premature stop codon or insertion of regulatory repressor sequence), or a change in an epigenetic modification important for expression of a gene) correcting or counteracting a disease-related gene defect present in a subject. In some embodiments, the cells comprise a transgene. In some embodiments, the cells comprise or express a therapeutic molecule, such as a therapeutic protein or enzyme, an immunoglobulin (e.g., antibody or antigen-binding fragment thereof), a chimeric antigen receptor (CAR), or an exogenous T cell receptor (TCR). In some embodiments, the cells comprise a bicistronic nucleic acid construct encoding the therapeutic molecule and the first isoform of the target protein. See, e.g., Yeku et al. (2017) Sci. Rep. 7 (1): 10541 and Rafiq et al. (2018) Nat. Biotechnol. 36 (9): 847-856, each of which is herein incorporated by reference in its entirety for all purposes, for examples of bicistronic constructs expressing CARs and another molecule. For example, the bicistronic construct can encode both a therapeutic protein (e.g., a CAR) and the first isoform (e.g., a modified isoform) of the target protein (e.g., IL2RG). In one embodiment, the bicistronic construct encodes a therapeutic protein (e.g., a CAR) and a modified isoform of IL2RG. In some embodiments, the therapeutic molecule, immunoglobulin, CAR, or exogenous TCR does not target the target protein (e.g., IL2RG). In some embodiments, the cells comprise or express an immunoglobulin, a CAR, or an exogenous TCR. In some embodiments, the cells comprise a CAR or an exogenous TCR. In some embodiments, the cells comprise a CAR or an exogenous TCR. For example, the donor cells or edited cells can be engineered to express a therapeutic molecule with therapeutic activity against any disease, such as any type of cancer (e.g., not dependent on whether the target protein is related to the disease or cancer), including disease or cancers that are unrelated to the target protein (e.g., the target protein discussed above is not what is being targeted to treat the disease or cancer, but the compositions and methods disclosed herein can provide a competitive advantage to the cells comprising or expressing the therapeutic molecule). For example, the disease or cancer can be a disease or cancer that is not associated with the target protein (e.g., the target protein does not cause the disease or cancer, and/or expression of the target protein is not correlated with the disease or cancer). Exemplary types of cancers and tumors that can be treated are described elsewhere herein. In some embodiments, the therapeutic molecule targets the diseased cells and/or an antigen expressed by the diseased cells (e.g., a tumor-associated antigen).

In some embodiments, the cells are autologous (i.e., from the subject). In some embodiments, the cells are allogeneic (i.e., not from the subject) or syngeneic (i.e., genetically identical, or sufficiently identical and immunologically compatible as to allow for transplantation). In some embodiments, the cells are mammalian cells or non-human mammalian cells (e.g., mouse or rat cells or non-human primate cells) (e.g., the subject is a mammal or a non-human mammal, and the cells are mammalian cells or non-human mammalian cells). In some embodiments, the cells are human cells (e.g., the subject is a human, and the cells are human cells).

In some embodiments, the cells can further comprise an exogenous donor nucleic acid (e.g., comprising the mutation) and/or a nuclease agent or one or more nucleic acids encoding the nuclease agent, wherein the nuclease agent targets a nuclease target sequence in a target genomic locus (e.g., an IL2RG locus). Such exogenous donor nucleic acids and nuclease agents are described above in the context of methods for generating donor cells or edited cells. In some embodiments, the cells can further comprise an exogenous donor nucleic acid (e.g., comprising the mutation) and/or a nuclease agent or one or more nucleic acids encoding the nuclease agent, wherein the nuclease agent targets a nuclease target sequence in the IL2RG genomic locus. Such exogenous donor nucleic acids and nuclease agents are described above in the context of methods for editing a target genomic locus encoding a target protein.

All patent filings, websites, other publications, accession numbers and the like cited above or below are incorporated by reference in their entirety for all purposes to the same extent as if each individual item were specifically and individually indicated to be so incorporated by reference. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. When a DNA sequence encoding an amino acid sequence is provided, it is understood that RNA sequences that encode the same amino acid sequence are also provided (by replacing the thymines with uracils). The amino acid sequences follow the standard convention of beginning at the amino terminus of the sequence and proceeding forward (i.e., from left to right in each line) to the carboxy terminus.

TABLE 1
Description of Sequences.

| SEQ | | |
|---|---|---|
| ID NO | Type | Description |
| 1 | DNA | REGN7257 V_{H} |
| 2 | Protein | REGN7257 V_{H} |
| 3 | DNA | REGN7257 V_{H} CDR1 |
| 4 | Protein | REGN7257 V_{H} CDR1 |
| 5 | DNA | REGN7257 V_{H} CDR2 |
| 6 | Protein | REGN7257 V_{H} CDR2 |
| 7 | DNA | REGN7257 V_{H} CDR3 |
| 8 | Protein | REGN7257 V_{H} CDR3 |
| 9 | DNA | REGN7257 V_{L} |
| 10 | Protein | REGN7257 V_{L} |
| 11 | DNA | REGN7257 V_{L} CDR1 |
| 12 | Protein | REGN7257 V_{L} CDR1 |
| 13 | DNA | REGN7257 V_{L} CDR2 |
| 14 | Protein | REGN7257 V_{L} CDR2 |
| 15 | DNA | REGN7257 V_{L} CDR3 |
| 16 | Protein | REGN7257 V_{L} CDR3 |
| 17 | DNA | REGN7257 HC |
| 18 | Protein | REGN7257 HC |
| 19 | DNA | REGN7257 LC |
| 20 | Protein | REGN7257 LC |
| 21 | Protein | Human IL2RG (P31785-1; NP_000197.1) |
| 22 | Protein | Human IL2RG M145K |
| 23 | DNA | Human IL2RG mRNA (cDNA) (NM_000206.3) |
| 24 | DNA | Human IL2RG CDS (CCDS14406.1) |
| 25 | Protein | Human IL2RG Fragment - |
| | | REGN7257 Binding Region 1 |
| 26 | Protein | Mouse IL2RG Fragment - Region 1 |
| 27-37 | Protein | Human IL2RG Fragment Variants |
| 38 | Protein | Human IL2RG Fragment v2 |
| 39 | Protein | Human IL2RG Fragment v2 with M145K Mutation |
| 40 | DNA | Human IL2RG Exon 3 Fragment |
| 41 | Protein | Cas9 Protein |
| 42 | DNA | Cas9 CDS |
| 43 | Protein | SV40 NLS v1 |
| 44 | Protein | SV40 NLS v2 |
| 45 | Protein | Nucleoplasmin NLS |
| 46 | RNA | crRNA Tail v1 |
| 47 | RNA | crRNA Tail v2 |
| 48 | RNA | TracrRNA v1 |
| 49 | RNA | TracrRNA v2 |
| 50 | RNA | TracrRNA v3 |
| 51 | RNA | gRNA Scaffold v1 |
| 52 | RNA | gRNA Scaffold v2 |
| 53 | RNA | gRNA Scaffold v3 |
| 54 | RNA | gRNA Scaffold v4 |
| 55 | RNA | gRNA Scaffold v5 |
| 56 | RNA | gRNA Scaffold v6 |
| 57 | RNA | gRNA Scaffold v7 |
| 58 | RNA | gRNA Scaffold v8 |
| 59 | DNA | Guide RNA Target Sequence Plus PAM v1 |
| 60 | DNA | Guide RNA Target Sequence Plus PAM v2 |
| 61 | DNA | Guide RNA Target Sequence Plus PAM v3 |
| 62 | Protein | Human IL2RG Fragment - |
| | | REGN7257 Binding Region 2 |
| 63 | Protein | Mouse IL2RG Fragment - Region 2 |
| 64-75 | DNA | Guide RNA Target Sequences |
| 76-87 | RNA | Guide RNA DNA-Targeting Segments |
| 88-117 | DNA | ssODN Sequences |
| 118- | DNA | Guide RNA Target Sequences |
| 135 | | |
| 136- | RNA | Guide RNA DNA-Targeting Segments |
| 153 | | |
| 154- | DNA | ssODN Sequences |
| 173 | | |
| 174 | Protein | Human IL2RG Fragment v3 |
| 175 | Protein | Human IL2RG Fragment v3 with W90Q Mutation |

### Examples

### Example 1. Antibody-Resistant Modified Receptor (ARMoR) Approach Enhances Lymphocyte Adoptive Transfer Efficiency In Vivo when Combined with Lymphosuppressive Conditioning

An obstacle to use of lymphosuppressive agents as conditioning therapies is the susceptibility of grafted cells, in addition to the target host cells, to their effects. Provided herein is a solution to this issue, by engineering resistance to conditioning agents into grafted cell therapies using antibody-resistant modified receptors (ARMoR). A schematic of this strategy is shown in FIG. 1, using the non-limiting example of an engineered interleukin 2 receptor subunit gamma (IL2RG) variant that is resistant to anti-IL2RG mediated blockade of cytokine signals essential for lymphocyte function. The goal is to introduce into a cell therapy product a fully functional form of the receptor that is not recognized by the conditioning agent. This can be achieved with alterations to the antibody recognition site that abolish binding but preserve receptor function. As a result, host cells remain susceptible to the conditioning agent, but engineered grafted cells are resistant and thus gain a competitive advantage in re-populating the host.

Blockade of immune cell activation and proliferation with the IL2Rg antibody REGN7257 can provide benefit in settings of T-cell-mediated diseases. In Il2rg^{hu/hu} mice, blockade with anti-IL2RG antibody (REGN 7257) reduces blood T and NK cell counts (data not shown). See, e.g., WO 2020/160242 A1 and US 2020/0247894 A1, each of which is herein incorporated by reference in its entirety for all purposes. The same was demonstrated in non-human primates (data not shown).

As initial proof of concept for the antibody-resistant modified receptor approach, humanized Il2rg (Il2rg^{hu/hu}) mice were administered a lymphosuppressive conditioning regimen of an anti-IL2RG antibody (REGN7257, IgG4 isotype; Table 2; see, e.g., WO 2020/160242 A1 and US 2020/0247894 A1, each of which is herein incorporated by reference in its entirety for all purposes) or an isotype control antibody (REGN1945), followed by adoptive transfer of pan splenic T cells from congenically-marked (CD45.1) wild-type (WT) donor mice (FIG. 2A). The WT donor mice express murine Il2rg, which is not recognized by REGN7257, and are therefore resistant to REGN7257. After conditioning and adoptive transfer, host and donor-derived T cells were then enumerated in peripheral blood of host mice by flow cytometric detection of congenic markers (CD45.1/CD45.2). Mice conditioned with REGN7257 showed greater proportions and numbers of “resistant” CD45.1 donor CD8+ and CD4+ T cells than animals pre-conditioned with an isotype control (FIG. 2B). This result is consistent with the notion that selective lymphosuppression (e.g., anti-IL2RG) of host cells, along with resistance of grafted cells to the same agent, confers a competitive advantage in vivo to antibody-resistant donor cells.

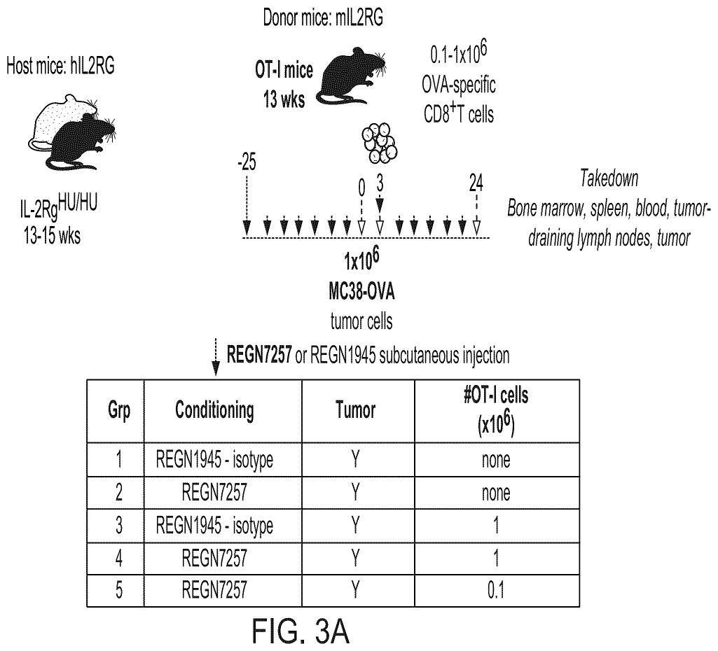

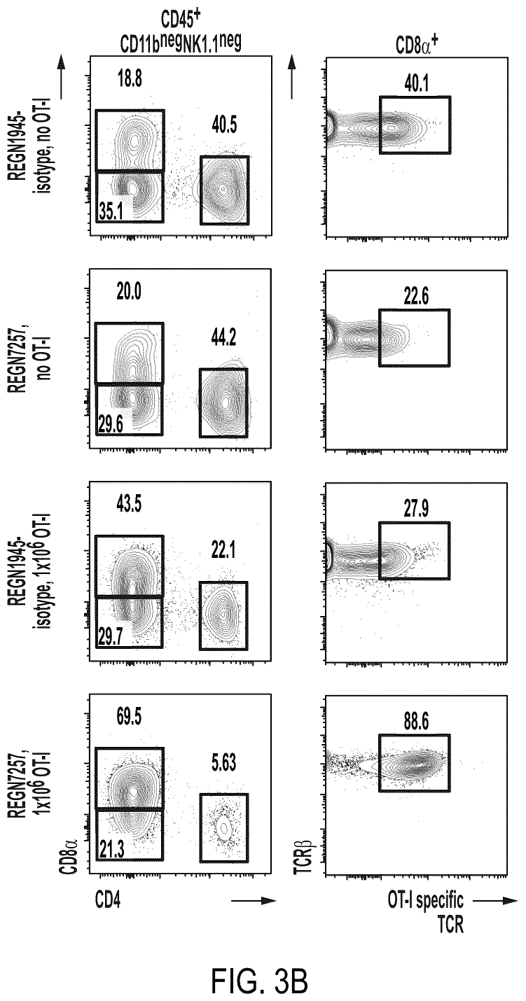

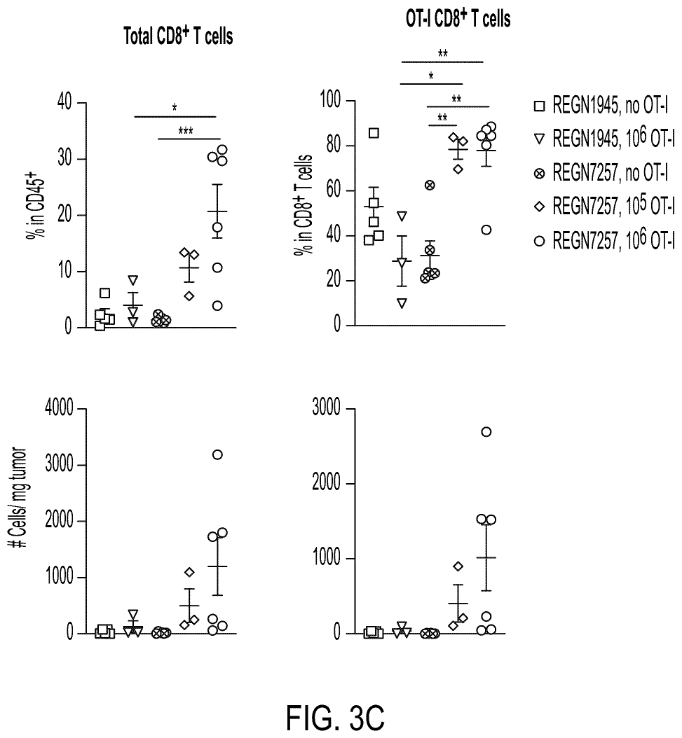

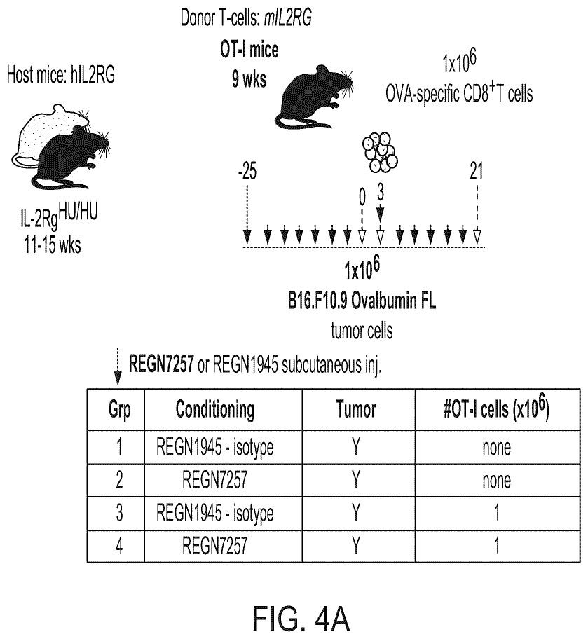

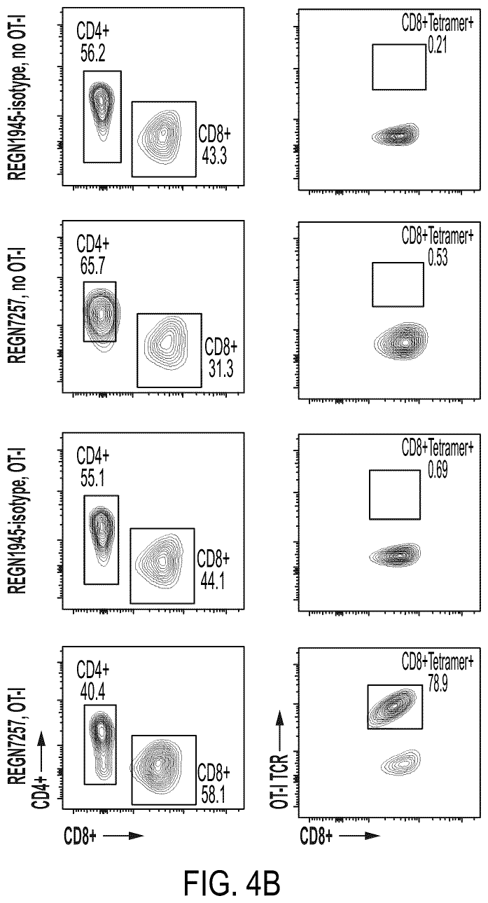

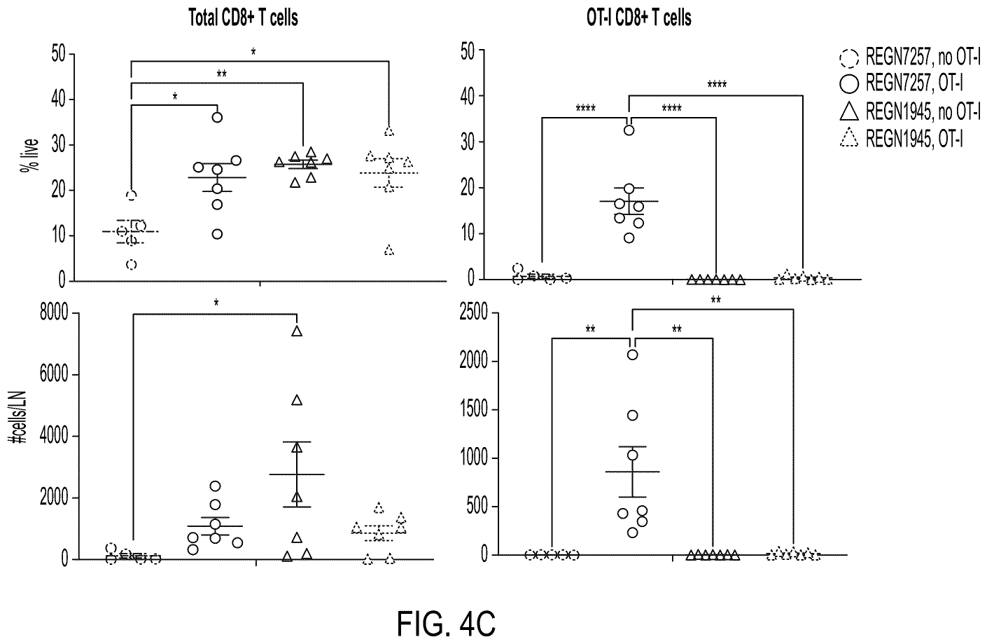

A therapeutic application of adoptively transferred lymphocytes is administration of T cells engineered with anti-tumor reactivity (e.g., CAR-T, TCR-T). To test the antibody-resistant modified receptor concept in a model of this clinical paradigm, 112rg^{hu/hu} mice were conditioned with anti-IL2RG (or isotype control) and then implanted with MC38 (adenocarcinoma) tumors expressing the ovalbumin (OVA) antigen (FIG. 3A). Tumor-bearing mice were then administered T-cells derived from OT-I mice, a transgenic line that expresses a single OVA-specific TCR in all their T cells. See Hogquist et al. (1994) Cell 76 (1): 17-27, herein incorporated by reference in its entirety for all purposes. The OT-I cells expressed murine Il2rg, and are therefore resistant to REGN7257. As compared to isotype-control-treated mice, REGN7257-treated mice showed an enhanced infiltration of OT-I T cells into tumors (FIGS. 3B-3C). These observations were corroborated in similarly designed studies with a second tumor type: B16.F10.9 (melanoma) tumors, also expressing the OVA antigen (FIG. 4A). Compared to isotype-control-treated animals, REGN7257-conditioned mice showed an enhanced infiltration of tumor-specific OT-I T cells into the tumor draining lymph node (FIGS. 4B-4C). Together, these studies indicate that conditioning treatments that selectively suppress host lymphocytes can confer a selective advantage, and hence enhanced therapeutic potential, to grafted cellular therapies resistant to the conditioning agents.

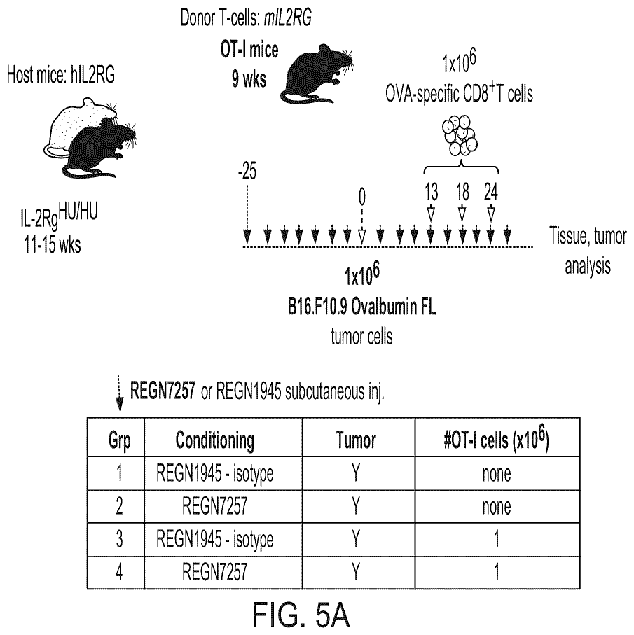

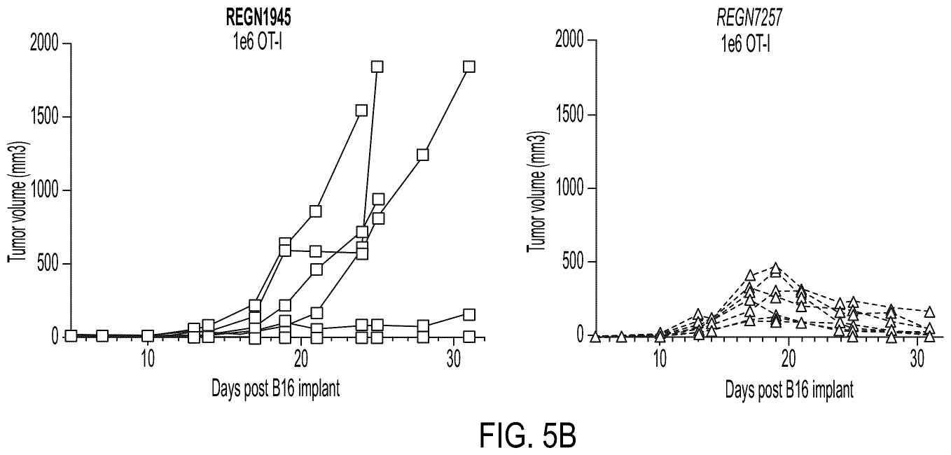

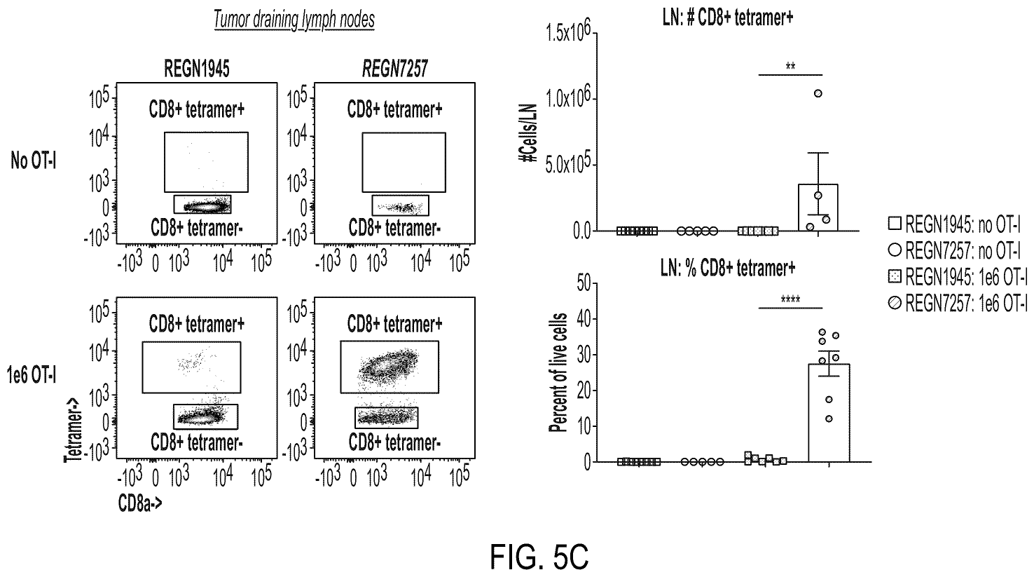

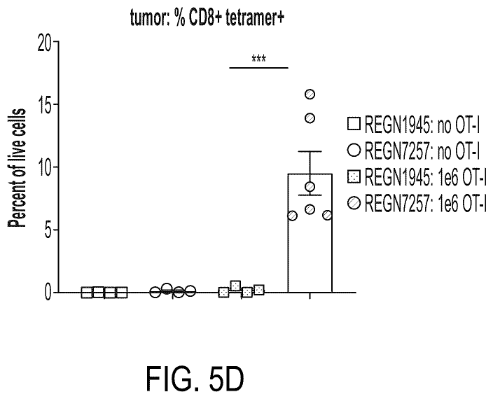

As shown in FIGS. 5A-5D, REGN7257 conditioning dramatically enhanced the ability of adoptively transferred “ARMoR′d” T cells to regress established B16.F10.9-OVA tumors (solid tumors) concurrent with enhanced tumor infiltration. REGN7257-conditioned mice showed enhanced tumor regression compared to isotype (REGN1945)-conditioned mice (FIG. 5B). Infiltration of OVA-specific OT-I T cells, identified by pMHC tetramer staining, was enhanced in REGN7257-conditioned mice (FIGS. 5C-5D).

TABLE 2
REGN7257.

| | DNA - | Protein - |
|---|---|---|
| REGN7257 | SEQ ID NO | SEQ ID NO |
| V_{H} | 1 | 2 |
| V_{H} CDR1 | 3 | 4 |
| V_{H} CDR2 | 5 | 6 |
| V_{H} CDR3 | 7 | 8 |
| V_{L} | 9 | 10 |
| V_{L} CDR1 | 11 | 12 |
| V_{L} CDR2 | 13 | 14 |
| V_{L} CDR3 | 15 | 16 |
| HC | 17 | 18 |
| LC | 19 | 20 |

The amino acid sequences of anti-IL2RG (REGN7257) antibody immunoglobulin heavy and light chains are set forth below (CDRs in lower case, variable regions in bold font in the amino acid sequences for the REGN7257 V_{H} and V_{L}).

SEQ ID NO: 1: REGN7257 V_{H} (Nucleic Acid)
GAGGTGCAGCTGGTGGAGTCTGGGGGAGGCTTGGTACAGCCTGGAGGGTCCCTGAGACTCTCCTGTGC
AGCCTCTGGATTCATCTTCAGTAGTTATGAAATGCATTGGGTCCGCCAGGCTCCAGGGAAGGGGCTGG
AGTGGATTTCATACATTAGTAGTAGTGGTACTACCATATACTACGCAGACTCTGTGAAGGGCCGATTC
ACCATCTCCAGAGACAACGCCAAGAACTCACTGTATCTGCACATGAACAGCCTGAGAGCCGAGGACA
CGGCTGTTTATTACTGTACGAGAGCCCGTATAACTGGAACTTTCGATGTTTTTGATATCTGGGGCCAAG
GGACAATGGTCACCGTCTCTTCA
SEQ ID NO: 2: REGN7257 V_{H} (Amino Acid)
EVQLVESGGGLVQPGGSLRLSCAASGFIFSSYEMHWVRQAPGKGLEWISYISSSGTTIYYADSVKGRFTISR
DNAKNSLYLHMNSLRAEDTAVYYCTRARITGTFDVFDIWGQGTMVTVSS
SEQ ID NO: 3: REGN7257 V_{H} CDR1 (Nucleic Acid)
GGATTCATCTTCAGTAGTTATGAA
SEQ ID NO: 4: REGN7257 V_{H} CDR1 (Amino Acid)
GFIFSSYE
SEQ ID NO: 5: REGN7257 V_{H} CDR2 (Nucleic Acid)
ATTAGTAGTAGTGGTACTACCATA
SEQ ID NO: 6: REGN7257 V_{H} CDR2 (Amino Acid)
ISSSGTTI
SEQ ID NO: 7: REGN7257 V_{H} CDR3 (Nucleic Acid)
ACGAGAGCCCGTATAACTGGAACTTTCGATGTTTTTGATATC
SEQ ID NO: 8: REGN7257 V_{H} CDR3 (Amino Acid)
TRARITGTFDVFDI
SEQ ID NO: 9: REGN7257 V_{L} (Nucleic Acid)
GACATCCAGATGACCCAGTCTCCATCCTCCCTGTCTGCATCTGTGGGAGACAGAGTCACCATCACTTGC
CGGGCAAGTCAGAGCATTAGCAGCTATTTAAATTGGTATCAGCAGAAACCAGGGAAAGCCCCTAAGC
TCCTGATCTTTGCTGCATCCAATTTACAGAGTGGGGTCCCATCAAGGTTCAGTGGCAGTAGATCTGGGA
CAGATTTCACTCTCACCATCAGCAGTCTGCAACCTGAAGATTTTGCAACTTACTACTGTCAACAGAATT
ACAATATCCCGTACACTTTTGGCCAGGGGACCAAGCTGGAGATCAAA
SEQ ID NO: 10: REGN7257 V_{L} (Amino Acid)
DIQMTQSPSSLSASVGDRVTITCRASQSISSYLNWYQQKPGKAPKLLIFAASNLQSGVPSRFSGSRSGTDFTL
TISSLQPEDFATYYCQQNYNIPYTFGQGTKLEIK
SEQ ID NO: 11: REGN7257 V_{L} CDR1 (Nucleic Acid)
CAGAGCATTAGCAGCTAT
SEQ ID NO: 12: REGN7257 V_{L} CDR1 (Amino Acid)
QSISSY
SEQ ID NO: 13: REGN7257 V_{L} CDR2 (Nucleic Acid)
GCTGCATCC
SEQ ID NO: 14: REGN7257 V_{L} CDR2 (Amino Acid)
AAS
SEQ ID NO: 15: REGN7257 V_{L} CDR3 (Nucleic Acid)
CAACAGAATTACAATATCCCGTACACT
SEQ ID NO: 16: REGN7257 V_{L} CDR3 (Amino Acid)
QQNYNIPYT
SEQ ID NO: 17: REGN7257 HC (Nucleic Acid)
GAGGTGCAGCTGGTGGAGTCTGGGGGAGGCTTGGTACAGCCTGGAGGGTCCCTGAGACTCTCCTGTGC
AGCCTCTGGATTCATCTTCAGTAGTTATGAAATGCATTGGGTCCGCCAGGCTCCAGGGAAGGGGCTGG
AGTGGATTTCATACATTAGTAGTAGTGGTACTACCATATACTACGCAGACTCTGTGAAGGGCCGATTC
ACCATCTCCAGAGACAACGCCAAGAACTCACTGTATCTGCACATGAACAGCCTGAGAGCCGAGGACA
CGGCTGTTTATTACTGTACGAGAGCCCGTATAACTGGAACTTTCGATGTTTTTGATATCTGGGGCCAAG
GGACAATGGTCACCGTCTCTTCAGCCTCCACCAAGGGCCCATCGGTCTTCCCCCTGGCGCCCTGCTCCA
GGAGCACCTCCGAGAGCACAGCCGCCCTGGGCTGCCTGGTCAAGGACTACTTCCCCGAACCGGTGACG
GTGTCGTGGAACTCAGGCGCCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGCACGAAGACCTACACCTGCA
ACGTAGATCACAAGCCCAGCAACACCAAGGTGGACAAGAGAGTTGAGTCCAAATATGGTCCCCCATG
CCCACCCTGCCCAGCACCTGAGTTCCTGGGGGGACCATCAGTCTTCCTGTTCCCCCCAAAACCCAAGG
ACACTCTCATGATCTCCCGGACCCCTGAGGTCACGTGCGTGGTGGTGGACGTGAGCCAGGAAGACCCC
GAGGTCCAGTTCAACTGGTACGTGGATGGCGTGGAGGTGCATAATGCCAAGACAAAGCCGCGGGAGG
AGCAGTTCAACAGCACGTACCGTGTGGTCAGCGTCCTCACCGTCCTGCACCAGGACTGGCTGAACGGC
AAGGAGTACAAGTGCAAGGTCTCCAACAAAGGCCTCCCGTCCTCCATCGAGAAAACCATCTCCAAAGC
CAAAGGGCAGCCCCGAGAGCCACAGGTGTACACCCTGCCCCCATCCCAGGAGGAGATGACCAAGAAC
CAGGTCAGCCTGACCTGCCTGGTCAAAGGCTTCTACCCCAGCGACATCGCCGTGGAGTGGGAGAGCAA
TGGGCAGCCGGAGAACAACTACAAGACCACGCCTCCCGTGCTGGACTCCGACGGCTCCTTCTTCCTCT
ACAGCAGGCTCACCGTGGACAAGAGCAGGTGGCAGGAGGGGAATGTCTTCTCATGCTCCGTGATGCAT
GAGGCTCTGCACAACCACTACACACAGAAGTCCCTCTCCCTGTCTCTGGGTAAATGA
SEQ ID NO: 18: REGN7257 HC (Amino Acid)
EVQLVESGGGLVQPGGSLRLSCAASgfifssyeMHWVRQAPGKGLEWISYisssgttiYYADSVKGRFTISRD
NAKNSLYLHMNSLRAEDTAVYYCtraritgtfdvfdiWGQGTMVTVSSASTKGPSVFPLAPCSRSTSESTAALG
CLVKDYFPEPVTVSWNSGALTSGVHTFPAVLQSSGLYSLSSVVTVPSSSLGTKTYTCNVDHKPSNTKVDKR
VESKYGPPCPPCPAPEFLGGPSVFLFPPKPKDTLMISRTPEVTCVVVDVSQEDPEVQFNWYVDGVEVHNAK
TKPREEQFNSTYRVVSVLTVLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTVDKSRWQEGNVFSCSVMH
EALHNHYTQKSLSLSLGK
SEQ ID NO: 19: REGN7257 LC (Nucleic Acid)
GACATCCAGATGACCCAGTCTCCATCCTCCCTGTCTGCATCTGTGGGAGACAGAGTCACCATCACTTGC
CGGGCAAGTCAGAGCATTAGCAGCTATTTAAATTGGTATCAGCAGAAACCAGGGAAAGCCCCTAAGC
TCCTGATCTTTGCTGCATCCAATTTACAGAGTGGGGTCCCATCAAGGTTCAGTGGCAGTAGATCTGGGA
CAGATTTCACTCTCACCATCAGCAGTCTGCAACCTGAAGATTTTGCAACTTACTACTGTCAACAGAATT
ACAATATCCCGTACACTTTTGGCCAGGGGACCAAGCTGGAGATCAAACGAACTGTGGCTGCACCATCT
GTCTTCATCTTCCCGCCATCTGATGAGCAGTTGAAATCTGGAACTGCCTCTGTTGTGTGCCTGCTGAAT
AACTTCTATCCCAGAGAGGCCAAAGTACAGTGGAAGGTGGATAACGCCCTCCAATCGGGTAACTCCCA
GGAGAGTGTCACAGAGCAGGACAGCAAGGACAGCACCTACAGCCTCAGCAGCACCCTGACGCTGAGC
AAAGCAGACTACGAGAAACACAAAGTCTACGCCTGCGAAGTCACCCATCAGGGCCTGAGCTCGCCCG
TCACAAAGAGCTTCAACAGGGGAGAGTGTTAG
SEQ ID NO: 20: REGN7257 LC (Amino Acid)
DIQMTQSPSSLSASVGDRVTITCRASqsissyLNWYQQKPGKAPKLLIFaasNLQSGVPSRFSGSRSGTDFT
LTISSLQPEDFATYYCqqnynipytFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSFNRGEC

### Example 2. Minimally Altered Antibody-Resistant Modified Receptors Retain Optimal Function in Engineered Cell Products

2.1 Identification of ARMoR modifications by cross species alignment and analysis.

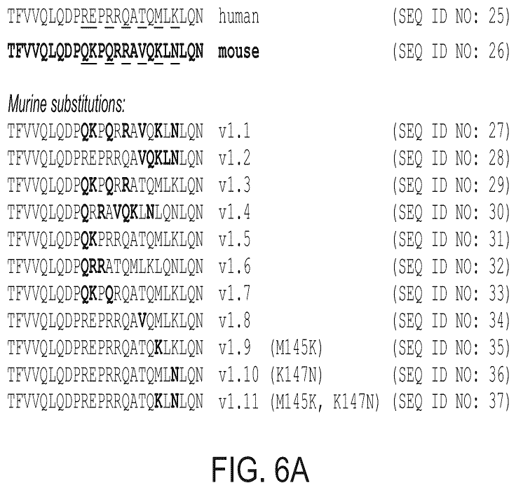

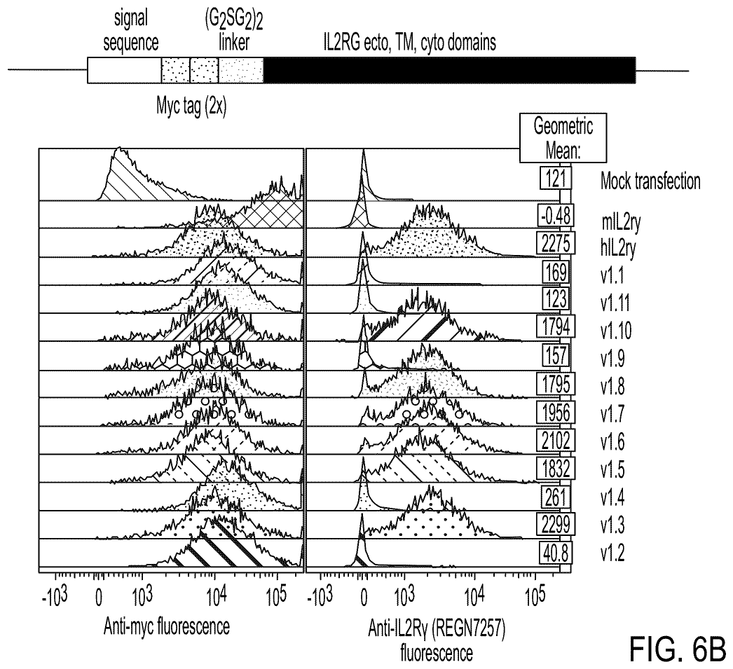

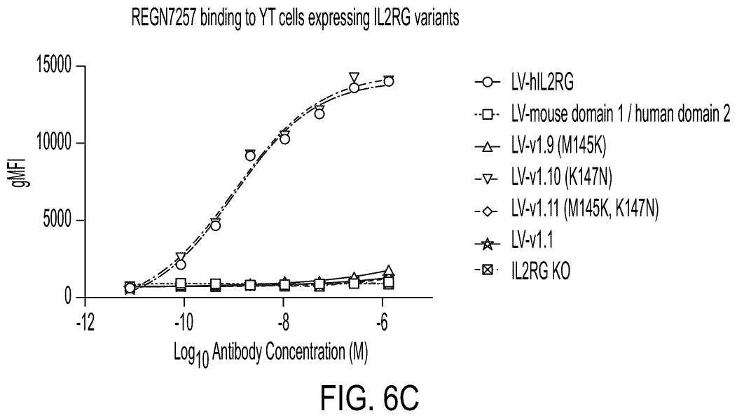

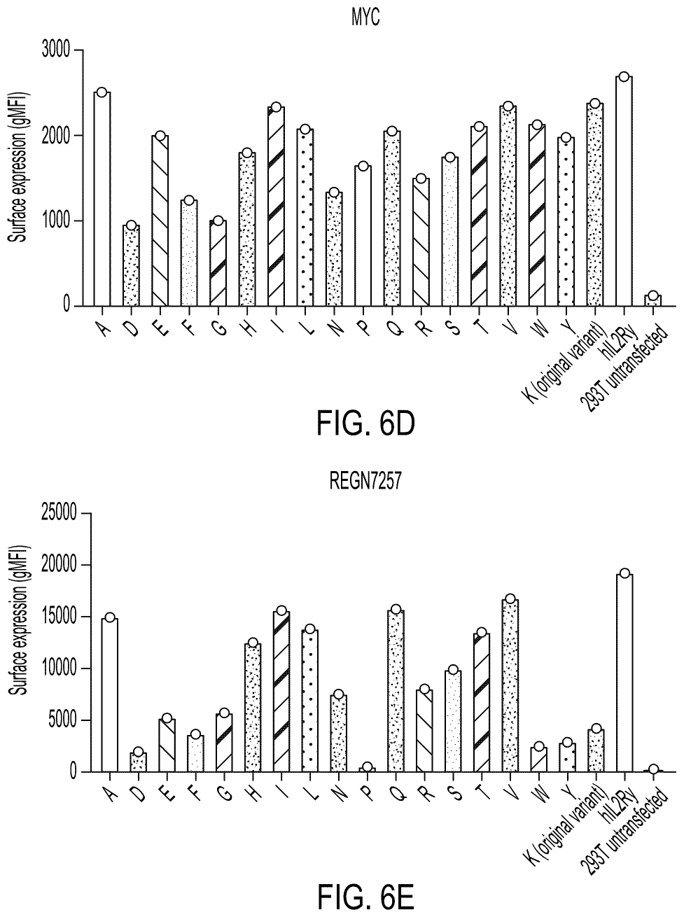

Studies utilizing cellular grafts expressing antibody-resistant murine IL2RG provided a key proof-of-concept for the antibody-resistant modified receptor approach. However, the fully murine IL2rg receptor is not ideal for use in a human cellular therapy because it is unlikely to fully replicate the function of the human receptor in the donor cells, and may also be immunogenic in human hosts. To identify a region of human IL2RG to focus finer mutational analyses, the likely REGN7257 binding region was mapped by hydrogen-deuterium exchange mass spectrometry. Kochert et al. (2018) Methods Mol. Biol. 1764:153-171, herein incorporated by reference in its entirety for all purposes. This mapped epitope region in human IL2RG was aligned to the corresponding murine region, and a series of amino acid substitutions were made (FIG. 6A). These variants were expressed as myc-tagged constructs in 293T cells, and several variants showed greatly reduced binding to anti-IL2RG (FIG. 6B). Notably, antibody-resistant variants all shared a methionine 145 to lysine (M145K) substitution, and this substitution on its own was sufficient to reduce REGN7257 binding. Loss of REGN7257 binding to M145K-bearing variants was confirmed by flow cytometric quantification across a broad range of REGN7257 concentrations tested on YT (human NK lymphoma) cells engineered to express specific modified variants (FIG. 6C).

Because human IL2RG residue M145 is critical for REGN7257 binding (FIGS. 6B-6C), saturating mutagenesis testing other amino acid substitutions was performed at this site. Expression of myc-tagged IL2RG constructs in 293T cells identified several additional M145 substitution variants that retained surface expression by flow cytometry (FIG. 6D) while strongly reducing REGN7257 binding (M145D, M145E, M145P, M145W, and M145Y, FIG. 6E).

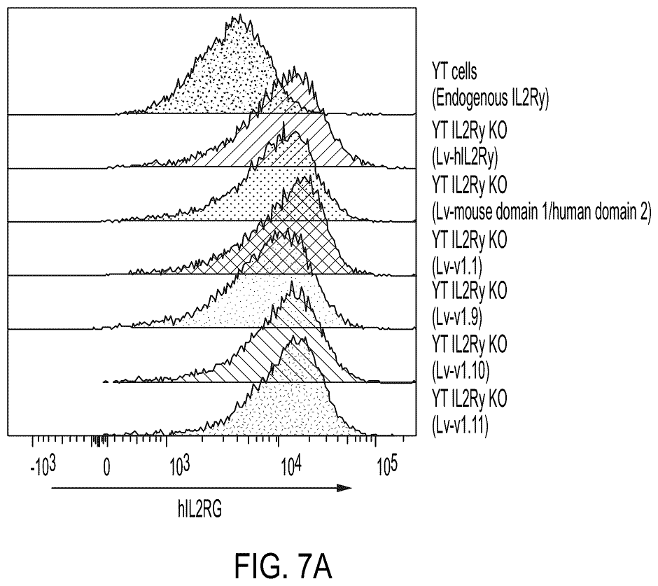

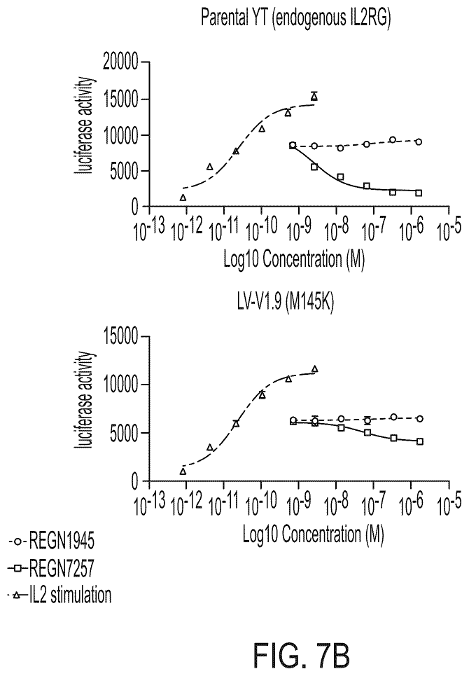

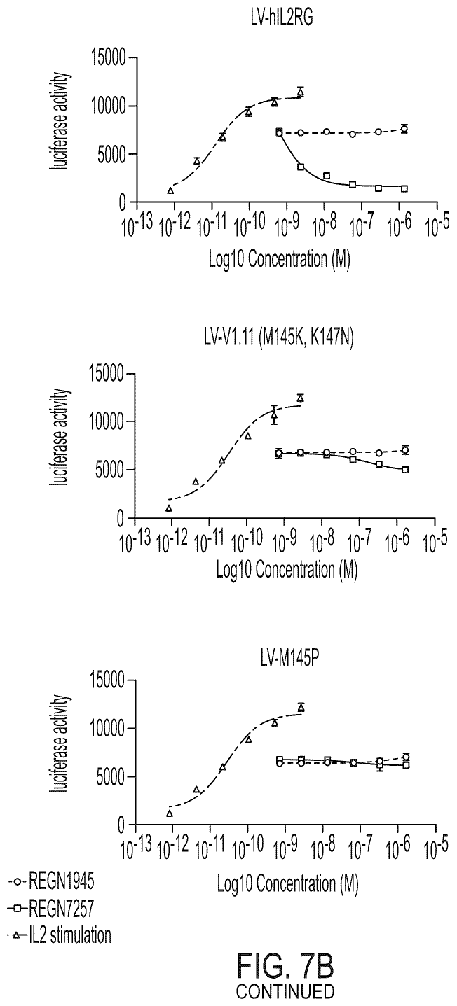

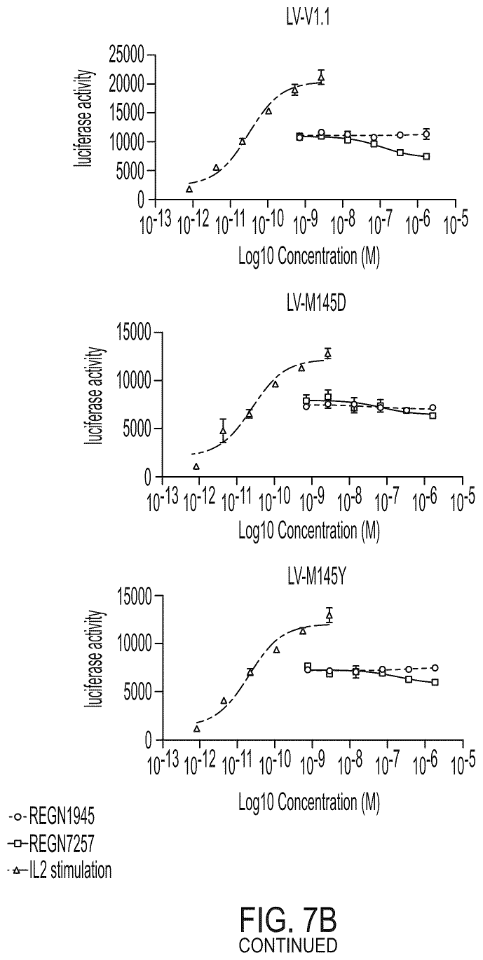

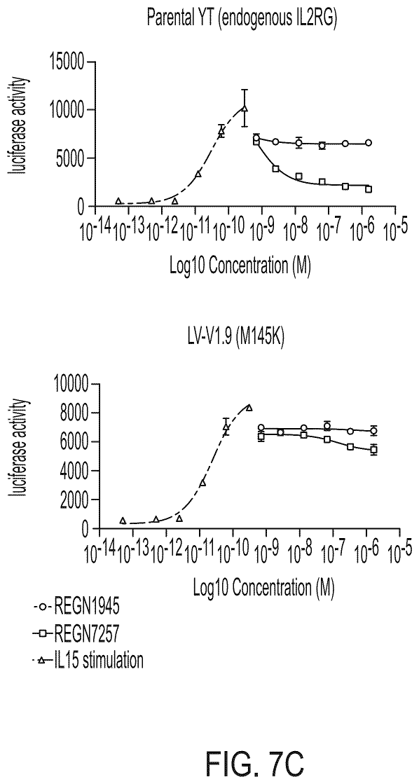

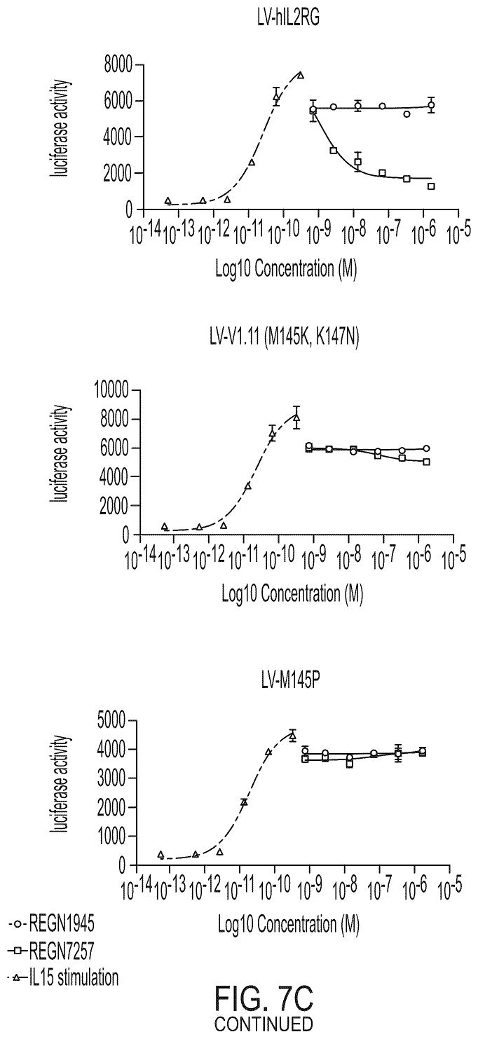

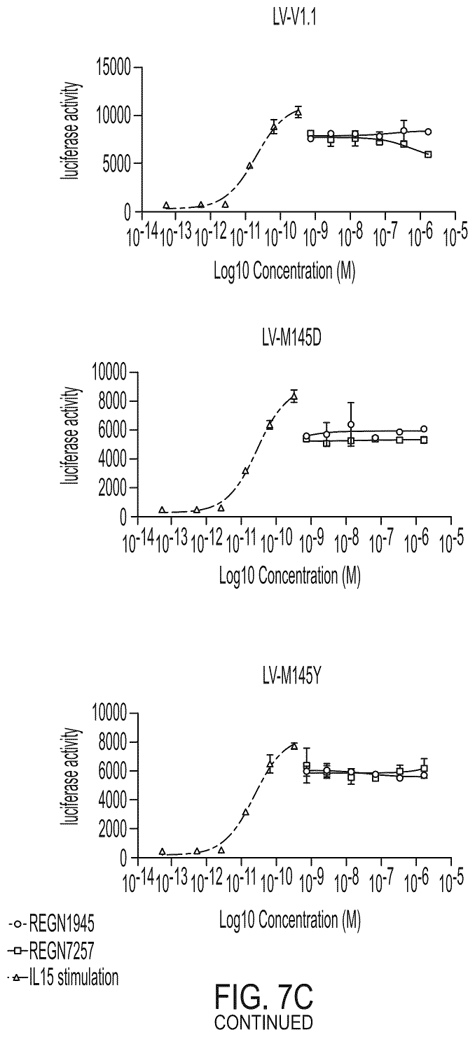

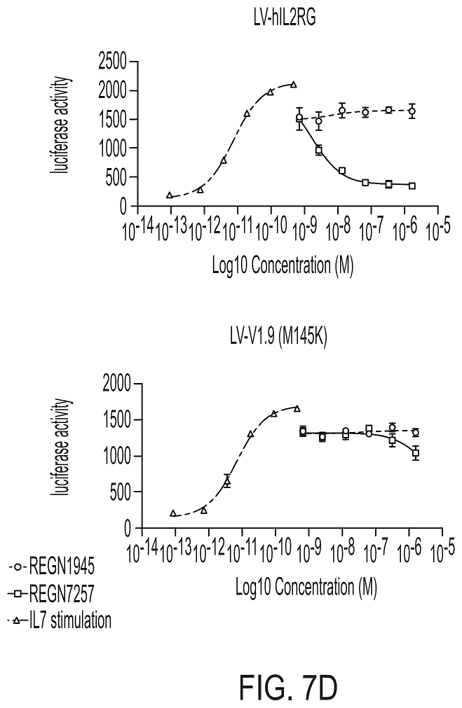

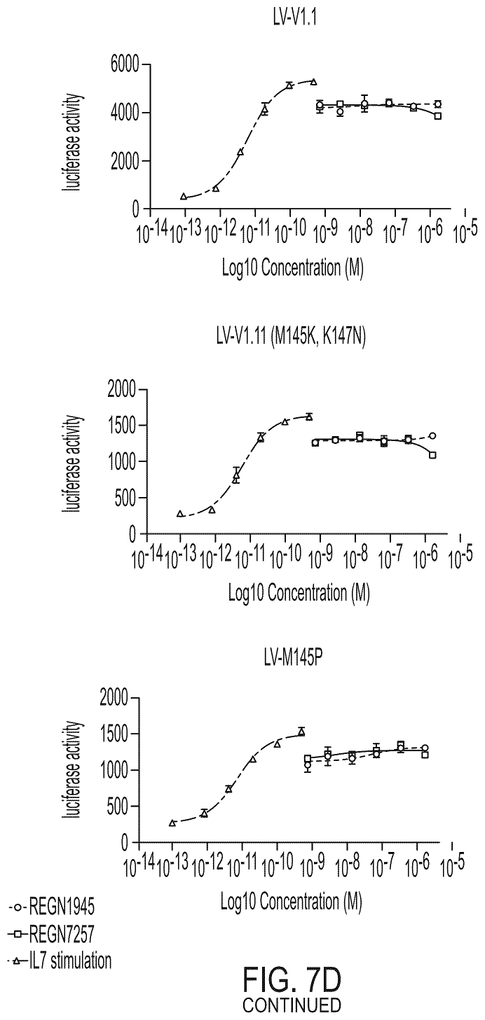

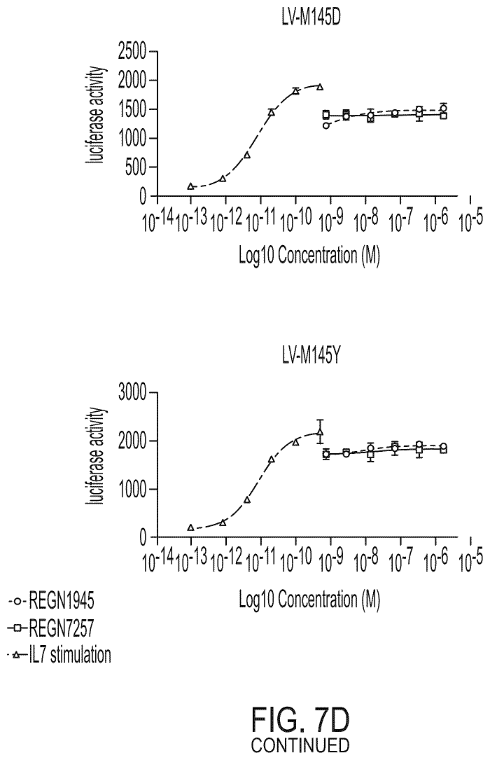

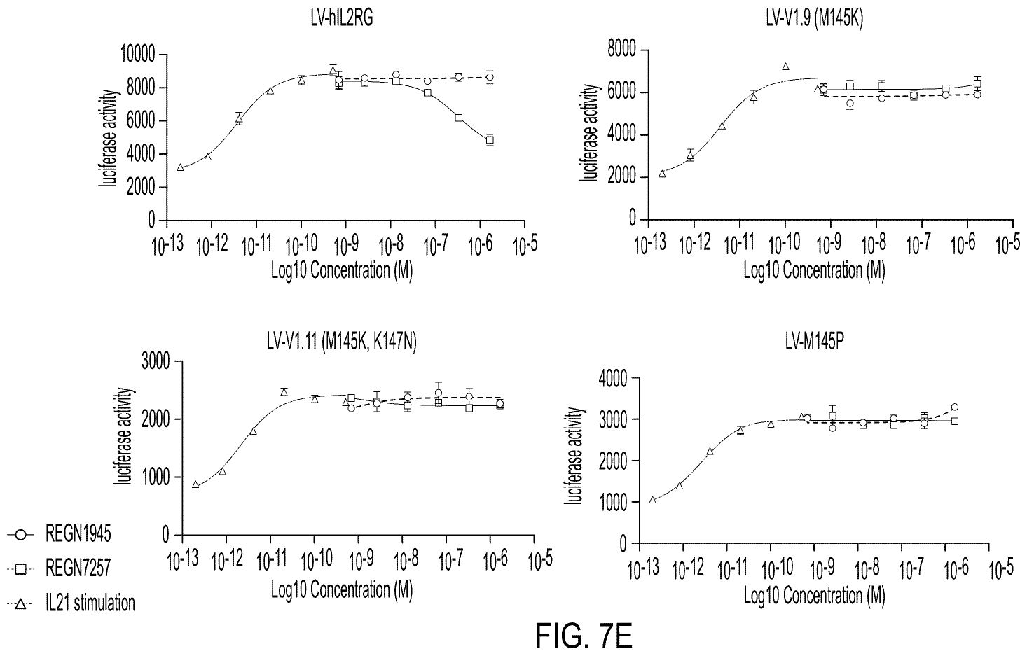

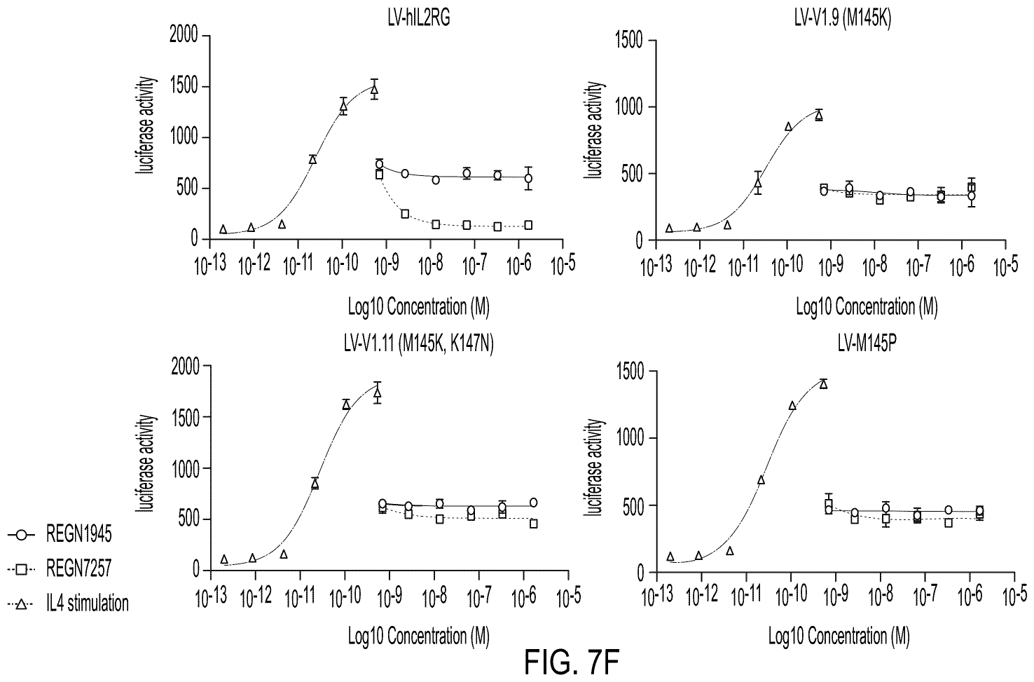

Bioassays for cytokine response were used to assess the function of a subset of antibody-resistant IL2RG variants. YT cells were engineered with a STAT5 response-element driven luciferase (STAT5.RE.luc) reporter, and endogenous IL2RG expression was knocked out using CRISPR/Cas9-mediated disruption of the gene locus. Individual IL2RG variants were re-introduced into these parental YT/STAT5.RE.luc/IL2RGD cells via lentiviral (LV) transduction (FIG. 7A). STAT5-driven luciferase activity in response to human IL2 was similar for antibody-resistant IL2RG variants compared to wild type human IL2RG (FIG. 7B, triangles). In addition, variants that showed a loss of binding to REGN7257 by flow cytometry (FIGS. 7B-7D) also showed, to varying degrees, escape from the REGN7257-mediated block of IL2 mediated signaling (FIG. 7B, squares and circles). Bioassay studies analyzing responses to human IL15, IL7, IL21, and IL4 showed a similar preservation of cytokine-induced signaling, as well as resistance to REGN7257-mediated blockade (FIGS. 7C-7F, squares and circles). Collectively, these studies illustrate that potential IL2RG ARMoR mutants respond efficiently to key gamma-chain family cytokines and are resistant to in vitro blockade by REGN7257.

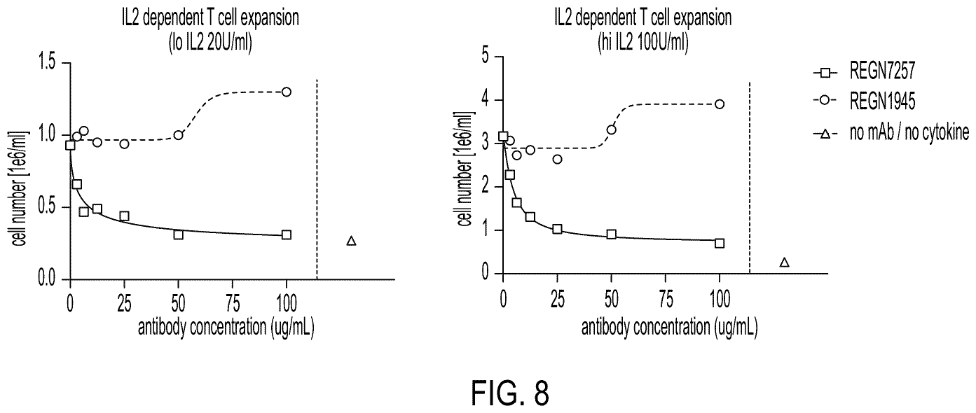

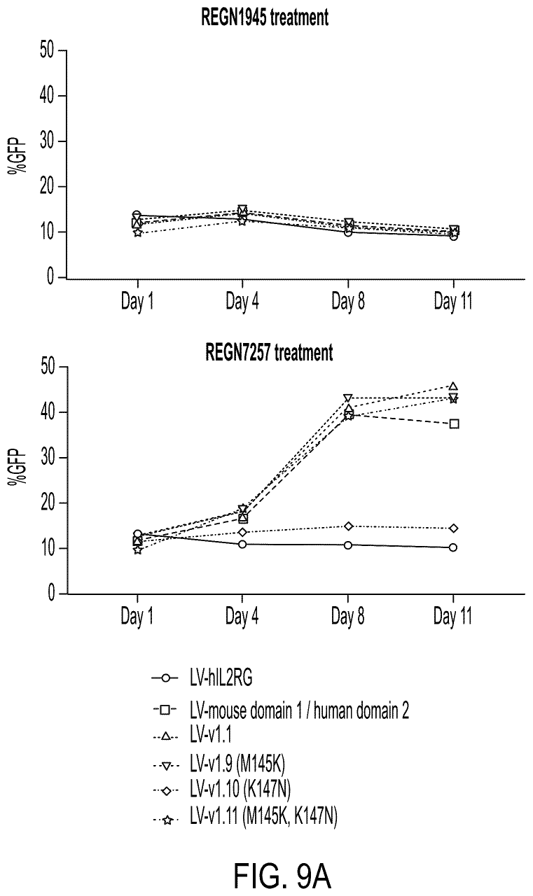

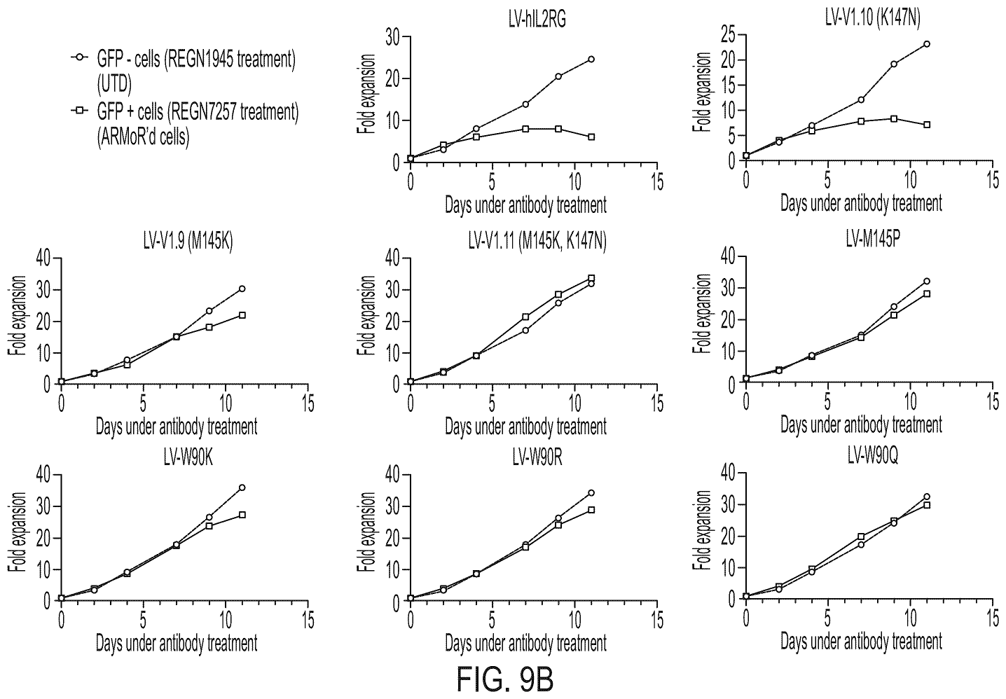

To test the function of potential IL2RG antibody-resistant modified receptors in a physiological system, select variants were tested for their ability to support IL2-dependent growth of primary human T cells in vitro. Human pan T cells activated with anti-CD3/anti-CD28 beads and grown in the presence of human IL2 showed significant, dose-dependent reduction of growth upon IL2RG blockade with REGN7257 (FIG. 8). To examine whether T cells expressing antibody-resistant IL2RG can escape the effects of REGN7257 blockade, cells were transduced with lentiviral (LV)-encoded IL2RG variants (with IRES-driven green fluorescent protein (GFP) to track transduction). LV transductions were done at low multiplicity of infection (MOI), achieving ˜10% GFP+ cells. Subsequently, treatment with REGN7257, but not isotype control antibody, led to an increased proportion of GFP+ cells in cultures transduced with antibody-resistant variants with the M145K substitution (FIG. 9A). In contrast, cells transduced with antibody-susceptible variants (e.g., wild type human IL2RG) showed no increase in GFP+ cells, and thus no competitive enrichment, with REGN7257 treatment. FIG. 9B shows that cells expressing REGN7257-resistant IL2RG variants cultured in the presence of REGN7257 (squares) grow at rate indistinguishable from non-transduced cells expressing endogenous WT IL2RG cultured in the presence of isotype REGN1945 (circles). These results show that introduction of antibody-resistant receptor variants confers a competitive growth advantage to primary lymphocytes in the presence of receptor blockade in vitro. In addition, the results indicate that REGN7257-resistant variants support identical IL2-dependent growth as WT IL2RG, and can function efficiently in cells still expressing endogenous IL2RG.

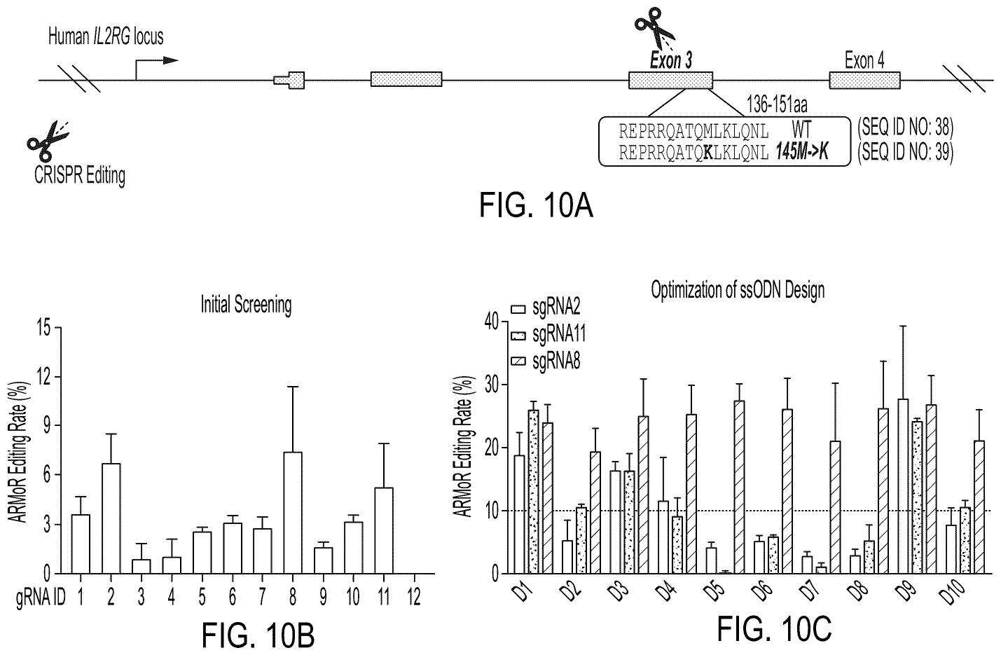

Given the importance of human IL2RG residue M145 to REGN7257 binding (FIGS. 6B-6E), CRISPR/Cas9-based gene editing strategies were developed to introduce modifications to the corresponding region of the human IL2RG gene (FIG. 10A). A panel of Cas9 single guide RNAs in exon 3, which encodes M145 and surrounding residues, was introduced into primary human T cells by electroporation of Cas9/sgRNA ribonucleoprotein complexes (RNPs). See Table 3. Editing frequencies were determined by PCR amplification of the corresponding genomic region and sequencing analysis to quantify indels and substitutions at predicted Cas9 cut sites, an indicator of error-prone non-homologous end joining (NHEJ) at the site of Cas9-induced double-stranded breaks (DSBs) (FIG. 10B). The three sgRNA sequences (sgRNAs 2, 8, and 11) showing the highest frequency of gene editing were then tested in combination with a series of single-strand oligodeoxynucleotide (ssODN) donor repair sequences, to introduce nucleotide edits encoding the M145K substitution (FIG. 10C). See Table 4. Ten ssODNs were designed for use with sgRNA 2, ten ssODNs were designed for use with sgRNA 8, and ten ssODNs were designed for use with sgRNA 11. Several sgRNA-ssODN combinations were identified that efficiently introduce the desired gene edit into primary human T cells genomic DNA, an approach readily extendable to other immune cells and their progenitors.

TABLE 3
IL2RG guide RNAs.

| | | | SEQ ID NOS |
|---|---|---|---|
| gRNA | Target Sequence | Guide Sequence | (Target, Guide) |
| 1 | CCTGTCTCCTGGGTTCCCGT | CCUGUCUCCUGGGUUCCCGU | 64, 76 |
| 2 | ACTGCAGAATCTGGGTAATT | ACUGCAGAAUCUGGGUAAUU | 65, 77 |
| 3 | GAACAACAAATGTTTGGTAG | GAACAACAAAUGUUUGGUAG | 66, 78 |
| 4 | AACATTTGTTGTTCAGCTCC | AACAUUUGUUGUUCAGCUCC | 67, 79 |
| 5 | TCCAGGACCCACGGGAACCC | UCCAGGACCCACGGGAACCC | 68, 80 |
| 6 | TCCTGGGTTCCCGTGGGTCC | UCCUGGGUUCCCGUGGGUCC | 69, 81 |
| 7 | TTGTTCAGCTCCAGGACCCA | UUGUUCAGCUCCAGGACCCA | 70, 82 |
| 8 | CTGCAGTTTTAGCATCTGTG | CUGCAGUUUUAGCAUCUGUG | 71, 83 |
| 9 | TGTTCAGCTCCAGGACCCAC | UGUUCAGCUCCAGGACCCAC | 72, 84 |
| 10 | GATGCTAAAACTGCAGAATC | GAUGCUAAAACUGCAGAAUC | 73, 85 |
| 11 | GCCTGTCTCCTGGGTTCCCG | GCCUGUCUCCUGGGUUCCCG | 74, 86 |
| 12 | GGAGCTGAACAACAAATGTT | GGAGCUGAACAACAAAUGUU | 75, 87 |

TABLE 4
IL2RG M145K ssODNs.

| | | SEQ |
|---|---|---|
| SSODN | Sequence | ID NO |
| 2-D1 | A*G*GACCCACGGGAACCCAGGAGACAGGCCACACAGAAGCTAAAACTGCAGA | 88 |
| | ATCTGGGTAATTTGAAAAGAAAGGGTCAAGAGACCAGGGATACTGTGGGACAT | |
| | TGGAGTCTACAGAG*T*A | |
| 2-D2 | T*A*CTCTGTAGACTCCAATGTCCCACAGTATCCCTGGTCTCTTGACCCTTTCTTT | 89 |
| | TCAAATTACCCAGATTCTGCAGTTTTAGCTTCTGTGTGGCCTGTCTCCTGGGTTC | |
| | CCGTGGGTC*C*T | |
| 2-D3 | C*C*AGGAGACAGGCCACACAGAAGCTAAAACTGCAGAATCTGGGTAATTTGAA | 90 |
| | AAGAAAGGGTCAAGAGACCAGGGATACTGTGGGACA*T*T | |
| 2-D4 | A*A*TGTCCCACAGTATCCCTGGTCTCTTGACCCTTTCTTTTCAAATTACCCAGAT | 91 |
| | TCTGCAGTTTTAGCTTCTGTGTGGCCTGTCTCCT*G*G | |
| 2-D5 | C*A*CAGAAGCTAAAACTGCAGAATCTGGGTAATTTGAAAAGAAAGGGTCAAG | 92 |
| | AGACCAG*G*G | |
| 2-D6 | C*C*CTGGTCTCTTGACCCTTTCTTTTCAAATTACCCAGATTCTGCAGTTTTAGCT | 93 |
| | TCTG*T*G | |
| 2-D7 | C*A*CAGAAGCTAAAACTGCAGAATCTGGGTAATTTGAAAAGAAAGGGTCAAG | 94 |
| | AGACCAGGGATACTGTGGGACATTGGAGTCTACAGAG*T*A | |
| 2-D8 | T*A*CTCTGTAGACTCCAATGTCCCACAGTATCCCTGGTCTCTTGACCCTTTCTTT | 95 |
| | TCAAATTACCCAGATTCTGCAGTTTTAGCTTCTG*T*G | |
| 2-D9 | A*G*GACCCACGGGAACCCAGGAGACAGGCCACACAGAAGCTAAAACTGCAGA | 96 |
| | ATCTGGGTAATTTGAAAAGAAAGGGTCAAGAGACCAG*G*G | |
| 2-D10 | C*C*CTGGTCTCTTGACCCTTTCTTTTCAAATTACCCAGATTCTGCAGTTTTAGCT | 97 |
| | TCTGTGTGGCCTGTCTCCTGGGTTCCCGTGGGTC*C*T | |
| 8-D1 | T*A*TCCCTGGTCTCTTGACCCTTTCTTTCCAAATTACCCAGATTCTGCAGTTTTA | 98 |
| | GCTTCTGTGTTGCCTGTCTCCTGGGTTCCCGTGGGTCCTGGAGCTGAACAACAA | |
| | ATGTTTGGTA*G*A | |
| 8-D2 | T*C*TACCAAACATTTGTTGTTCAGCTCCAGGACCCACGGGAACCCAGGAGACA | 99 |
| | GGCAACACAGAAGCTAAAACTGCAGAATCTGGGTAATTTGGAAAGAAAGGGTC | |
| | AAGAGACCAGGGA*T*A | |
| 8-D3 | T*G*ACCCTTTCTTTCCAAATTACCCAGATTCTGCAGTTTTAGCTTCTGTGTTGCC | 100 |
| | TGTCTCCTGGGTTCCCGTGGGTCCTGGAGCTGAA*C*A | |
| 8-D4 | T*G*TTCAGCTCCAGGACCCACGGGAACCCAGGAGACAGGCAACACAGAAGCT | 101 |
| | AAAACTGCAGAATCTGGGTAATTTGGAAAGAAAGGGT*C*A | |
| 8-D5 | A*A*ATTACCCAGATTCTGCAGTTTTAGCTTCTGTGTTGCCTGTCTCCTGGGTTCC | 102 |
| | CGTG*G*G | |
| 8-D6 | C*C*CACGGGAACCCAGGAGACAGGCAACACAGAAGCTAAAACTGCAGAATCT | 103 |
| | GGGTAAT*T*T | |
| 8-D7 | T*G*CAGTTTTAGCTTCTGTGTTGCCTGTC*T*C | 104 |
| 8-D8 | G*A*GACAGGCAACACAGAAGCTAAAACTG*C*A | 105 |
| 8-D9 | A*A*ATTACCCAGATTCTGCAGTTTTAGCTTCTGTGTTGCCTGTCTCCTGGGTTCC | 106 |
| | CGTGGGTCCTGGAGCTGAACAACAAATGTTTGGT*A*G | |
| 8-D10 | C*T*ACCAAACATTTGTTGTTCAGCTCCAGGACCCACGGGAACCCAGGAGACAG | 107 |
| | GCAACACAGAAGCTAAAACTGCAGAATCTGGGTAAT*T*T | |
| 11-D1 | T*A*TCCCTGGTCTCTTGACCCTTTCTTTCCAAATTACCCAGATTCTGCAGTTTTA | 108 |
| | GCTTCTGTGTTGCCTGTCTCCTGGGTTCCCGTGG*G*T | |
| 11-D2 | A*C*CCACGGGAACCCAGGAGACAGGCAACACAGAAGCTAAAACTGCAGAATC | 109 |
| | TGGGTAATTTGGAAAGAAAGGGTCAAGAGACCAGGGA*T*A | |
| 11-D3 | T*C*TTTCCAAATTACCCAGATTCTGCAGTTTTAGCTTCTGTGTGGCCTGTCTCCT | 110 |
| | GGGCTCTCGTGGGTCCTGGAGCTGAACAACAAATGTTTGGTAGAGGTGGATCTC | |
| | CTTTTTTTGC*A*A | |
| 11-D4 | T*T*GCAAAAAAAGGAGATCCACCTCTACCAAACATTTGTTGTTCAGCTCCAGG | 111 |
| | ACCCACGAGAGCCCAGGAGACAGGCCACACAGAAGCTAAAACTGCAGAATCTG | |
| | GGTAATTTGGAAA*G*A | |
| 11-D5 | C*A*GATTCTGCAGTTTTAGCTTCTGTGTGGCCTGTCTCCTGGGCTCTCGTGGGTC | 112 |
| | CTGGAGCTGAACAACAAATGTTTGGTAGAGGTGG*A*T | |
| 11-D6 | A*T*CCACCTCTACCAAACATTTGTTGTTCAGCTCCAGGACCCACGAGAGCCCAG | 113 |
| | GAGACAGGCCACACAGAAGCTAAAACTGCAGAATC*T*G | |
| 11-D7 | T*A*GCTTCTGTGTGGCCTGTCTCCTGGGCTCTCGTGGGTCCTGGAGCTGAACAA | 114 |
| | CAAAT*G*T | |
| 11-D8 | A*C*ATTTGTTGTTCAGCTCCAGGACCCACGAGAGCCCAGGAGACAGGCCACAC | 115 |
| | AGAAGC*T*A | |
| 11-D9 | T*A*GCTTCTGTGTGGCCTGTCTCCTGGGCTCTCGTGGGTCCTGGAGCTGAACAA | 116 |
| | CAAATGTTTGGTAGAGGTGGATCTCCTTTTTTTGC*A*A | |
| 11-D10 | T*T*GCAAAAAAAGGAGATCCACCTCTACCAAACATTTGTTGTTCAGCTCCAGG | 117 |
| | ACCCACGAGAGCCCAGGAGACAGGCCACACAGAAGC*T*A | |

*denotes phosphorothioate linkage

2.2 Identification of ARMoR modifications by high resolution structural analysis.

High-resolution cryogenic electron microscopy (cryoEM) analysis of the REGN7257 Fab region complexed with recombinant human IL2RG extracellular domain (ectodomain; ECD) was conducted to gain greater insights into residue contacts between antibody and antigen (data not shown). This structural data revealed that residue M145 of IL2RG sits at the center of the REGN7257 epitope, making hydrophobic contacts with both the heavy and light chains. The residue is not situated near the IL2 interaction site (closest distance is 7 Å), nor the IL2RA or IL2RB subunits of the full trimeric IL2 receptor. Wang et al. (2005) Science 310 (5751): 1159-1163, herein incorporated by reference in its entirety for all purposes. Together, these data explain why mutating M145 can strongly impact REGN7257 binding, while preserving the function of the receptor complex.

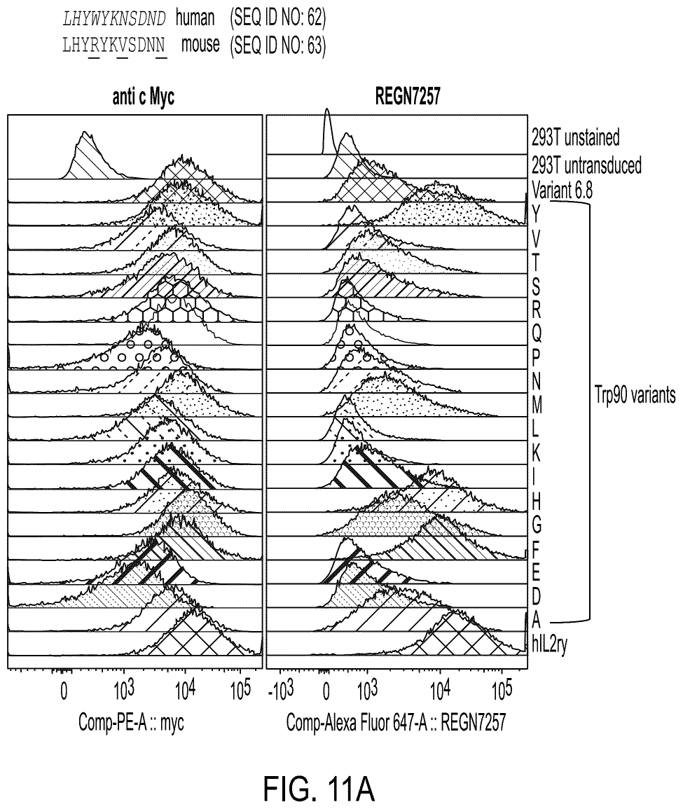

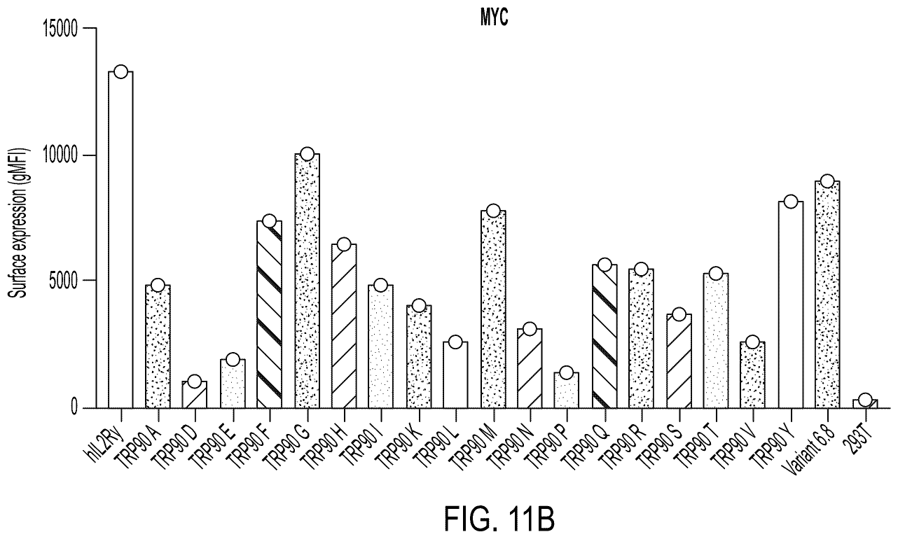

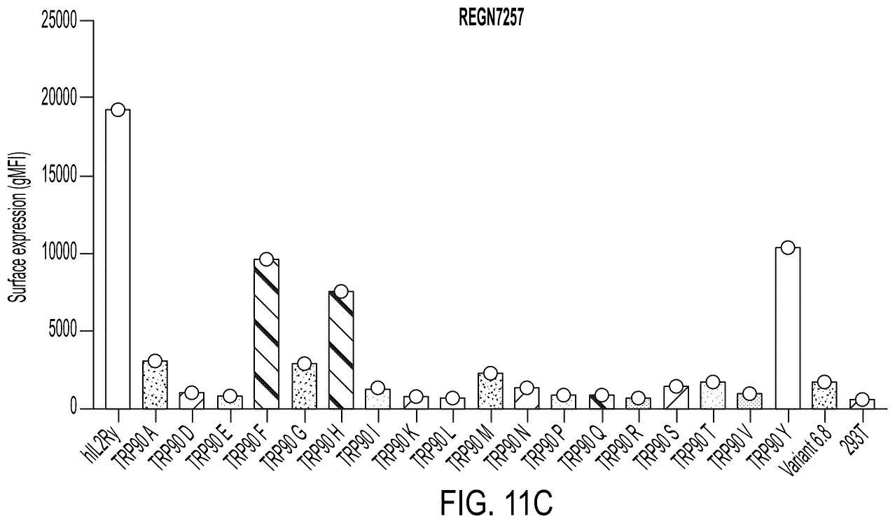

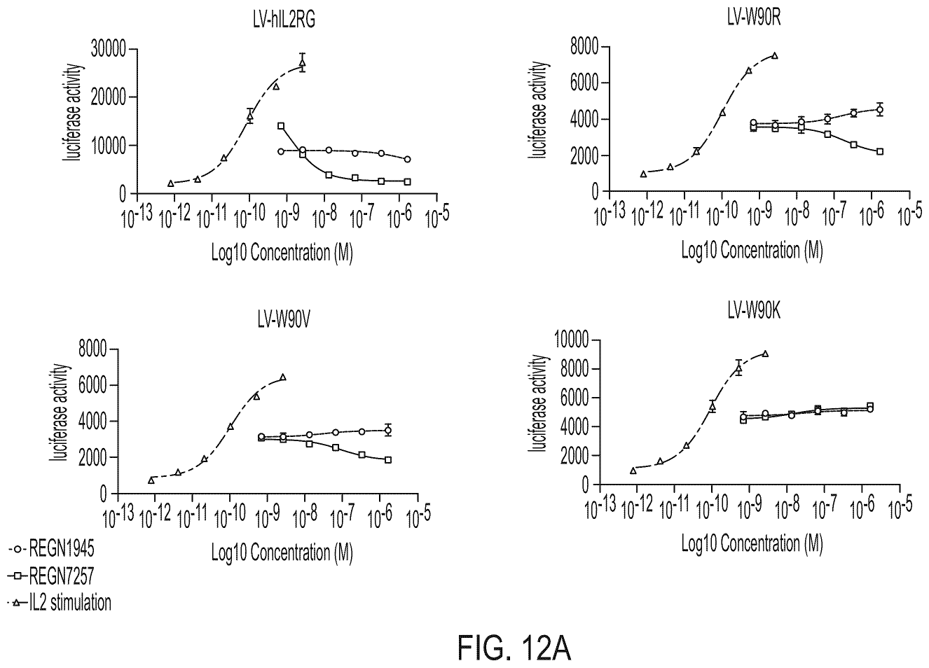

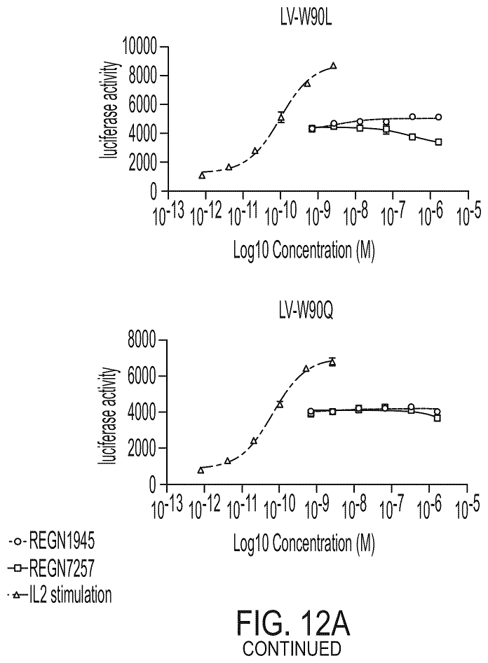

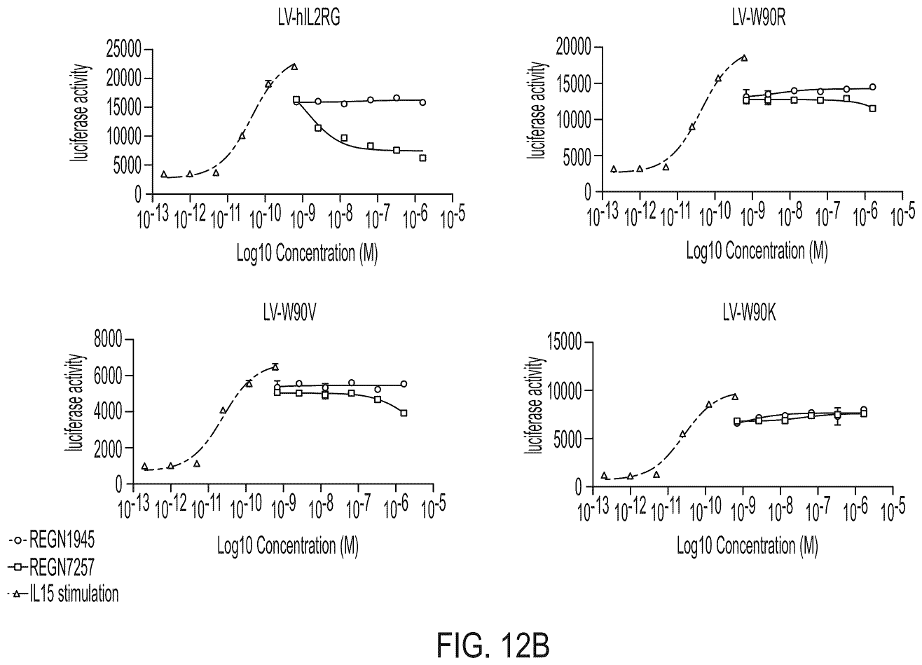

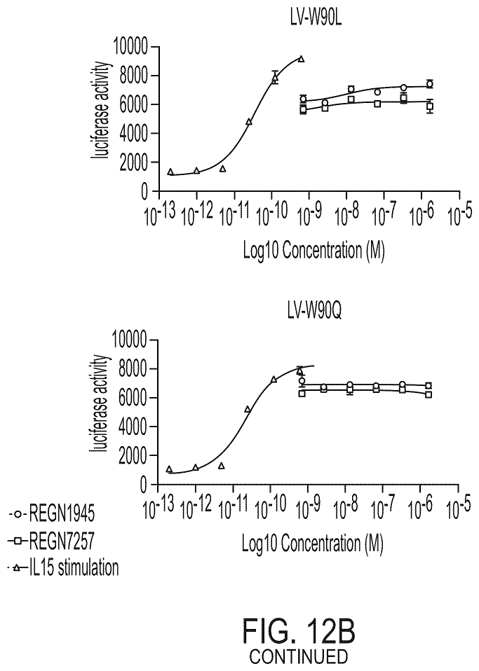

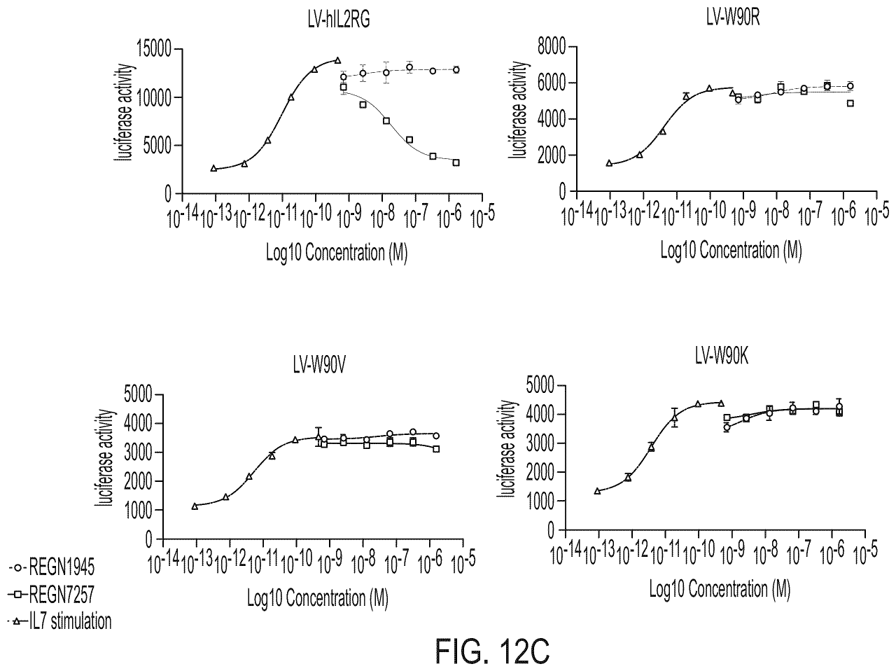

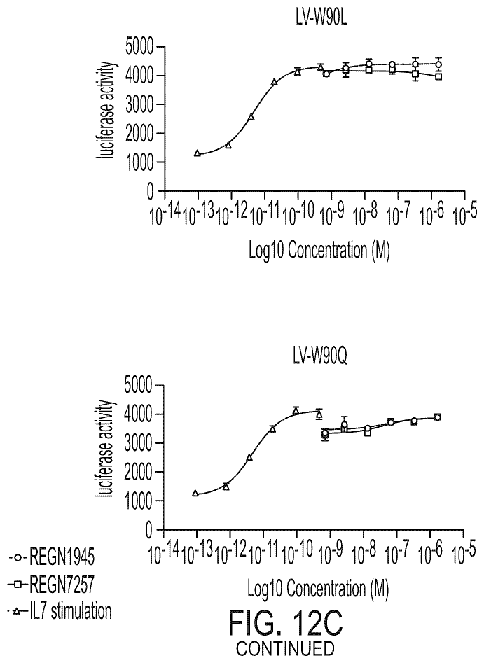

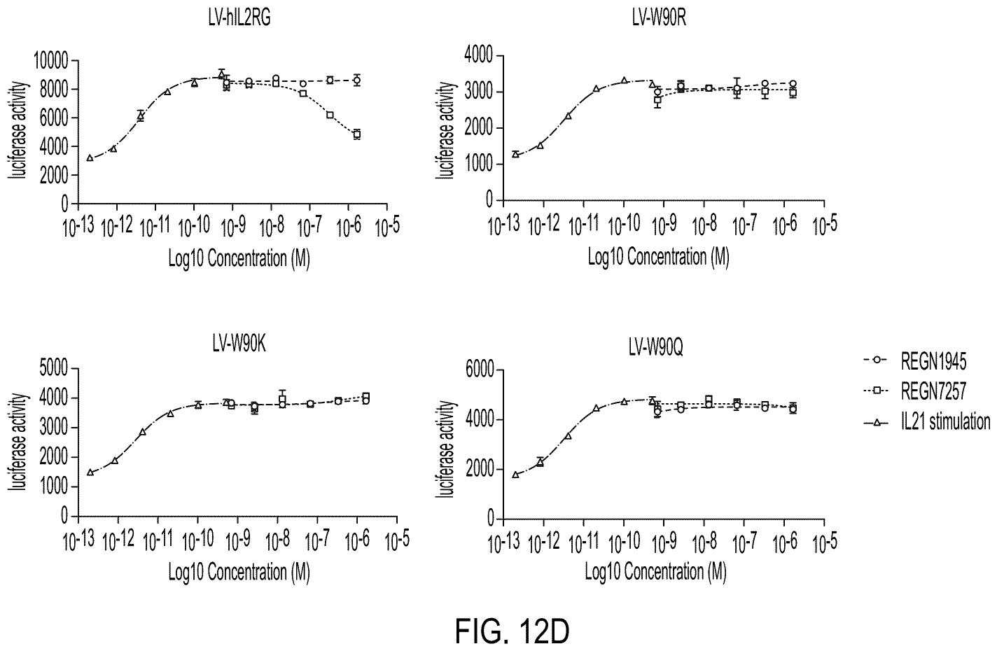

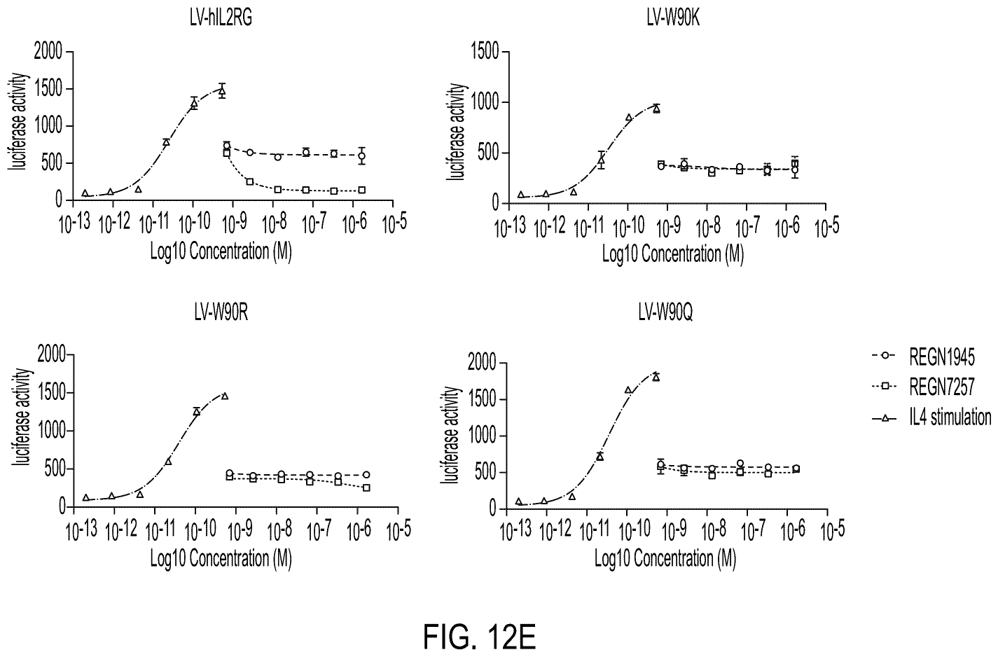

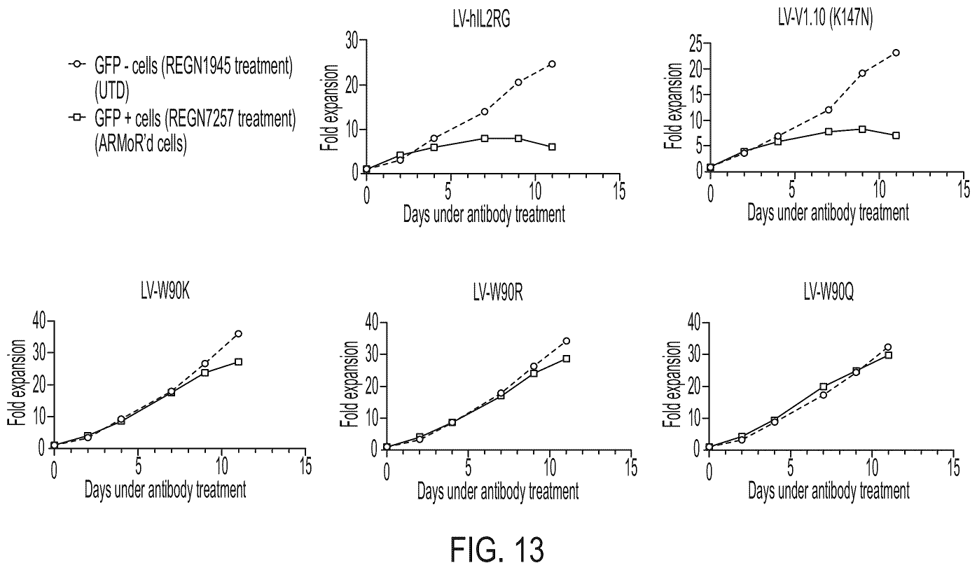

CryoEM studies identified several additional residues of human IL2RG that make potentially crucial molecular contacts with REGN7257, including W90, K92, N93, D95, D97, T127, R139, R140, Q141, T143, and K147 (data not shown). Because W90 contacts REGN7257 via its side chain, saturating mutagenesis testing amino acid substitutions was performed at this site. Expression of myc-tagged IL2RG constructs in 293T cells identified several W90 substitution variants that retained surface expression by flow cytometry (FIGS. 11A-11B) while strongly reducing REGN7257 binding (W90V, W90R, W90Q, W90L, W90K, W90E, and W90D; FIGS. 11A and 11C). These IL2RG variants with mutations in REGN7257 binding region 2 signaled efficiently in response to y-chain cytokines and evaded signaling blockade by REGN7257 (FIGS. 12A-12E). In addition, these IL2RG variants with mutations in REGN7257 binding region 2 supported IL2-dependent growth of primary T cells and conferred resistance to REGN7257 growth suppression (FIG. 13).

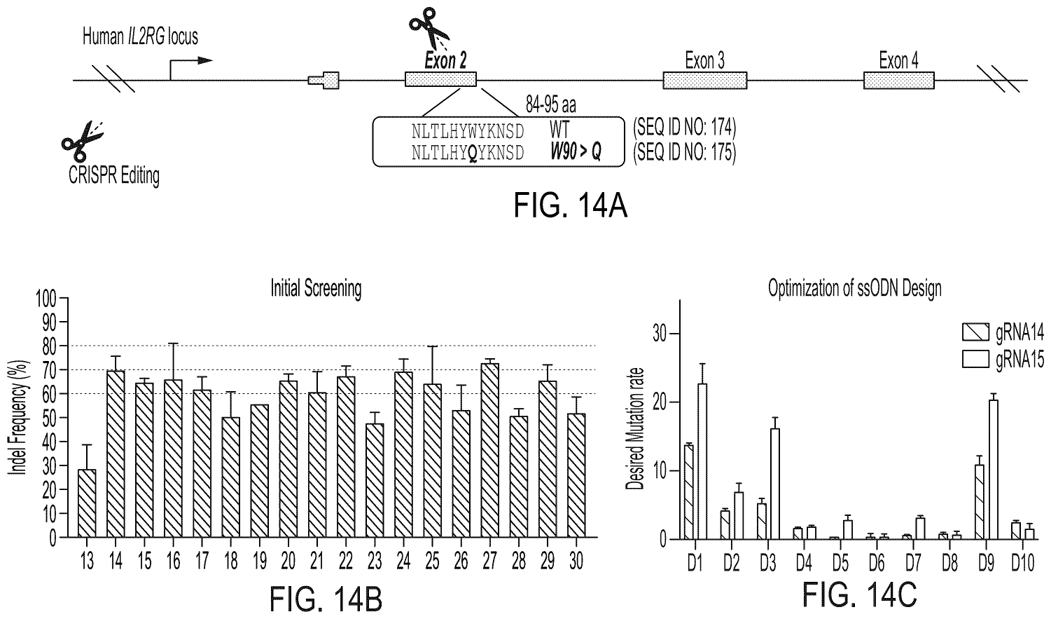

Given the importance of human IL2RG residue W90 to REGN7257 binding (FIGS. 11A-11C), CRISPR/Cas9-based gene editing strategies were developed to introduce modifications to the corresponding region of the human IL2RG gene (FIG. 14A). A panel of Cas9 single guide RNAs in exon 2, which encodes W90 and surrounding residues, was introduced into primary human T cells by electroporation of Cas9/sgRNA ribonucleoprotein complexes (RNPs). See Table 5. Editing frequencies were determined by PCR amplification of the corresponding genomic region and sequencing analysis to quantify indels and substitutions at predicted Cas9 cut sites, an indicator of error-prone non-homologous end joining (NHEJ) at the site of Cas9-induced double-stranded breaks (DSBs) (FIG. 14B). The two sgRNA sequences (sgRNAs 14 and 15) targeting the region encoding W90 and showing the highest frequency of gene editing were then tested in combination with a series of single-strand oligodeoxynucleotide (ssODN) donor repair sequences, to introduce nucleotide edits encoding the W90Q substitution (FIG. 14C). See Table 6. Ten ssODNs were designed for use with sgRNA14, and ten ssODNs were designed for use with sgRNA15. Several sgRNA-ssODN combinations were identified that efficiently introduce the desired gene edit into primary human T cells genomic DNA, an approach readily extendable to other immune cells and their progenitors.

TABLE 5
IL2RG guide RNAs.

| | | | SEQ ID NOS |
|---|---|---|---|
| gRNA | Target Sequence | Guide Sequence | (Target, Guide) |
| 13 | TCGAGTACATGAATTGCACT | UCGAGUACAUGAAUUGCACU | 118, 136 |
| 14 | CAGAGTGAGGTTGGTAGGCT | CAGAGUGAGGUUGGUAGGCU | 119, 137 |
| 15 | TAATGCAGAGTGAGGTTGGT | UAAUGCAGAGUGAGGUUGGU | 120, 138 |
| 16 | GGTCAGGAAGAAATCTAGAT | GGUCAGGAAGAAAUCUAGAU | 121, 139 |
| 17 | GCAGAGTGAGGTTGGTAGGC | GCAGAGUGAGGUUGGUAGGC | 122, 140 |
| 18 | GAAACACTGAGGGAGTCAGT | GAAACACUGAGGGAGUCAGU | 123, 141 |
| 19 | AGTCAGTGGGCATAGTGGTC | AGUCAGUGGGCAUAGUGGUC | 124, 142 |
| 20 | CAAAACACTGAACCTCTGGG | CAAAACACUGAACCUCUGGG | 125, 143 |
| 21 | TCAGGAAGAAATCTAGATTG | UCAGGAAGAAAUCUAGAUUG | 126, 144 |
| 22 | GTCAGGAAGAAATCTAGATT | GUCAGGAAGAAAUCUAGAUU | 127, 145 |
| 23 | AACACAAAACACTGAACCTC | AACACAAAACACUGAACCUC | 128, 146 |
| 24 | GAGGGAGTCAGTGGGCATAG | GAGGGAGUCAGUGGGCAUAG | 129, 147 |
| 25 | AAAACACTGAACCTCTGGGA | AAAACACUGAACCUCUGGGA | 130, 148 |
| 26 | AAACACTGAACCTCTGGGAG | AAACACUGAACCUCUGGGAG | 131, 149 |
| 27 | AGAGTGAGGTTGGTAGGCTG | AGAGUGAGGUUGGUAGGCUG | 132, 150 |
| 28 | GGGCAGAGTGGAAACACTGA | GGGCAGAGUGGAAACACUGA | 133, 151 |
| 29 | ACACAAAACACTGAACCTCT | ACACAAAACACUGAACCUCU | 134, 152 |
| 30 | GGAAACACTGAGGGAGTCAG | GGAAACACUGAGGGAGUCAG | 135, 153 |

TABLE 6
IL2RG W90Q ssODNs.

| | | SEQ |
|---|---|---|
| SSODN | Sequence | ID NO |
| 14-D1 | C*T*TCTTCATCCCCTCCCCCTCGTCCCTTCTCATACTGATAATGCAGAGTGAGGT | 154 |
| | TGGTAGGCTGTGGCTCAGAGCTGCTGTTCCAAGTGCAATTCATGTACTCGACAT | |
| | TGAACACAAA*A*C | |
| 14-D2 | G*T*TTTGTGTTCAATGTCGAGTACATGAATTGCACTTGGAACAGCAGCTCTGAG | 155 |
| | CCACAGCCTACCAACCTCACTCTGCATTATCAGTATGAGAAGGGACGAGGGGG | |
| | AGGGGATGAAGA*A*G | |
| 14-D3 | C*C*CCTCGTCCCTTCTCATACTGATAATGCAGAGTGAGGTTGGTAGGCTGTGGC | 156 |
| | TCAGAGCTGCTGTTCCAAGTGCAATTCATGTACTC*G*A | |
| 14-D4 | T*C*GAGTACATGAATTGCACTTGGAACAGCAGCTCTGAGCCACAGCCTACCAA | 157 |
| | CCTCACTCTGCATTATCAGTATGAGAAGGGACGAGG*G*G | |
| 14-D5 | C*A*TACTGATAATGCAGAGTGAGGTTGGTAGGCTGTGGCTCAGAGCTGCTGTT | 158 |
| | CCAAGT*G*C | |
| 14-D6 | G*C*ACTTGGAACAGCAGCTCTGAGCCACAGCCTACCAACCTCACTCTGCATTAT | 159 |
| | CAGTA*T*G | |
| 14-D7 | C*A*TACTGATAATGCAGAGTGAGGTTGGTAGGCTGTGGCTCAGAGCTGCTGTT | 160 |
| | CCAAGTGCAATTCATGTACTCGACATTGAACACAAA*A*C | |
| 14-D8 | G*T*TTTGTGTTCAATGTCGAGTACATGAATTGCACTTGGAACAGCAGCTCTGAG | 161 |
| | CCACAGCCTACCAACCTCACTCTGCATTATCAGTA*T*G | |
| 14-D9 | C*T*TCTTCATCCCCTCCCCCTCGTCCCTTCTCATACTGATAATGCAGAGTGAGGT | 162 |
| | TGGTAGGCTGTGGCTCAGAGCTGCTGTTCCAAGT*G*C | |
| 14-D10 | G*C*ACTTGGAACAGCAGCTCTGAGCCACAGCCTACCAACCTCACTCTGCATTAT | 163 |
| | CAGTATGAGAAGGGACGAGGGGGAGGGGATGAAGA*A*G | |
| 15-D1 | C*A*CCTCTTCTTCATCCCCTCCCCCTCGTCCCTTCTCATACTGATAGTGTAGTGT | 164 |
| | TAGGTTGGTAGGCTGGGGCTCAGAGCTGCTGTTCCAAGTGCAATTCATGTACTC | |
| | GACATTGAAC*A*C | |
| 15-D2 | G*T*GTTCAATGTCGAGTACATGAATTGCACTTGGAACAGCAGCTCTGAGCCCC | 165 |
| | AGCCTACCAACCTAACACTACACTATCAGTATGAGAAGGGACGAGGGGGAGGG | |
| | GATGAAGAAGAGG*T*G | |
| 15-D3 | C*C*CTCCCCCTCGTCCCTTCTCATACTGATAGTGTAGTGTTAGGTTGGTAGGCT | 166 |
| | GGGGCTCAGAGCTGCTGTTCCAAGTGCAATTCATG*T*A | |
| 15-D4 | T*A*CATGAATTGCACTTGGAACAGCAGCTCTGAGCCCCAGCCTACCAACCTAA | 167 |
| | CACTACACTATCAGTATGAGAAGGGACGAGGGGGAG*G*G | |
| 15-D5 | C*T*TCTCATACTGATAGTGTAGTGTTAGGTTGGTAGGCTGGGGCTCAGAGCTGC | 168 |
| | TGTTC*C*A | |
| 15-D6 | T*G*GAACAGCAGCTCTGAGCCCCAGCCTACCAACCTAACACTACACTATCAGT | 169 |
| | ATGAGA*A*G | |
| 15-D7 | C*T*TCTCATACTGATAGTGTAGTGTTAGGTTGGTAGGCTGGGGCTCAGAGCTGC | 170 |
| | TGTTCCAAGTGCAATTCATGTACTCGACATTGAAC*A*C | |
| 15-D8 | G*T*GTTCAATGTCGAGTACATGAATTGCACTTGGAACAGCAGCTCTGAGCCCC | 171 |
| | AGCCTACCAACCTAACACTACACTATCAGTATGAGA*A*G | |
| 15-D9 | C*A*CCTCTTCTTCATCCCCTCCCCCTCGTCCCTTCTCATACTGATAGTGTAGTGT | 172 |
| | TAGGTTGGTAGGCTGGGGCTCAGAGCTGCTGTTC*C*A | |
| 15-D10 | T*G*GAACAGCAGCTCTGAGCCCCAGCCTACCAACCTAACACTACACTATCAGT | 173 |
| | ATGAGAAGGGACGAGGGGGAGGGGATGAAGAAGAGG*T*G | |

*denotes phosphorothioate linkage

### Example 3. Antibody-Resistant Modified Receptors for Engineered NK Therapies

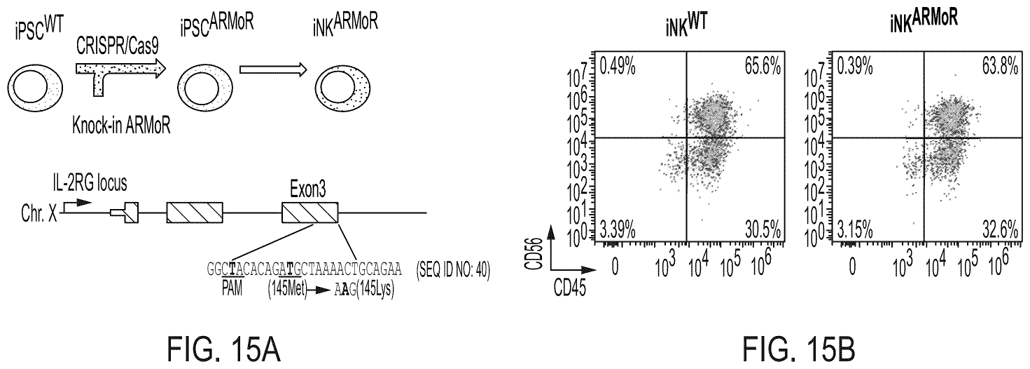

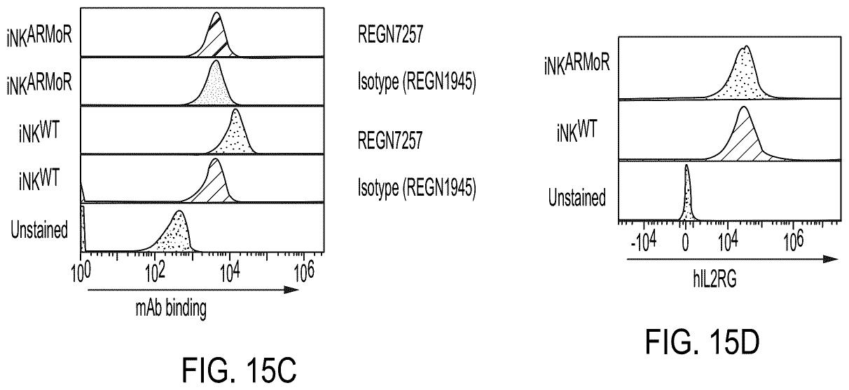

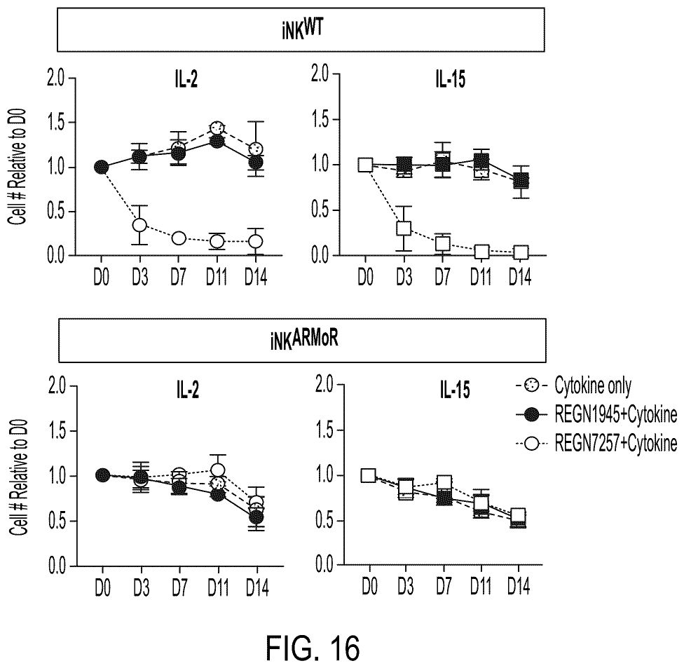

Engineered natural killer (NK) cells are promising cancer therapies due to their intrinsic cytotoxic function and lower risk of graft-versus-host reactivity compared to T cells. Moreover, highly potent NK cells can be derived from induced pluripotent stem cells (iPSCs), which are highly amenable to genetic manipulation. Goldenson et al. (2022) Front. Immunol. 13:841107, herein incorporated by reference in its entirety for all purposes. To test the IL2RG antibody-resistant modified receptor (ARMoR) approach in NK cells, the previously characterized M145K substitution was introduced into the IL2RG gene of human iPSCs, which were then used to generate iPSC-derived NK cells (iNKs, FIG. 15A). The NK differentiation potential of iPSCs engineered with the M145K substitution (iPSC^{ARMoR}) was similar to iPSCs lacking the mutation (iPSC^{WT}), indicating preserved IL2, IL7, and IL15 signaling by the M145K IL2RG variant during NK differentiation (FIG. 15B). Flow cytometry measurements on iNK cells engineered with the M145K IL2RG mutation (INK^{ARMoR}) confirmed a loss of binding to anti-IL2RG REGN7257 (FIG. 15C). Measurements with a second anti-IL2RG antibody against a different epitope confirmed that iNK^{ARMoR} and iNKWT cells express similar surface levels of IL2RG overall (FIG. 15D). Accordingly, while iNKWT cells showed suppressed growth in vitro when treated with REGN7257, iNK^{ARMoR} cells were resistant to suppression and showed similar growth to isotype-treated or untreated cells (FIG. 16).

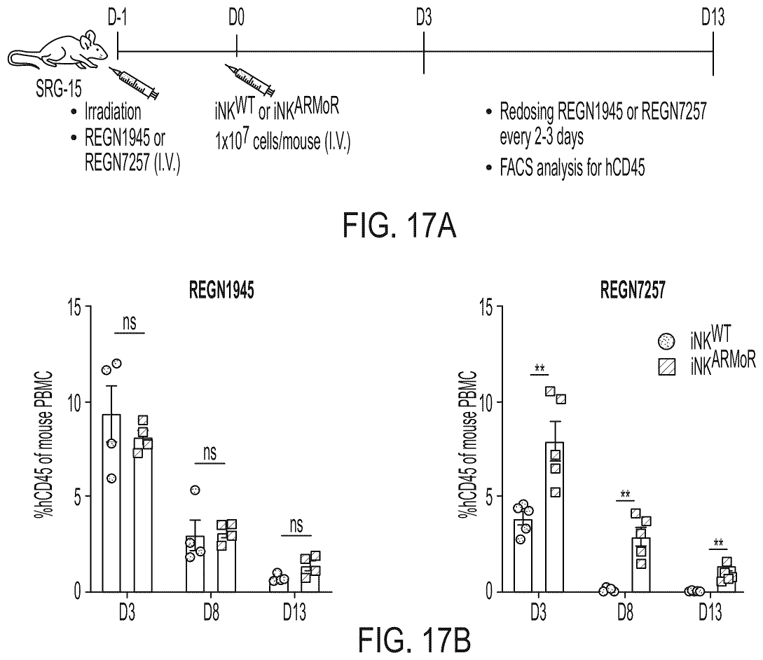

The effects of IL2RG antibody-resistant engineered receptor engineering were assessed in vivo by testing the engraftment and persistence of human iNK cells in immunodeficient mice (FIG. 17A). In mice treated with an isotype control antibody (REGN1945), iNKWT and iNK^{ARMoR} cells showed similar persistence, remaining detectable at similar levels though the two-week study (FIG. 17B). However, in mice treated with REGN7257, iNKWT cells were suppressed and virtually absent by day 8, while iNK^{ARMoR} cells persisted through the end of the study at levels similar to isotype-treated mice. Therefore, NK cells with antibody-resistant engineered receptors showed resistance to suppression in vivo, and a competitive advantage compared to non-edited NK cells.

We claim:

1. A method for improving engraftment of donor cells in a subject in need thereof, comprising:
(a) providing donor cells that have been modified to express a first isoform of a target protein, wherein the target protein is a protein expressed on the cell surface of hematopoietic cells, wherein the first isoform of the target protein is different from a second isoform of the target protein, wherein the second isoform is expressed in host cells of the subject;
(b) administering the donor cells to the subject, and
(c) selectively inhibiting host cells in the subject based on their expression of the second isoform of the target protein, thereby improving engraftment of donor cells in the subject.

2. The method of claim 1, wherein the target protein is a receptor.

3. The method of claim 1 or 2, wherein the target protein is a cytokine receptor or a chemokine receptor, optionally wherein the target protein is the cytokine receptor.

4. The method of any preceding claim, wherein the target protein is a protein expressed on the cell surface of lymphocytes.

5. The method of any preceding claim, wherein the target protein is a cytokine receptor sub-unit of an interleukin-2 (IL-2) receptor, an IL-4 receptor, an IL-7 receptor, an IL-9 receptor, an IL-15 receptor, or an IL-21 receptor.

6. The method of any preceding claim, wherein the target protein is interleukin-2 receptor subunit gamma (IL2RG).

7. The method of any preceding claim, wherein the selective inhibition of host cells in step (c) does not comprise ablation of host cells by an active killing mechanism.

8. The method of any preceding claim, wherein the selective inhibition in step (c) comprises: (1) blocking growth of the host cells to provide a competitive growth advantage to the donor cells; (2) blocking localization or trafficking of the host cells to provide a competitive homing advantage to the donor cells; (3) blocking a cell-cell interaction or adhesion of the host cells to provide a competitive tissue infiltration advantage to the donor cells; or (4) blocking immune cell activation in the host cells to provide a competitive advantage to the donor cells.

9. The method of any preceding claim, wherein the selective inhibition in step (c) comprises blocking growth of the host cells and/or blocking immune cell activation in the host cells to provide a competitive growth advantage to the donor cells.

10. The method of any preceding claim, wherein the first isoform and the second isoform are functionally indistinguishable but immunologically distinguishable.

11. The method of any preceding claim, wherein the donor cells express both the first isoform of the target protein and the second isoform of the target protein.

12. The method of any one of claims 1-10, wherein the donor cells express only the first isoform of the target protein.

13. The method of any preceding claim, wherein the first isoform of the target protein is expressed from an expression vector in the donor cells, or wherein a genomic locus has been edited to express the first isoform of the target protein in the donor cells.

14. The method of claim 13, wherein the genomic locus is an endogenous genomic locus encoding the target protein, optionally wherein the target protein is IL2RG and the genomic locus is an IL2RG genomic locus.

15. The method of claim 13, wherein the genomic locus is not an endogenous genomic locus encoding the target protein, optionally wherein the target protein is IL2RG, and the genomic locus is not an IL2RG genomic locus.

16. The method of any preceding claim, wherein the first isoform of the target protein is a genetically engineered isoform of the target protein.

17. The method of any preceding claim, wherein the first isoform of the target protein is genetically engineered to comprise a mutation to provide an altered epitope, optionally wherein the mutation is an artificial mutation.

18. The method of claim 17, wherein the altered epitope is a binding region of a target protein antagonist such that the target protein antagonist exhibits reduced or abolished ability to bind and/or inhibit the first isoform of the target protein compared to its ability to bind and/or inhibit the second isoform of the target protein.

19. The method of claim 18, wherein both the first isoform of the target protein and the second isoform of the target protein retain the ability to bind to an endogenous ligand, optionally wherein the target protein antagonist blocks the binding of the endogenous ligand to the second isoform of the target protein but not the first isoform of the target protein.

20. The method claim 18 or 19, wherein the target protein is IL2RG,
wherein the altered epitope is in a binding region of the target protein antagonist, wherein the target protein antagonist is an antibody comprising an immunoglobulin light chain or variable region thereof comprising three light chain CDRs and an immunoglobulin heavy chain or variable region thereof comprising three heavy chain CDRs,
wherein the three light chain CDRs comprise, consist essentially of, or consist of the sequences set forth in SEQ ID NOS: 12, 14, and 16, respectively, and
wherein the three heavy chain CDRs comprise, consist essentially of, or consist of the sequences set forth in SEQ ID NOS: 4, 6, and 8, respectively.

21. The method of any one of claims 17-20, wherein the target protein is IL2RG, and wherein the mutation comprises a mutation encoded by nucleotides within exon 2 and/or exon 3 of the IL2RG gene at the IL2RG genomic locus.

22. The method of any one of claims 17-21, wherein the target protein is IL2RG, and wherein the mutation comprises a mutation or substitution within a region from position T127 to position N150 and/or within a region from position L87 to position D97.

23. The method of any one of claims 17-22, wherein the target protein is IL2RG, and wherein the mutation comprises a mutation or substitution at position M145, position W90, position K92, position N93, position D95, position D97, position T127, position R139, position R140, position Q141, position T143, and/or position K147.

24. The method of any one of claims 17-23, wherein the target protein is IL2RG, and wherein the mutation comprises a mutation or substitution at position M145, optionally wherein the substitution is a M145K substitution, a M145D substitution, a M145E substitution, a M145P substitution, a M145W substitution, or an M145Y substitution.

25. The method of any one of claims 17-24, wherein the target protein is IL2RG, and wherein the mutation comprises a M145K substitution.

26. The method of any one of claims 17-25, wherein the target protein is IL2RG, and wherein the mutation comprises a mutation or substitution at position W90, optionally wherein the substitution is a W90V substitution, a W90R substitution, a W90Q substitution, a W90L substitution, a W90K substitution, a W90E substitution, or a W90D substitution, optionally wherein the mutation comprises a W90Q substitution.

27. The method of any one of claims 17-26, wherein the target protein is IL2RG, and wherein the mutation comprises a mutation or substitution at position M145 and a mutation or substitution at position W90.

28. The method of any preceding claim, wherein the selective inhibition in step (c) comprises administering a target protein antagonist to the subject, wherein the target protein antagonist specifically binds to the second isoform of the target protein but does not specifically bind to the first isoform of the target protein, optionally wherein step (c) comprises multiple administrations of the target protein antagonist.

29. The method of any one of claims 18-20 and 28, wherein the target protein antagonist is an antigen-binding protein.

30. The method of claim 29, wherein the antigen-binding protein is an antibody or an antigen-binding fragment thereof.

31. The method of claim 29 or 30, wherein the target protein is IL2RG,
wherein the antigen-binding protein comprises an immunoglobulin light chain or variable region thereof comprising three light chain CDRs and an immunoglobulin heavy chain or variable region thereof comprising three heavy chain CDRs,
wherein the three light chain CDRs comprise, consist essentially of, or consist of sequences at least 90% identical to the sequences set forth in SEQ ID NOS: 12, 14, and 16, respectively, and
wherein the three heavy chain CDRs comprise, consist essentially of, or consist of sequences at least 90% identical to the sequences set forth in SEQ ID NOS: 4, 6, and 8, respectively.

32. The method of claim 31,
wherein the three light chain CDRs comprise, consist essentially of, or consist of the sequences set forth in SEQ ID NOS: 12, 14, and 16, respectively, and
wherein the three heavy chain CDRs comprise, consist essentially of, or consist of the sequences set forth in SEQ ID NOS: 4, 6, and 8, respectively.

33. The method of claim 29 or 30, wherein the target protein is IL2RG,
wherein the antigen-binding protein comprises an immunoglobulin light chain variable region that comprises, consists essentially of, or consists of a sequence at least 90% identical to the sequence set forth in SEQ ID NO: 10, and
wherein the antigen-binding protein comprises an immunoglobulin heavy chain variable region that comprises, consists essentially of, or consists of a sequence at least 90% identical to the sequence set forth in SEQ ID NO: 2.

34. The method of claim 33, wherein the target protein is IL2RG,
wherein the immunoglobulin light chain variable region comprises, consists essentially of, or consists of the sequence set forth in SEQ ID NO: 10, and
wherein the immunoglobulin heavy chain variable region comprises, consists essentially of, or consists of the sequence set forth in SEQ ID NO: 2.

35. The method of 29 or 30, wherein the target protein is IL2RG,
wherein the antigen-binding protein comprises an immunoglobulin light chain that comprises, consists essentially of, or consists of the sequence set forth in SEQ ID NO: 20, and
wherein the antigen-binding protein comprises an immunoglobulin heavy chain that comprises, consists essentially of, or consists of the sequence set forth in SEQ ID NO: 18.

36. The method of any preceding claim, wherein the donor cells and/or the host cells are hematopoietic cells, optionally wherein the donor cells and/or the host cells are immune cells.

37. The method of any preceding claim, wherein the donor cells and/or the host cells are lymphocytes or lymphoid progenitor cells.

38. The method of any preceding claim, wherein the donor cells and/or the host cells are T cells.

39. The method of any preceding claim, wherein the donor cells and/or the host cells are tumor infiltrating lymphocytes (TILs).

40. The method of any one of claims 1-37, wherein the donor cells and/or the host cells are B cells.

41. The method of any one of claims 1-37, wherein the donor cells and/or the host cells are NK cells.

42. The method of any one of claims 1-36, wherein the donor cells and/or the host cells are hematopoietic stem and progenitor cells.

43. The method of any one of claims 1-41, wherein the donor cells and/or the host cells are derived from hematopoietic stem cells or hematopoietic stem and progenitor cells.

44. The method of any one of claims 1-42, wherein the donor cells are derived from induced pluripotent stem cells.

45. The method of any preceding claim, wherein the subject is a mammal or a non-human mammal, and the donor cells are mammalian cells or non-human mammalian cells.

46. The method of any preceding claim, wherein the subject is a human, and the donor cells are human cells.

47. The method of any preceding claim, wherein the donor cells comprise or express a therapeutic molecule.

48. The method of claim 47, wherein the therapeutic molecule does not target the target protein.

49. The method of any preceding claim, wherein the donor cells comprise or express an immunoglobulin.

50. The method of claim 49, wherein the immunoglobulin does not target the target protein.

51. The method of any preceding claim, wherein the donor cells comprise a chimeric antigen receptor (CAR) or an exogenous T cell receptor (TCR).

52. The method of claim 51, wherein the CAR or the exogenous TCR does not target the target protein.

53. The method of any preceding claim, wherein the donor cells are autologous.

54. The method of any one of claims 1-52, wherein the donor cells are allogeneic or syngeneic.

55. The method of any preceding claim, wherein the subject has a disease or disorder, and the method is for treating the disease or disorder.

56. The method of any preceding claim, wherein the subject has cancer.

57. The method of claim 56, wherein the cancer is a solid tumor cancer.

58. The method of claim 56, wherein the cancer is a hematologic cancer.

59. The method of any preceding claim, wherein the subject has a hematopoietic malignancy, and the method is for treating the hematopoietic malignancy in the subject.

60. The method of any preceding claim, wherein the subject has defective immune cells or a genetic deficiency in hematopoiesis.

61. The method of claim 60, wherein the genetic deficiency in hematopoiesis is sickle cell disease or severe combined immunodeficiency (SCID).

62. The method of any preceding claim, wherein steps (b) and (c) occur simultaneously.

63. The method of any one of claims 1-61, wherein step (b) occurs prior to step (c), optionally wherein step (c) comprises multiple administrations of a target protein antagonist subsequent to step (b).

64. The method of any one of claims 1-61, wherein step (b) occurs subsequent to step (c), optionally wherein step (c) comprises multiple administrations of a target protein antagonist prior to step (b).

65. The method of any one of claims 1-64, wherein step (c) occurs both prior to and subsequent to step (b), optionally wherein step (c) comprises multiple administrations of a target protein antagonist prior to step (b) and/or multiple administrations of the target protein antagonist subsequent to step (b).

66. The method of any preceding claim, further comprising generating the donor cells by modifying a population of cells to express the first isoform of the target protein prior to step (a).

67. The method of claim 66, wherein the population of cells is a population of induced pluripotent stem cells, and the method further comprises differentiating the induced pluripotent stem cells prior to step (a) into the donor cells that are administered in step (a), optionally wherein the induced pluripotent stem cells are differentiated into hematopoietic cells, lymphocytes or lymphoid progenitor cells, T cells, B cells, NK cells, hematopoietic stem cells, or hematopoietic stem and progenitor cells.

68. The method of claim 66, wherein the population of cells is a population of hematopoietic stem cells or hematopoietic stem and progenitor cells, and the method further comprises differentiating the hematopoietic stem cells or hematopoietic stem and progenitor cells prior to step (a) into the donor cells that are administered in step (a), optionally wherein the hematopoietic stem cells or hematopoietic stem and progenitor cells are differentiated into differentiated hematopoietic cells, lymphocytes or lymphoid progenitor cells, T cells, B cells, or NK cells.

69. The method of any one of claims 66-68, wherein generating the donor cells comprises introducing an expression vector encoding the first isoform of the target protein to express the first isoform of the target protein prior to step (a), or wherein generating the donor cells comprises editing a genomic locus in the population of cells to express the first isoform of the target protein prior to step (a).

70. The method of claim 69, wherein the genomic locus is an endogenous genomic locus encoding the target protein, optionally wherein the target protein is IL2RG, and the genomic locus is an IL2RG genomic locus.

71. The method of claim 69, wherein the genomic locus is not an endogenous genomic locus encoding the target protein, optionally wherein the target protein is IL2RG, and the genomic locus is not an IL2RG genomic locus.

72. The method of any one of claims 69-71, wherein the editing comprises introducing into the population of cells:
(1) a nuclease agent or one or more nucleic acids encoding the nuclease agent, wherein the nuclease agent targets a nuclease target sequence in the genomic locus; and
(2) an exogenous donor nucleic acid,
wherein the nuclease agent cleaves the genomic locus and the exogenous donor nucleic acid is inserted into the genomic locus or recombines with the genomic locus to generate the donor cells that express the first isoform of the target protein.

73. The method of claim 72, wherein the nuclease agent comprises:
(a) a zinc finger nuclease (ZFN);
(b) a transcription activator-like effector nuclease (TALEN); or
(c) (i) a Cas protein; and
(ii) a guide RNA, wherein the guide RNA comprises a DNA-targeting segment that targets a guide RNA target sequence that is the nuclease target sequence, and wherein the guide RNA binds to the Cas protein and targets the Cas protein to the guide RNA target sequence.

74. The method of claim 73, wherein the nuclease agent comprises the Cas protein and the guide RNA, optionally wherein the DNA-targeting segment comprises the sequence set forth in any one of SEQ ID NOS: 76-87 or the guide RNA target sequence comprises the sequence set forth in any one of SEQ ID NOS: 64-75, or optionally wherein the DNA-targeting segment comprises the sequence set forth in any one of SEQ ID NOS: 136-153 or the guide RNA target sequence comprises the sequence set forth in any one of SEQ ID NOS:
118-135.

75. The method of claim 73 or 74, wherein the Cas protein is a Cas9 protein.

76. The method of any one of claims 72-75, wherein the exogenous donor nucleic acid comprises homology arms.

77. The method of any one of claims 72-76, wherein the exogenous donor nucleic acid is a single-stranded oligodeoxynucleotide (ssODN), optionally wherein the ssODN comprises the nucleic acid sequence set forth in any one of SEQ ID NOS: 88-117 or optionally wherein the ssODN comprises the nucleic acid sequence set forth in any one of SEQ ID NOS: 154-173.

78. The method of claim 77, wherein:
(I) the DNA-targeting segment comprises the sequence set forth in SEQ ID NO: 77 or the guide RNA target sequence comprises the sequence set forth in SEQ ID NO: 65, and wherein the ssODN comprises the nucleic acid sequence set forth in any one of SEQ ID NOS: 88-97;
(II) the DNA-targeting segment comprises the sequence set forth in SEQ ID NO: 83 or the guide RNA target sequence comprises the sequence set forth in SEQ ID NO: 71, and wherein the ssODN comprises the nucleic acid sequence set forth in any one of SEQ ID NOS: 98-10⁷;
(III) the DNA-targeting segment comprises the sequence set forth in SEQ ID NO: 86 or the guide RNA target sequence comprises the sequence set forth in SEQ ID NO: 74, and wherein the ssODN comprises the nucleic acid sequence set forth in any one of SEQ ID NOS: 108-117;
(IV) the DNA-targeting segment comprises the sequence set forth in SEQ ID NO: 137 or the guide RNA target sequence comprises the sequence set forth in SEQ ID NO: 119, and wherein the ssODN comprises the nucleic acid sequence set forth in any one of SEQ ID NOS: 154-163; or
(V) the DNA-targeting segment comprises the sequence set forth in SEQ ID NO: 138 or the guide RNA target sequence comprises the sequence set forth in SEQ ID NO: 120, and wherein the ssODN comprises the nucleic acid sequence set forth in any one of SEQ ID NOS: 164-173.

79. The method of any one of claims 66-78, further comprising isolating the population of cells from the subject or from a different subject prior to modifying the population of cells.

80. A combination medicament for administration to a subject in need thereof, comprising:
(a) a population of donor cells modified to express a first isoform of a target protein, wherein the target protein is a protein expressed on the cell surface of hematopoietic cells, wherein the first isoform of the target protein is different from a second isoform of the target protein; and
(b) a target protein antagonist that specifically binds to the second isoform of the target protein but does not specifically bind to the first isoform of the target protein.

81. The combination medicament of claim 80, wherein the target protein is a receptor.

82. The combination medicament of claim 80 or 81, wherein the target protein is a cytokine receptor or a chemokine receptor, optionally wherein the target protein is the cytokine receptor.

83. The combination medicament of any one of claims 80-82, wherein the target protein is a protein expressed on the cell surface of lymphocytes.

84. The combination medicament of any one of claims 80-83, wherein the target protein is a cytokine receptor sub-unit of an interleukin-2 (IL-2) receptor, an IL-4 receptor, an IL-7 receptor, an IL-9 receptor, an IL-15 receptor, or an IL-21 receptor.

85. The combination medicament of any one of claims 80-84, wherein the target protein is interleukin-2 receptor subunit gamma (IL2RG).

86. The combination medicament of any one of claims 80-85, wherein the target protein antagonist selectively inhibits host cells in the subject based on their expression of the second isoform of the target protein.

87. The combination medicament of claim 86, wherein the selective inhibition of host cells does not comprise ablation of host cells by an active killing mechanism.

88. The combination medicament of claim 86 or 87, wherein the selective inhibition comprises: (1) blocking growth of the host cells to provide a competitive growth advantage to the donor cells; (2) blocking localization or trafficking of the host cells to provide a competitive homing advantage to the donor cells; (3) blocking a cell-cell interaction or adhesion of the host cells to provide a competitive tissue infiltration advantage to the donor cells; or (4) blocking immune cell activation in the host cells to provide a competitive advantage to the donor cells.

89. The combination medicament of any one of claims 86-88, wherein the selective inhibition comprises blocking growth of the host cells and/or blocking immune cell activation in the host cells to provide a competitive growth advantage to the donor cells.

90. The combination medicament of any one of claims 80-89, wherein the first isoform and the second isoform are functionally indistinguishable but immunologically distinguishable.

91. The combination medicament of any one of claims 80-90, wherein the donor cells express both the first isoform of the target protein and the second isoform of the target protein.

92. The combination medicament of any one of claims 80-90, wherein the donor cells express only the first isoform of the target protein.

93. The combination medicament of any one of claims 80-92, wherein the first isoform of the target protein is expressed from an expression vector in the population of donor cells, or wherein a genomic locus has been edited to express the first isoform of the target protein in the population of donor cells.

94. The combination medicament of claim 93, wherein the genomic locus is an endogenous genomic locus encoding the target protein, optionally wherein the target protein is IL2RG, and the genomic locus is an IL2RG genomic locus.

95. The combination medicament of claim 93, wherein the genomic locus is not an endogenous genomic locus encoding the target protein, optionally wherein the target protein is IL2RG, and the genomic locus is not an IL2RG genomic locus.

96. The combination medicament of any one of claims 80-95, wherein the first isoform of the target protein is a genetically engineered isoform of the target protein.

97. The combination medicament of any one of claims 80-96, wherein the first isoform of the target protein is genetically engineered to comprise a mutation to provide an altered epitope, optionally wherein the mutation is an artificial mutation.

98. The combination medicament of claim 97, wherein the altered epitope is a binding region of the target protein antagonist such that the target protein antagonist exhibits reduced or abolished ability to bind and/or inhibit the first isoform of the target protein compared to its ability to bind and/or inhibit the second isoform of the target protein.

99. The combination medicament of claim 98, wherein both the first isoform of the target protein and the second isoform of the target protein retain the ability to bind to an endogenous ligand, optionally wherein the target protein antagonist blocks the binding of the endogenous ligand to the second isoform of the target protein but not the first isoform of the target protein.

100. The combination medicament of any one of claims 97-99, wherein the target protein is IL2RG,
wherein the altered epitope is in a binding region of the target protein antagonist, wherein the target protein antagonist is an antibody comprising an immunoglobulin light chain or variable region thereof comprising three light chain CDRs and an immunoglobulin heavy chain or variable region thereof comprising three heavy chain CDRs,
wherein the three light chain CDRs comprise, consist essentially of, or consist of the sequences set forth in SEQ ID NOS: 12, 14, and 16, respectively, and
wherein the three heavy chain CDRs comprise, consist essentially of, or consist of the sequences set forth in SEQ ID NOS: 4, 6, and 8, respectively.

101. The combination medicament of any one of claims 97-100, wherein the target protein is IL2RG, and wherein the mutation comprises a mutation encoded by nucleotides within exon 2 and/or exon 3 of the IL2RG gene at the IL2RG genomic locus.

102. The combination medicament of any one of claims 97-101, wherein the target protein is IL2RG, and wherein the mutation comprises a mutation or substitution within a region from position T127 to position N150 and/or within a region from position L87 to position D97.

103. The combination medicament of any one of claims 97-102, wherein the target protein is IL2RG, and wherein the mutation comprises a mutation or substitution at position M145, position W90, position K92, position N93, position D95, position D97, position T127, position R139, position R140, position Q141, position T143, and/or position K147.

104. The combination medicament of any one of claims 97-103, wherein the target protein is IL2RG, and wherein the mutation comprises a mutation or substitution at position M145, optionally wherein the substitution is a M145K substitution, a M145D substitution, a M145E substitution, a M145P substitution, a M145W substitution, or an M145Y substitution.

105. The combination medicament of any one of claims 97-104, wherein the target protein is IL2RG, and wherein the mutation comprises a M145K substitution.

106. The combination medicament of any one of claims 97-105, wherein the target protein is IL2RG, and wherein the mutation comprises a mutation or substitution at position W90, optionally wherein the substitution is a W90V substitution, a W90R substitution, a W90Q substitution, a W90L substitution, a W90K substitution, a W90E substitution, or a W90D substitution, optionally wherein the mutation comprises a W90Q substitution.

107. The combination medicament of any one of claims 97-106, wherein the target protein is IL2RG, and wherein the mutation comprises a mutation or substitution at position M145 and a mutation or substitution at position W90.

108. The combination medicament of any one of claims 80-107, wherein the target protein antagonist is an antigen-binding protein.

109. The combination medicament of claim 108, wherein the antigen-binding protein is an antibody or an antigen-binding fragment thereof.

110. The combination medicament of claim 108 or 109, wherein the target protein is IL2RG,
wherein the antigen-binding protein comprises an immunoglobulin light chain or variable region thereof comprising three light chain CDRs and an immunoglobulin heavy chain or variable region thereof comprising three heavy chain CDRs,
wherein the three light chain CDRs comprise, consist essentially of, or consist of sequences at least 90% identical to the sequences set forth in SEQ ID NOS: 12, 14, and 16, respectively, and
wherein the three heavy chain CDRs comprise, consist essentially of, or consist of sequences at least 90% identical to the sequences set forth in SEQ ID NOS: 4, 6, and 8, respectively.

111. The combination medicament of claim 110,
wherein the three light chain CDRs comprise, consist essentially of, or consist of the sequences set forth in SEQ ID NOS: 12, 14, and 16, respectively, and
wherein the three heavy chain CDRs comprise, consist essentially of, or consist of the sequences set forth in SEQ ID NOS: 4, 6, and 8, respectively.

112. The combination medicament of claim 108 or 109, wherein the target protein is IL2RG,
wherein the antigen-binding protein comprises an immunoglobulin light chain variable region that comprises, consists essentially of, or consists of a sequence at least 90% identical to the sequence set forth in SEQ ID NO: 10, and
wherein the antigen-binding protein comprises an immunoglobulin heavy chain variable region that comprises, consists essentially of, or consists of a sequence at least 90% identical to the sequence set forth in SEQ ID NO: 2.

113. The combination medicament of claim 112, wherein the target protein is IL2RG,
wherein the immunoglobulin light chain variable region comprises, consists essentially of, or consists of the sequence set forth in SEQ ID NO: 10, and
wherein the immunoglobulin heavy chain variable region comprises, consists essentially of, or consists of the sequence set forth in SEQ ID NO: 2.

114. The combination medicament of claim 108 or 109, wherein the target protein is IL2RG,
wherein the antigen-binding protein comprises an immunoglobulin light chain that comprises, consists essentially of, or consists of the sequence set forth in SEQ ID NO: 20, and
wherein the antigen-binding protein comprises an immunoglobulin heavy chain that comprises, consists essentially of, or consists of the sequence set forth in SEQ ID NO: 18.

115. The combination medicament of any one of claims 80-114, wherein the donor cells are hematopoietic cells, optionally wherein the donor cells are immune cells.

116. The combination medicament of any one of claims 80-115, wherein the donor cells are lymphocytes or lymphoid progenitor cells.

117. The combination medicament of any one of claims 80-116, wherein the donor cells are T cells.

118. The combination medicament of any one of claims 80-117, wherein the donor cells are tumor infiltrating lymphocytes (TILs).

119. The combination medicament of any one of claims 80-116, wherein the donor cells are B cells.

120. The combination medicament of any one of claims 80-116, wherein the donor cells are NK cells.

121. The combination medicament of any one of claims 80-115, wherein the donor cells are hematopoietic stem cells or hematopoietic stem and progenitor cells.

122. The combination medicament of any one of claims 80-121, wherein the donor cells are derived from induced pluripotent stem cells or are derived from hematopoietic stem cells or hematopoietic stem and progenitor cells.

123. The combination medicament of any one of claims 80-122, wherein the subject is a mammal or a non-human mammal, and the donor cells are mammalian cells or non-human mammalian cells.

124. The combination medicament of any one of claims 80-123, wherein the subject is a human, and the donor cells are human cells.

125. The combination medicament of any one of claims 80-124, wherein the donor cells comprise or express a therapeutic molecule.

126. The combination medicament of claim 125, wherein the therapeutic molecule does not target the target protein.

127. The combination medicament of any one of claims 80-126, wherein the donor cells comprise or express an immunoglobulin.

128. The combination medicament of claim 127, wherein the immunoglobulin does not target the target protein.

129. The combination medicament of any one of claims 80-128, wherein the donor cells comprise a chimeric antigen receptor (CAR) or an exogenous T cell receptor (TCR).

130. The combination medicament of claim 129, wherein the CAR or the exogenous TCR does not target the target protein.

131. The combination medicament of any one of claims 80-130, wherein the donor cells are autologous.

132. The combination medicament of any one of claims 80-130, wherein the donor cells are allogeneic or syngeneic.

133. The combination medicament of any one of claims 80-132, wherein the subject has a disease or disorder, and the combination medicament is for treating the disease or disorder.

134. The combination medicament of any one of claims 80-133, wherein the subject has cancer.

135. The combination medicament of claim 134, wherein the cancer is a solid tumor cancer.

136. The combination medicament of claim 134, wherein the cancer is a hematologic cancer.

137. The combination medicament of any one of claims 80-132, wherein the subject has a hematopoietic malignancy, and the combination medicament is for treating the hematopoietic malignancy in the subject.

138. The combination medicament of any one of claims 80-137, wherein the subject has defective immune cells or a genetic deficiency in hematopoiesis.

139. The combination medicament of claim 138, wherein the genetic deficiency in hematopoiesis is sickle cell disease or severe combined immunodeficiency (SCID).

140. An isolated cell or population of cells modified to express a first isoform of interleukin-2 receptor subunit gamma (IL2RG) that is different from a second isoform of IL2RG, wherein the first isoform of IL2RG is genetically engineered to comprise a mutation to provide an altered epitope, wherein the altered epitope is a binding region of an IL2RG antagonist such that the IL2RG antagonist exhibits reduced or abolished ability to bind and/or inhibit the first isoform of IL2RG compared to its ability to bind and/or inhibit the second isoform of IL2RG, and wherein the first isoform of IL2RG retains binding to its endogenous ligands.

141. The isolated cell or population of cells of claim 140, wherein the mutation is an artificial mutation.

142. The isolated cell or population of cells of claim 140 or 141, wherein both the first isoform of IL2RG and the second isoform of IL2RG retain the ability to bind to an endogenous ligand, optionally wherein the IL2RG antagonist blocks the binding of the endogenous ligand to the second isoform of IL2RG but not the first isoform of IL2RG.

143. The isolated cell or population of cells of any one of claims 140-142, wherein the first isoform and the second isoform are functionally indistinguishable but immunologically distinguishable.

144. The isolated cell or population of cells of any one of claims 140-143, wherein the cell or cells express both the first isoform of IL2RG and the second isoform of IL2RG.

145. The isolated cell or population of cells of any one of claims 140-143, wherein the cell or cells express only the first isoform of IL2RG.

146. The isolated cell or population of cells of any one of claims 140-145, wherein the first isoform of IL2RG is expressed from an expression vector in the cell or cells, or wherein a genomic locus has been edited to express the first isoform of IL2RG in the cell or cells.

147. The isolated cell or population of cells of claim 146, wherein the genomic locus is an endogenous IL2RG genomic locus.

148. The isolated cell or population of cells of claim 146, wherein the genomic locus is not an endogenous IL2RG genomic locus.

149. The isolated cell or population of cells of any one of claims 140-148, wherein the altered epitope is in a binding region of the IL2RG antagonist, wherein the IL2RG antagonist is an antibody comprising an immunoglobulin light chain or variable region thereof comprising three light chain CDRs and an immunoglobulin heavy chain or variable region thereof comprising three heavy chain CDRs,
wherein the three light chain CDRs comprise, consist essentially of, or consist of the sequences set forth in SEQ ID NOS: 12, 14, and 16, respectively, and
wherein the three heavy chain CDRs comprise, consist essentially of, or consist of the sequences set forth in SEQ ID NOS: 4, 6, and 8, respectively.

150. The isolated cell or population of cells of any one of claims 140-149, wherein the mutation comprises a mutation encoded by nucleotides within exon 2 and/or exon 3 of the IL2RG gene at the IL2RG genomic locus.

151. The isolated cell or population of cells of any one of claims 140-150, wherein the mutation comprises a mutation or substitution within a region from position T127 to position N150 and/or within a region from position L87 to position D97.

152. The isolated cell or population of cells of any one of claims 140-151, wherein the mutation comprises a mutation or substitution at position M145, position W90, position K92, position N93, position D95, position D97, position T127, position R139, position R140, position Q141, position T143, and/or position K147.

153. The isolated cell or population of cells of any one of claims 140-152, wherein the mutation comprises a mutation or substitution at position M145, optionally wherein the substitution is a M145K substitution, a M145D substitution, a M145E substitution, a M145P substitution, a M145W substitution, or an M145Y substitution.

154. The isolated cell or population of cells of any one of claims 140-153, wherein the mutation comprises a M145K substitution.

155. The isolated cell or population of cells of any one of claims 140-154, wherein the mutation comprises a mutation or substitution at position W90, optionally wherein the substitution is a W90V substitution, a W90R substitution, a W90Q substitution, a W90L substitution, a W90K substitution, a W90E substitution, or a W90D substitution, optionally wherein the mutation comprises a W90Q substitution.

156. The isolated cell or population of cells of any one of claims 140-155, wherein the mutation comprises a mutation or substitution at position M145 and a mutation or substitution at position W90.

157. The isolated cell or population of cells of any one of claims 140-156, wherein the first isoform of IL2RG and the second isoform of IL2RG are immunologically distinguishable by the IL2RG antagonist, wherein the IL2RG antagonist specifically binds to the second isoform of IL2RG but does not specifically bind to the first isoform of IL2RG.

158. The isolated cell or population of cells of any one of claims 140-157, wherein the IL2RG antagonist is an antigen-binding protein.

159. The isolated cell or population of cells of claim 158, wherein the antigen-binding protein is an antibody or an antigen-binding fragment thereof.

160. The isolated cell or population of cells of claim 158 or 159, wherein the antigen-binding protein comprises an immunoglobulin light chain or variable region thereof comprising three light chain CDRs and an immunoglobulin heavy chain or variable region thereof comprising three heavy chain CDRs,
wherein the three light chain CDRs comprise, consist essentially of, or consist of sequences at least 90% identical to the sequences set forth in SEQ ID NOS: 12, 14, and 16, respectively, and
wherein the three heavy chain CDRs comprise, consist essentially of, or consist of sequences at least 90% identical to the sequences set forth in SEQ ID NOS: 4, 6, and 8, respectively.

161. The isolated cell or population of cells of claim 160, wherein the three light chain CDRs comprise, consist essentially of, or consist of the sequences set forth in SEQ ID NOS: 12, 14, and 16, respectively, and
wherein the three heavy chain CDRs comprise, consist essentially of, or consist of the sequences set forth in SEQ ID NOS: 4, 6, and 8, respectively.

162. The isolated cell or population of cells of claim 158 or 159, wherein the antigen-binding protein comprises an immunoglobulin light chain variable region that comprises, consists essentially of, or consists of a sequence at least 90% identical to the sequence set forth in SEQ ID NO: 10, and
wherein the antigen-binding protein comprises an immunoglobulin heavy chain variable region that comprises, consists essentially of, or consists of a sequence at least 90% identical to the sequence set forth in SEQ ID NO: 2.

163. The isolated cell or population of cells of claim 162, wherein the immunoglobulin light chain variable region comprises, consists essentially of, or consists of the sequence set forth in SEQ ID NO: 10, and
wherein the immunoglobulin heavy chain variable region comprises, consists essentially of, or consists of the sequence set forth in SEQ ID NO: 2.

164. The isolated cell or population of cells of claim 158 or 159, wherein the antigen-binding protein comprises an immunoglobulin light chain that comprises, consists essentially of, or consists of the sequence set forth in SEQ ID NO: 20, and
wherein the antigen-binding protein comprises an immunoglobulin heavy chain that comprises, consists essentially of, or consists of the sequence set forth in SEQ ID NO: 18.

165. The isolated cell or population of cells of any one of claims 140-164, wherein the cell or cells are hematopoietic cell(s), optionally wherein the cell or cells are immune cell(s).

166. The isolated cell or population of cells of any one of claims 140-165, wherein the cell or cells are lymphocytes or lymphoid progenitor cell(s).

167. The isolated cell or population of cells of any one of claims 140-166, wherein the cell or cells are T cell(s).

168. The isolated cell or population of cells of any one of claims 140-167, wherein the cell or cells are tumor infiltrating lymphocyte(s) (TILs).

169. The isolated cell or population of cells of any one of claims 140-166, wherein the cell or cells are B cell(s).

170. The isolated cell or population of cells of any one of claims 140-166, wherein the cell or cells are NK cell(s).

171. The isolated cell or population of cells of any one of claims 140-165, wherein the cell or cells are hematopoietic stem cell(s) or hematopoietic stem and progenitor cell(s).

172. The isolated cell or population of cells of any one of claims 140-171, wherein the cell or cells are induced pluripotent stem cell(s).

173. The isolated cell or population of cells of any one of claims 140-172, wherein the cell or cells are mammalian cell(s) or non-human mammalian cell(s).

174. The isolated cell or population of cells of any one of claims 140-173, wherein the cell or cells are human cell(s).

175. The isolated cell or population of cells of any one of claims 140-174, wherein the cell or cells comprise or express a therapeutic molecule.

176. The isolated cell or population of cells of claim 175, wherein the therapeutic molecule does not target IL2RG.

177. The isolated cell or population of cells of any one of claims 140-176, wherein the cell or cells comprise or express an immunoglobulin.

178. The isolated cell or population of cells of claim 177, wherein the immunoglobulin does not target IL2RG.

179. The isolated cell or population of cells of any one of claims 140-178, wherein the cell or cells comprise a chimeric antigen receptor (CAR) or an exogenous T cell receptor (TCR).

180. The isolated cell or population of cells of claim 179, wherein the CAR or the exogenous TCR does not target IL2RG.

181. The isolated cell or population of cells of any one of claims 140-180, wherein the cell or cells are isolated from a subject.

182. The isolated cell or population of cells of any one of claims 140-181 for use in treatment of a subject having cells expressing the second isoform of IL2RG.

183. The isolated cell or population of cells for use of claim 182, wherein the cell or cells are isolated from the subject.

184. A method of making the isolated cell or population of cells of any one of claims 140-183, comprising modifying a cell or population of cells to express the first isoform of IL2RG.

185. The method of claim 184, wherein the modifying comprises introducing an expression vector encoding the first isoform of IL2RG, or wherein the modifying comprises editing a genomic locus to express the first isoform of IL2RG.

186. The method of claim 185, wherein the genomic locus is an endogenous IL2RG genomic locus.

187. The method of claim 185, wherein the genomic locus is not an endogenous IL2RG genomic locus.

188. The method of any one of claims 185-187, wherein the editing comprises introducing into the cells:
(1) a nuclease agent or one or more nucleic acids encoding the nuclease agent, wherein the nuclease agent targets a nuclease target sequence in the genomic locus; and
(2) an exogenous donor nucleic acid,
wherein the nuclease agent cleaves the genomic locus and the exogenous donor nucleic acid is inserted into the genomic locus or recombines with the genomic locus to generate the donor cells that express the first isoform of IL2RG.

189. The method of claim 188, wherein the nuclease agent comprises:
(a) a zinc finger nuclease (ZFN);
(b) a transcription activator-like effector nuclease (TALEN); or
(c) (i) a Cas protein; and
(ii) a guide RNA, wherein the guide RNA comprises a DNA-targeting segment that targets a guide RNA target sequence that is the nuclease target sequence, and wherein the guide RNA binds to the Cas protein and targets the Cas protein to the guide RNA target sequence.

190. The method of claim 189, wherein the nuclease agent comprises the Cas protein and the guide RNA, optionally wherein the DNA-targeting segment comprises the sequence set forth in any one of SEQ ID NOS: 76-87 or the guide RNA target sequence comprises the sequence set forth in any one of SEQ ID NOS: 64-75, or optionally wherein the DNA-targeting segment comprises the sequence set forth in any one of SEQ ID NOS: 136-153 or the guide RNA target sequence comprises the sequence set forth in any one of SEQ ID NOS:
118-135.

191. The method of claim 189 or 190, wherein the Cas protein is a Cas9 protein.

192. The method of any one of claims 188-191, wherein the exogenous donor nucleic acid comprises homology arms.

193. The method of any one of claims 188-192, wherein the exogenous donor nucleic acid is a single-stranded oligodeoxynucleotide (ssODN), optionally wherein the ssODN comprises the nucleic acid sequence set forth in any one of SEQ ID NOS: 88-117 or optionally wherein the ssODN comprises the nucleic acid sequence set forth in any one of SEQ ID NOS: 154-173.

194. The method of claim 193, wherein:
(I) the DNA-targeting segment comprises the sequence set forth in SEQ ID NO: 77 or the guide RNA target sequence comprises the sequence set forth in SEQ ID NO: 65, and wherein the ssODN comprises the nucleic acid sequence set forth in any one of SEQ ID NOS: 88-97;
(II) the DNA-targeting segment comprises the sequence set forth in SEQ ID NO: 83 or the guide RNA target sequence comprises the sequence set forth in SEQ ID NO: 71, and wherein the ssODN comprises the nucleic acid sequence set forth in any one of SEQ ID NOS: 98-10⁷;
(III) the DNA-targeting segment comprises the sequence set forth in SEQ ID NO: 86 or the guide RNA target sequence comprises the sequence set forth in SEQ ID NO: 74, and wherein the ssODN comprises the nucleic acid sequence set forth in any one of SEQ ID NOS: 108-117;
(IV) the DNA-targeting segment comprises the sequence set forth in SEQ ID NO: 137 or the guide RNA target sequence comprises the sequence set forth in SEQ ID NO: 119, and wherein the ssODN comprises the nucleic acid sequence set forth in any one of SEQ ID NOS: 154-163; or
(V) the DNA-targeting segment comprises the sequence set forth in SEQ ID NO: 138 or the guide RNA target sequence comprises the sequence set forth in SEQ ID NO: 120, and wherein the ssODN comprises the nucleic acid sequence set forth in any one of SEQ ID NOS: 164-173.

195. A genetically engineered human interleukin-2 receptor subunit gamma (IL2RG) protein comprising an artificial mutation to provide an altered epitope, wherein the altered epitope is a binding region of an IL2RG antagonist such that the IL2RG antagonist exhibits reduced or abolished ability to bind and/or inhibit the genetically engineered IL2RG protein compared to its ability to bind and/or inhibit a wild type human IL2RG protein, and wherein the genetically engineered IL2RG protein retains binding to its endogenous ligands.

196. The genetically engineered human IL2RG protein of claim 195, wherein the genetically engineered IL2RG protein is functionally indistinguishable but immunologically distinguishable from a native IL2RG protein.

197. The genetically engineered human IL2RG protein of claim 195 or 196, wherein the altered epitope is in a binding region of the IL2RG antagonist, wherein the IL2RG antagonist is an antibody comprising an immunoglobulin light chain or variable region thereof comprising three light chain CDRs and an immunoglobulin heavy chain or variable region thereof comprising three heavy chain CDRs,
wherein the three light chain CDRs comprise, consist essentially of, or consist of the sequences set forth in SEQ ID NOS: 12, 14, and 16, respectively, and
wherein the three heavy chain CDRs comprise, consist essentially of, or consist of the sequences set forth in SEQ ID NOS: 4, 6, and 8, respectively.

198. The genetically engineered human IL2RG protein of any one of claims 195-197, wherein the mutation comprises a mutation encoded by nucleotides within exon 2 and/or exon 3 of the IL2RG gene at the IL2RG genomic locus.

199. The genetically engineered human IL2RG protein of any one of claims 195-198, wherein the mutation comprises a mutation or substitution within a region from position T127 to position N150 and/or within a region from position L87 to position D97.

200. The genetically engineered human IL2RG protein of any one of claims 195-199, wherein the artificial mutation comprises a mutation or substitution at position M145, position W90, position K92, position N93, position D95, position D97, position T127, position R139, position R140, position Q141, position T143, and/or position K147.

201. The genetically engineered human IL2RG protein of any one of claims 195-200, wherein the artificial mutation comprises a mutation or substitution at position M145, optionally wherein the substitution is a M145K substitution, a M145D substitution, a M145E substitution, a M145P substitution, a M145W substitution, or an M145Y substitution.

202. The genetically engineered human IL2RG protein of any one of claims 195-201, wherein the artificial mutation comprises a M145K substitution.

203. The genetically engineered human IL2RG protein of any one of claims 195-202, wherein the mutation comprises a mutation or substitution at position W90, optionally wherein the substitution is a W90V substitution, a W90R substitution, a W90Q substitution, a W90L substitution, a W90K substitution, a W90E substitution, or a W90D substitution, optionally wherein the mutation comprises a W90Q substitution.

204. The genetically engineered human IL2RG protein of any one of claims 195-203, wherein the mutation comprises a mutation or substitution at position M145 and a mutation or substitution at position W90.

205. The genetically engineered human IL2RG protein of any one of claims 195-204, wherein the genetically engineered IL2RG protein and the native IL2RG protein are functionally indistinguishable but immunologically distinguishable by the IL2RG antagonist.

206. The genetically engineered human IL2RG protein of any one of claims 195-205, wherein the IL2RG antagonist is an antigen-binding protein.

207. The genetically engineered human IL2RG protein of claim 206, wherein the antigen-binding protein is an antibody or an antigen-binding fragment thereof.

208. The genetically engineered human IL2RG protein of claim 206 or 207, wherein the antigen-binding protein comprises an immunoglobulin light chain or variable region thereof comprising three light chain CDRs and an immunoglobulin heavy chain or variable region thereof comprising three heavy chain CDRs,
wherein the three light chain CDRs comprise, consist essentially of, or consist of sequences at least 90% identical to the sequences set forth in SEQ ID NOS: 12, 14, and 16, respectively, and
wherein the three heavy chain CDRs comprise, consist essentially of, or consist of sequences at least 90% identical to the sequences set forth in SEQ ID NOS: 4, 6, and 8, respectively.

209. The genetically engineered human IL2RG protein of claim 208, wherein the three light chain CDRs comprise, consist essentially of, or consist of the sequences set forth in SEQ ID NOS: 12, 14, and 16, respectively, and
wherein the three heavy chain CDRs comprise, consist essentially of, or consist of the sequences set forth in SEQ ID NOS: 4, 6, and 8, respectively.

210. The genetically engineered human IL2RG protein of claim 206 or 207, wherein the antigen-binding protein comprises an immunoglobulin light chain variable region that comprises, consists essentially of, or consists of a sequence at least 90% identical to the sequence set forth in SEQ ID NO: 10, and
wherein the antigen-binding protein comprises an immunoglobulin heavy chain or variable region that comprises, consists essentially of, or consists of a sequence at least 90% identical to the sequence set forth in SEQ ID NO: 2.

211. The genetically engineered human IL2RG protein of claim 210, wherein the immunoglobulin light chain variable region comprises, consists essentially of, or consists of the sequence set forth in SEQ ID NO: 10, and
wherein the immunoglobulin heavy chain variable region comprises, consists essentially of, or consists of the sequence set forth in SEQ ID NO: 2.

212. The genetically engineered human IL2RG protein of claim 206 or 207, wherein the antigen-binding protein comprises an immunoglobulin light chain that comprises, consists essentially of, or consists of the sequence set forth in SEQ ID NO: 20, and
wherein the antigen-binding protein comprises an immunoglobulin heavy chain that comprises, consists essentially of, or consists of the sequence set forth in SEQ ID NO: 18.

213. A nucleic acid encoding the genetically engineered human IL2RG protein of any one of claims 195-212, optionally wherein the nucleic acid is an expression vector encoding the genetically engineered human IL2RG protein.
